(19) United States
(12) Patent Application Publication
(10) Pub. No.: US 2012/0135912 A1
(43) Pub. Date: May 31, 2012

(54) POLYPEPTIDE INHIBITORS OF VLA4

(75) Inventors: MADAN M. PAIDHUNGAT, SAN FRANCISCO, CA (US); AMULYA NANISETTI, FREMONT, CA (US); THOMAS BOUQUIN, ALLEROED (DK); KIM VILBOUR ANDERSEN, BROENSHOEJ (DK); STEVEN J. CHAPIN, FREMONT, CA (US); CLAUS KREBBER, PALO ALTO, CA (US); RONG A. FAN, REDWOOD CITY, CA (US); DANIEL MALASHOCK, REDWOOD CITY, CA (US); AMY BRIDEAU-ANDERSEN, SAN CLEMENTE, CA (US); ERIK KARRER, MOUNTAIN VIEW, CA (US); BRUCE DEVENS, OAKLAND, CA (US); STEVEN H. BASS, HILLSBOROUGH, CA (US)

(73) Assignee: PERSEID THERAPEUTICS LLC, REDWOOD CITY, CA (US)

(21) Appl. No.: 13/104,956

(22) Filed: May 10, 2011

Related U.S. Application Data
(60) Provisional application No. 61/333,210, filed on May 10, 2010.

(51) Int. Cl. A61K 38/00; C12N 5/10; C12N 1/21; C12N 1/19; A61P 29/00; C07K 9/00; C07K 14/00; C07K 14/765; C07K 16/18; C07H 21/00; C12P 21/06; C12N 15/63

(52) U.S. Cl. 514/1.1; 435/69.1; 435/325; 435/352; 435/358; 435/367; 435/419; 435/252.3; 435/252.31; 435/252.33; 435/252.35; 435/254.2; 435/320.1; 530/322; 530/324; 530/350; 530/363; 530/387.3; 536/23.1

(57) ABSTRACT

The invention provides novel 2D-VCAM-1 variant polypeptides and conjugates thereof that bind human VLA4. The invention also provides related polynucleotides, compositions, vectors, host cells, and methods.

### CROSS-REFERENCE TO RELATED APPLICATIONS

This application claims the benefit of provisional application U.S. Ser. No. 61/333,210, filed May 10, 2010, pursuant 35 U.S.C. §119(e), which is incorporated by reference in its entirety.

### FIELD OF THE INVENTION

The present invention relates to novel polypeptide inhibitors of the interaction of VLA4 (integrin α4β1) and VCAM-1, and the polynucleotides which encode them.

### REFERENCE TO SEQUENCE LISTING

The “Sequence Listing” submitted electronically concurrently herewith pursuant 37 C.F.R. §1.821 in computer readable form (CRF) via EFS-Web as file name 0376US_ST25_shifted.txt is incorporated herein by reference. The electronic copy of the Sequence Listing was created on May 10, 2011, and the size on disk is 364 kilobytes.

### BACKGROUND

The inflammatory process is made up of a complex set of interactions among soluble factors and cells that can arise in tissue as a response to traumatic, infectious, post-ischemic, toxic or autoimmune injury. Under normal circumstances, inflammation facilitates recovery from infection and eventual healing. However, persistent inflammation can lead to tissue damage by leukocytes, lymphocytes or collagen, and eventually may oxidize DNA badly enough to promote neoplastic transformation (Nathan C., Nature 420:846-852 (2002)).

There are a number of diseases in Which inflammation is considered to have either a primary or significant secondary pathogenic role. For example, inflammation is believed to play an important role in the pathology of Alzheimer's disease, anaphylaxis, ankylosing spondylitis, asthma, atherosclerosis, atopic dermatitis, chronic obstructive pulmonary disease, Crohn's disease, gout, Hashimoto's thyroiditis, ischaemia-reperfusion injury, multiple sclerosis, osteoarthritis, pemphigus, periodic fever syndromes, psoriasis, rheumatoid arthritis, sarcoidosis, systemic lupus erthematosus, type I diabetes mellitus, ulcerative colitis, vasculitides (such as Wegener's syndrome, Goodpasture's syndrome, giant cell arteritis, polyarteritis nodosa), and xenograft rejection (Nathan, supra). Similarly, inflammation is believed to be as significant as microbial toxicity in the pathology of infectious diseases such as bacterial dysentery, Chagas disease, cystic fibrosis pneumonitis, bilariasis, Helicobacter pylori gastritis, hepatitis C, influenza virus pneumonia, leprosy (tuberculoid form), neisserial or pneumococcal meningitis, post-streptococcal glomerulonephritis, sepsis syndrome, and tuberculosis. Accordingly, inflammation has become one of the main therapeutic targets in a number of diverse disorders (Nathan, supra).

Increased leukocyte adherence to endothelium is a characteristic of the acute inflammatory response (Harlan J. M., Blood 65(3):513-525(1985)). At sites of inflammation, leukocyte adhesion receptor activity is modulated and the cells become capable of interacting with ligands expressed on the luminal surface of the vasculature (Newham P. et al., J. Biol. Chem. 272(31):19429-19440(1997)). The fundamental events in leukocyte-endothelial interactions in the acute inflammatory response are: initial adherence, maintained sticking, diapedesis, and extravascular migration (Harlan, supra; Mayrovitz H. et al., Thromb. Haemost. 38:823-830(1997)). Leukocyte integrin receptors and endothelial immunoglobulin superfamily cell adhesion molecules (IgCAMs) are believed to contribute to the events that facilitate leukocyte emigration into the tissues (Newham et al., supra; Butcher E. C., Adv. Exp. Med. Biol. 323:181-194(1992); Springer T. A. Cell 76:301-314(1994)).

Integrins form a large family of transmembrane proteins which consist of two non-covalently bound α and β subunits of 120-180 kDa and 90-120 kDA, respectively (Ghosh S., Ann. Rhem. Dis. 62(Suppl II):ii70-ii72 (2003)). The non-covalently bound α and β subunits exist as αβ heterodimers of different combinations of α and β chains that share extensive structural homology (Pepinsky R. B. et al., J. Pharmacol. Exp. Ther. 312(2):742-750 (2005)). The integrin very late antigen-4 (VLA4, also referred to as CD49d/CD29 or integrin α4β1), a heterodimer of α4 and β1 subunits, is expressed predominantly on cells belonging to the hematopoietic lineage, such as mononuclear cells and eosinophils. VLA4 is believed to play a major role in the regulation of leukocyte extravasation in response to inflammation (Hemler M. E. et al., Immum. Rev. 114:45-65 (1990)). Integrin α4β1 is also expressed by melanoma and some other tumor cell types. Rice and Bevilacqua (Rice G. E. and Bevilacqua M. P., Science 246:1303-1306 (1989)) have proposed that VLA4 and its co-receptor, VCAM-1, mediate the hematogenous metastatic spread of these cells.

VLA4 is expressed on neural crest cells, lymphocytes, monocytes, eosinophils, myoblasts, and, at low levels, on polymorphonuclear cells (Newham P. et al., J. Immunol. 160:4508-4517 (1998)). VLA4 has been reported to regulate leukocyte migration into damaged tissue (Pepinsky et supra). It is a key receptor for fibronectin and for the cell surface Ig superfamily member, vascular cell-adhesion molecule-1 (VCAM-1). VLA4 also hinds to some extent to mucosal vascular addressin cell adhesion molecule (MADCAM-1; Pepinsky et al., supra).

VCAM-1, the primary co-receptor of VLA4, is expressed on endothelial surface in response to cytokine stimulation, in forms containing either six or seven extracellular domains (Hession C. et al., J. Biol. Chem. 266:6682-6685 (1991)). The predominant form on cytokine-stimulated endothelium is the seven-domain form. It has been reported that there is 63% sequence identity for the alignment of the regions containing domains 1-3 with that containing domains 4-6, whereas the sequence identity of other pairwise comparisons of the VCAM-1 domains averages 24% (Dudgeon T. J. et al, Eur. J. Biochem. 226:517-523 (1994)). It is believed that the first six domains of VCAM-1 arose from a tandem duplication of a unit consisting of three domains (Dudgeon et at, supra).

There are two binding sites in VCAM-1 for VLA4, located in domain-1 and domain-4 of VCAM-1. The domain-4 site is absent from the six domain form of VCAM-1. The residues which mediate VLA4 binding have been mapped to a conserved Gln-Ile-Asp-Ser-Pro motif in domains 1 and 4 of VCAM-1 (Osborn L. et al., J. Cell. Biol. 124:601-608 (1994); Vonderheide R. H. et al., J. Cell. Biol. 125:215-222 (1994); Renz M. E. et al., J. Cell. Biol. 125:1395-4306 (1994); Clements J. M. et al., J. Cell. Sci. 107:2127-2135 (1994)). Newham et al., J. Biol. Chem. 272(31):19429-19440 (1997), describe VCAM-1/MAdCAM-1 chimeras and mutant VCAM-1 constructs containing amino acid substitutions within the predicted integrin adhesion face and characterized their binding to integrins α4β1 and α4β7. Among other observations made, the authors reported that Glu³⁸ mutated to either glycine or a leucine demonstrated a super-adhesive phenotype with respect to α4β1 binding.

Antagonists of α4 integrin have been reported as being effective in inhibiting a variety of experimental models of inflammatory diseases. Leone and coworkers (Leone D. R. et al., J. Pharmacol. Exp. Ther. 305:1150-1156 (2003)) report that two types of α4β1 inhibitors, an anti-rat α4 monoclonal antibody TA-2 and a small molecule inhibitor BIO5192, were efficacious in delaying paralysis associated with experimental autoimmune encephalomyelitis (EAE) in rats. EAE is an inflammatory condition of the central nervous system, with similarities to multiple sclerosis (Yednock T. A. et al., Nature 356:63-66 (1992)). Circulating leukocytes in both EAE and in multiple sclerosis penetrate the blood-brain barrier and damage myelin, resulting in impaired nerve conduction and paralysis. Yednock, et al. (supra) report that an anti-α4 integrin antibody was effective at preventing the accumulation of leukocytes in the central nervous system and the development of EAE in experimental animals.

Another integrin, α4β7 (lymphocyte Peyer patch adhesion molecule, or LPAM-1), is present on most lamina propria T cells and IgA-secreting B cells. The main co-receptor of α4β7 is mucosal vascular addressin cell adhesion molecule (“MADCAM-1”). Other ligands for α4β7 include fibronectin, VCAM-1, and α4 integrin itself (Rice and Bevilacqua, supra). VLA4 and LPAM-1, as well as other integrin receptors, exist in both a low affinity conformational state (or inactive state) and a high affinity state (or activated state), allowing cells to modulate the binding affinity of their integrin receptors (Dustin, M. L. and Springer, T. A., Nature, 34:619-624 (1989)). Transition from the low affinity to high affinity state is due to conformational changes in the integrin receptor following divalent cation binding such as magnesium (Mg²⁺) and manganese (Mn²⁺), which increases the affinity of VLA4 and LPAM-1 to their ligands, VCAM-1 and MADCAM-1, respectively (Dransfield, I., et al., J. Cell Biol. 116:219-226 (1992); Mould, A. P. et al., J. Biol. Chem 277:19800-19806 (2002)). During the inflammatory adhesion mechanism, activated integrins halt rolling leukocytes and attach them firmly to the vascular endothelium.

The recombinant monoclonal anti-α4 antibody natalizumab (TYSABRI®) blocks the ability of integrins α4β1 (VLA4) and α4β7 (LPAM-1) to bind to their respective endothelial co-receptors, VCAM-1 and MADCAM-1. Natalizumab was approved by the Food and Drug Administration (FDA) for the treatment of relapsing forms of multiple sclerosis in November 2004 and is being investigated for the treatment of Crohn's disease and rheumatoid arthritis (Yousry T. A. et al., New Engl. J. Med. 354(9):924-933 (2006)). Miller and coworkers (Miller D. H. et al., New Engl. J. Med. 348(1):15-23 (2003)) have reported that, in a placebo-controlled trial, treatment with natalizumab led to fewer inflammatory brain lesions and fewer relapses over a six-month period in patients with relapsing multiple sclerosis. Treatment with natalizumab was shown to increase the rates of clinical remission and response and C-reactive protein levels, and was well tolerated in patients with active Crohn's disease (Ghosh S. et al., New Engl. J. Med. 348(1):24-32 (2003)).

Although natalizumab has appeared to be a promising therapeutic in the treatment of multiple sclerosis (MS) and Crohn's disease, Yousry et al. (supra), and more recently Wenning et al. (N. Eng. J. Med. 361:1075-1080 (2009)) and Linda et al. (N. Eng. J. Med. 361:1081-1087 (2009)) have reported that progressive multifocal leukoencephalopathy (PML), a demyelinating disease of the central new system caused by the JC virus, developed in some patients treated with natalizumab. Studies in MS patients have shown that the pharmacodynamic effect of natalizumab significantly outlasts its serum half-life with some immunesuppressing effects lasting up to 6 months after the last drug administration (Stuve, O., et al., Ann. Neurol, 59:743-747 (2006)). Owing to the long pharmacokinetic and pharmacodynamic half-lives of this monoclonal antibody therapeutic, it has been suggested that accelerated clearance of natalizumab via plasma exchange procedures may improve the clinical outcomes of natalizumab treated patients who develop PML (Khatri B. O. et al. Neurology 72:402-409 (2009)). The plasma exchange procedure, which while only partially effective in reducing natalizumab levels, nevertheless requires the patient to undergo an invasive procedure of two to three hours duration thrice weekly for as many as several weeks (Khatri et al., supra).

Natalizumab is a humanized IgG4 monoclonal antibody. IgG4 antibodies naturally undergo half-antibody exchange (or chain swapping) with other IgG4 antibodies in vivo. This process leads to randomization of the Fab arms and renders the chain swapped IgG4 population essentially monovalent towards its original antigen (Aalberse R. C. and Schuurman J. Immunology 105:9-19 (2002); Van der Neut Kolfschoten, M. et al., Science. 317: 1554-1557 (2007)). IgG4 chain swapping is mediated by reduction of the IgG4 hinge sequence (Bloom, J. W. et al., Protein Sci. 6:407-415 (1997); Schuurman, J., et al., Mol. Immunol. 38:1-8 (2001)). Several lines of evidence support the notion that natalizumab also undergoes IgG4 chain swapping in vivo. First, studies in rats, in which natalizumab was coninjected with a human IgG4 monoclonal antibody, demonstrated that only 13% of natalizumab detected in the serum was intact after 24 hours (Shapiro, R. I. et al., J. Pharm. Biomed. Anal. 55(1):168-175 (2011)). Second, Fab arm exchanged natalizumab (based on light chain differences between natalizumab and endogenous IgG4) was detected within hours of administration of natalizumab in multiple schlerosis (MS) patients (Labrijn A. F. et al., Nat. Biotechnol. 27(8):767-771 (2009)). The extent of natalizumab chain swapping in people is dependent upon the level of endogenous IgG4, which can vary significantly among and between people (Aucouturier, P. et al., J. Immunol. Methods, 74:151-162 (1984)). Phase 3 trials of natalizumab demonstrated that greater than or equal to 70% of α4 integrin receptors were saturated when 300 mg of natalizumab was administered every 4 weeks (Rudick R. A. and Sandrock A. Expert Rev Neurotherapeutics 4:571-580 (2004); Biogen Idec Data on File). A universal natalizumab dosing regimen applicable to all MS patients regardless of their endogenous IgG4 levels may lead to underdosing and reduced efficacy in patients with high endogenous IgG4 levels and safety to issues in people with low endogenous IgG4 levels (due to over immune suppression).

The binding of natalizumab and other anti-α4 monoclonal antibodies has been reported to down-regulate the amount of VLA4 on the cell surface of lymphocytes whereas a small molecule inhibitor of VLA4, BIO5192, was demonstrated to have no effect on VLA4 receptor internalization (Putzki, N. et al. Eur. Neurology 63:311-317 (2010); Leone, D. R, et al. J. Pharmacol. Exp. Ther. 305:1150-1162 (2003)). Additionally, Wipfler et al. (Mult. Scler. 17:16-23 (2011)) and Harrer et al. (J. Neuroimmunol. (2011)), recently reported that natalizumab has secondary effects on other cellular adhesion molecules in patients leading to the cell surface down regulation of leukocyte function antigen-1 (LFA-1) on T cells and β1 integrin on T-cells, B-cells, natural killer cells and natural killer T cells, respectively. LFA-1 is important in leukocyte trafficking and activation and plays a key role in the development of EAE (Dugger, K. J. et al. J. Neuroimmunol. 206:22-27 (2009). β1 integrin forms heterodimeric integrin adhesion receptors with at least twelve different subunits (Byron, A., et al. J. Cell Sci. 122:4007-4011 (2009)). Secondary effects of natalizumab on LEA-1 and β1 containing integrin adhesion molecules, along with the long pharmacodynamic effect of the monoclonal antibody, may lead to significant defects in cell migration, immune homeostasis, and immune surveillance and an increased risk of opportunistic infections, such as that caused by JC virus.

Accordingly it is clear that effective alternative therapies for the treatment of disorders associated with inflammation such as multiple schlerosis, Crohn's disease, and the like with reduced potential for side effects remain highly desirable.

### SUMMARY OF THE INVENTION

The present invention provides novel 2D-VCAM-1 variant polypeptides that bind VLA4 (integrin α4β1) and inhibit the interaction of VLA4 with VCAM-1. In a specific embodiment, the present invention provides a recombinant 2D-VCAM-1 variant polypeptide comprising a sequence which differs in 0-8 amino acid positions from SEQ ID NO:18, and contains at least two amino acid residues selected from the group consisting of: phenylalanine or tyrosine at position 34 relative to SEQ ID NO:18 (F34 or P34Y); proline at position 37 relative to SEQ ID NO:18 (P37); leucine at position 39 relative to SEQ ID NO:18 (L39); and arginine at position 74 relative to SEQ ID NO:18 (R74), wherein the polypeptide has a binding affinity for a human VLA4 (integrin α4β1) protein that is greater than the binding affinity of Q38L-2D-VCAM-1. (SEQ ID NO:10) for the human VLA4 protein. The invention further provides recombinant 2D-VCAM1 variant polypeptides, wherein the variant exhibits greater binding to activated VLA4 as compared inactive VLA4. In another embodiment, the invention provides recombinant 2D-VCAM-1 variant polypeptides, wherein the variant exhibits greater binding to activated VLA4 as compared to Q38L-2D-VCAM-1 variant (SEQ ID NO:10).

In another embodiment, the present invention provides a fusion protein comprising a first polypeptide wherein the first polypeptide is a 2D-VCAM-1 variant polypeptide of the present invention and a second polypeptide or peptide fused (covalently attached) to the N- or C-terminus of the 2D-VCAM-1 variant. In a further embodiment, the present invention provides a fusion protein comprising a first polypeptide, wherein the first polypeptide is a 2D-VCAM-1 variant polypeptide of the present invention, a second polypeptide or peptide, wherein the second polypeptide or peptide is fused to the N-terminus of the 2D-VCAM variant polypeptide, and a third polypeptide or peptide is fused to the C-terminus of the 2D-VCAM variant polypeptide, and wherein the second and third polypeptide or peptide may be identical or different. Exemplary second and third polypeptides/peptides include, for example, poly-histidine tags and variants thereof, all or part of an Fc region of an immunoglobulin, human serum albumin, and the like.

The present invention also provides a 2D-VCAM-1 variant conjugate which exhibits VLA4 binding activity. The conjugate comprises a recombinant 2D-VCAM-1 variant polypeptide of the invention, or fusion protein thereof, covalently linked to one or more non-polypeptide conjugation moieties, wherein the conjugation moiety is a non-polypeptide polymer moiety, a sugar moiety, or a non-polypeptide lipophilic moiety. In some instances, the non-polypeptide polymer moiety is a polyethylene glycol (PEG) moiety. In another aspect, the present invention provides polypeptide fusions comprising 2D-VCAM-1 variant polypeptides and another polypeptide, such as a 2D-VCAM-1 valiant-Fc fusion polypeptides which exhibit VLA4-binding activity.

The invention also includes a composition comprising a 2D-VCAM-1 variant polypeptide or conjugate or fusion polypeptide of the invention, plus an excipient or carrier, such as a pharmaceutically acceptable excipient or carrier.

In another aspect, the invention includes a method of producing a recombinant 2D-VCAM-1 variant polypeptide of the invention, comprising culturing host cells comprising a polynucleotide of the invention and recovering the expressed polypeptide. The invention also includes a method of producing a 2D-VCAM-1 variant conjugate of the invention, comprising attaching a conjugation moiety (such as a PEG) to a 2D-VCAM-1 variant polypeptide of the invention.

In a further aspect, the invention includes a method of treating an inflammatory disease in a subject, the method comprising administering to the subject a therapeutically effective amount of a 2D-VCAM-1 variant polypeptide of the present invention, or conjugate or pharmaceutical composition thereof.

Additional aspects of the invention are described below.

### BRIEF DESCRIPTION OF THE FIGURES

FIG. 1A depicts a polynucleotide sequence (SEQ ID NO:1) encoding the first two domains of human VCAM-1 (i.e., human 2D-VCAM-1).

FIG. 1B depicts the amino amino acid sequence (SEQ ID NO:2) encoded by the polynucleotide sequence of FIG. 1A, and corresponds to the first two domains of human VCAM-1 (i.e., human 2D-VCAM-1).

FIG. 2 depicts an alignment of amino acid sequences of exemplary 2D-VCAM-1 variant polypeptides of the present invention as compared to the amino acid sequence of human 2D-VCAM-1 (SEQ ID NO:2) and a variant of human 2D-VCAM-1 having the single substitution Q38L, (Q38L-2D-VCAM-1, SEQ ID NO:10).

FIG. 3 depicts sensorgram traces from a BIACORE analysis of the binding of a 2D-VCAM-1 variant polypeptide of the present invention, Clone 146 (SEQ NO:18), as compared to Q38L-2D-VCAM-1 (SEQ ID NO:10), and human 2D-VCAM-1 (SEQ ID NO:2). The corresponding experiment is described in Example 12.

FIG. 4 demonstrates the association phase, reflecting the binding of the 2D-VCAM-1 analyte and the VLA4-Fc ligand, which is represented by the curves at times prior to the time marked by the arrow, and the dissociation phase, represented by the curves at times after the time marked by the arrow, as described in Example 12. Results are depicted for a 2D-VCAM-1 variant polypeptide of the present invention, Clone 146 (SEQ ID NO:18), as compared to Q38L -2D-VCAM-1 (SEQ ID NO:10), and human 2D-VCAM-1 (SEQ ID NO:2)

FIG. 5 depicts inhibition of U937 cell adhesion to immobilized 7D-VCAM-1 by anti-VLA4 antibody and by various soluble 2D-VCAM-1 polypeptides as described in Example 13. Results are depicted for an anti-VLA-4 antibody (positive control), dilution buffer alone (“no treatment”), 2D-VCAM-1 variant Clone 46 (SEQ ID NO:12), Q38L-2D-VCAM-1 (SEQ ID NO:10), and human 2D-VCAM-1 (SEQ ID NO:2)

FIG. 6 depicts titration curves of U937 cell adhesion to immobilized 7D-VCAM-1 in the presence of varying concentrations of variant Clone 46 (SEQ ID NO:12), variant Clone 146 (SEQ ID NO:18), and Q38L-2D-VCAM-1 (SEQ ID NO:10) as described in Example 13.

FIG. A is a plot of paralysis score vs. number of days post immunization of female SJL mice with 1000 μg/ml PLP and 2 mg/ml Mycobacterium tuberculosis in Incomplete Freund's Adjuvant. Starting at day 7 until day 15, mice were either left untreated or injected intravenously with either rat anti-murine α4 integrin monoclonal antibody PS/2 every day (control), 2D-VCAM-1 variant Clone 59 (SEQ ID NO:16) every day, or 2D-VCAM-1 variant Clone 146 (SEQ ID NO:18) every day. The results demonstrate that 2D-VCAM-1 variants of the invention are effective in vivo in delaying the onset of paralysis and reducing the severity of disease symptoms in a murine experimental autoimmune encephalomyelitis (EAE) model as described in Example 14.

FIG. 7B is a a plot of paralysis score vs. number of days post immunization of female SJL mice with 1000 μg/ml PLP and 2 mg/ml Mycobacterium tuberculosis in Incomplete Freund's Adjuvant. Mice were treated with either rat anti-murine α4 integrin monoclonal antibody PS/2 or 2D-VCAM-1 variant clone 146 (SEQ ID NO:18) N-terminally PEGylated with a 50K branched PEG-aldehyde reagent (“PEG50-146”) at 3 mg/kg on days 6, 8, 10 and 13 after PLP administration. The results demonstrate that PEGylated 2D-VCAM-1 variants of the invention are effective in vivo in delaying the onset of paralysis and reducing the severity of disease symptoms in a murine experimental autoimmune encephalomyelitis (EAE) model as described in Example 16.

FIG. 8 is a plot of clinical score vs. number of days after immunization of female Hartley guinea pigs with whole guinea pig brain homogenates and Mycobacterium tuberculosis in complete Freund adjuvant. Starting on day 13, guinea pigs were either left untreated or injected subcutaneously on days 13, 15 and 17 with 2D-VCAM-1 variant clone 146 (SEQ ID NO: 18) N-terminally PEGylated with a 50K branched PEG-aldehyde reagent (“PEG50-146”) or with a 2D-VCAM-1 variant Clone 146 N-terminally PEGylated with an 80K branched PEG-aldehyde reagent (“PEG80-146”). The humanized anti-α4 integrin monoclonal antibody natalizumab was administered on day 13 (control). The results demonstrate that PEGylated 2D-VCAM-1 variants of the invention are effective in vivo in delaying the onset of paralysis in a guinea pig experimental autoimmune encephalomyelitis (EAE) model as described in Example 17.

FIG. 9A is a plot of drug serum concentration (ng/ml) vs. time (hours) after subcutaneous injection of PEG50-146 at a dose of either 0.3 mg/kg or 3 mg/kg. The plot depicts the concentration of 2D-VCAM-1 variants of this invention in the serum of cynomolgus monkeys following subcutaneous administration of the polypeptides as described in Example 18.

FIG. 9B is a plot of drug serum concentration (ng/ml) vs. time (hours) after subcutaneous injection of natalizumab at a dose 3 mg/kg. The plot depicts the concentration of natalizumab in the serum of cynomolgus monkeys following a single subcutaneous administration of the polypeptides as described in Example 18.

FIG. 10 is a plot of % of peripheral B cells that are CD49+ (α4 integrin) vs. day, where on day 0 Female Balb/c mice were injected subcutaneously on day 0, day 2 and day 4 with either PEG50-146, a rat anti-murine α4 integrin monoclonal antibody PS/2, or PBS. The results demonstrate that the 2D-VCAM-1 variants of the invention do not decrease the percentage of CD49d+ (α4 integrin)/B220+ B cells in the peripheral blood of mice as described in Example 19A.

FIGS. 11A and 11B are a plot of % peripheral B cells that are CD49d+ (α4 integrin) vs. day and a plot of % peripheral T cells that are CD49d+ (α4 integrin) vs. day, respectively, where male cynomolgus monkeys were injected subcutaneously on day 1 with PEG50-146 or humanized anti-α4 integrin monoclonal antibody natalizumab (control). The results demonstrate that the 2D-VCAM-1 variants do not significantly decrease the percentage of CD49+ (α4 integrin)/CD20+ B cells or CD49+ (α4 integrin)/CD3+ T cells in the peripheral blood of monkeys as described in Example 19B.

FIGS. 12A and 12B are a plot of % peripheral B cells that are CD29+ (β1 integrin) vs. day and a plot of % peripheral T cells that are CD29+ (β1 integrin) vs. day, respectively, where male cynomolgus monkeys were injected subcutaneously on day 1 with PEG50-146 or humanized anti-α4 integrin monoclonal antibody natalizumab (control). The results demonstrate that the 2D-VCAM-1 variants of the invention do not significantly decrease the percentage of CD29+ (β1 integrin)/CD20+ B cells in the peripheral blood of mice as described in Example 19B.

FIG. 13 is a bar graph that illustrates the VLA4 binding characteristics of sequence-optimized variants of clone 146 (SEQ ID NO:18). A description of the experiment is provided in Example 20.

FIG. 14 is a bar graph that illustrates the preferential binding of the 2D-VCAM-1 variant polypeptides to high affinity (or activated) integrin on guinea pig spleen cells where conversion of VLA4 to the activated format was induced by adding 1 mM MnCl₂ to the assay buffer as described in Example 21.

FIG. 15 depicts sensorgram traces from a BIACORE analysis of the binding of 2D-VCAM-1 Clone 146 (SEQ ID NO:18) to human VLA4-Fc molecule under different integrin receptor activation states as described in Example 21.

FIG. 16A illustrates the linkage between PEG50-146 pharmacokinetics and pharmacodynamics in guinea pigs as described in Example 23.

FIG. 16B illustrates the linkage between PEG50-146 pharmacokinetics and pharmacodynamics in cynomolgus monkeys as described in Example 23.

FIG. 17A depicts sensorgram traces from a BIACORE analysis of the binding of Fc fusion proteins of Clone 146 (146-Fc; Panel A) and Clone 046 (046-Fc; Panel B) to human VLA4 as described in Example 23.

FIG. 17B depicts sensorgram traces from a BIACORE analysis of the binding of Fc fusion protein of Clone 146 (146-Fc) to human VLA4-Fc wherein 146-Fc was immobilized On the chimp and human VLA4-Fc was injected at various concentrations. The dissociation curve of 146-Fc binding to human VLA4-Fc is qualitatively similar to that of the non-Fc version of Clone 146 to human VLA4-Fc in a Biacore assay in the reverse orientation (inset). A description of the experiment is provided in Example 23.

FIG. 17B depicts sensorgram traces from a BIACORE analysis of the binding of Fc fusion protein of Clone 046 (046-Fc) to human VLA4-Fc wherein 046-Fc was immobilized on the chip arid human VLA4-Fc was injected at various concentrations. The dissociation curve of 046-Fc binding to human VLA4-Fc is qualitatively similar to that of the non-Fc version of Clone 046 to human VLA4-Fc in a Biacore assay in the reverse orientation (inset). A description of the experiment is provided in Example 23.

FIG. 18 depicts sensorgram traces from a BIACORE analysis of the binding bifunctionally PEGylated 2D-VCAM-1 Clone 146 (SEQ ID NO:18) and monomeric, non-PEGylated 2D-VCAM-1 Clone 146 (SEQ ID NO:18) to human VLA4 as described in Example 24.

### DETAILED DESCRIPTION

### Definitions

Unless otherwise defined herein or in the remainder of the specification, all technical and scientific terms used herein have the same meaning as commonly understood by those of ordinary skill in the art to which the invention belongs.

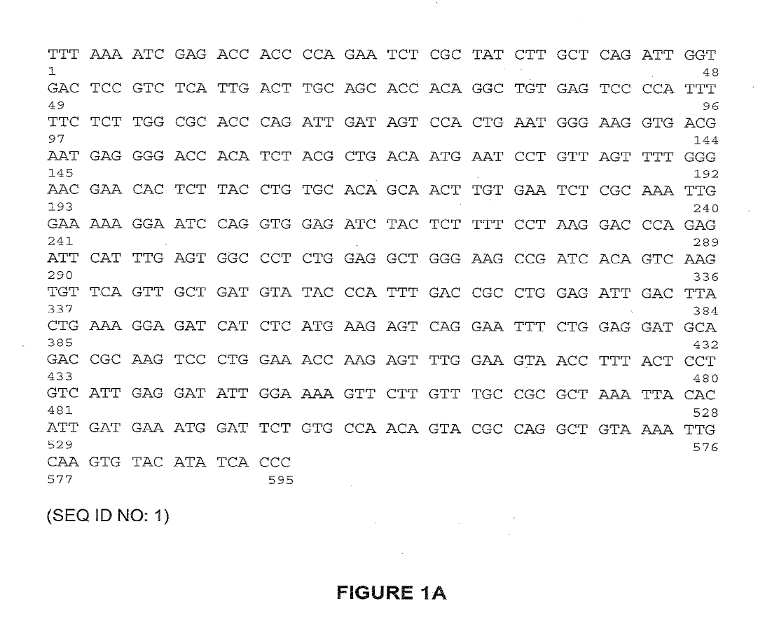

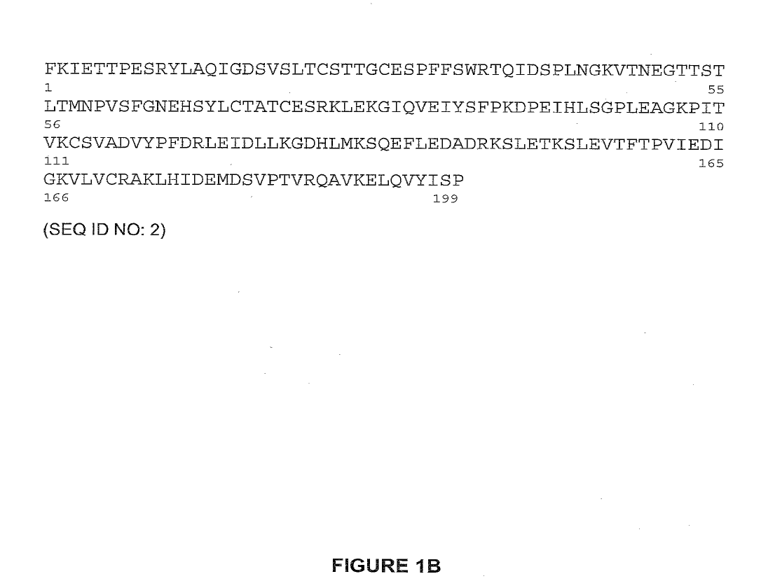

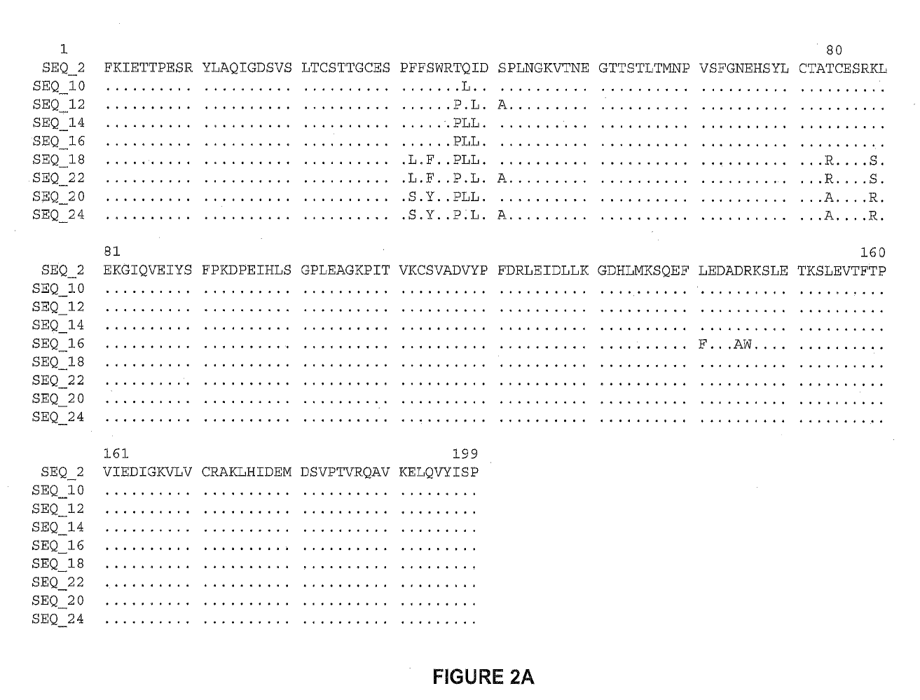

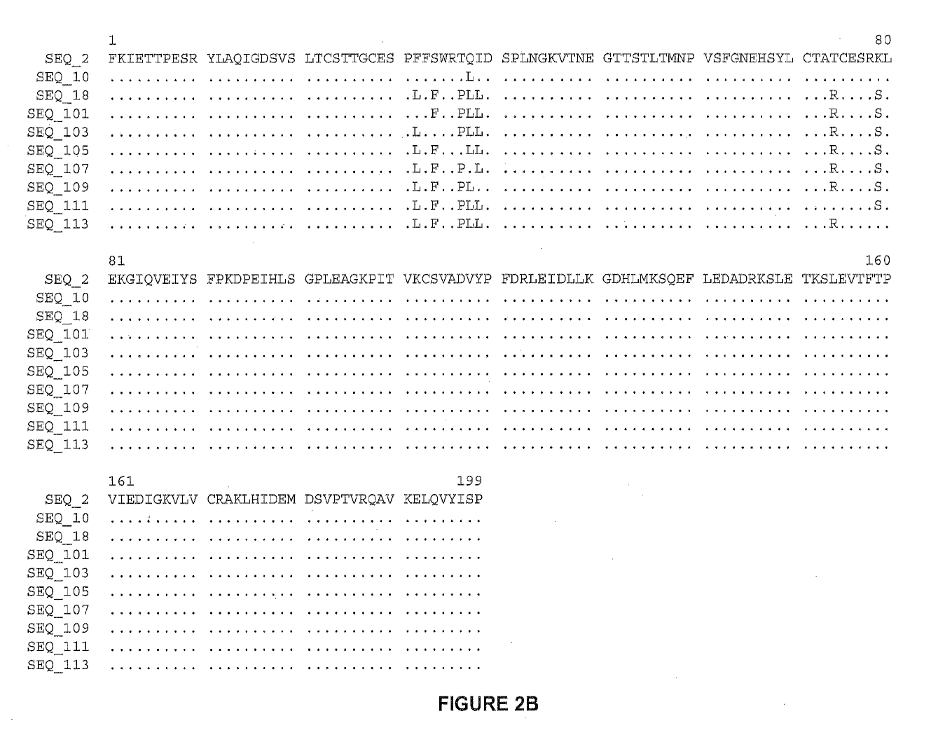

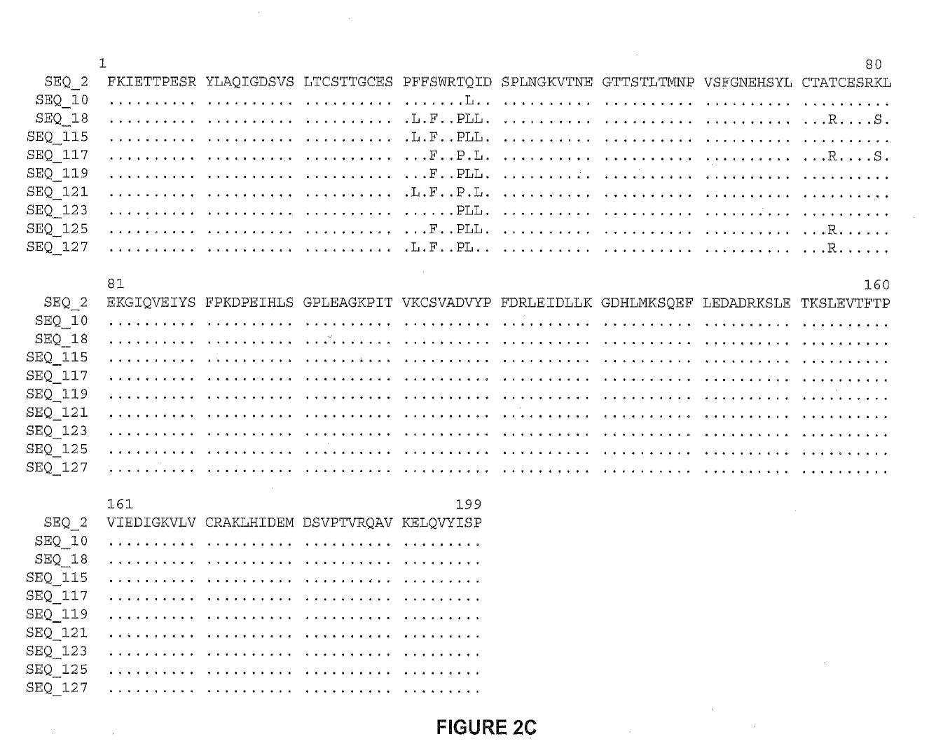

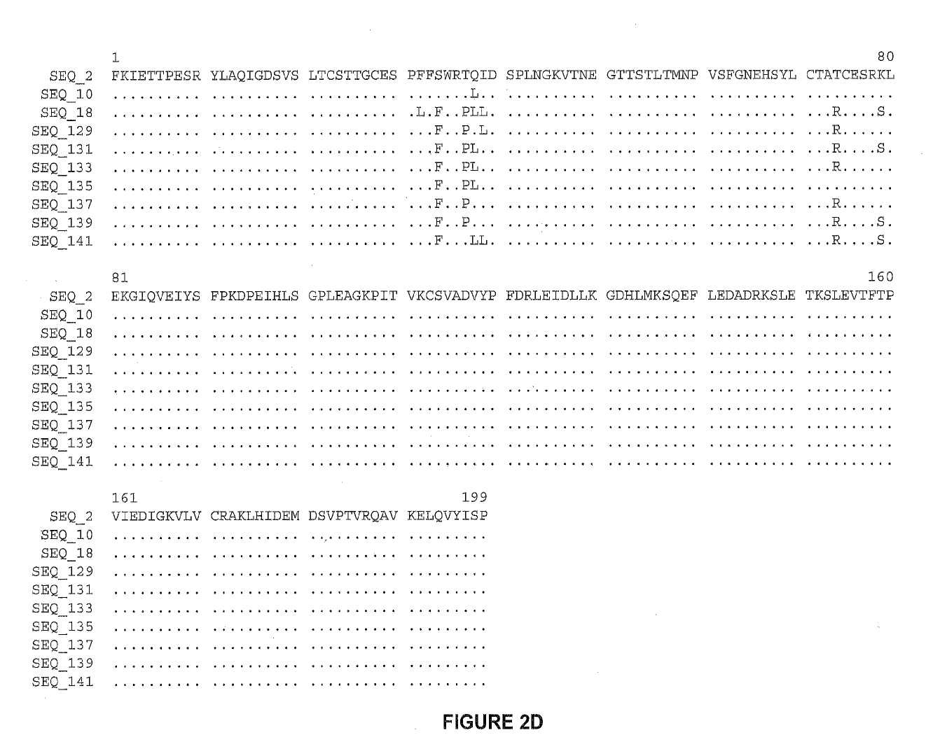

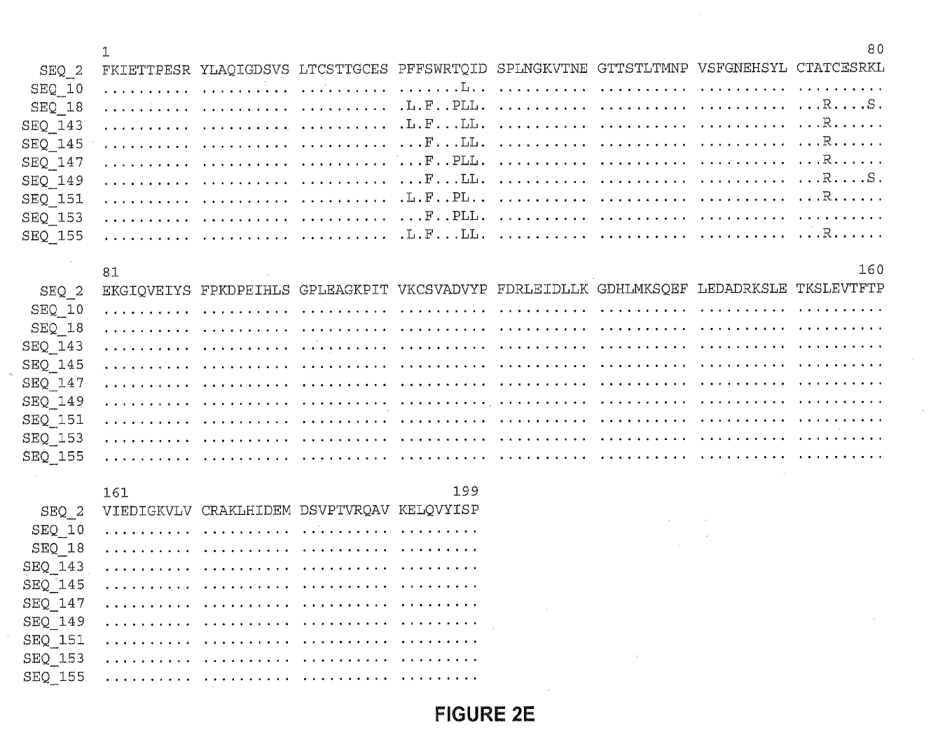

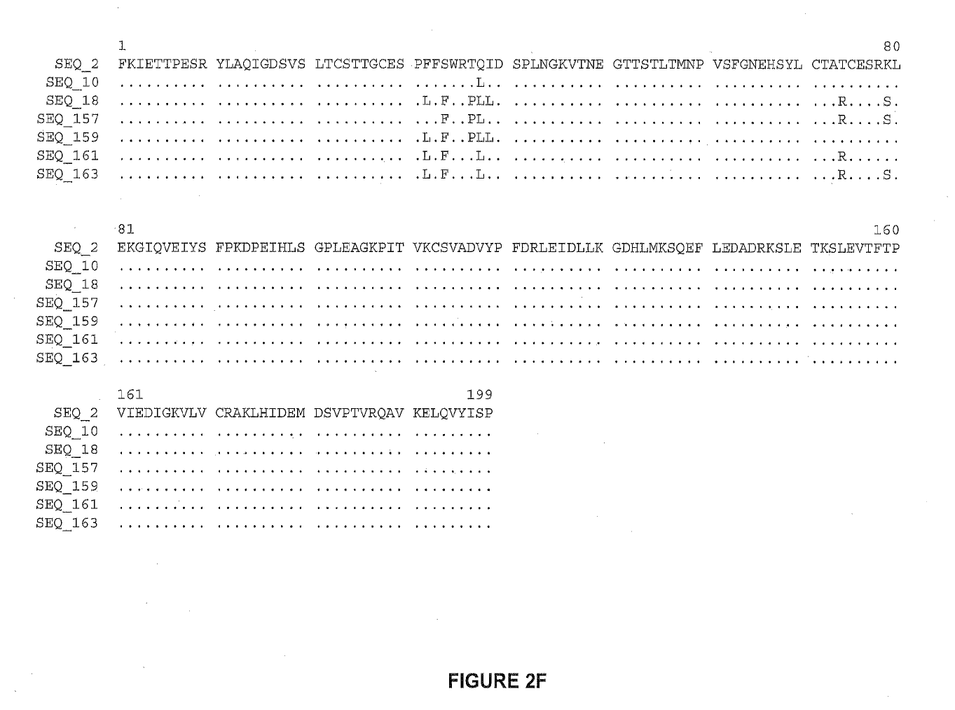

As used herein, the term “human 2D-VCAM-1” (or simply, “2D-VCAM-1”) refers to an artificial construct of the first two domains of the six-domain or the seven-dormain forms of native human VCAM-1; this two-domain form of VCAM-1 does not actually occur in nature (in other words, it is non-naturally occurring). SEQ ED NO:1 and SEQ ID NO:2 (FIGS. 1 and 2, respectively) provide the nucleic acid and amino acid sequences, respectively, of human 2D-VCAM-1. Amino acid residues 1-88 and 96-199 of SEQ ID NO:2 correspond to the sequences of Domain-1 and Domain-2 of human 2D-VCAM-1, respectively, and amino acid residues 89-95 of SEQ ID NO:2 correspond to the sequence of a linker peptide Which joins Domain-1 and Domain-2. The human 2D-VCAM-1 sequence corresponds to amino acid residues 1-199 of the mature six-domain form of human VCAM-1 (“6D-VCAM-1”, SEQ ID NO:4; encoded by the nucleic acid sequence SEQ ID NO:3) as well as residues 1-199 of the mature seven-domain form of human VCAM-1 (“7D-VCAM-1”, SEQ ID NO:6; encoded by the nucleic acid sequence SEQ ID NO:5), both of which do occur in nature (i.e., are naturally occurring).

As used herein, the term “2D-VCAM-1 variant polypeptide” (or “2D-VCAM-1 variant”) refers to a polypeptide comprising a sequence comprising the Domain-1 sequence and the Domain-2 sequence of human 2D-VCAM-1 (amino acids 1-88 and 96-199, respectively, of SEQ ID NO:2) wherein the variant sequence differs from the Domain-1 and/or the Domain-2 sequence of human 2D-VCAM-1 in one or more amino acid position(s), and which exhibits VLA4 binding activity.

A “polypeptide” is a polymer of amino acids comprising naturally occurring amino acids or artificial amino acid analogues, or a character string representing an amino acid polymer, depending on context. Given the degeneracy of the genetic cod; one or more nucleic acids, or the complementary nucleic acids thereof; that encode a specific polypeptide sequence can be determined from the polypeptide sequence.

A “polynucleotide” (or a “nucleic acid”) is a polymer of nucleotides comprising nucleotides A,C,T,U,G, or other naturally occurring nucleotides or artificial nucleotide analogues, or a character string representing a nucleic acid, depending on context. Either the given nucleic acid or the complementary nucleic acid can be determined from any specified polynucleotide sequence.

Numbering of a given amino acid polymer or nucleic acid polymer “corresponds to” or is “relative to” the numbering of a selected amino acid polymer or nucleic acid polymer when the position of any given polymer component (e.g., amino acid, nucleotide, also referred to generically as a “residue”) is designated by reference to the same or to an equivalent position in the selected amino acid or nucleic acid polymer, rather than by the actual numerical position of the component in the given polymer. Thus, for example, the numbering of a given amino acid position in a given polypeptide sequence corresponds to the same or equivalent amino acid position in a selected polypeptide sequence used as a reference sequence.

An “equivalent position” (for example, an “equivalent amino acid position” or “equivalent nucleic acid position” or “equivalent residue position”) is defined herein as a position (such as, an amino acid position or nucleic acid position or residue position) of a test polypeptide (or test polynucleotide) sequence which aligns with a corresponding position of a reference polypeptide (or reference polynucleotide) sequence, when optimally aligned using an alignment algorithm as described herein. The equivalent amino acid position of the test polypeptide need not have the same numerical position number as the corresponding position of the reference polypeptide; likewise, the equivalent nucleic acid position of the test polynucleotide need not have the same numerical position number as the corresponding position of the reference polynucleotide.

Two polypeptide sequences are “optimally aligned” when they are aligned using defined parameters, i,e., a defined amino acid substitution matrix, gap existence penalty (also termed gap open penalty), and gap extension penalty, so as to arrive at the highest similarity score possible for that pair of sequences. The BLOSUM62 matrix (Henikoff and Henikoff (1992) Proc. Natl. Acad. Sci. USA 89(22):10915-10919) is often used as a default scoring substitution matrix in polypeptide sequence alignment algorithms (such as BLASTP). The gap existence penalty is imposed for the introduction of a single amino acid gap in one of the aligned sequences, and the gap extension penalty is imposed for each residue position in the gap. Unless otherwise stated, alignment parameters employed herein are: BLOSUM62 scoring matrix, gap existence penalty=11, and gap extension penalty=1. The alignment score is defined by the amino acid positions of each sequence at which the alignment begins and ends (e.g. the alignment window), and optionally by the insertion of a gap or multiple gaps into one or both sequences, so as to arrive at the highest possible similarity score.

The terminology used for identifying amino acid positions and amino acid substitutions is illustrated as follows: T37 indicates position number 37 is occupied by a threonine (Thr) residue in a reference amino acid sequence, such as SEQ ID NO:2. T37P indicates that the threonine residue of position 37 has been substituted with a proline (Pro) residue. Alternative substitutions are indicated with a “/”, e.g., T37M/P means an amino acid sequence in which the threonine residue at position 37 is substituted with a methionine (Met) residue or a praline (Pro) residue. Multiple substitutions may sometimes be indicated with a “+”, e.g. T37M/P+139L indicates an amino acid sequence which contains a substitution of the threonine residue at position 37 with an a methionine residue or a praline residue, and a substitution of the isoleucine residue at position 39 with a leucine residue. Deletions are indicated by an asterisk. For example, S100* indicates that the serine residue at position 100 has been deleted. Deletions of two or more continuous amino acids may be indicated as follows, e.g., P95*-S100* indicates the deletion of residues P95 to 5100 inclusive (that is, residues 95, 96, 97, 98, 99, and 100 are deleted). Insertions are indicated the following manner: Insertion of an additional serine residue after the proline residue located at position 95 is indicated as P95PS. Combined substitutions and insertions are indicated in the following way: Substitution of the praline residue at position 95 with a serine residue and insertion of an alanine residue after the position 95 amino acid residue is indicated as P955A,

Unless otherwise indicated, the position numbering of amino acid residues of the 2D-VCAM-1 variant polypeptides recited herein is relative to the amino acid sequence SEQ ID NO:2, the sequence of human 2D-VCAM-1. It is to be understood that while the examples and modifications to the parent polypeptide may be provided herein relative to the sequence SEQ ID NO:2 (or relative to another specified sequence), the examples pertain to other polypeptides of the invention, and the modifications described herein may be made in equivalent amino acid positions (as described above) of any of the other polypeptides described herein. Thus, as an example, the substitution Q38L relative to SEQ ID NO:2 is understood to correspond to amino acid position L38 in. SEQ ID NO:18. As another example, the substitution F32L relative to SEQ ID NO:2 corresponds to the substitution S32L relative to SEQ ID NO:20, and corresponds to amino acid position L32 in SEQ ID NO:18.

VLA4 exists in nature as a non-covalent heterodimer of integrin subunits α4 and β1. A “VLA4 protein”, as defined herein, is intended to include naturally occurring and non-naturally occurring forms of integrin α4β1. Example 3 herein describes the recombinant expression and purification of one such VLA4 protein, a soluble fusion protein comprising human integrin α4 and β1 subunits each fused to an Fc domain (referred to herein as a “human VLA4-Fc” or simply as a “VLA4-Fc”), which may be used, for example, in the assay of Example 10 to determine the VLA4 binding activities of the 2D-VCAM-1 variant polypeptides of the present invention.

LPAM-1 similarly exists in nature as a non-covalent heterodimer of integrin subunits α4 and β7. An “LPAM-1 protein”, as defined herein, is intended to include naturally occurring and non-naturally occurring forms of integrin α4β7, such as for example a soluble fusion protein comprising human integrin α4 and β7 subunits each fused to an Fc domain (referred to herein as a “human LPAM-1-Fc” or simply as a “LPAM-1-Fc”), as described in Examples 4 and 11.

The term “exhibiting (or e.g., exhibits, having, or has) VLA4 binding activity” is intended to indicate that the polypeptide or conjugate of the invention has measurable binding activity to a VLA4 protein. The VLA4 protein may, for example, be expressed on the surface of a cell, such as a human U937 cell, in which case binding to VLA4 protein (in this instance, human VLA4 protein) on the cell surface may be determined using a cell adhesion assay, such as the cell adhesion assay described in Example 13 herein. Alternatively, the VLA4 protein may be produced in a soluble form, e.g., as a fusion protein, such as a human. VLA4-Fc fusion protein described in Example 3 herein, and binding to the VLA4-Fc may be determined, for example, using an ELISA assay as described in Example 10 or a Surface Plasmon Resonance (BIACORE) assay as described in Example 12. Other assays which measure VLA4 binding activity, including binding activities of polypeptides and conjugates of the invention to non-human (e.g., murine,rodent, non-human primate) VLA4 proteins are known in the art and/or are described in the Examples.

One of skill in the art recognizes that what constitutes a “measurable binding activity” depends in part on the nature of the assay being undertaken, but, as a general guideline, a measurable activity is one in which the assay signal generated in the presence of the test compound (e.g., a polypeptide of the invention) is quantifiably different than the assay signal generated in the absence of the test compound. In some instances the activity exhibited by a polypeptide or conjugate of the invention (as evidenced, e.g., by an EC50, adherence index, or other value related to activity) may be about equal to, be less than, or be greater than that of the particular activity exhibited by a reference 2D-VCAM-1 polypeptide (such as, human 2D-VCAM-1 polypeptide, SEQ ID NO:2).

A “variant” is a polypeptide comprising a sequence which differs in one or more amino acid position(s) from that of a parent polypeptide sequence (e.g., by substitution, deletion, or insertion). A variant may comprise a sequence which differs from the parent polypeptides sequence in up to 10% of the total number of residues of the parent polypeptide sequence, such as in up to 8% of the residues, e.g.,in up to 6%, 5%, 4%, 3% 2% or 1% of the total number of residue of the parent polypeptide sequence. For example, a variant of the 199 amino acid polypeptide sequence SEQ ID NO:2 comprises a sequence which differs in up to 10% of the total number of residues of the parent polypeptide sequence, that is, in up to 19 amino acid positions within the 199 amino acid polypeptide sequence SEQ ID NO:2 (such as in 1, 2, 3, 4, 5, 6, 7, 8, 9, 10, 11, 12, 13, 14, 15, 16, 17, 18, or 19 amino acid positions), e.g. in 1-19 amino acid positions, in 1-18 amino acid positions, in 1-17 amino acid positions, in 1-16 amino acid positions, in 1-15 amino acid positions, in 1-14 amino acid positions, in 1-13 amino acid positions, in 1-12 amino acid positions, in 1-11 amino acid positions, in 1-10 amino acid positions, in 1-9 amino acid positions, in 1-8 amino acid positions, in 1-7 amino acid positions, in 1-6 amino acid positions, in 1-5 amino acid positions, in 1-4 amino acid positions, in 1-3 amino acid positions, or in 1-2 amino acid positions within SEQ ID NO:2.

The term “parent polypeptide” is intended to indicate the polypeptide sequence to be modified in accordance with the present invention. The parent polypeptide sequence may, for example, be that of the human 2D-VCAM-1 polypeptide identified herein as SEQ ID NO:2, or the Q38L-2D-VCAM-1 polypeptide identified herein as SEQ ID NO:10. Alternatively, the parent polypeptide sequence may be that of a 2D-VCAM-1 variant disclosed herein, such as, for example, one of SEQ ID NOs:12, 14, 16, 18, 20, 22, or 24.

“Naturally occurring” as applied to an object refers to the fact that the object can be found in nature as distinct from being artificially produced by man. For example, a polypeptide or polynucleotide sequence that is present in an organism (including viruses, bacteria, protozoa, insects, plants or mammalian tissue) that can be isolated from a source in nature and which has not been intentionally modified by man in the laboratory is naturally occurring. “Non-naturally occurring” (also termed “synthetic” or “artificial”) as applied to an object means that the object is not naturally-occurring—i.e., the object cannot be found in nature as distinct from being artificially produced by man.

A “fragment” or “subsequence” is any portion of an entire sequence, up to but not including the entire sequence. Thus, a fragment or subsequence refers to a sequence of amino acids or nucleic acids that comprises a part of a longer sequence of amino acids (e.g., polypeptide) or nucleic acids (e.g., polynucleotide).

A “specific binding affinity” between two molecules, e.g., a ligand and a receptor, means a preferential binding of one molecule for another in a mixture of molecules. The binding of the molecules is typically considered specific if the binding affinity is about 1×104 M-1 to about 1×109 M-1 or greater (i.e., KD of about 10-4 M to 10-9 M or less). Binding affinity of a ligand and a receptor may be measured by standard techniques known to those of skill in the art. Non-limiting examples of well-known techniques for measuring binding affinities include Biacore® technology (Biacore AB, Sweden), isothermal titration microcalorimetry (MicroCal LLC, Northampton, Mass. USA), ELISA, and flow cytometry (e.g., FACS). For example, flow cytometric methods may be used to select for populations of molecules (such as, for example, cell surface-displayed ligands) which specifically bind to the associated binding pair member (such as a receptor). Ligand-receptor complexes may be detected and sorted by fluorescence (for example, by reacting the complex with a fluorescent antibody that recognizes the complex, or by reacting a complex comprising a biotinylated ligand with a streptavidin-conjugated fluroescent probe). Molecules of interest which bind an associated binding pair member (e.g., receptor) are pooled and re-sorted in the presence of lower concentrations of receptor. By performing multiple rounds of cell sorting in the presence of decreasing concentrations of receptor (an exemplary concentration range being on the order of 10-6 M down to 10-9 M, i.e., 1 micromolar (μM) down to 1 nanomolar (nM), or less, depending on the nature of the ligand-receptor interaction), enriched populations of molecules exhibiting specific binding affinity for the receptor may be obtained.

A polypeptide, nucleic acid, or other component is “isolated” when it is partially or completely separated from components with which it is normally associated (other peptides, polypeptides, proteins (including complexes, e.g., polymerases and ribosomes which may accompany a native sequence), nucleic acids, cells, synthetic reagents, cellular contaminants, cellular components, etc.), e.g., such as from other components with which it is normally associated in the cell from which it was originally derived. A polypeptide, nucleic acid, or other component is isolated when it is partially or completely recovered or separated from other components of its natural environment such that it is the predominant species present in a composition, mixture, or collection of components (i.e., on a molar basis it is more abundant than any other individual species in the composition). In some instances, the preparation consists of more than about 60%, 70% or 75%, typically more than about 80%, or preferably more than about 90% of the isolated species.

A “substantially pure” or “isolated” nucleic acid (e.g., RNA or DNA), polypeptide, protein, or composition also means where the object species (e.g., nucleic acid or polypeptide) comprises at least about 50, 60, or 70 percent by weight (on a molar basis) of all macromolecular species present. A substantially pure or isolated composition can also comprise at least about 80, 90, or 95 percent by weight of all macromolecular species present in the composition. An isolated object species can also be purified to essential homogeneity (contaminant species cannot be detected in the composition by conventional detection methods) wherein the composition consists essentially of derivatives of a single macromolecular species. The term “purified” generally denotes that a nucleic acid, polypeptide, or protein gives rise to essentially one band in an electrophoretic gel. It typically means that the nucleic acid, polypeptide, or protein is at least about 50% pure, 60% pure, 70% pure, 75% pure, more preferably at least about 85% pure, and most preferably at least about 99% pure.

The term “isolated nucleic acid” may refer to a nucleic acid (e.g., DNA or RNA) that is not immediately contiguous with both of the coding sequences with which it is immediately contiguous (i.e., one at the 5′ and one at the 3′ end) in the naturally occurring genome of the organism from which the nucleic acid of the invention is derived. Thus, this term includes, e.g., a cDNA or a genomic DNA fragment produced by polymerase chain reaction (PCR) or restriction endonuclease treatment, whether such cDNA or genomic DNA fragment is incorporated into a vector, integrated into the genome of the same or a different species than the organism, including, e.g., a virus, from which it was originally derived, linked to an additional coding sequence to form a hybrid gene encoding a chimeric polypeptide, or independent of any other DNA sequences. The DNA may be double-stranded or single-stranded, sense or antisense.

A “recombinant polynucleotide” or a “recombinant polypeptide” is a non-naturally occurring polynucleotide or polypeptide which may include nucleic acid or amino acid sequences, respectively, from more than one source polynucleotide or polypeptide, which source polynucleotide or polypeptide may be a naturally occurring polynucleotide or polypeptide, or can itself have been subjected to mutagenesis or other type of modification. A polynucleotide or polypeptide may be deemed “recombinant” when it is synthetic or artificial or engineered, or derived from a synthetic or artificial or engineered polypeptide or nucleic acid. A recombinant polynucleotide (e.g., DNA or RNA) can be made by the combination (e.g., artificial combination) of at least two segments of sequence that are not typically included together, not typically associated with one another, or are otherwise typically separated from one another. A recombinant polynucleotide can comprise a nucleic acid molecule formed by the joining together or combination of polynucleotide segments from different sources and/or artificially synthesized. A “recombinant polypeptide” often refers to a polypeptide that results from a cloned or recombinant polynucleotide.

The term “recombinant” when used with reference, e.g., to a cell, polynucleotide, vector, or polypeptide typically indicates that the cell, polynucleotide, vector, or polypeptide has been modified by the introduction of a heterologous (i.e., non-native, foreign) nucleic acid or the alteration of a native nucleic acid, or that the protein or polypeptide has been modified by the introduction of a heterologous amino acid, or that the cell is derived from a cell so modified. Recombinant cells express nucleic acid sequences that are not found in the native (non-recombinant) form of the cell or express native nucleic acid sequences that would otherwise be abnormally expressed, under-expressed, or not expressed at all. The term “recombinant” when used with reference to a cell indicates that the cell replicates a heterologous nucleic acid, or expresses a polypeptide encoded by a heterologous nucleic acid. Recombinant cells can contain coding sequences that are not found within the native (non-recombinant) form of the cell. Recombinant cells can also contain coding sequences found in the native form of the cell wherein the coding sequences are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, recombination, and related techniques.

A “vector” is a component or composition for facilitating cell transduction or transfection by a selected nucleic acid, or expression of the nucleic acid in the cell. Vectors include, e.g., plasmids, cosmids, viruses, YACs, etc. An “expression vector” is a nucleic acid construct or sequence, generated recombinantly or synthetically, with a series of specific nucleic acid elements that permit transcription of a particular nucleic acid in a host cell. The expression vector can be part of a plasmid, virus, or nucleic acid fragment. The expression vector typically includes a nucleic acid to he transcribed operably linked to a promoter. The nucleic acid to be transcribed is typically under the direction or control of the promoter.

The term “immunoassay” includes an assay that uses an antibody or immunogen to bind or specifically bind an antigen. The immunoassay is typically characterized by the use of specific binding properties of a particular antibody to isolate, target, and/or quantify the antigen.

The term “subject” as used herein includes, but is not limited to, an organism; a mammal, including, e.g., human, non-human primate (e.g., baboon, orangutan, monkey), mouse, pig, cow, goat, cat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

The term “pharmaceutical composition” means a composition which is suitable for pharmaceutical use in a subject, including an animal or human. A pharmaceutical composition generally comprises an effective amount of an active agent, and an excipient or carrier, including, e.g., a pharmaceutically acceptable excipient or carrier.

The tenor “effective amount” means a dosage or amount sufficient to produce a desired result. The desired result may comprise an objective or subjective improvement in the recipient of the dosage or amount.

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as a nucleic acid, vector, gene, polypeptide, protein, substance, or composition thereof that, when administered to a subject who does not display signs or symptoms of pathology, disease or disorder, or who displays only early signs or symptoms of pathology, disease, or disorder, diminishes, prevents, or decreases the risk of the subject developing a pathology, disease, or disorder. A “prophylactically useful” agent or compound (e.g., nucleic acid or polypeptide) refers to an agent or compound that is useful in diminishing, preventing, treating, or decreasing development of pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such as a nucleic acid, vector, gene, polypeptide, protein, substance, or composition thereof, that eliminates or diminishes signs or symptoms of pathology, disease or disorder, when administered to a subject suffering from such signs or symptoms. A “therapeutically useful” agent or compound (e.g., nucleic acid or polypeptide) indicates that an agent or compound is useful in diminishing, treating, or eliminating such signs or symptoms of a pathology, disease or disorder.

Generally, the nomenclature used hereafter and the laboratory procedures in cell culture, molecular genetics, molecular biology, nucleic acid chemistry, and protein chemistry described below are those well known and commonly employed by those of ordinary skill in the art. Standard techniques, such as those described in Sambrook et al., Molecular Cloning: A Laboratory Manual (3nd Ed.), Vols. 1-3, Cold Spring Harbor Laboratory, Cold Spring Harbor, N.Y., 2001 (hereinafter “Sambrook”) and Current Protocols in Molecular Biology, F. M. Ausubel et al, eds., Current Protocols, John Wiley & Sons, Inc. (updated through 2010) (hereinafter “Ausubel”) are used for recombinant nucleic acid methods, nucleic acid synthesis, cell culture methods, and transgene incorporation, e.g., electroporation, injection, gene gun, impressing through the skin, and lipofection. Generally, oligonucleotide synthesis and purification steps are performed according to specifications. The techniques and procedures are generally performed according to conventional methods in the art and various general references which are provided throughout this document. The procedures therein are believed to be well known to those of ordinary skill in the art and are provided for the convenience of the reader.

The term “screening” describes, in general, a process that identifies optimal molecules of the present invention, such as, e.g., polypeptides of the invention, and nucleic acids encoding such molecules. Several properties of these respective molecules can be used in screening, for example: ability of a molecule to bind a ligand or to a receptor, to inhibit cell proliferation, to inhibit viral replication in virus-infected cells, to induce or inhibit cellular cytokine production, to alter an immune response (e.g., induce or inhibit a desired immune response), in a test system or an in vitro, ex vivo or in vivo application. In the case of antigens, several properties of the antigen can be used in selection and screening including antigen expression, folding, stability, immunogenicity and presence of epitopes from several related antigens.

“Selection” is a form of screening in which identification and physical separation are achieved simultaneously. One mode of selection is genetic selection, which may be accomplished, for example, by expression of a selection marker, which in some circumstances allows cells expressing the marker to survive while other cells die (or vice versa). Selection markers include drug and toxin resistance genes, and the like. Screening markers include, for example, luciferase, β-galactosidase and green fluorescent protein, and the like. Another mode of selection involves physical sorting based on a detectable event, such as binding of a ligand to a receptor, reaction of a substrate with an enzyme, or any other physical process which can generate a detectable signal either directly (e.g., by utilizing a chromogenic/fluorogenic substrate ligand) or indirectly (e.g., by reacting with a chromogenic/fluorogenic secondary antibody or chromogenic/fluorogenic ligand).

Selection by physical sorting may by accomplished by a variety of methods, such as by flow cytometry, e.g. in whole cell or microdroplet formats. For example, libraries may be subcloned into a surface display vector to permit expression of polypeptides on the cell surface. Libraries of surface-expressed polypeptides may then be pre-enriched for those polypeptides which bind to a receptor, using flow cytometry. For instance, cells displaying polypeptides which bind the receptor may be detected using a fluorescent-labeled anti-receptor antibody, which, when bound to the receptor, indirectly labels the cells owing to the interaction of the receptor with the surface-displayed polypeptide. Cells which are fluorescently labeled in this manner may then be sorted by flow cytometry, which pre-enriches the library for members which encode expressed and folded polypeptides that bind the receptor. Libraries pre-enriched for receptor binders may then be subjected to a selection using a competition assay to further enrich for members encoding polypeptides that bind tightly to the receptor and/or exhibit a slow off-rate from the receptor, relative to a reference polypeptide. In this approach, receptor is added to a population of cells expressing polypeptides on the cell surface. The cells are washed, and then a reference polypeptide is added. Cell populations are sorted by flow cytometry after various times to select for surface-displayed polypeptides which remained bound to the receptor for increasing periods of time in the presence of the reference polypeptide. Cell populations selected in this manner are expected to be enriched for library members which encode polypeptides exhibiting tighter binding to the cognate receptor, and permits the isolation and identification of polypeptides with activities associated with tight receptor binding.

In the present description and claims, any reference to “a” component, e.g. the context of a non-polypeptide moiety, an amino acid residue, a substitution, a buffet, etc., is intended to refer to one or more of such components, unless stated otherwise or unless it is clear from the particular context that this is not the case. For example, the expression “a component selected from A, B and C” is intended to include all combinations of A, B and C, e.g., A, B, A+C, B+C, or A+B+C.

Various additional terms are defined or otherwise characterized herein.

### Molecules and Methods of the Invention

The present invention provides recombinant 2D-VCAM-1 variant polypeptides and fusion proteins and conjugates thereof, which exhibit VLA4 binding activity. Such 2D-VCAM-1 variant polypeptides and fusion proteins and conjugates thereof may be useful in inhibiting (antagonizing) the binding of native VCAM-1 to VLA4.

Although the present invention is not intended to be limited by a particular theory of underlying mechanism, it is proposed that VLA4 binding activity in surrogate assay systems, such as those described in more detail herein, may be predictive of the efficacy of 2D-VCAM-1 variant polypeptides and fusion proteins and conjugates thereof (also referred to collectively as “molecules of the invention”) in, for example, inhibiting the binding of native VLA4 to native VCAM-1 in a subject. The ability of molecules of the invention to antagonize the VCAM-1:VLA4 interaction makes them useful in preventing or treating disorders, diseases, or symptoms associated with the binding of VLA4 to VCAM-1. Such antagonists may inhibit cell adhesion processes including cell activation, migration, proliferation and differentiation,

### 2D-VCAM-1 Variant Polypeptides

2D-VCAM-1 variant polypeptides of the present invention inhibit the binding of VLA4 and VCAM-1, as demonstrated, e.g., in the cell-based assay of Example 13. Because VLA4 has been implicated as a key mediator in the inflammatory response, inhibitors of the VLA-4:VCAM-1 binding interaction (also referred to as “VLA4 antagonists”) are potentially useful for the treatment, (i.e., therapeutic treatment or prophylactic treatment) of inflammation and for the treatment, (i.e., therapeutic treatment or prophylactic treatment) of conditions associated with inflammatory diseases and disorders.

in one embodiment, the present invention provides a recombinant 2D-VCAM-1 variant polypeptide comprising a sequence which differs in 0-8 amino acid positions from SEQ ID NO: 18, and contains at least two amino acid residues selected from the group consisting of a phenylalanine or tyrosine at position 34 relative to SEQ ID NO:18 (F34 or F34Y); proline at position 37 relative to SEQ ID NO: 18 (P37); leucine at position 39 relative to SEQ NO:18 (L39); and arginine at position 74 relative to SEQ ID NO:18 (R74), wherein the polypeptide has a binding affinity for a human VLA4 (integrin α4β1) protein that is greater than the binding affinity of Q38L-2D-VCAM-1 (SEQ ID NO:10) for the human VLA4 protein.

2D-VCAM-1 variant polypeptides of the present invention may have a combination of any two, three, or four of the above-described amino acid residues, with numbering relative to SEQ ID NO:18. Exemplary combinations include P37+L39; P37+R74; F34+P37; F34+R74; P37+L39+R74; F34+L39+R74; F34+P37+L39; S34+P37+R74; S34+L39+R74; Y34+P37+L39; as well as a combination of four of the above-described amino acid residues, i.e., F34+P37+L39+R74 and Y34+P37+L39+R74, where amino acid position numbering is relative to SEQ ID NO:18.

The polypeptide sequences of the 2D-VCAM-1 variant polypeptides of the present invention often contain phenylalanine at position 34 relative to SEQ ID NO:18 (F34) or a tyrosine at position 34 relative to SEQ ID NO:18 (Y34), and usually contain phenylalanine at position 34 relative to SEQ ID NO:18 (F34). In addition to the sequence features described above, 2D-VCAM-1 variant polypeptides also often contain leucine at position 32 relative to SEQ ID NO:18 (L32) and/or leucine at position 38 relative to SEQ ID NO:18 (L38) and/or serine at position 79 relative to SEQ ID NO:18 (S79).

In the above-described embodiments, 2D-VCAM-1 variant polypeptides may comprise an amino acid sequence that differs from 0-7, 0-6, 0-5, 0-4, 0-3, 0-2, or 0-1 positions relative to SEQ ID NO:18, Suitable substitutions include, L32F, F34S, L38Q, S41A, R74A/I/L/M/F/W/Y/V, S79K/R, L141F, D145A, and R146W.

In a specific embodiment, the recombinant 2D-VCAM-1 variant polypeptide comprises a sequence which differs in 0-8 amino acid positions from SEQ ID NO:18 and which contains the amino acid proline at position 37 (P37) and/or the amino acid leucine at position 39 (L39) relative to SEQ ID NO: 18. Typically, the 2D-VCAM-1 variant polypeptide contains both P37 and L39. Some such 2D-VCAM-1 variant polypeptides also contain a leucine at position 38 (L38) relative to SEQ ID NO:18. Some 2D-VCAM-1 variant polypeptides according to this aspect of the invention comprise one or more substitution selected from L32F/S, F34S/Y, L38Q, S41A, R74T/A, S79K/R, L141F/W, D145A, and R146W relative to SEQ ID NO:18.

2D-VCAM-1 variant polypeptides of the present the invention include a recombinant 2D-VCAM-1 variant polypeptide comprising a sequence which differs in 0-8 amino acid positions from SEQ ID NO:12 and which contains the amino acid proline at position 37 (P37) and the amino acid leucine at position 39 (L39) relative to SEQ ID NO:12. In this embodiment, the 2D-VCAM-1 variant polypeptides may comprise an amino acid sequence that differs from 0-7, 0-6, 0-5, 0-4, 0-3, 0-2, or 0-1 positions relative to SEQ ID NO:12. Some such 2D-VCAM-1 variant polypeptides also contain an alanine at position 41 (A41) relative to SEQ ID NO:12, Some 2D-VCAM-1 variant polypeptides according to this aspect of the invention comprise one or more substitution selected from F32L/S, S34F/Y, Q38L, A41S, T74R/A, K79S/R, L141F/W, D145A, and R146W relative to SEQ ID NO:12.

In addition to the specific substitutions described above, which are recited relative to SEQ ID NOs: 12 and 18, 2D-VCAM-1 variant polypeptides of the present invention may additionally contain conservative substitutions. Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine, praline, cysteine and methionine). The most commonly occurring exchanges are Ala/Ser, Val/Ile, Asp/Glu, Thr/Ser, Ala/Gly, Ala/Thr, Ala/Val, Ser/Gly, Tyr/Phe, Ala/Pro, Lys/Arg, Asp/Asn, Leu/Ile, and Leu/Val as well as these in reverse.

In another aspect, the invention includes a recombinant 2D-VCAM-1 variant polypeptide having a first domain sequence and a second domain sequence (which are also referred to herein as “domain-1” and “domain-2”, respectively) which are linked either directly by a peptide bond or indirectly via a linker (such as, for example, a linker peptide). The domain-1 sequence differs in 2-8 amino acid positions (that is, differs in 2 positions, in 3 positions, in 4 positions, in 5 positions, in 6 positions, in 7 positions, or in 8 positions) from the Domain-1 amino acid sequence of human 2D-VCAM-1 (amino acids 1-88 of SEQ ID NO:2) and comprises the substitutions T37P and I39I, relative to SEQ ID NO:2. The domain-1 sequence of the variant may fluffier comprise one or more domain-1 substitutions selected from F32L/S, S34F/Y, Q38L, S41A, T74R/A, and K79S/R relative to SEQ ID NO:2. The domain-2 sequence of the variant differs in 0-3 amino acid positions (that is, is the same as, or, differs in 1 position, in 2 positions, or in 3 positions) from the Domain-2 amino acid sequence of human. 2D-VCAM-1 (amino acids 96499 of SEQ ID NO:2), and optionally comprises one or more domain-2 substitution selected from L141F, D145A, and R146W relative to SEQ ID NO:2. Some such 2D-VCAM-1 variant polypeptides according to this aspect of the invention contain a linker peptide, such as a linker peptide corresponding to amino acids 89-95 of SEQ ID NO:2, which indirectly links the domain-1 sequence and the domain-2 sequence.

In addition to one or more of the domain-1 substitutions described above, some 2D-VCAM-1 variant polypeptides of the present invention may contain one or more of the following amino acid residues relative to human 2D-VCAM-1 (SEQ ID NO:2): arginine at position 36 (R36), aspartic acid at position 40 (D40), and proline at position 42 (P42), where amino acid position is determined by optimal alignment with SEQ ID NO:2. Some such 2D-VCAM-1 variant polypeptides of the invention contain at least the amino acid residue aspartic acid at position 40 (D40). For example, 2D-VCAM-1 variant polypeptides of the invention may contain any one or more of the above identified domain-I substitutions, in combination with D40 and one or both of R36 and P42. For instance, some 2D-VCAM-1 variant polypeptides of the present invention contain the residues R36, D40 and P42 plus the substitutions T37P and I39I, relative to SEQ ID NO:2, and may further comprise one or more of the domain-1 substitutions F32L/S, S34F/Y, Q38L, S41A, T74R/A, and K79S/R relative to SEQ ID NO:2.

The 2D-VCAM-1 variant polypeptides of the invention bind a VLA4 protein (integrin α4β1), for example, a human VLA4 protein, such as a human VLA4-Fc fusion described in Example 3 herein. In other words, the 2D-VCAM-1 variant polypeptides of the invention “exhibit VLA4 binding activity”. In some instances, a 2D-VCAM-1 variant polypeptide of the invention has a binding affinity for a VLA4 protein, such as a human VLA4 protein, e.g., a human VLA4-Fc fusion described in Example 3 herein, that is greater than the binding affinity of human 2D-VCAM-1 (SEQ ID NO:2) for the VLA4 protein. Some such 2D-VCAM-1 variant polypeptides of the invention have a binding affinity for a VLA4 protein that is greater than the binding affinity of Q38L-2D-VCAM-1 (SEQ ID NO:10) for the VLA4 protein.

Some 2D-VCAM-1 variant sequences of the present invention may comprise one of the following motifs: RPQLDAP (SEQ ID NO:7), or RPLLDSP (SEQ ID NO:8). Generally these motif sequences are located at or about amino acid positions 36-42 relative to the human 2D-VCAM-1 sequence (SEQ ID NO:2). The present invention therefore also provides a recombinant 2D-VCAM-1 variant polypeptide having a variant sequence which differs in up to 10 amino acid positions from SEQ ID NO:2 and which comprises a motif selected from RPQLDAP (SEQ ID NO:7) and RPLLDSP (SEQ ID NO:8). Typically, such a 2D-VCAM-1 variant polypeptide sequence comprising one of the above motifs differs in up to 8, in up to 6, or in up to 4 amino acid positions from SEQ ID NO:2, and has a binding affinity for a VLA4 protein that is greater than the binding affinity of human 2D-VCAM-1 (SEQ ID NO:2), or, in some instances, greater than the binding affinity of of Q38L-2D-VCAM-1 (SEQ ID NO:10), for the VLA4 protein.

Exemplary 2D-VCAM-1 variants of of the present invention are provided herein as SEQ ID NO:12, SEQ NO:14, SEQ ID NO:16, SEQ ID NO:18, SEQ ID NO:20, SEQ ID NO:22, SEQ ID NO:24, SEQ ID NO: 101, SEQ ID NO: 103, SEQ ID NO: 105, SEQ ID NO: 107, SEQ ID NO: 109, SEQ ID NO:111, SEQ ID NO: 113, SEQ ID NO: 115, SEQ ID NO:117, SEQ ID NO:119, SEQ ID NO:121, SEQ ID NO:123, SEQ ID NO:125, SEQ NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ NO:145, SEQ ID NO:147, SEQ NO:149, SEQ ID NO:151, SEQ ID NO:153, SEQ ID NO:155, SEQ ID NO:157, SEQ ID NO:159, SEQ ID NO:161, and SEQ ID NO:163.

2D-VCAM-1 variant polypeptides of the present invention may contain additional amino acid substitutions, deletions, and/or insertions of amino acid residues relative to the reference (or parent) polypeptide sequence, such as SEQ ID NO:2 or one of the exemplary 2D-VCAM-1 variant sequences provided herein, such as SEQ ID NO:12 or SEQ ID NO:18. Such 2D-VCAM-1 variant polypeptides may be readily identified using mutagenesis methods and other methods for generating variant libraries known in the art, together with, for example, the ELISA assay of Example 10 and/or the BIACORE assay of Example 12 to determine binding to VLA4.

Other positions suitable for substitution, deletions, and/or insertions may be located in the natural linker between domains 1 and 2 of 2D-VCAM. Amino acid residues. 89-95 of SEQ ID NO:2 form a natural linker peptide between Domain-1 and Domain-2 of human 2D-VCAM-1. Some 2D-VCAM-1 variants of the present invention contain this linker (corresponding to residues 89-95 of SEQ ID NO:2), indirectly joining the domain-1 and domain-2 sequence of the variant polypeptide. The present invention also contemplates the use of this and other suitable linkers to join domain-1 and domain-2, as well as to join a 2D-VCAM-1 variant polypeptide with an Fc region in a 2D-VCAM-1 variant-Fc fusion polypeptide. Other suitable linkers include linkers made up of any of the 20 naturally occurring amino acids (i.e., Ala, Arg, Asn, Asp, Cys, Gln, Glu, Gly, His, Ile, Leu, Lys, Met, Phe, Pro, Ser, Thr, Trp, Tyr, and Val) or any non-naturally occurring amino acids known in the art, as well as combinations thereof. The use of linkers made up of either naturally occurring amino acid residues or non-naturally occurring amino acid residues to connect polypeptides into novel fusion polypeptides is well known in the art and has been reported in the following references, each of which is incorporated herein by reference; Hallewell, et al. (1989) J. Biol. Chem. 264:5260-5268; Alfthan, et al. (1995) Protein Eng 8:725-731; Robinson & Suer (1996) Biochemistry 35:109-116; Khandekar, et al. (1997) J. Biol. Chem., 272:32190-32197; Fares, et at (1998) Endocrinology 139:2459-2464; Smallshaw et al. (1999) Protein Eng. 12:623-630; and U.S. Pat. No. 5,856,456. The use of linker peptides has also been demonstrated in the production of single-chain antibodies where the variable regions of a light chain (VL) and a heavy chain (VH) are joined through a linker peptide. A widely used linker peptide is a 15-mer consisting of three repeats of a Gly-Gly-Gly-Gly-Ser amino acid sequence ((Gly4 Ser)3; SEQ NO:25). Phage display technology has been used to diversify and select appropriate linker sequences (Tang, et al. (1996) J. Biol. Chem. 271:15682-45686; Hennecke, et al. (1998) Protein Eng. 11:405-410, both of which are incorporated herein by reference).

In some embodiments, the linker peptide which indirectly joins the domain-1 and domain-2 sequences may contain at least 50% glycine residues, and in some instances at least 75% glycine residues. The linker peptide may also be made up of only glycine residues. For example, the linker may contain 1-20 glycine residues, 2-16 glycine residues, 3-15 glycine residues, 4-12 glycine residues or 5-10 glycine residues. In addition to the glycine residues, the linker may comprise other residues, in particular, residues selected from the group consisting of Ser, Ala and Thr. Linker peptides employed in 2D-VCAM-1 variants of the invention may contain only glycine and serine residues in their sequences. For example, the linker peptide may be of the form (Gly₃Ser)ₙ, where n is from 1 to 5, inclusive, or from 1 to 3, inclusive. Other suitable linker peptides having only glycine and serine residues may be of the form (Gly₄Ser)ₙ, wherein n is from 1 to 4, inclusive, or from 1 to 3, inclusive.

Other suitable linker peptides include those having the amino acid sequence Glyₓ-Xaa-Gly_{y}-Xaa-Gly_{z}, wherein each Xaa is independently selected from the group consisting of Ala, Val, Leu, Ile, Met, Phe, Trp, Pro, Gly, Ser, Thr, Cys, Tyr, Asn, Gln, Lys, Arg, His, Asp and Glu, and wherein x, y and z are each integers from 1 to 5, inclusive. In some embodiments, each Xaa is independently selected from the group consisting of Ser, Ala and Thr. More particularly, the linker peptide has the amino acid sequence Gly-Gly-Xaa-Gly-Gly-Gly-Xaa-Gly-Gly-Gly (SEQ ID NO:26), wherein each Xaa is independently selected from the group consisting of Ala, Val, Leu, Ile, Met, Phe, Trp, Pro, Gly, Ser, Thr, Cys, Tyr, Asn, Gln, Lys, Arg, His, Asp and Glu. In some embodiments, each Xaa is independently selected from the group consisting of Ser, Ala and Thr. Typically, Xaa is Ser.

Suitable linker peptides include those comprising at least one proline residue in the amino acid sequence of the linker peptide. Linker peptides for use in 2D-VCAM-1 variants of the present invention also may comprise at least one cysteine residue and/or at least one lysine residue. Thus, in some embodiments of the present invention the linker peptide comprises amino acid residues selected from the group consisting of Gly, Ser, Ala, Thr, Cys, Lys, and Pro.

Other sequence modifications may be made for the purpose of altering the serum half life characteristic of the 2D-VCAM-1 variants when administered in vivo. For example, sequence modifications for introducing or removing glycosylation or PEGylation sites may be made in the 2D-VCAM-1 variants of the present invention. This is described in more detail hereinbelow. In one embodiment, the present invention also provides 2D-VCAM-1 variant polypeptide sequences with a substitution, insertion, or deletion, of an amino acid to which a non-polypeptide conjugation moiety may be covalently bound. The specific amino acid to be removed or introduced is selected based on the nature of the non-polypeptide conjugation moiety to be removed or introduced. For example, when the non-polypeptide conjugation moiety is a non-polypeptide polymer, such as, for example, a polyalkylene oxide derivative such as a polyethylene glycol (PEG) activated with a suitable reactive functional group, the attachment group may he cysteine, lysine, the N-terminal amino acid residue (i.e., via the terminal α-amino group), aspartic acid, glutamic acid, histidine, or arginine, or any combination of two or more thereof. Lysine and cysteine are typical attachment groups which are introduced or removed in the context of adding or removing attachment sites for conjugation to a non-polycpeptide conjugation moiety.

As used herein, the term “non-polypeptide conjugation moiety” refers to a non-polypeptide polymer moiety, sugar moiety, or non-polymeric lipophilic moiety. Exemplary non-polypeptide conjugation moieties are described in further detail below. The amino acid residue to which a non-polypeptide conjugation moiety covalently binds is referred to herein as an “attachment group”. The non-polypeptide conjugation moieties react with specific attachment sites on the attachment groups. The term “attachment site” refers to the specific functional group involved in the conjugation reaction,

Exemplary substitutions (relative to either SEQ ID NOs:2, 12, or 18) for introducing lysine-reactive attachment sites into the sequences of the 2D-VCAM-1 variant polypeptides of the invention include R10K, R36K, R78K, R123K, R146K, R172K, R187K, as well as combinations of any two or more thereof. Exemplary substitutions (relative to either SEQ ID NOs:2, 12, or 18, except where noted) for removing lysine-reactive attachment sites from the 2D-VCAM-1 variant polypeptide sequences of the invention include: K2R/Q, K46R/Q, K79R/Q (note that position 79 is not a lysine-reactive attachment site in SEQ ID NO:18), K82R/Q, K93R/Q, K107R/Q, K112R/Q, K130R/Q, K136R/Q, K147R/Q, K152R/Q, K167R/Q, as well as combinations of any two or more thereof. Exemplary non-polypeptide conjugation moieties are described in further detail below.

b 2D-VCAM-1 variant polypeptides of the present invention are typically from about 190 to about 230 amino acid residues in length, more typically from about 185 to about 210 amino acid residues in length, such as from about 190 to about 205 amino acid residues in length, generally from about 195 to about 200 amino acid residues in length, and often about 199 amino acid residues in length.

In another embodiment, the present invention provides a fusion protein comprising a first polypeptide, wherein the first polypeptide is a 2D-VCAM-1 variant polypeptide of the present invention and a second polypeptide or peptide, wherein the second polypeptide or peptide is used to either the N or C-terminus of the 2D-VCAM variant polypeptide. In another embodiment, the present invention provides a fusion protein comprising a first polypeptide, wherein the first polypeptide is a 2D-VCAM-1 variant polypeptide of the present invention, a second polypeptide or peptide, wherein the second polypeptide or peptide is fused to the N-terminus of the 2D-VCAM variant polypeptide, and a third polypeptide or peptide is fused to the C-terminus of the 2D-VCAM variant polypeptide, wherein the second and third polypeptide or peptide may be identical or different. Polypeptides and peptides that are suitable for use in the context of a fusion protein of the present invention include those which impart desirable characteristics to 2D-VCAM-1 variant polypeptide, such as, for example, extended half life, binding to a molecular entity other than VLA4 which may be desirable for targeting or purification, and the like. Exemplary second and third polypeptides/peptides include, for example, poly-histidine tags and variants thereof, all or part of an Fc region of an immunoglobulin, human serum albumin, and the like.

For example, the 2D-VCAM-1 variant polypeptide may be expressed as a fusion protein comprising a tag peptide or a linker peptide of, e.g., 1-5 or 1-10 or 1-20 or 1-30 amino acid residues. The tag peptide or linker peptide may be attached (i.e., fused) to the N-terminus or to the C-terminus of the 2D-VCAM-1 variant polypeptide. The tag peptide or linker peptide may, for example, be designed to facilitate purification of the polypeptide, or may facilitate conjugation to a non-polypeptide polymeric moiety (as described in more detail below). A number of suitable tags are commercially available from, for example, Unizyme. Laboratories, Denmark. Exemplary tags include: His-His-His-His-His-His (SEQ ID NO:27); Met-Lys-His-His-His-His-His-His (SEQ ID NO:28); Met-Lys-His-His-Ala-His-His-Gln-His-His (SEQ ID NO:29); Met-Lys-His-Gln-His-Gln-His-Gln-His-Gln-His-Gln-His-Gln (SEQ ID NO:30); Gln-Gln-Lys-Leu-Ile-Ser-Glu-Glu-Asp-Leu (SEQ ID NO:31; a C-terminal tag described by Evan, G. I. et al., Mol. Cell. Biol. 5:3610-16 (1985), which is incorporated by reference herein). Additional suitable linker peptides may be obtained by optimizing the linker peptides described herein or in the literature by using well known mutagenesis techniques, such as random mutagenesis.

In another example, the fusion protein may be a 2D-VCAM-1 variant-Fc fusion polypeptide comprising a 2D-VCAM-1 variant polypeptide of the present invention linked, either directly or indirectly via a linker, to part or all of an Fc region of an immunoglobulin (Ig), Fc regions suitable for use in the practice of the present invention include part or all of the second and third constant domains of the heavy chains (i.e., CH2 and CH3) of antibody isotypes IgG, IgA, or IgD, or the second, third, and/or fourth constant domains (i.e., CH2, CH3, and CH4) of the heavy chains of IgM or IgE. Suitable linkers include those described in more detail hereinbelow. Example 22 illustrates the construction of a 2D-VCAM-1 variant-Fc fusion polypeptide of the present invention.

### Properties of 2D-VCAM-1 Variants

Sonic 2D-VCAM-1 variant polypeptides of the present invention have greater binding affinities (i.e., lower EC50 values) for a VLA4 protein (such as, human VLA4-Fc) compared to the binding affinity of human 2D-VCAM-1 (SEQ ID NO:2) for the VLA4 protein as determined by the ELISA assay of Example 10. In the assay of Example 10, 2D-VCAM-1 variant polypeptides of the present invention typically exhibit at least a 5-fold, such as at least a 10-fold greater affinity for human VLA4-Fc, relative to the affinity of human 2D-VCAM-1 (SEQ ID NO:2) for human VLA4-Fc. Some such 2D-VCAM-1 variant polypeptides of the invention exhibit at least a 15-fold, at least a 20-fold, at least a 50-fold, at least a 100-fold, at least a 150-fold, at least a 200-fold, at least a 300-fold, up to a 500-fold greater affinity for human VLA4-Fc relative to the affinity of human 2D-VCAM-1 (SEQ ID NO:2) for human VLA4-Fc in the assay of Example 10.

Some 2D-VCAM-1. variant polypeptides of the present invention also have at least equivalent, or typically greater, binding affinities for a VLA4 protein compared to the binding affinity of Q38L-2D-VCAM-1 polypeptide (SEQ ID NO:10) for the VLA4 protein, as determined by either the ELISA assay of Example 10 and/or the BIACORE assay of Example 12. In the assay of Example 10, 2D-VCAM-1 variant polypeptides of the present invention typically exhibit at least a 2-fold greater affinity for human VLA4-Fc, relative to that of the Q38L-2D-VCAM-1 polypeptide (SEQ ID NO:10), for human VLA4-Fc. More typically, 2D-VCAM-1 variant polypeptides of the invention exhibit at least a 3-fold, at least a 5-fold, at least a 10-fold, at least a 15-fold, at least a 20-fold, at least a 30-fold, up to a 50-fold greater affinity for human VLA4-Fc, relative to that of the Q38L-2D-VCAM-1 polypeptide (SEQ ID NO:10) in the assay of Example 10.

2D-VCAM-1 variant polypeptides of the present invention exhibit greater or lesser binding affinity for an LPAM-1 protein (integrin α4β7). i.e., where the ratio of VLA4/LPAM-1 binding affinity is greater or less than 1, respectively). Certain 2D-VCAM-1 variant polypeptides of the present invention exhibit greater or lesser binding affinity for an. LPAM-1 protein (integrin α4β7), compared to that of human 2D-VCAM-1 (SEQ ID NO:2) or that of the Q38L-2D-VCAM-1 polypeptide (SEQ NO:10), as determined in the ELISA assay of Example 11. As with the recombinant monoclonal antibody natalizumab (TYSABRI®) and wild type human VCAM-1, some 2D-VCAM-1 variant polypeptides of the present invention exhibit binding activity with respect to both human VLA4 (α4β1) and LPAM-1 (α4β7).

Accordingly, some 2D-VCAM-1 variant polypeptides of the present invention exhibit greater binding affinity to an LPAM-1 protein relative to the Q38L-2D-VCAM-1 polypeptide. Such variants typically exhibit at least a 2-fold, at least a 3-fold, at least a 5-fold, at least a 10-fold, at least a 15-fold, at least a 20-fold, at least a 30-fold, up to a 50-fold greater affinity for an LPAM-1. protein relative to that of the Q38L-2D-VCAM-1 polypeptide (SEQ ID NO:10) in the assay of Example 11.

Furthermore, some 2D-VCAM-1 variant polypeptides of the present invention exhibit equivalent or reduced binding affinity to an LPAM-1. protein relative to that of the human 2D-VCAM-1 polypeptide or the Q38L-2D-VCAM-1 polypeptide. Such variants typically exhibit less than a 2-fold improvement in binding, relative to that of the Q38L-2D-VCAM-1 polypeptide (SEQ ID NO:10), to LPAM-1-Fc in the assay of Example 11. More typically, such 2D-VCAM-1 variant polypeptides exhibit less than a 1.5-fold, less than a 1-fold, less than a 0.5-fold, less than a 0.2-fold, to a 0.1-fold improvement in binding to LPAM-1-Fc relative to that of the Q38L-2D-VCAM-1 polypeptide (SEQ ID NO:10) in the assay of Example 11.

Some 2D-VCAM-1 variant polypeptides of the present invention have equilibrium dissociation constants (KD) for a VLA4 protein (e.g., human VLA4-Fc) that are less than that of human 2D-VCAM-1, In other words, some 2D-VCAM-1 variant polypeptides have binding affinities for the VLA4 protein that are greater than that of human 2D-VCAM-1 (SEQ ID NO:2), as determined, e.g., by the kinetic binding assay of Example 12. In the assay of Example 12, 2D-VCAM-1 variant polypeptides of the present invention typically exhibit at least a 5-fold greater affinity for VLA4-Fc than human 2D-VCAM-1 (SEQ ID NO:2). More typically, they exhibit at least a 10-fold, at least a 15-fold, at least a 20-fold, at least a 30-fold, at least a 50-fold, at least a 100-fold, at least a 150-fold, at least a 200-fold, at least a 300-fold, at least a 400-fold, up to a 500-fold greater binding affinity for VLA4-Fc relative to that of human 2D-VCAM-1 (SEQ ID NO:2) in the assay of Example 12.

Some 2D-VCAM-1 variant polypeptides of the present invention have binding affinities for a VLA4 protein, e.g. VLA4-Fc, that are about equal to, or typically greater than, that of the Q38L-2D-VCAM-1 polypeptide (SEQ ID NO:10). Some such 2D-VCAM-1 variant polypeptides of the present invention exhibit at least a 2-fold, at least a 4-fold, at least an 8-fold, at least a 10-fold, at least a 15-fold, at least a 20-fold, at least a 25-fold, at least a 30-fold, at least a 35-fold, at least a 40-fold, up to a 50-fold greater binding affinity for VLA4-Fc than that of the Q38L-2D-VCAM-1 polypeptide (SEQ ID NO:10) in the assay of Example 12.

Certain 2D-VCAM-1 variant polypeptides of the present invention exhibit a binding affinity for an LPAM-1 protein (α4β7), e.g. LPAM-1-Fc, that is greater than that of wild type 2D-VCAM-1. Such variants typically exhibit at least a 2-fold, at least a 5-fold, at least a 10-fold, at least a 15-fold, at least a 20-fold, at least a 30-fold, at least a 50-fold, at least a 100-fold, at least a 150-fold, at least a 200-fold, at least a 300-fold, at least a 400-fold, up to a 500-fold greater binding affinity for LPAM-1-Fc than human 2D-VCAM-1 (SEQ ID NO:2) in the assay of Example 12.

Other 2D-VCAM-1 variant polypeptides of the present invention exhibit about equal binding affinity, or lower binding affinity, for an LPAM-1 protein (α4β7), e.g. LPAM-1-Fc, relative to that of wild type 2D-VCAM-1 or Q38L-2D-VCAM-1. Such variants typically exhibit less than a 2-fold greater binding affinity, relative to that of the Q38L-2D-VCAM-1 polypeptide, for LPAM-1-Fc in the assay of Example 12. More typically, such 2D-VCAM-1 variant polypeptides exhibit less than about a 1.5-fold, less than about a 1-fold, less than about a 0.5-fold, less than about a 0.2-fold, to about a 0.1-fold greater binding affinity to LPAM-1-Fc relative to that of the Q38L-2D-VCAM-1 polypeptide (SEQ ID NO:10) in the assay of Example 12.

In addition, as shown in Example 12, some 2D-VCAM-1 variant polypeptides of the invention exhibit ratios of VLA4/LPAM-1 binding affinity which are greater than that of the ratio of VLA4/LPAM-1. binding affinity exhibited by human 2D-VCAM-1 or by Q38L-2D-VCAM-1. In other words, some variants of the invention exhibit a greater improvement in binding affinity to a VLA4 (α4β1) protein than to an LPAM-1 (α4 β7) protein, as compared to the relative binding affinities of either human 2D-VCAM-1 or Q38L-2D-VCAM-1 for VLA4 versus LPAM-1. A 2D-VCAM-1 variant polypeptide having a high VLA4/LPAM-1 binding affinity ratio, relative to that of human 2D-VCAM-1 or Q38L-2D-VCAM-1, may be especially advantageous if LPAM-1 binding is not desired, for example, if LPAM-1 binding is correlated to adverse side effects such as progressive multifocal leukoencephalopathy (PML).

2D-VCAM-1 variant polypeptides of the present invention have been demonstrated to exhibit preferential binding to activated. VLA4 as compared to inactive VLA4 as shown in FIGS. 14 and 15. The corresponding experiments are described in Example 21 hereinbelow. As explained in the Background section, VLA4, like other integrin receptors, exists in both a low affinity conformational state (or inactive state) and a high affinity conformational state (or activated state). The transition from the low affinity to high affinity state is due to conformational changes in the integrin receptor following divalent cation binding such as magnesium (Mg²⁺) and manganese (Mn²⁺), which increases the affinity of VLA4 and LPAM-1 to their ligands. As shown in FIG. 14, 2D-VCAM-1 variant polypeptides of the present invention (illustrated by SEQ ID NOs:12, 14, and 18) exhibit greater binding to activated VLA4 as compared to inactive VLA4. The 2D-VCAM-1 variant polypeptides also exhibit greater binding to activated VLA4 as compared to wildtype 2D-VCAM-1 (SEQ NO:2) and/or the Q38L-2D-VCAM-1 variant (SEQ ID NO:10).

FIG. 15 illustrates the effect of VLA4 activation state on the binding of Clone 146 (SEQ ID NO:18). The figure confirms that the 2D-VCAM-1 variant polypeptide binds to activated VLA4, but does not bind tightly to unactivated VLA4. This preference of 2D-VCAM-1 polypeptides to activated VLA4 may lead to selective blocking of the activated receptors that are relevant to disease and reducing the potential pool of polypeptide bound to irrelevant integrin receptors. Selectively targeting the relevant VLA4 receptors may also allow optimal 2D-VCAM-1 variant polypeptide dose levels that enable prevention of the disease without excessive immune suppression. Therefore, 2D-VCAM-1 variant polypeptides of the present invention may be beneficial in providing a reduced risk of opportunistic infection, such as by JC virus, and diseases caused by such infections, such as PML, compared to other α4β1 antagonists, such as natalizumab, that bind equally well to activated and non-activated VLA4.

When administered to a mammal, variant polypeptides of the present invention appear not to downregulate surface expression of α4 and β1 integrins on peripheral blood lymphocytes as demonstrated in Examples 19A and 19B. In Example 19A, female Balb/c mice were injected subcutaneously with either a pegylated form of Clone 146 (SEQ NO:18) (“PEG50-146”), a rat anti-murine α4 integrin monoclonal antibody PS/2 or phosphate buffer solution (PBS). Whole blood collected from the mice was analyzed for α4 and β1 integrin levels on B220° cells. The results are depicted in FIG. 10. In contrast to the increase observed in cell surface levels of a4 integrin in the PS/2-treated mice, the cell surface levels of □4 integrin appeared unchanged in the PEG50-146-treated mice. Similar results were obtained with respect to b1 integrin levels (Table 12) in Example 19B, male cynomolgus monkeys were injected subcutaneously with either PEG50-146 or humanized anti-α4 integrin monoclonal antibody natalizumab. Whole blood collected from the monkeys was analyzed for α4 integrin and β1 integrin cell surface levels on CD20+ peripheral B cells and CD3+ peripheral T cells. As shown in FIG. 11A, there was a significant reduction in the natalizumab treated monkeys in percentage of CD49d+ B cells as compared to the PEG50-146 variant. As shown in FIG. 11B, a similar reduction in T cells that were CD49d+ was observed with the natalizumab treated monkeys, relative to the PEG50-146 variant-treated monkeys. As shown in FIGS. 12A and 12B, there was also a significant reduction in the natalizumab treated monkeys in percentage of CD29+ B cells and CD29+ T cells, respectively, as compared to the PEG50-146 variant.

Accordingly, in a further embodiment, the present invention provides 2D-VCAM-1 variant polypeptides that when administered to mammal (a human or non-human mammal), do not induce a decrease in the percentage of CD49d+ or CD29+ B cells or T cells in peripheral blood as compared to an untreated control mammal using the assay in Example 19. Without wishing to be bound by any theory, it is believed that the variants' property of not inducing a decrease in surface expression of α4 and β1 integrins on peripheral blood lymphocytes when administered in vivo may have profound effects on immunological activities dependent upon these adhesion molecules. These activities include immune surveillance and migration of immune cells to sites of inflammation. It is believed that this lack of impact on adhesion cell surface expression may translate to a reduced effect on immune surveillance, immune cell trafficking, immune homeostasis, and risk of opportunistic infections, such as that caused by It virus, leading to an overall reduced risk of PML, compared to other VLA4 antagonists that affect the cell surface expression of cell adhesion molecules.

It is believed that protein fusions and conjugates comprising the 2D-VCAM-1 variant polypeptides of the present invention also exhibit the properties described above for the 2D-VCAM-1 variant polypeptides. In view of the properties observed with respect to the molecules of the present invention as described above and in the Examples hereinbelow, it is believed that molecules of the present invention may be beneficial as a therapeutic or prophylactic treatment of an inflammatory disease or disorder with a reduction in risk of PML associated with other VLA4 antagonists.

2D-VCAM-1 variant polypeptides having the properties described herein which are not specifically described, can be readily identified based on the key amino acid residue information provided hereinabove coupled with mutagenesis, as described in the section entitled “Polynucleotides of the Invention” below, and the assays described in the Examples.

### Polynucleotides of the Invention

The present invention provides polynucleotides comprising nucleic acid sequences that encode 2D-VCAM-1 variant polypeptides of the invention and protein fusions thereof as described above. Exemplary polynucleotides of the invention include SEQ ID NOS:11, 13, 15, 17, 19, 21, 23, 100, 102, 104, 106, 108, 110, 112, 114, 116, 118, 120, 122, 124, 126, 128, 130, 132, 134, 136, 138, 140, 142, 144, 146, 148, 150, 152, 154, 156, 1.58, 160, and 162. These polynucleotides encode exemplary polypeptides of the invention corresponding to SEQ ID NOS:12, 14, 16, 18, 20, 22, 24, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, and 163 respectively.

Those having ordinary skill in the art will readily appreciate that due to the degeneracy of the genetic code, a multitude of polynucleotide sequences encoding 2D-VCAM-1 variants of the present invention exist. Table 1 is a Codon Table that provides the synonymous codons for each amino acid. For example, the codons AGA, AGG, CGA, CGC, CGG, and CGU all encode the amino acid arginine. Thus, at every position in the polynucleotides of the invention where an arginine is specified by a codon, the codon can be altered, to any of the corresponding codons described above without altering the encoded polypeptide. It is understood that U in an RNA sequence corresponds to T in a DNA sequence.

TABLE 1
Codon Table

| Amino acids | | | Codon |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Gln | P | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Such “silent variations” are one species of “conservative” variation. One of ordinary skill in the art will recognize that each codon in a polynucleotide sequence (except AUG, which is ordinarily the only codon for methionine, and UGG, which is ordinarily the only codon for tryptophan) can be modified by standard techniques to encode a functionally identical polypeptide. Accordingly, each silent variation of a polynucleotide which encodes a polypeptide is implicit in any described sequence. The invention contemplates and provides each and every possible variation of polynucleotide sequence encoding a polypeptide of the invention that could be made by selecting combinations based on possible codon choices. These combinations are made in accordance with the standard triplet genetic code (set forth in Table 1), as applied to the polynucleotide sequences of the present invention.

A group of two or more different codons that, when translated in the same context, all encode the same amino acid, are referred to herein as “synonymous codons.” 2D-VCAM-1 variants of the present invention may be codon optimized for expression in a particular host organism by modifying the polynucleotides to conform with the optimum codon usage of the desired host organism. Those having ordinary skill in the art will recognize that tables and other references providing preference information for a wide range of organisms are readily available. See e.g., Henaut and Danchin in “Escherichia coli and Salmonella,” Neidhardt, et al, Eds., ASM Press, Washington D.C. (1996), pp. 2047-2066, which is incorporated herein by reference. An example of a polynucleotide optimized for expression by a particular host organism is SEQ ID NO:55, which is an E, coli optimized human 2D-VCAM-1 polynucleotide sequence. The human 2D-VCAM-1 polynucleotide sequence is provided as SEQ ID NO:1.

The terms “conservatively modified variations” and “conservative variations” are used interchangeably herein to refer to those polynucleotides that encode identical or essentially identical amino acid sequences, or in the situation where the polynucleotides are not coding sequences, the term refers to polynucleotides that are identical. One of ordinary skill in the art will recognize that individual substitutions, deletions or additions which alter, add or delete a single amino acid or a small percentage of amino acids in an encoded sequence are considered conservatively modified variations where the alteration results in one or more of the following: the deletion of an amino acid, addition of an amino acid, or substitution of an amino acid with a chemically similar amino acid. When more than one amino acid is affected, the percentage is typically less than 5% of amino acid residues over the length of the encoded sequence, and more typically less than 2%. References providing amino acids that are considered conservative substitutions for one another are well known in the art.

Polynucleotides of the present invention can be prepared using methods that are well known in the art. Typically, oligonucleotides of up to about 50 to 120 bases are individually synthesized, then joined (e.g., by enzymatic or chemical ligation methods, or polymerase-mediated methods) to form essentially any desired continuous sequence. Polynucleotides of the present invention can be prepared by chemical synthesis using, for example, the classical phosphoramidite method described by Beaucage, et al, (1981) Tetrahedron Letters 22:1859-69, or the method described by Matthes, et al, (1984) EMBO J. 3:801-05, both of which are incorporated herein by reference. According to the phosphoramidite method, oligonucleotides are synthesized, purified, annealed, ligated and cloned in appropriate vectors. In addition, essentially any oligonucleotide can be custom ordered from any of a variety of commercial sources, such as, for example. The Midland Certified Reagent Company (Midland, Tex.). The Great American Gene Company (Ramona, Calif.), ExpreasGen Inc. (Chicago, Ill.), and others.

Polynucleotides may also be synthesized by well-known techniques as described in, for example, Caruthers, et al., Cold Spring Harbor Symp, Quant. Biol, 47:411-418 (1982) and Adams, et al., J. Am. Chem. Soc. 105:661 (1983), both of which are incorporated herein by reference. Double stranded DNA fragments may then be obtained either by synthesizing the complementary strand and annealing the strands together under appropriate conditions, or by generating the complementary strand in a polymerase chain reaction using DNA polymerase with an appropriate primer sequence.

2D-VCAM-1 variant polypeptides not specifically detailed herein may be readily identified using known methods for generating variant libraries followed by screening to detect VLA4 binding activity using, for example, the assay methods of Examples 6 or 7. Methods for generating variant libraries are well known in the art. For example, mutagenesis and directed evolution methods can be readily applied to polynucleotides (such as, for example, polynuckotides encoding human 2D-VCAM-1 (e.g., SEQ ID NO:1) or any of the 2D-VCAM-1 variant polypeptide-encoding polynucleotides of the present invention, described herein) to generate variant libraries that can be expressed, screened, and assayed using the methods described herein. Mutagenesis and directed evolution methods are well known in the art. See, e.g., Ling, et al., “Approaches to DNA mutagenesis; an overview,” Anal. Biochem., 254(2);157-78 (1997); Dale, et al, “Oligonucleotide-directed random mutagenesis using the phosphorothioate method,” Methods Mol. Biol., 57:369-74 (1996); Smith, “In vitro mutagenesis,” Ann. Rev. Genet., 19:423-462 (1985); Botstein, et al., “Strategies and applications of in vitro mutagenesis,” Science, 229:1193-1201 (1985); Carter, “Site-directed mutagenesis,”Biochem. J., 237:1-7 (1986); Kramer, et al, “Point Mismatch Repair,” Cell, 38:879-887 (1984); Wells, et al., “Cassette mutagenesis: an efficient method for generation of multiple mutations at defined sites,” Gene, 34:315-323 (1985); Minshull, et al., “Protein evolution by molecular breeding,” Current Opinion in Chemical Biology, 3:284-290 (1999); Christians, et al., “Directed evolution of thymidine kinase for AZT phosphorylation using DNA family shuffling,” Nature Biotechnology, 17:259-264 (1999); Crameri, et al., “DNA shuffling of a family of genes from diverse species accelerates directed evolution,” Nature, 391:288-291; Crameri, et at, “Molecular evolution of an arsenate detoxification pathway by DNA shuffling,” Nature Biotechnology, 15;436-438 (1997); Zhang, et al., “Directed evolution of an effective fucosidase from a galactosidase by DNA shuffling and screening,” Proceedings of the National Academy of Sciences, U.S.A., 94:45-4-4509; Crameri, et al., “Improved green fluorescent protein by molecular evolution using DNA shuffling,” Nature Biotechnology, 14:315-319 (1996); Stemmer, “Rapid evolution of a protein in vitro by DNA shuffling,” Nature, 370:389-391 (1994); Stemmer, “DNA shuffling by random fragmentation and reassembly: In vitro recombination for molecular evolution,” Proceedings of the National Academy of Sciences, U.S.A., 91:10747-10751 (1994); WO 95/22625; WO 97/0078; WO 97/35966; WO 98/27230; WO 00/42651; and WO 01/75767, all of which are incorporated herein by reference.

General texts which describe molecular biological techniques useful herein, including the use of vectors, promoters and many other relevant topics, include Berger and Kimmel, Guide to Molecular Cloning Techniques, Methods in Enzymology, vol. 152, Academic Press, Inc., San Diego, Calif. (“Berger”); Sambrook et al., Molecular Cloning—A Laboratory Manual (3rd Ed.), Vol. 1-3, Cold Spring Harbor Laboratory, Cold Spring Harbor, N.Y., 2001 (“Sambrook”), and Current Protocols in Molecular Biology, F. M. Ausubel, et al., Eds., Current Protocols, John Wiley & Sons, Inc. (supplemented through 2010) (“Ausubel”), all of which are incorporated herein by reference. Examples of protocols sufficient to direct persons of skill through in vitro amplification methods, including the polymerase chain reaction (PCR), the ligase chain reaction (LCR), Qβ-replicase amplification and other RNA polymerase mediated techniques (e.g., NASBA) are found in Berger, Sambrook, and Ausdhel, as well as Mullis, et al. (1987) U.S. Pat. No. 4,683,202; PCR. Protocols: A Guide to Methods and Applications (Innis, et al., eds.) Academic Press Inc., San Diego, Calif. (1990) (Innis); Arnheim & Levinson ((Oct. 1, 1990) C&EN36-47; The Journal of NIH Research (1991) 3:81-94; Kwoh, et al. (1989) Proc. Natl. Acad. Sci. USA, 86:1173; Guatelli, et al. (1990) Proc. Natl. Acad. Sci. USA 87:1874; Lomeli, et al. (1989) J. Clin. Chem. 35:1826; Landegren. et al. (1988) Science 241:1077-1080; Van Brunt (1990) i 8:291-294; Wu and Wallace (1989) Gene 4:560; Barringer, et al. (1990) Gene 89:117, and Sooknanan and Malek (1995) Biotechnology 13:563-564, all of which are incorporated herein by reference. Improved methods for cloning in vitro amplified nucleic acids are described in Wallace, et al., U.S. Pat. No. 5,426,039, which is incorporated herein by reference. Improved methods for amplifying large nucleic acids by PCB are summarized in Cheng, et al. (1994) Nature 369:684-685, which is incorporated herein by reference. One of ordinary skill in the art will readily appreciate that essentially any RNA can be converted into a double stranded DNA suitable for restriction digestion, PCR expansion and sequencing using reverse transcriptase and a polymerase as described in Ausubel, Sambrook, and Berger, supra, which is incorporated herein by reference.

### Vectors, Promoters, and Expression Systems

The present invention also includes recombinant constructs comprising one or more of the polynucleotides that encode the 2D-VCAM-1 variants of the present invention, which are described above. The term. “construct.” or “nucleic acid construct” refers herein to a nucleic acid, either single- or double-stranded, which is isolated from a naturally occurring gene or which has been modified to contain segments of nucleic acids in a manner that would not otherwise exist in nature. The term “nucleic acid construct” is synonymous with the term “expression cassette” when the nucleic acid construct contains the control sequences required for expression of a polynucleotide sequence of the present invention.

In a specific embodiment, the present invention also provides an expression vector comprising a polynucleotide of the present invention operably linked to a promoter. The term “expression vector” refers herein to a DNA molecule, linear or circular that comprises a segment encoding a polypeptide of the invention, which is operably linked to additional segments that provide for its transcription. As used herein, the term “expression” includes any step involved in the production of the polypeptide including, but not limited to, transcription, post-transcriptional modification, translation, post-translational modification, and secretion.

Nucleic acid constructs of the present invention typically include a control sequence, such as a promoter. The term “control sequences” refers herein to all of the components that are necessary or advantageous for the expression of a 2D-VCAM-1 variant of the present invention. Each control sequence may be native or foreign to the nucleotide sequence encoding the polypeptide. Such control sequences include, but are not limited to, a leader, a polyadenylation sequence, a propeptide sequence, a promoter, a signal peptide sequence, and a transcription terminator. At a minimum, the control sequences include a promoter and a transcriptional and a translational stop signals. The control sequences may be provided with additional sequences that introduce specific restriction sites, which facilitate ligation of the control sequences with, the coding sequence of the nucleotide sequence encoding the polypeptide.

The term “operably linked” refers herein to a configuration in which a control sequence is appropriately placed at a position relative to the coding sequence of the DNA sequence such that the control sequence directs the expression of a polypeptide.

When used herein, the term “coding sequence” refers to a polynucleotide sequence that directly specifies the amino acid sequence of its protein product. The boundaries of the coding sequence are generally determined by an open reading frame, which usually begins with the ATG start codon. The coding sequence typically includes a DNA, cDNA, and/or recombinant polynucleotide sequence.

Polynucleotides encoding 2D-VCAM-1 variant polypeptides of the present invention can be incorporated into any one of a variety of expression vectors that are well known in the art.

Expression vectors compatible with prokaryotic host cells may be used, such as prokaryotic expression vectors that are known in the art. These include, for example, BLUESCRIPT vector (Stratagene), T7 expression vector (Invitrogen), pET vector (Novagen), multifunctional E. coli cloning and expression vectors, and the like.

Expression vectors compatible with eukaryotic host cells may alternatively be used, such as eukaryotic expression vectors that are known in the art. These include, for example, pCMV vectors (e.g., Invtrogen), pIRES vector (Clontech), pSG5 vector (Stratagene), pCDNA3.1 (Invitrogen Life Technologies), pCDNA3 (Invitrogen Life Technologies), Ubiquitous Chromatin Opening Element (UCOE™) expression vector (Millipore), and the like.

Suitable vectors include chromosomal, nonchromosomal, and synthetic DNA sequences. Exemplary vectors include a bacterial artificial chromosome (BAC), a yeast artificial chromosome (YAC), a plasmid, such as, for example, a bacterial plasmid or a yeast plasmid, a cosmid, a phage, vectors derived from viral DNA, such as, for example, vaccinia, adenovirus, fowl pox virus, pseudorabies, adenovirus, adeno-associated virus, retroviruses, and the like, as well as vectors derived from combinations of plasmids and phage DNA. Any vector that transduces genetic material into a cell, and if replication is desired, which is replicable and viable in the relevant host can be used.

When incorporated into an expression vector, a polynucleotide of the invention is operatively linked to an appropriate transcription control sequence (promoter) to direct mRNA synthesis, such as, for example, T5 promoter. Other promoters known to control expression of genes in prokaryotic or eukaryotic cells or their viruses and which can be used in some embodiments of the invention include SV40 promoter, E. coli lac or trp promoter, phage lambda PL promoter, tac promoter, T7 promoter, and the like. An expression vector optionally contains a ribosome binding site for translation initiation, and a transcription terminator, such as Pin II. The vector also optionally includes appropriate sequences for amplifying expression, such as, for example, an enhancer.

In addition, the expression vectors of the present invention optionally contain one or more selectable marker genes to provide a phenotypic trait for selection of transformed host cells. Suitable marker genes include those coding for resistance to the antibiotic spectinomycin or streptomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance. Additional selectable marker genes include dihydrofolate reductase or neomycin resistance for eukaryotic cell culture, and tetracycline or ampicillin resistance in E. coli.

Vectors of the present invention can be employed to transform an appropriate host to permit the host to express a 2D-VCAM-1 variant described herein. Examples of appropriate expression hosts include those described hereinbelow in the section entitled “Expression Hosts”.

Polynucleotides encoding a 2D-VCAM-1 variant of the present invention can also be fused, for example, in-frame to nucleic acids encoding a secretion/localization sequence, to target polypeptide expression to a desired cellular compartment, membrane, or organelle of a cell, or to direct polypeptide secretion to the periplasmic space or into the cell culture media. Such sequences are known to those of skill in the art, and include secretion leader peptides, organelle targeting sequences (e.g., nuclear localization sequences, endoplasmic reticulum (ER) retention signals, mitochondrial transit sequences, chloroplast transit sequences), membrane localization/anchor sequences (e.g., stop transfer sequences, GPI anchor sequences), and the like.

### Expression Hosts

The present invention also provides engineered host cells that are transduced (transformed or transfected) with a vector or construct of the invention, as well as a method of production of polypeptides of the invention by, recombinant techniques. As used herein, the term “host cell” refers to any cell type which is susceptible to transformation with a nucleic acid construct of the present invention. Therefore, the present invention includes isolated host cells comprising any polynucleotide of the present invention that is described hereinabove. Typically, the polynucleotide is operably connected to one or more promoters and/or enhancers that provide for expression of the polynucleotide of the host cell.

The isolated host cell can be a eukaryotic cell (e.g., a CHO cell), such as a mammalian cell, a yeast cell, or a plant cell, or the host cell can be a prokaryotic cell, such as a bacterial cell (e.g., E. coil, Bacillus sp., Streptomyces, and the like). Introduction of the nucleic acid construct into the host cell can be effected by calcium phosphate transfection, DEAE-Dextran mediated transfection, electroporation, gene it vaccine gun, injection, or other common techniques (see, e.g., Davis, L., Dibner, M., and Battey, I. (1986) Basic Methods in Molecular Biology), which is incorporated herein by reference, for in vivo, ex vivo or in vitro methods.

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre” or a “prepro” form of the protein may also be important for correct insertion, folding and/or function. Different host cells such as E. coli, Bacillus sp., yeast or mammalian cells such as CHO, HeLa, BHK, MDCK, HEK 293, W138, and the like, have specific cellular machinery and characteristic mechanisms for such post-translational activities and may be chosen to ensure the correct modification and processing of the introduced foreign protein.

Stable expression can be used for long-term, high-yield production of recombinant proteins. For example, cell lines which stably express a polypeptide of the present invention are transduced using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. For example, resistant clumps of stably transformed cells can be proliferated using tissue culture techniques appropriate to the cell type.

Host cells transformed with a polynucleotide sequence encoding a polypeptide of the invention are optionally cultured under conditions suitable for the expression and recovery of the encoded protein from cell culture. The polypeptide produced by a recombinant cell may be secreted, membrane-bound, or contained intracellularly, depending on the sequence and/or the vector used. As will be understood by those of skill in the art, expression vectors containing polynucleotides encoding polypeptides of the invention can be designed to include signal sequences which direct secretion of mature polypeptides through a prokaryotic or eukaryotic cell membrane.

### Production and Recovery of 2D-VCAM-1 Variants

In another embodiment, the present invention provides a method of making a 2D-VCAM-1 variant of the present invention, the method comprising: culturing a host cell transformed with a polynucleotide of the present invention under conditions suitable for expression of the encoded polypeptide; and recovering the polypeptide from the culture medium or from the transformed and cultured host cells. Host cells employed in the production and recovery of 2D-VCAM-1 variants are typically isolated host cells, as compared to nigher order organisms, such as plants or animals.

Following transduction of a suitable host strain and growth of the host strain to an appropriate cell density, the selected promoter is induced by appropriate means, such as, for example, by temperature shift or chemical induction, and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well known to those skilled in the art.

As noted, many references are available for the culture and production of many cells, including cells of bacterial, plant, animal (especially mammalian) and archebacterial origin. These references include Sambrook, Austibel, and Berger (supra), as well as Freshney (1994) Culture of Animal Cells, a Manual of Basic Technique, third edition, Wiley-Liss, New York (and the references cited therein); Doyle and Griffiths (1997) Mammalian Cell Culture: Essential Techniques John Wiley and Sons, NY; Humason (1979). Animal Tissue Techniques, fourth edition, W.H. Freeman and Company; and Ricciardelli, et at (1989) In vitro Cell Dev. Biol. 25:1016-1024, all of which are incorporated herein by reference. References that describe plant cell culture and regeneration include, Payne, et al. (1992) Plant Cell and Tissue Culture in Liquid Systems, John Wiley & Sons, Inc. New York, N.Y.; Gamborg and Phillips (Eds) (1995) Plant Cell, Tissue and Organ Culture; Fundamental Methods Springer Lab Manual, Springer-Verlag (Berlin Heidelberg, New York); Jones, Ed. (1984) Plant Gene Transfer and Expression Protocols, Hummana Press, Totowa, N.J. and Plant Molecular Biology (1993) R. R. D. Croy, Ed. Bios Scientific Publishers, Oxford, U.K. ISBN 0 12 198370 6, all of which are incorporated herein by reference.

Cell culture media in general is described in Atlas and Parks (Eds.) The Handbook of Microbiological Media (1993) CRC Press, Boca Raton, Fla., which is incorporated herein by reference. Additional information for cell culture is found in available commercial literature such as the Life Science Research Cell Culture Catalogue (1998) from Sigma-Adrich, (St. Louis, Mo.) and The Plant Culture Catalogue and supplement (1997) from Sigma-Aldrich, Inc. (St. Louis, Mo.), both of which are incorporated herein by reference.

2D-VCAM-1 variants of the present invention may be recovered/isolated and optionally purified from recombinant cell cultures by any of a number of methods well known in the art, such as, for example, ammonium sulfate or solvent precipitation (such as, for example, by using a solvent like ethanol, acetone, and the like), acid extraction, ion (anion or cation) exchange chromatography, high performance liquid Chromatography (HPLC), phosphocellulose chromatography, hydrophobic interaction chromatography, affinity chromatography, hydroxylapatite chromatography, lectin chromatography and size-exclusion chromatography. Suitable protein purification methods are described in Sandana (1997) Bioseparation of Proteins, Academic Press, Inc.; Bollag et al. (1996) Protein Methods, 2nd Edition, Wiley-Liss, NY; Walker (1996) The Protein Protocols Handbook, Humana Press, NJ; Harris and Angal (1990) Protein Purification Applications: A Practical Approach, IRL Press at Oxford, Oxford, England; Harris and Angal, Protein Purification Methods: A Practical Approach, IRL Press at Oxford, Oxford, England; Scopes (1993) Protein Purification: Principles and Practice, 3rd Edition, Springer Verlag, NY; Janson and Ryden (1998) Protein Purification: Principles, High Resolution Methods and Applications, Second Edition, Wiley-VCH, NY; and Walker (1998) Protein Protocols on CD-ROM, Humana Press, NJ, all of which are incorporated herein by reference. Typically, the purification step comprises purification by a chromatography method, employing an eluant mixture that comprises arginine.

Bacterially-produced 2D-VCAM-1 variants may form inclusion bodies (IBs), which require further processing steps to generate active polypeptides. This further processing may entail the isolation and solubilization of the inclusion bodies, unfolding the polypeptide, then refolding the polypeptide into the correct biologically active tertiary structure. The present invention provides a method of producing a polypeptide of the present invention, said method comprising: culturing a host cell transformed with a polynucleotide encoding a polypeptide of the present invention under conditions suitable for expression the polypeptide as inclusion bodies; recovering inclusion bodies comprising the encoded polypeptide from the transformed and cultured host cells; solubilizing (denaturing) the recovered inclusion bodies comprising the polypeptide with a solubilizing agent; purifying the solubilized polypeptide; allowing the polypeptide to refold; and purifying the refolded polypeptide.

Inclusion bodies are typically solubilized in solvents such as urea. Refolding may be accomplished, for example, by incubating solubilized polypeptide in a solution of dilute urea and glutathione.

### Polypeptide Variant Conjugates

The present invention provides a polypeptide variant conjugate comprising a 2D-VCAM-1 variant polypeptide of the present invention or fusion protein thereof covalently bound to at least one non-polypeptide conjugation moiety. Typically, the polypeptide variant conjugate comprises a 2D-VCAM-1 variant polypeptide of the present invention covalently bound to one or more non-polypeptide conjugation moieties.

As used herein, the term “non-polypeptide conjugation moiety” refers to a non-polypeptide polymer, a sugar moiety, or a non-polymeric lipophilic moiety. The term “non-polypeptide polymer” refers herein to a water soluble polymer that may be a natural or synthetic polymer (homopolymer, copolymer, terpolymer, and the like), that is not a peptide, polypeptide, or protein. As used herein, the term “sugar moiety” refers to a carbohydrate molecule attached by an in vivo or in vitro glycosylation process, such as an N- or O-glycosylation process.

Non-polypeptide conjugation moieties are typically selected to alter specific attributes of the 2D-VCAM-1, variant polypeptides, such as, for example, in vivo serum half life or functional in vivo half life, stability, immunogenicity, and the like. As used herein, the term “in vivo serum half-life” refers to the time at which 50% of the compound of interest circulates in the bloodstream of a non-human mammal such as a rat, mouse, rabbit, or monkey. The term “serum” is used herein to refer to its normal meaning, i.e., as blood plasma without fibrinogen and other clotting factors. The term “functional in vivo half-life” refers herein to the time at which 50% of the biological activity of the compound of interest is still present in the body or target organ, or the time at which the activity of the compound of interest is 50% of the initial value. The functional in vivo half-life may be determined in a non-human mammal, such as a rat, mouse, rabbit, dog, or monkey. Methods for determining both in vivo serum half-life and functional in viva half-life are well known in the art. For example, a 2D-VCAM-1 variant polypeptide or conjugate thereof may he administered to a non-human mammal, and blood samples collected at fixed time intervals. The blood samples may be analyzed for levels of 2D-VCAM-1 variant by, for example, an ELISA assay using an anti-VCAM-1 antibody, or by assaying with a soluble or insoluble VLA4 protein preparation (e.g., binding to a soluble VLA4-Fc or a membrane-bound VLA4 and detecting the presence of the complex). The half life can be determined from a plot of 2D-VCAM-1 variant concentration versus time.

2D-VCAM-1 variant conjugates of the present invention typically exhibit greater functional in vivo half-life and/or greater serum half-life as compared to the corresponding non-conjugated 2D-VCAM-1 variant polypeptides. These 2D-VCAM-1 variant conjugates usually have a non-polypeptide polymer or a sugar moiety conjugated to the 2D-VCAM-1 variant polypeptide. For example, each non-polypeptide polymer and/or sugar moiety may be conjugated either directly or indirectly via a linker, to the 2D-VCAM-1 variant polypeptide. The non-polypeptide conjugation moiety is typically bound, either directly or indirectly via a linker, to an attachment group in the 2D-VCAM-1 variant polypeptide.

The teen “greater” as it is used in connection with the functional in vivo half-life or serum half-life is used herein to indicate that the relevant half life of the 2D-VCAM-1 variant conjugate is statistically significantly greater than a reference molecule, such as the corresponding non-conjugated 2D-VCAM-1, variant polypeptide or a component thereof, when determined under comparable conditions. Thus, 2D-VCAM-1 variant conjugates include those that have a functional in vivo half-life or a serum half-life that is greater than that of the corresponding non-conjugated 2D-VCAM-1 variant polypeptide.

2D-VCAM-1 variant conjugates include those where the ratio between the functional in vivo half-life (or serum half-life) of the conjugate and that of the corresponding non-conjugated 2D-VCAM-1 variant polypeptide is at least 1.25, at least 1.5, at least 1.75, at least 2, at least 3, at least 4, at least 5, at least 6, at least 7, or at least 8.

In a further aspect, a 2D-VCAM-1 variant conjugate may exhibit greater bioavailability than the corresponding non-conjugated 2D-VCAM-1 variant polypeptide. An indication of bioavailability is provided, by the “Area. Under the Curve when administered subcutaneously” parameter or “AUCsc”. The term “AUCsc” or “Area Under the Curve when administered subcutaneously” is used in its normal meaning to refer to the area under the activity-in-serum vs. time curve, where the 2D-VCAM-1. variant has been administered subcutaneously to an experimental animal. Once the experimental activity time points have been determined, the AUCsc may conveniently be calculated by a computer program, such as GraphPad Prism 3.01, GraphPad Software Inc., La Jolla, Calif. The term “greater” as it is used in connection with AUCsc is used to indicate that the Area Under the Curve for a 2D-VCAM-1 variant conjugate, when administered subcutaneously, is statistically significantly greater than the corresponding non-conjugated 2D-VCAM-1. variant polypeptide, when determined under comparable conditions. In order to make direct comparisons between different molecules, the AUCsc values should typically be normalized, e.g., expressed as AUCsc/dose administered.

Exemplary 2D-VCAM-1 variant conjugates include those in which the ratio between the AUCsc of the conjugate and the AUCsc of the corresponding non-conjugated 2D-VCAM-1 variant polypeptide is at least 1.25, at least, 1.5, at least 2, at least 3, at least 4, at least 5, at least 6, at least 7, at least 8, at least 9, at least 10, at least 12, at least 14, at least 16, at least 18, or at least 20 when administered under comparable conditions to the same species of experimental animal (e.g., rat, monkey, and the like).

Certain 2D-VCAM-1 variant conjugates of the present invention exhibit greater activity after a longer duration post-administration than the corresponding non-conjugated 2D-VCAM-1 variant polypeptide. These 2D-VCAM-1 variant conjugates exhibit a Tmax that is greater than the Tmax for the corresponding non-conjugated 2D-VCAM-1 variant polypeptide. As used herein, the term “Tmax” refers to the time point in the activity-in-serum vs. time curve where the highest activity in serum is observed. The ratio of Tmax for such a 2D-VCAM-1 variant conjugate to the Tmax of the corresponding non-conjugated 2D-VCAM-1 variant polypeptide is at least 1.2, at least 1.4, at least 1.6, at least 1.8, at least 2, at least 2.5, at least 3, at least 4, at least 5, at least 6, at least 7, at least 8, at least 9, or at least 10, when administered subcutaneously, in particular when administered subcutaneously in an experimental animal such as a rat or a monkey.

The conjugation moiety may be covalently bound to the polypeptide either directly or indirectly via a linker. Suitable linker moieties include a peptide, a non-peptidic, non-polymeric aliphatic moiety, an oligonucleotide, and the like. Suitable linkers include those described hereinabove in the section entitled “2D-VCAM-1 Variant Polypeptides.” In some embodiments, a linker peptide is covalently attached to the N-terminus of the 2D-VCAM-1 variant, and a non-polypeptide conjugation moiety is covalently attached to the N-terminus of the attached linker peptide. In some embodiments, a linker peptide is attached to the N-terminus or the C-terminus of the 2D-VCAM-1 variant, and a non-polypeptide conjugation moiety is covalently attached to an attachment group (such as, a Cys or a Lys residue) in the linker peptide. Glycosylation sites, which are also discussed in more detail below, may also be incorporated into the linker peptide sequence. Exemplary attachment groups for attaching a non-polypeptide conjugation moiety are described below in Table 2.

TABLE 2
Useful attachment groups and examples of corresponding
non-polypeptide conjugation moieties

| | | Examples of non- | | |
|---|---|---|---|---|
| Attachment | Attachment | polypeptide | Conjugation | |
| site | group | conjugation moieties | method/activated PEG | Reference |
| —NH₂ | N-terminal | Polymer, e.g. PEG | mPEG-SPA | Nektar, Inc. 2003 |
| | amino | | mPEG2-NHS | and 2005-2006 |
| | group, Lys | | mPEG2-butryALD | Catalogs*; |
| | | | (Nektar, Inc.) | |
| | | | SUNBRIGHT series: | NOF Corp. 2005 |
| | | | ME-020CS, ME-250CS, | Catalogue; PEG |
| | | | ME-100CS, ME-200CS; | Derivatives, |
| | | | MEGC-20HS, MEGC- | Phospholipids and |
| | | | 50HS, MEGC-10TS, | Drug Delivery |
| | | | MEGC-20TX, MEGC- | Materials for |
| | | | 30TS (mPEG N- | Pharmaceutical |
| | | | hydroxysuccinimide | Products and |
| | | | active esters); ME- | Formulations* |
| | | | 020AS, ME-050AS, | |
| | | | ME-050HS, ME-050AL, | |
| | | | ME-100AL, ME- | |
| | | | 200AL, ME-300AL | |
| | | | (mPEG aldehydes); HO- | |
| | | | 050AL, HO-100AL, | |
| | | | HO-200AL, HO-300AL | |
| | | | (Hydroxy-PEG- | |
| | | | aldehydes); GL2- | |
| | | | 200GS2, GL2-400GS2, | |
| | | | GL2-200HS, GL2- | |
| | | | 400HS (Branched PEG | |
| | | | N-hydroxysuccinimide | |
| | | | esters) (NOF Corp.) | |
| —COOH | C-terminal | Polymer, e.g. PEG | mPEG-Hz | Nektar, Inc. 2003 |
| | carboxy | | | and 2005-2006 |
| | group, Asp, | | | Catalogs* |
| | Glu | Sugar moiety | In vitro coupling | |
| —SH | Cys | Polymer, e.g. PEG, | mPEG-VS | Nektar, Inc. 2003 |
| | | | mPEG2-MAL | Catalog*; |
| | | | (Nektar, Inc.) | |
| | | | SUNBRIGHT series: | NOF Corp. 2005 |
| | | | ME-020MA, ME- | Catalogue: PEG |
| | | | 050MA, ME-120MA, | Derivatives, |
| | | | ME-200MA, ME- | Phospholipids and |
| | | | 300MA (Maleimido- | Drug Delivery |
| | | | mPEGs); GL2-200MA, | Materials for |
| | | | GL2-400MA (Branched | Pharmaceutical |
| | | | PEG maleimides) | Products and |
| | | | (NOF Corp.) | Formulations*; |
| | | Sugar moiety | In vitro coupling | Delgado et al, |
| | | | | Critical Reviews |
| | | | | in Therapeutic |
| | | | | Drug Carrier |
| | | | | Systems |
| | | | | 9(3, 4): 249-304 |
| | | | | (1992)*; |
| —OH | Ser, Thr, | Sugar moiety | In vivo O-linked | |
| | OH—, Lys | | glycosylation | |
| —CONH₂ | Asn as part | Sugar moiety | In vivo glycosylation | |
| | of an N- | | | |
| | glycosylation | | | |
| | site | | | |
| Aromatic | Phe, Tyr, | Sugar moiety | In vitro coupling | |
| residue | Trp | | | |
| —CONH₂ | Gln | Sugar moiety | In vitro coupling | Yan and Wold, |
| | | | | Biochemistry, |
| | | | | 1984, Jul. 31; |
| | | | | 23(16): 3759-65* |
| Guanidino | Arg | Sugar moiety | In vitro coupling | Lundblad and |
| | | | | Noyes, Chemical |
| | | | | Reagents for |
| | | | | Protein |
| | | | | Modification, |
| | | | | CRC Press Inc. |
| | | | | Boca Raton, FI* |
| Imidazole | His | Sugar moiety | In vitro coupling | Same as for |
| ring | | | | guanidino |

*Each publication is incorporated herein by reference.

Polymers suitable for use in the practice of the present invention may be branched (i.e., having two or more linear polymer chains linked together by a linker group) or linear and typically have an average molecular weight in the range of from about 300 to about 100,000 daltons and typically from about 1,000 daltons to about 80,000 daltons, or from about 2,000 to about 60,000 or 50,000 or 40,000 or 30,000 or 20,000, or 10,000 daltons, and in some embodiments from about 1,000 daltons to about 5,000 daltons. More particularly, the polymer molecule will typically have an average molecular weight of about 2,000 daltons, 5,000 daltons, 10,000 daltons, 1.2,000 daltons, 15,000 daltons, 20,000 daltons, 30,000 daltons, 40,000 daltons, 50,000 daltons, 60,000 daltons or 80,000 daltons.

When used in the context of describing the molecular weight of a polymer, the term “about” indicates an approximate average molecular weight and reflects the fact that there will normally be a certain molecular weight distribution in a given polymer preparation.

Exemplary polymers that are suitable for use in the conjugates of the present invention include polyalkylene oxides (PAO), such as a polyalkylene glycol (PAG) which may, for example, be a polyethylene glycol (PEG), a monomethoxypolyethylene glycol (mPEG), a polypropylene glycol (PPG), a branched polyethylene glycol having two or more polyethylene glycol chains linked together by a linker group (a linker, such as, for example, lysine, glycerol, and the like), polyvinyl alcohol (PVA), polycarboxylate, poly(vinylpyrrolidone), polyethylene-co-maleic acid anhydride, a dextran (such as, for example, carboxymethyl dextran), and other like polymers.

Polyalkylene glycol-derived polymers are typically employed in the conjugates of the present invention because they are generally biocompatible, non-toxic, non-antigenic, non-immunogenic, water soluble, and are easily excreted from living organisms. Polyethylene glycol (PEG) in particular is favored because it has only a few reactive groups that are capable of cross-linking to other compounds, such as polysacchararides (e.g., dextran). Monofunctional PEG, such as, for example, a monomethoxypolyethylene glycol (mPEG), is one example of a suitable polymer for use in the conjugates of the present invention.

To effect covalent attachment of a hydroxylated polymer like polyethylene glycol to the 2D-VCAM-1 variant polypeptide, at least one terminal hydroxyl group of the polymer molecule is typically provided in activated form, i.e., derivatized with functional groups that are reactive with the target attachment group in the 2D-VCAM-1 variant polypeptide Exemplary reactive functional groups include primary amino groups, hydrazide (HZ), thiol, succinate (SUC), succinimidyl succinate (SS), succinimidyl succinimide (SSA), succinimidyl proprionate (SPA), succinimidyl carboxymethylate (SCM), benzotriazole carbonate (BTC), N-hydroxysuccinimide (NHS), aldehyde, nitrophenylcarbonate (NPC), maleimide (MAL) and tresylate (TRES). 2D-VCAM-1 conjugates of the present invention may comprise two 2D-VCAM-1 variant polypeptides of the present invention covalently bonded to one bi-functional PEG. As used herein, the term “bi-functional peg” refers to a polyethylene glycol moiety that is derivatized with two functional groups that are reactive with the target attachment group in the 2D-VCAM-1 variant polypeptide.

Suitably activated polymer molecules are commercially available, e.g., from Nektar Therapeutics, Inc., San Francisco, Calif., NOF Corporation, Japan, and DowPharma, Midland, Mich. Alternatively, the polymer molecules can be activated by conventional methods known in the art, such as those disclosed in WO 90/13540, which is incorporated herein by reference. Specific examples of activated linear and branched mono- and bi-functional polymer molecules that are suitable for use in the 2D-VCAM-1 variant conjugates of the present invention are described in the Nektar 2005-2006 Advanced Pegylation Catalogue and the NOF DDS Catalogue Ver. 12, both of which are incorporated herein by reference. Specific examples of these activated polyethylene glycol polymers include the following linear PEGs: NHS-PEG (e.g., SPA-PEG, SSPA-PEG, SBA-PEG, SS-PEG, SSA-PEG, SC-PET, SG-PEG, and, SCM-PET), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, bi-functional PEGs such as MAL-PEG-MAL, NHS-PEG-NHS, SS-PEG-SS, and the like, and branched. PEGs such as mPEG2-NHS, disclosed in the Nektar 2005-2006 “Advanced Pegylation” Catalogue (which is incorporated herein by reference) and those disclosed in the NOF DDS Catalogue Ver. 12, U.S. Pat. Nos. 5,932,462 and 5643,575, all of which are incorporated herein by reference.

Other examples of activated linear and branched polymer molecules that are suitable for use in the 2D-VCAM-1 variant conjugates of the present invention are described in NOF Corp. 2005 Catalogue; “PEG Derivatives, Phospholipids and Drug Delivery Materials for Pharmaceutical Products and Formulations” and NOF Corp, Catalogue Ver. 12 DDS, both of which is incorporated herein by reference. Additional examples of activated polyethylene glycol polymers include the following PEGs: NHS-PEG. (e.g., SUNBRIGHT ME-020 (-050, -100, -200)-CS (CH₃O)CH₂CHO)ₙ—CO—CH₂CH₂—COO—NHS); SUNBRIGHT MEGC-20 (-50)-HS and SUNBRIGHT MEGC-10 (-20, -30)-TS (CH₃O(CH₂CH₂O)ₙ—CO—CH₂CH₂CH₂—COO—NHS); SUNBRIGHT ME-020(-050)-AS (CH₃O(CH₂CH₂O )ₙ—CH₂—COO—NHS); SUNBRIGHT ME-050HS (CH₃O(CH₂CH₂O)ₙ—(CH₂)₅—COO—NHS)); Aldehyde PEG (e.g., SUNBRIGHT ME-050 (-100, -200, -300)-AL (CH₃O(CH₂CH₂O)ₙ—CH₂CH₂CHO)); Maleimido-PEGs (e.g., SUNBRIGHT ME-020 (-050, -120, -200, -200)-MA (CH₃O(CH₂CH₂O)ₙ—(CH₂)₃NHCO(CH₂)₂—(C₄H₂NO₂)); Branched PEG Maleimides (e.g., SUNBRIGHT GL2-200 (-400)-MA); Branched PEG NHS-glutaryl (e.g., SUNBRIGHT GL2-200 (-400)-GS2); Branched PEG NHS-carboxymethyl (e.g., SUNBRIGHT GL2-200 (-400)-HS); Branched PEG aldehyde (e.g., SUNBRIGHT GL3-200 (400)AL2); all disclosed in NOF Corp. 2005 Catalogue; “PEG Derivatives, Phospholipids and Drug Delivery Materials for Pharmaceutical Products and Formulations” (which is incorporated herein by reference) and those disclosed in NOF Corp. Catalogue Ver. 12 DDS, U.S. Pat. No. 6,875,841 and U.S. Patent Application Publications US 2005/0058620 and US 2005/0288490, all of which are incorporated herein by reference.

The following publications disclose farther useful polymers and/or PEGylation chemistries that are suitable for use in connection with the 2D-VCAM-1 variant conjugates of the present invention: U.S. Pat. No. 5,824,778, U.S. Pat. No. 5,476,653, U.S. Pat. No. 6,875,841, U.S. Pat. No. 5,872,191, U.S. Pat. No. 5,767,284, EP 0 839 850, WO 97/32607, EP 229,108, EP 402,378, U.S. Pat. No. 4,902,502, U.S. Pat. No. 5,281,698, U.S. Pat. No. 5,122,614, U.S. Pat. No. 5,219,564, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. No. 5,473,034, U.S. Pat. No. 5,516,673, EP 605 963, U.S. Pat. No. 5,382,657, EP 510 356, EP 400 472, EP 183 503 and EP 154 316, all of which are incorporated herein by reference.

Specific examples of activated PEG polymers particularly preferred for coupling to cysteine residues include the following linear PEGs: Vinyl sulfone-PEG (VS-PEG), preferably vinylsulfone-mPEG 9VS-mPEG); maleimide-PEG (MAL-PEG, as well as other maleimide PEGs described herein) preferably maleimide-mPEG (MAL-mPEG, as well as other maleimide mPEGs described herein) and orthopyridyl disulfide-PEG (OPS -PEG), preferably orthopyridyl-disulfide-mPEG (OPSS-mPEG).

The conjugation of the 2D-VCAM-1 variant polypeptide and the activated polymer molecule(s) is conducted in accordance with any conventional method, e.g. as described in the following references (which also describe suitable methods for activation of polymer molecules): Harris and Zalipsky, eds., Polyethylene glycol) Chemistry and Biological Applications, AZC, Washington; R. F. Taylor, (1991), “Protein Immobilisation: Fundamentals and Applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y., all of which are incorporated herein by reference.

The process of conjugating an activated polyethylene glycol to the 2D-VCAM-1 variant polypeptide is referred to herein as “PEGylation”. Covalent coupling of a polyethylene glycol moiety to the 2D-VCAM-1 variant polypeptide can be targeted to a specific attachment site by selection of the appropriate activated polyethylene glycol and reaction conditions. These are well known in the art and are described in more detail hereinbelow. Furthermore, the conjugation may be achieved in one step or in a stepwise manner using known methods, such as those described in WO 99/55377, which is incorporated herein by reference.

For PEGylation of cysteine residues the polypeptide is usually treated with a reducing agent, such as dithiothreitol (DTT) prior to PEGylation. The reducing agent is subsequently removed by any conventional method, such as by desalting. Conjugation of PEG to a cysteine residue typically takes place in a suitable buffer at pH 6-9 at temperatures varying from 4° C. to 25° C. for periods up to about 16 hours.

PEGylation of lysines often employs PEG moieties activated with active esters, such as, for example, N-hydroxysuccinimidyl (NHS) ester (e.g., mPEG-N-hydroxylsuccinimide (e.g., mPEG-NHS or mPEG2-NHS), esters such as PEG succinimidyl propionate (e.g., mPEG-SPA) or PEG succinimidyl butanoate (e.g., mPEG-SBA), and the like, including other activated PEG NHS esters described herein). One or more PEG moieties can be attached to a protein within 30 minutes at pH 8-9.5 at room temperature if about equimolar amounts of PEG and protein are mixed. A molar ratio of PEG to protein amino groups of 1-5 to 1 will usually suffice. Increasing pH increases the rate of reaction, while lowering pH reduces the rate of reaction. These highly reactive active esters can couple at physiological pH, but less reactive derivatives typically require higher pH. Low temperatures may also be employed if a labile protein is being used. Under low temperature conditions, a longer reaction time may be used.

Covalent attachment of a PEG moiety to the N-terminal amino group of a 2D-VCAM-1 variant polypeptide is referred to herein as “N-terminal PEGylation”. N-terminal PEGylation is facilitated by the difference between the pKa values of the α-amino group of the N-terminal amino acid (˜7.6 to 8.0) and the ε-amino group of lysine (˜10). PEGylation of the N-terminal amino group often employs PEG-aldehydes, which are more selective for amines and thus are less likely to react with the imidazole group of histidine; in addition, PEG reagents used for lysine conjugation (e.g., as noted above) may also be used for conjugation of the N-terminal amine. Conjugation of a PEG-aldehyde to the N-terminal amino group typically takes place in a suitable buffer (such as, 100 mM sodium acetate or 100 mM sodium bisphosphate buffer with 20 mM sodium cyanoborohydride) at pH ˜5.0 overnight at temperatures varying from about 4° C. to 25° C. Useful N-terminal PEGylation methods and chemistries are also described in U.S. Pat. No. 5,985,265 and U.S. Pat. No. 6,077,939, both of which are incorporated herein by reference.

In some instances, the N-terminally PEGylated 2D-VCAM-1 variant conjugates of the invention have a PEG directly attached to the α-amino group of the N-terminal amino acid of the 2D-VCAM-1 variant polypeptide. In some instances, the PEG is indirectly attached to the N-terminus of the polypeptide via a linker peptide; that is, the linker peptide is attached (fused) to the N-terminus of the 2D-VCAM-1 variant polypeptide, and the PEG is then covalently attached to the the α-amino group of the N-terminal amino acid of the linker peptide. Suitable linker peptides are described above. The PEG may be linear or branched, and typically has an average molecular weight of about 20 kdal to about 80 kdal, such as about 30 kdal to about 60 kdal, for example about 50 kdal.

A 2D-VCAM-1 variant conjugate may have one or more polyethylene glycol moietie attached to a lysine residue in the 2D-VCAM-1 variant polypeptide of the present invention. In some instances, at least one or more PEG moieties is conjugated to the 2D-VCAM-1 variant polypeptide at one or more of the following lysine attachment groups (with reference to the amino acid position number corresponding to that of SEQ NO:2, 12, or 18, except where noted): K2, K46, K79 (note that position 79 is not a lysine-reactive attachment site in SEQ ID NO:18), K82, K93, K107, K112, K130, K136, K147, K152, K167, the α amino group on the N-terminal amino acid residue of the 2D-VCAM-1 variant polypeptide, and any combination of two or more thereof. PEG moieties may also be conjugated to a 2D-VCAM-1 variant polypeptide at one or more of the following attachment groups introduced by substitution: R10K, R36K, R78K, R123K, R146K, R172K, R187K, (with reference to the amino acid position number corresponding to that of SEQ ID NOs:2, 12, 18) as well as any combination of any two or more thereof.

If cysteine pegylation methods are utilized, a 2D-VCAM-1 variant conjugate of the present invention may comprise a PEG moiety bound to the 2D-VCAM-1 variant polypeptide at a cysteine attachment group, such as, for example: C23, C28, C71, C75, C113, C171 (with reference to the amino acid position number corresponding to that of SEQ ID NO:2), and any combination of two or more thereof. PEG moieties may be bound to any combination of the aforedescribed attachment groups, as well as any introduced attachment groups described herein,

In some instances, it may be desirable to conjugate as many of the available polymer attachment groups as possible with polymer. This may be achieved by utilizing a molar excess of polymer relative to the 2D-VCAM-1 variant polypeptide. Such molar excess may be achieved by utilizing a molar ratio of activated polymer to 2D-VCAM-1 variant polypeptide of up to about 100:1 or 200:1 to about 1000:1. In some cases, the molar ratio may be somewhat lower, such as up to about 50:1, 10:1 or 5:1. An equimolar ratio of activated polymer to 2D-VCAM-1 variant polypeptide may also be used.

As described above, 2D-VCAM-1 variant conjugates of the present invention also include conjugates having one or more sugar moieties (i.e., carbohydrate molecules) bound to the 2D-VCAM-1 variant polypeptide. These conjugates are also referred to herein as “glycosylated” 2D-VCAM-1 variant polypeptides. The glycosylation sites on the 2D-VCAM-1 variant polypeptide are typically either an N- or O-glycosylation site. As used herein, the term “N-glycosylation site” refers to the sequence N-X-S/T/C”, wherein X is any amino acid residue except proline, N is asparagines and S/T/C is either serine, threonine or cysteine, preferably serine or threonine, and most preferably threonine. An “O-glycosylation site” refers herein to the —OH group of a serine or threonine residue.

2D-VCAM-1 variant polypeptides may be glycosylated by either an in vitro or in vivo process. Depending on the coupling method employed, the carbohydrate(s) may be attached to the following attachment groups: a) arginine and histidine, as described in Lundhlad and Noyes, “Chemical Reagents for Protein Modification”, CRC Press Inc. Boca Raton, Fla., which is incorporated herein by reference, b) free carboxyl groups (e.g. of the C-terminal amino acid residue, asparagine or glutamine), c) free sulfhydryl groups such as that of cysteine, d) free hydroxyl groups such as those of serine, threonine, tyrosine or hydroxyproline, e) aromatic residues such as those of phenylalanine or tryptophan, or f) the amide group of glutamine. Such groups may be introduced and/or removed in the polypeptide of the invention. Suitable methods of in vitro coupling are described in WO 87/05330 and in Aplin et al.,“CRC Crit Rev. Biochem.” pp. 259-306, 1981, both of which are incorporated herein by reference. The in vitro coupling of sugar moieties to protein- and peptide-bound Gln-residues can also be carried out by transglutaminases (TGases), e.g. as described by Sato et al., 1996 Biochemistry 35:1307-13080 and EP 725145, both of which are incorporated herein by reference.

2D-VCAM-1 variant polypeptides of the present invention may be glycosylated in vivo by introducing a polynucleotide encoding a 2D-VCAM-1 variant polypeptide leaving one or more N- or O-glycosylation sites into a glycosylating eukaryotic expression host cell. The glycosylating eukaryotic expression host cell may be selected from a fungal cell (e.g., a filamentous fungal cell or a yeast cell), an insect cell, a mammalian cells, a plant cell, or any other glycosylating eukaryotic expression host cell known in the art.

Non-polypeptide lipophilic moieties that are suitable for conjugation to the 2D-VCAM-1 variant polypeptides of the present invention include a natural compound such as a saturated or an unsaturated fatty acid, a fatty acid diketone, a terpene, a prostaglandin, a vitamin, a carotenoid or a steroid, a phospholipid, or alternatively, a synthetic compound, such as a linear or branched aliphatic, aryl, alkaryl acid (e.g., carboxylic, sulphonic, and the like), alcohol, amine, and the like. Conjugation to non-polypeptide lipophilic moieties may take place at any one of the following exemplary attachment sites: the N-terminus or the C-terminus of the 2D-VCAM-1 variant polypeptide, the hydroxyl groups of the amino acid residues Ser, Thr or Tyr, the ε-amino group of Lys, the SH group of Cys or the carboxyl group of Asp and Gin. The 2D-VCAM-1 variant polypeptide and the non-polypeptide lipophilic moiety may be conjugated to each other either directly or indirectly via a linker in accordance with methods known in the art, such as those described in Bodanszky, “Peptide Synthesis”, John Wiley, New York (1976) and WO 96/12505, both of which are incorporated herein by reference.

Non-polypeptide conjugation moieties may he bound, either directly or indirectly via a linker moiety, to the 2D-VCAM-1 variant polypeptide of the present invention. For example, the non-polypeptide polymer may be conjugated to the 2D-VCAM-1 variant polypeptide via a cyanuric chloride linker as described Abuchowski et al., (1977), J. Biol. Chem., 252, 3578-3581; and U.S. Pat. No. 4,179,337, both of which are incorporated herein by reference. Other suitable linkers are well known in the art.

### Pharmaceutical Compositions

The present invention provides a composition comprising a 2D-VCAM-1 variant polypeptide of the present invention or protein fusion or conjugate thereof as described hereinabove, together with a carrier or excipient, such as a pharmaceutically acceptable carrier or excipient.

Suitable pharmaceutically acceptable excipients include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidone, low melting waxes, ion exchange resins, and the like, as well as combinations of any two or more thereof. Other suitable pharmaceutically acceptable excipients are described in “Remington's Pharmaceutical Sciences”, 18th edition, A. R. Gennaro), Ed., Mack Pub, Co. New Jersey (1991), “Pharmaceutical Formulation Development of Peptides and Proteins, S. Frokjaer and L. Hovgaard, Eds., Taylor & Francis (2000), and “Handbook of Pharmaceutical Excipients, 3rd edition, A. Kibbe, Ed., Pharmaceutical Press (2000), all of which are incorporated herein by reference.

Pharmaceutical compositions containing a 2D-VCAM-1 variant polypeptide of the present invention or protein fusion or conjugate thereof may he in any form suitable for the intended method of administration, including, for example, a solution, a suspension, or an emulsion. Liquid carriers contemplated for use in the practice of the present invention include, for example, water, saline, pharmaceutically acceptable organic solvent(s), pharmaceutically acceptable oils and fats, and the like, as well as mixtures of any two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

2D-VCAM-1 variant polypeptides of the present invention and protein fusions and conjugates thereof may be administered orally, parenterally, sublingually, by inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or iontophoretic devices. The term “parenteral” as used herein includes subcutaneous injections, intravenous administration, intramuscular administration, intrasternal injections, transdermal or transuaucosal administration, or infusion techniques.

Injectable preparations (such as, for example, sterile injectable aqueous or oleaginous suspensions) may be formulated using standard methods and materials known in the art, such as, for example, suitable dispersing, wetting, and suspension agents. The sterile injectable preparation may also be a solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono-or diglycerides. In addition, fatty acids such as oleic acid find use in the preparation of injectables.

Solid dosage forms fur oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, such as, for example, lubricating agents (e.g., magnesium stearate), and the like. In the case of capsules, tablets, and pills, the dosage forms may also comprise buffering agents. Tablets and pills can additionally be prepared with enteric coatings.

2D-VCAM-1 variant polypeptides of the present invention or protein fusion or conjugate thereof may also be administered in the form of liposomes. As is known in the art, liposomes are generally derived from phospholipids or other lipid substances. Liposomes are formed by mono- or multicellular hydrated liquid crystals that are dispersed in an aqueous medium. Any non-toxic, physiologically acceptable and metabolizable lipid capable of forming liposomes can be used. The present compositions in lipid form can contain, in addition to a compound of the present invention, stabilizers, preservatives, excipients, and the like. Typical lipids are the phospholipids and phosphatidyl cholines (lecithins), both natural and synthetic. Methods of forming liposomes are known in the art and are described in Prescott, Ed., “Methods in Cell Biology”, Volume XIV, Academic Press, New York, N.W., p. 33 et seq. (1976), which is incorporated herein by reference.

### Methods of Using 2D-VCAM-1 Variant Polypeptides, Conjugates, and Compositions Thereof

The present invention provides methods of using the 2D-VCAM-1 variant polypeptides of the present invention, and conjugates and pharmaceutical compositions thereof, to inhibit interactions of VLA4 (α4β1 integrin) with VCAM-1 which are implicated in the mechanism for inflammation. More specifically, the present invention provides a method of treating an inflammatory disease in a subject comprising administering to the subject (a human or a non-human mammal) a therapeutically effective amount of a 2D-VCAM-1 variant polypeptide of the present invention or a conjugate or pharmaceutical composition thereof. The treatment may be either a therapeutic treatment or a prophylactic treatment of an inflammatory disease, including those described herein. In another embodiment, the present invention provides use of a 2D-VCAM-1 variant polypeptide of the present invention or fusion protein or conjugate thereof for the manufacture of a medicament for the treatment (therapeutic or prophylactic) of an inflammatory disease.

Antagonists to α4 integrin have been reported as being effective in inhibiting a wide variety of experimental models of inflammatory diseases and autoimmunity because they inhibit the recruitment of lymphocytes and monocytes to sites of inflammation (see Féral, C. C. et al., J. Clin. Invest., 116(3): 715-723 (2006); Ransohoff; R. M. et al., Nat. Rev. Immunol., 3:569-581 (2003); Smolen, J. S., et al., Nat. Rev. Drug Discov., 2:473-488 (2003); James, W. G., et al., J. Immunol. 170:520-527 (2003); von Andrian, U. H., et al. N. Engl. J. Med. 348:68-72 (2003), all of which are incorporated herein by reference). As used herein, the term “inflammatory disease” refers to a disease or disorder in which inflammation plays a significant pathological role, including, for example, an autoimmune disease or disorder. These include diseases such as Alzheimer's disease, anaphylaxis, ankylosing spondylitis, asthma, atherosclerosis, atopic dermatitis, chronic obstructive pulmonary disease, Crohn's disease, gout, Hashimoto's thyroiditis, ischaemia-reperfusion injury, multiple sclerosis, osteoarthritis, pemphigus, periodic fever syndromes, psoriasis, rheumatoid arthritis, sarcoidosis, systemic lupus erthematosus, type I diabetes mellitus, ulcerative colitis, vasculitides (Wegener's syndrome, Goodpasture's syndrome, giant cell arteritis, polyarteritis nodosa), and xenograft rejection, bacterial dysentery, Chagas disease, cystic fibrosis pneumonitis, bilariasis, Helicobacter pylori gastritis, hepatitis C, influenza virus pneumonia, leprosy (tuberculoid form), neisserial or pneumococcal meningitis, post-streptococcal glomerulonephritis, sepsis syndrome, and tuberculosis. Nathan, Nature, 420:846 (2002). In particular, the present invention provides methods for the treatment, prevention, alleviation, or suppression of diseases or disorders mediated by the VLA4 pathway. 2D-VCAM-1 variant polypeptides of the present invention and protein fusions and conjugates thereof may be used for the manufacture of a medicament for the therapeutic or prophylactic treatment of an inflammatory disease or disorder. Such diseases and disorders include, for example, asthma, multiple sclerosis, allergic rhinitis, allergic conjunctivitis, inflammatory lung diseases, rheumatoid arthritis, septic arthritis, type I diabetes, organ transplant rejection, inflammatory bowel disease, and others mediated by adhesion molecules such as, for example, Alzheimer's disease, atherosclerosis, AIDs dementia and tumor metastasis.

A characteristic of acute inflammation is increased leukocyte adherence to endothelium (Harlan, Blood, 65(3):5130525 (1985)). The present invention provides a method of inhibiting adhesion of a leukocyte to an endothelial cell, the method comprising administering a therapeutically effective amount of a 2D-VCAM-1, variant polypeptide of the present invention, or a conjugate or a composition thereof, to a human or non-human mammalian subject. 2D-VCAM-1 variant polypeptides of the present invention have been shown to have little effect on the steady state expression of VLA4 or cell surface levels of −4 or β1 integrin containing adhesion molecules as shown in Example 19, hereinbelow. Therefore, in one embodiment, the present invention provides a method of inhibiting adhesion of a leukocyte to an endothelial cell with minimal impact on cell surface expression of α4 or β1 containing cell adhesion molecules such as α4β1 (VLA4), α4β7 (LPAM-1), α0β1 (VLA1), α2β1 (VLA2), α3β1 (VLA3), α5β1 (VLA5), α6β1 (VLA6), α7β1, α8β1, and α9β1.

In view of the properties of the 2D-VCAM-1 molecules described hereinabove and in the Examples, it is believed that the molecules of the present invention may provide a therapeutic and prophylactic benefit in reducing the PML risk associated with other VLA4 antagonists.

Those having ordinary skill in the art will appreciate that the specific dose level for any particular subject or patient may depend upon a variety of factors including the activity of the specific compound employed, the age, body weight, general health, route of administration, severity of the disorder, rate of excretion, and the like. The therapeutically effective amount for a given situation can be readily determined by routine experimentation and is within the skill and judgment of the ordinary clinician.

For purposes of the present invention, a therapeutically effective dose will be generally be from about 0.1 μg/kg/day to about 100 mg/kg/day of a 2D-VCAM-1 variant polypeptide or conjugate of the present invention, sometimes from about 10 μg/kg/day to about 500 μg/kg/day, and sometimes from about 10 μg/kg/day to about 100 μg/kd/day, which may be administered in one or multiple doses.

The 2D-VCAM-1 variant polypeptides of the present invention and conjugates and pharmaceutical compositions thereof can be administered at the recommended maximum clinical dosage or at lower dosages. Dosage levels of the active compounds and conjugates in the compositions of the invention may be varied so as to obtain a desired therapeutic response depending on the route of administration, severity of the disease, the response of the patient, and the like.

While the 2D-VCAM-1 variant polypeptides and conjugates of the present invention can be administered, as the sole active pharmaceutical agent, they may also be used in combination with one or more other agents The combination can be administered as separate compositions or as a single dosage form containing both agents. When administered as a combination, the therapeutic agents can be formulated as separate compositions that are given at the same time or different times, or the therapeutic agents can be given as a single composition.

2D-VCAM-1 variant polypeptides and conjugates of the present invention are also useful for detecting the presence of cells bearing VLA4. The presence of such cells in the brain may indicate the need to begin a therapeutic treatment. 2D-VCAM-1 variant polypeptides or conjugates thereof may be labeled with a detectable label, such as a fluorescent label, a radioisotope, a paramagnetic isotope, and the like. The labeled 2D-VCAM-1 variant polypeptide or conjugate thereof is contacted with the cells, in vitro (a sample drawn from the subject) or in vivo. Labelled 2D-VCAM-1 variant polypeptides or conjugates thereof that bind to the subject cells are detected and quantified. A change in VLA4 levels in the subject may signal an undesirable inflammatory response. The present invention thus provides a method for detecting VLA4, the method comprising contacting a 2D-VCAM-1 variant polypeptide or conjugate thereof with a tissue sample from a human or non-human mammalian subject; and detecting complexes formed by specific binding between the 2D-VCAM-1 variant polypeptide or conjugate thereof and the VLA4 in the tissue sample.

Embodiments of the invention include the following:
1. A recombinant 2D-VCAM-1 variant polypeptide comprising a sequence which differs in 0-8 amino acid positions from SEQ ID NO:12, and contains a proline at position 37 (P37) and a leucine at position 39 (L39) relative to SEQ ID NO:12,
   wherein the polypeptide has a binding affinity for a human VLA4 (integrin α4β1) protein that is greater than the binding affinity of Q38L -2D-VCAM-1 (SEQ ID NO:10) for the human VLA4 protein.
2. The recombinant 2D-VCAM-1 variant polypeptide of embodiment 1, wherein the sequence contains an alanine at position 41 (A41) relative to SEQ ID NO:12.
3. The recombinant 2D-VCAM-1 variant polypeptide of embodiment 1, wherein the sequence comprises one or more substitution selected from F32L/S, S34F/Y, Q38L, A41S, T74R/A, K79S/R, L141F/W, D145A, and R146W relative to SEQ ID NO:12.
4. The recombinant 2D-VCAM-1 variant polypeptide of embodiment 1, wherein the sequence is selected from SEQ ID NO:12, SEQ ID NO:14, SEQ ID NO:16, SEQ ID NO:18, SEQ ID NO:20, SEQ ID NO:22, and SEQ ID NO:24.
5. The recombinant 2D-VCAM-1 variant polypeptide of embodiment 1, wherein the sequence differs in 0-5 amino acid positions from SEQ ID NO:12.
6. The recombinant 2D-VCAM-1 variant polypeptide of claim 5, wherein the sequence is selected from SEQ ID NO:12, SEQ ID NO:14, SEQ ID NO:16, SEQ ID NO:22, and SEQ ID NO:24.
7. The recombinant 2D-VCAM-1 variant polypeptide of embodiment 1, wherein the sequence contains a leucine at position 38 (L38) relative to SEQ ID NO:12.
8. A recombinant 2D-VCAM-1 variant polypeptide comprising a sequence which differs in 0-8 amino acid positions from. SEQ ID NO:18, and contains a proline at position 37 (P37) and a leucine at position 39 (L39) relative to SEQ ID NO:1.8,
   wherein the polypeptide has a binding affinity for a human VLA4 (integrin α4β1) protein that is greater than the binding affinity of Q38L-2D-VCAM-1 (SEQ ID NO:10) for the human VLA4 protein.
9. The recombinant 2D-VCAM-1 variant polypeptide of embodiment 8, wherein the sequence is selected from SEQ ID NO:18, SEQ ID NO:12, SEQ ID NO:14, SEQ ID NO:16, SEQ ID NO:20, SEQ ID NO:22, and SEQ ID NO:24.
   10. The recombinant 2D-VCAM-1 variant polypeptide of embodiment 8, wherein the variant sequence comprises one or more substitution selected from L32F/S, F34S/Y, L38Q, S41A, R74T/A, S79K/R, L141F/W, D145A, and R146W relative to SEQ ID NO:18.
   11. The recombinant 2D-VCAM-1 variant polypeptide of embodiment 8, wherein the sequence differs in 0-5 amino acid positions from SEQ ID NO:18.
   12. The recombinant 2D-VCAM-1 variant polypeptide of embodiment 11, wherein the sequence is selected from SEQ ID NO:18, SEQ ID NO:14, SEQ ID NO:20, and SEQ ID NO:22.
   13. The recombinant 2D-VCAM-1 variant polypeptide of embodiment 1 or embodiment 8, wherein the sequence comprises the native human. VCAM-1 Domain-2 sequence corresponding to amino acids 96-199 of SEQ ID NO:12 or SEQ ID NO:18.
   14. The recombinant 2D-VCAM-1 variant polypeptide of embodiment 1 or embodiment 8, wherein the sequence is 191-199 amino acids in length.
   15. The recombinant 2D-VCAM-1 variant polypeptide of embodiment 1 or embodiment 8, wherein the sequence comprises a motif selected from RPQLDAP (SEQ ID NO:7) and RPLLDSP (SEQ ID NO:8).
   16. The recombinant 2D-VCAM-1 variant polypeptide of embodiment 1 or embodiment 8, wherein the polypeptide has a binding affinity for human VLA4 protein that is at least 4-fold greater than the binding affinity of Q38L-2D-VCAM-1 (SEQ ID NO:10) for human VLA4 protein in the assay of Example 10.
   17. The recombinant 2D-VCAM-1 variant polypeptide of embodiment 1 or embodiment 8, wherein the polypeptide has a binding affinity for human LPAM-1 protein (integrin α4β7) that is less than the binding affinity of Q38L-2D-VCAM-1 (SEQ ID NO:10) for human LPAM-1 protein in the assay of Example 11.
   18. The recombinant 2D-VCAM-1 variant polypeptide of embodiment 1 or embodiment 8, wherein the polypeptide has a binding affinity for human LPAM-1 protein that is greater than the binding affinity of Q38L-2D-VCAM-1 (SEQ ID NO:10) for human LPAM-1 protein in the assay of Example 11.
   19. The recombinant 2D-VCAM-1 variant polypeptide of embodiment 1 or embodiment 8, wherein the polypeptide exhibits a VLA4/LPAM-1 binding affinity ratio of at least 2, relative to the VLA4/LPAM-1 binding affinity ratio of Q38L-2D-VCAM-1 (SEQ ID NO:10) in the assay of Example 12.
   20. The recombinant 2D-VCAM-1 variant polypeptide of embodiment 1 or embodiment 8, wherein the sequence comprises the human 2D-VCAM-1 linker sequence corresponding to amino acids 89-95 of SEQ ID NO:12 or SEQ ID NO:18.
   21. The recombinant 2D-VCAM-1 variant polypeptide of embodiment 1 or embodiment 8, further comprising a tag peptide fused to the N-terminus or to the C-terminus.
   22. The recombinant VLA4-binding polypeptide of embodiment 21, wherein the tag peptide is a polyhistidine tag having the sequence SEQ ID NO:25.
   23. A 2D-VCAM-1 variant conjugate, comprising a recombinant 2D-VCAM-1, variant polypeptide of embodiment 1 or embodiment 9 and at least one conjugation moiety covalently bound to the polypeptide, wherein the conjugation moiety is selected from a non-polypeptide polymer moiety, a sugar moiety, and a non-polypeptide lipophilic moiety.
   24. The conjugate of embodiment 23, wherein the non-polypeptide polymer moiety is a polyethylene glycol (PEG).
   25. The conjugate of embodiment 24, wherein the PEG is a branched PEG of about 20-80 kdal molecular weight.
   26. The conjugate of embodiment 24, wherein the PEG is covalently bound to a lysine or to the N-terminus of the polypeptide.
   27. The conjugate of embodiment 24, comprising two or more PEGS covalently bound to the polypeptide.
   28. The conjugate of embodiment 24, wherein the PEG is bound to the N-terminus of the polypeptide, either directly or indirectly via a linker peptide or a tag peptide.
   29. The conjugate of embodiment 24, wherein the PEG a is branched PEG.
   30. The conjugate of embodiment 24, comprising the sequence SEQ ID NO:12, SEQ NO:14, SEQ ID NO:16, or SEQ ID NO:18.
   31. A composition comprising the polypeptide of embodiment 1 or embodiment 8, or the conjugate of embodiment 23, and a pharmaceutically acceptable carrier or excipient.
   32. A recombinant polynucleotide comprising a nucleic acid sequence encoding the 2D-VCAM-1 variant polypeptide of embodiment 1 or embodiment 8.
   33. A vector comprising the polynucleotide of embodiment 32 operably linked to a promoter.
   34. A host cell transformed or transfected with the vector of embodiment 33.
   35. A method of producing a recombinant 2D-VCAM-1 variant polypeptide, the method comprising:
   (a) culturing a host cell transformed with the vector of embodiment 33 under conditions suitable for expression of the polypeptide; and
   (b) recovering the polypeptide from the culture medium or from the host cell.
   36. A method of producing a recombinant 2D-VCAM-1 variant polypeptide, the method comprising:
   (a) culturing a host cell transformed with the vector of embodiment 33 under conditions suitable for expression of the polypeptide as inclusion bodies;
   (b) recovering the inclusion bodies comprising the polypeptide;
   (c) solubilizing the inclusion bodies to obtain solubilized polypeptide;
   (d) incubating the solubilized polypeptide in a solution of urea and reduced/oxidized glutathione under conditions suitable for the solubilized polypeptide to refold, to obtain refolded polypeptide;
   (e) purifying the refolded polypeptide; and
   (f) recovering the polypeptide from step (e).
   37. The method of embodiment 36, wherein step (e) comprises purifying the refolded polypeptide by chromatography over a ceramic hydroxyapatite (CHT) column.
   38. The method of embodiment 35, 36, or 37, further comprising:
      covalently attaching at least one conjugation moiety to the recovered polypeptide.
   39. The method of embodiment 38,wherein the step of covalently attaching at least one conjugation moiety to the polypeptide comprises incubating the polypeptide with a branched PEG-aldehyde of 20-80 kdal molecular weight under conditions effective to conjugate the PEG-aldehyde to the N-terminal-amino group of the polypeptide, thereby producing a 2D-VCAM-1 conjugate comprising a branched PEG moiety covalently attached to the N-terminus of the polypeptide.
   40. A method of inhibiting the binding of VCAM-1 and VLA4 in a human or non-human mammalian subject, the method comprising: administering to the subject the polypeptide of embodiment 1 or embodiment 8, the conjugate of embodiment 23, or the composition of embodiment 31, in an amount effective to inhibit the binding of VCAM-1 and VLA4 in the subject.

The foregoing and other aspects of the invention may be better understood in connection with the following non-limiting examples.

### EXAMPLES

### Example 1

### Construction of 2D-VCAM-1 Plasmids, Cell Transfection for CHO Cell-Surface Display of 2D-VCAM-1, and Detection of Cell-Surface Displayed 2D-VCAM-1

To display 2D-VCAM-1 on the cell surface of CHO K1 cells, polynucleotides encoding the first two domains of human VCAM-1 and variants thereof (e.g., human 2D-VCAM-1, SEQ ID NO:1; Q38L-2D-VCAM-1, SEQ ID NO:9; and exemplary 2D-VCAM-1 variants of the invention, SEQ ID NOS:11, 13, 15, 17, 19, 21, and 23) were translationally fused to polynucleotides encoding human IgG kappa light chain constant region (“Cκ”; SEQ ID NO:32; the amino acid sequence of which is provided as SEQ NO:33), the V5 epitope derived from the Paramyxovirus SV5 (Southern, J. A. et al., J. Gen. Virol. 72:1551.-1557 (1991); SEQ ID NO:34; the amino acid sequence of which is provided as SEQ ID NO:35) and the glycosyl-phosphatidylinositol (GPI) membrane anchor (SEQ ID NO:36; the amino acid sequence of which is provided as SEQ ID NO:37). The rationale for using a fusion with Cκ was to facilitate proper folding and target the 2D-VCAM-1 variants to the secretory pathway. The V5 epitope was used to provide a convenient epitope tag to monitor the recombinant protein expression levels displayed at the cell surface.

To construct human 2D-VCAM-1 plasmids, the signal peptide and the first two domains of human VCAM-1 were amplified by PCR using oligonucleotides TBO380 (SEQ ID NO:38) and TBO381 (SEQ ID NO:39) from a leukocyte cDNA library as template (Human Leukocyte Quick-Clone cDNA, Clontech catalog no. 637240). The resulting 680 base pair (bp) amplicon was purified and used as template for two PCR reactions aimed at introducing restriction sites for subsequent cloning and destroying an internal BamHI restriction site as follows. The PCR product TBO380-TBO381 was re-amplified with the oligonucleotides TBO388 (SEQ ID NO:40) and TBO383 (SEQ ID NO:41), giving rise to 394 bp amplicon containing a BamHI restriction site for subsequent subcloning, the signal peptide and 303 bases from VCAM-1 and a silent mutation in Asp94 of VCAM-1 (contained in the oligonucleotide TBO383) to destroy the original internal BamHI restriction site present in the human VCAM-1 gene. The PCR product TBO380-TBO381 was re-amplified with oligonucleotides TBO382 (SEQ ID NO:42) and TBO385 (SEQ ID NO:43) to create a 46 bp overlapping 5′ end matching that of the PCR product TBO388-TBO383 and containing a silent mutation in Val195 of VCAM-1 (contained in the oligonucleotide TBO385) to create a BsrGI restriction site for subsequent subcloning. The two PCR products were purified and subsequently assembled by a PCR reaction with the oligonucleotides TBO388 and TBO385. The resulting 688 bp amplicon was used as template in a new PCR reaction with the oligonucleotides TBO388 and TBO392 (SEQ ID NO:44), giving rise to a 705 bp amplicon containing the first 17 nucleotides from the Cκ.

The human Cκ sequence was amplified by PCR from a leukocyte cDNA library as template (Human Leukocyte Quick-Clone cDNA, Clontech Catalog No. 637240) using oligonucleotides TBO389 (SEQ ID NO:45) and TBO390 (SEQ ID NO:46). The resulting 271 bp amplicon was purified and used as template in a new PCR reaction with oligonucleotides TBO391 (SEQ ID NO:47) and TBO393 (SEQ ID NO:48), giving rise to a 354 bp amplicon containing a 36 nucleotides overlapping 5′ end matching that of the PCR product TBO388-TBO392, human Cκ sequence, a UGA stop codon and a SbfI restriction site. The two PCR products TBO388-TBO392 and TBO391-TBO393 were purified and assembled by PCR, using oligonucleotides TBO388 and TBO393. The resulting 1023 bp amplicon was digested with the restriction enzymes BamHI and SbfI and subcloned at the corresponding sites in the vector pB183, giving pVM009. The pVM009 plasmid contains the 2D-VCAM-1-Cκ-UGA-V5-GPI construct (SEQ ID NO:49, the amino acid sequence of which is provided as SEQ ID NO:50) which allows expression of a translational fusion between human 2D-VCAM-1 and Cκ, whose subcellular localization (secreted, or membrane anchored via the GPI author) is controlled via Regulated Readthrough technology. Regulated Readthrough vectors allow alternative production of membrane-bound and soluble forms of a recombinant protein from the same vector which can he exploited to obtain high expressor transfectants. Individual transfectant cells that show high surface expression are selected and amplified to obtain clonal cell lines that can then be used for production of high levels of soluble protein. Methods for making and using Regulated Readthrough vectors are described in International Application Publication No. WO 2005/073375 and in Bouquin T. et al., J. Biotechnol. 125:516-528 (2006), which are incorporated herein by reference.

Plasmid pVM009 was used as template to create the plasmid pVM025, which was identical to pVM009, except that it did not contain the UGA stop codon between the Cκ and V5 sequences, Plasmid pVM009 was used as template in a PCR reaction with oligonucleotides TBO394 (SEQ ID NO:51) and TBO014 (SEQ ID NO:52) and the resulting 274 bp amplicon was digested with restriction enzymes BstEII and PmeI and subcloned at the corresponding sites in pVM009, giving pVM025. Plasmid pVM025 allowed targeting of a translational fusion between 2D-VCAM-1, Cκ and the V5 epitope (SEQ ID NO:53, the amino acid sequence of which provided as SEQ ID NO:54) to the plasma membrane of CHO K1 cells.

Retroviral versions of the plasmids pVM009 and pVM025were constructed as follows. To generate a retroviral version of pVM009, the plasmid was digested with restriction enzymes BamHI and PmeI to release the 2D-VCAM-1-Cκ-UGA-V5-GPI cassette, which was purified and subsequently subcloned into the corresponding sites in the lentiviral vector pC100 (Bouquin T. et al., J. Biotechnol. 125:516-528 (2006)). The resulting pVM010 plasmid allowed alternative secretion or membrane anchorage of the 2D-VCAM-1-Cκ protein fusion in retroviral mammalian cell lines using Regulated Readthrough technology.

To generate a retroviral version of the plasmid pVM025, the TBO394-TBO014 PCR product was digested with the restriction enzymes BstEII and PmeI and subcloned into pVM010 at the corresponding sites. The resulting pVM011 plasmid (containing the 2D-VCAM-1-Cκ-V5-GPI cassette without the UGA stop codon between the Cκ and V5 sequences) was used to generate retroviral CHO K1 cell lines displaying a translational fusion between 2D-VCAM-1, Cκ and the V5 epitope at the cell surface.

We also constructed a derivative Plasmid from pVM011, called pVM036, in which the 2D-VCAM-1. sequence was optimized for E. coli expression. Site-directed mutagenesis was used to replace codons at the following residues with ones that were more favorable for E. coil expression: Arg10, Arg36, Ile39,Arg78, Arg123, Arg126, Arg146, Arg172, Pro184, Arg187, Ile197, and Cys199 in the mature human VCAM-1. The E. coli optimized human 2D-VCAM-1 nucleic acid sequence is provided as SEQ ID NO:55. The E. coil optimized 2D-VCAM-1 construct and the original construct showed comparable expression in CHO K1 cells indicating that the codon optimization did not negatively affect expression in CHO K1 cells.

All adherent CHO K1-based cell lines were grown in 37° C. incubators under 5% CO2 in D-MEM Nut Mix F-12 (HAM) medium (Gibco, Catalog No. BRL 31330-038) supplemented with 100 U/mL Penicillin and 100 μg/mL Streptomycin (BioWhittaker, DE17-602E) and 10% heat-inactivated fetal bovine serum (Gibco, Catalog No. BRL 10091-148). Cells were harvested with Cell Dissociation solution (Sigma, Catalog No. C5914) according to manufacturer's recommendations. Transgenic cell lines were generated using retroviral- or non-retroviral-based vectors as follows.

Transgenic cells harboring non-retroviral vectors were generated by transfection using the plasmid of interest in the presence of the transfection agent LIPOFECTAMINE™ 2000 (Invitrogen, Catalog No. 11668-019) and following manufacturer's recommendations. After transfection, cells were grown for 2 or 3 days before adding the selection antibiotic Hygromycin (360 μg/mL; invitrogen, Catalog No. 10687-010) or Blasticidin (5 mg/L; Invitrogen, Catalog No. R210-01), respectively. The selection culture medium was replenished every 2-3 days until generation of a stable cell pool (7 to 12 ‘days).

For retroviral vectors derived from pLenti6/V5-D-TOPO® (pVM010, pVM011, pVM036 & 2D-VCAM-1 libraries), retrovirus was produced in 293 FT cells (Invitrogen) following recommendations included in the VIRAPOWER™ lentiviral kit (Invitrogen, Catalog No. K4950-00), Briefly, 293FT cells were co-transfected with the pLenti6/V5-D-TOPO®-based vectors and the packaging plasmids (included in the VIRAPOWER™ lentiviral kit) to produce retrovirus particles. After 48 hours, supernatants containing retroviral particles were harvested, filter-sterilized to remove cell debris, and subsequently used to infect CHO-K1 cells. Retrovirus titers were assessed by infecting CHO K1 cells with a series of virus dilutions and following manufacturer's recommendations. Selection with 5 mg/L Blasticidin (Invitrogen, Catalog No. R210-01) was started 3 days post-infection and the selection medium was replenished every 2-3 days until generation of a stable cell pool (12 days).

The codon-optimized human 2D-VCAM-1 nucleic acid sequence (SEQ ID NO:55) for dual organism expression was compared to the native human 2D-VCAM-1 nucleic acid sequence (SEQ ID NO:1) for protein expression via membrane display in CHO K1 cells. Both pVM011 (native human 2D-VCAM-1) and pVM036 (codon-optimized human 2D-VCAM-1) retroviral vectors were used to produce retrovirus in 293FT cells by means of the VIRAPOWER™ lentiviral kit (Invitrogen Catalog No. K4950-00). After generation of CHO K1 stable cell lines, which were respectively named pVM011-R0 and pVM036-R0, the cells were assayed for recombinant protein membrane display using fluorescence-activated cell sorting (FACS)-analysis using a FACSCalibur™ Flow Cytometer (BD Biosciences) equipped with an Argon laser at 488 nm, and data analysis using CellQuest™ Pro software, ver 5.1.1 (BD Biosciences). Because the membrane displayed recombinant protein is a translational fusion between the human 2D-VCAM-4, the human IgG Kappa chain, the V5 epitope and the GPI anchor, it was possible to perform dual-staining using conjugated antibodies targeted at different epitopes of the recombinant protein. For this purpose, the FITC-conjugated anti-V5 mAb (Invitrogen, Catalog No. 46-0308; diluted 1:500) and PECy5-conjugated anti-human VCAM-1 mAB (Becton Dickinson, Catalog No. 551148; diluted 1:10) were used to stain the pVM011-R0 and pVM036-R0 transgenic cell pools. The fluorescence profiles were very similar between the two cell lines. More specifically, the percentage of cells positive for both V5 and 2D-VCAM-1 staining represented 84% and 72% of the pVM011-R0 and pVM036-R0 transgenic pools, respectively. The average fluorescence signals for the P1 (FITC) channel were 113 and 112 in the p011-R0 and pVM036-R0 transgenic cell pools, respectively. Similarly, the average fluorescence signals for the P3 (PECy5) channel were 590 and 503 in the pVM011-R0 and pVM036-R0 transgenic cell pools, respectively. This result indicates that the recombinant mRNA containing the codon optimized 2D-VCAM-1 is translated as efficiently as the recombinant mRNA containing the original human 2D-VCAM-1 in CHO K1 cells. Furthermore, this result indicates that it is possible to perform a functional library screen in CHO K1 cells displaying variants based on the codon-optimized 2D-VCAM-1, followed by subsequent subcloning and production of 2D-VCAM-1 variants in E. coli.

### Example 2

### Construction of 2D-VCAM-1 Phagemids, Production of Phage Displaying 2D-VCAM-1, and Detection of Phage-Displayed 2D-VCAM-1

2D-VCAM-1 was displayed on the surface of filamentous phage using a monovalent-phage-display vector plasmid pS0124. Plasmid pS0124 is a phagemid E. coil vector that contains a colE1 origin for replication in E. coli, an M13 origin and packaging signal, the β lactamase gene fur ampicillin resistance selection in E. coli, and an arabinose (araBAD) operon that drives the expression of a translational fusion of the STII secretory signal, the polypeptide to be displayed (for example, human 2D-VCAM-1), a 6-His tag, a suppressible amber stop codon, and the M13 geneIII C-terminal domain. The nucleic acid sequence of an exemplary translational fusion with human 2D-VCAM-1 is provided as SEQ ID NO:56. Unique SfiI and NotI sites between the STII secretory signal sequence and the His tag permit cloning of the DNA sequence encoding the polypeptide to be displayed to generate an in-frame fusion. The araBAD promoter (pBAD) can be activated by arabinose interacting with the araC activator protein whose gene is also contained in the plasmid. When the plasmid is transformed into an amber-suppressor strain such as TG1 (Stratagem) or TOP10 (Invitrogen), basal expression from the araBAD promoter drives the expression and processing of a mature fusion protein consisting of the polypeptide to he displayed, the His tag, and the C-terminal domain of the M13 protein III.

DNA encoding 2D-VCAM-1 was amplified with primers to introduce flanking SfiI and NotI sites, which were subsequently used to subclone into pS0124 to generate plasmid pS0124-2DVCAM1. The plasmid was transformed into E. coli TG1 cells (Stratagene, Catalog No. 200123) and the transformants were used for phage production as described below.

For preparation of phage displaying 2D-VCAM-1 or its variants, a phagemid clone was inoculated into 3 ml of 2×YT+Carb50 (50 ug/mL, of Carbenicillin) and subsequently subcultured into 50 mls of 2×YT+Carb50 until mid-log phase at which time 1 mL of M13K07 helper phage (˜1 E+10 cfu/mL) was added and the culture was grown overnight at 37° C. at 250 rpm. The overnight culture was centrifuged at 6000 rpm for 20 min and the supernatant was collected and mixed with one-fourth volume of 5×PEG/NaCl (20% polyethylene glycol 8000, 2.5M NaCl) solution and incubated on ice for 30 min. Phage DNA was collected by centrifugation at 10,000 rpm for 30 min at 4 oC and resuspended in 1-2 ml of Tris buffered saline (TBS; 25 mM Tris, pH 7.5, 150 nM NaCl)+1% bovine serum albumin (BSA). The resuspension was clarified by centrifugation at maximum speed in a microftige for 5 min and the supernatant was stored at 4° C. for up to a week.

Phage were titered by making a ten-fold serial dilution series of the phage in TBS+1% BSA, adding 1 ul of each dilution to 100 ul of a log-phase TG1 culture and allowing phage to infect TG1 cells at 37° C. for 20 min. The infected culture was then spread on LB agar plates containing 50 ug/ml carbenicillin. Phagemid transfectant colonies obtained after overnight incubation at 37° C. were counted and used to calculate the phage titer. Typically, the titer was in the 1012-1013 cfu/ml range.

Phage-displayed 2D-VCAM-1 was tested for function using a phage ELISA. NUNC MaxiSorp™ plates were coated overnight at 4° C. with 50 ul/well of VLA4-Fc (see Example 3) at a concentration of 5 ug/ml in TBS-M (TBS plus 1 mM MnCl2). After washing with TBS-MT (TBS plus 1 snM MnCl2 and 0.02% TWEEN-20), the plates were blocked with TBS-M containing 3% milk (nonfat dried milk powder; Sigma-Aldrich). The block was washed and phage dilutions (2-fold titrations in TBS-MT plus 1% milk) were added to the coated plates and incubated at room temperature. After 2 h of incubation, the plates were washed with TBS-MT and bound phage was detected by incubating with horseradish peroxidase (HRP)-anti M13 antibody conjugate (GE Healthcare) for 30 min., washing with TBS-MT, and detecting the immobilized HRP using TMB-H2O2 reagent (Pierce). The reaction was stopped after color development using 2M H2S04 and the plate was read for absorbance at 450 nM using a spectrophotometer and SoftMaxPro™ software (Molecular Devices, Sunnyvale Calif.). The absorbance was plotted against phage dilution to obtain a binding curve to compare the binding of different variants.

### Example 3

### Cloning, Expression, Purification and Characterization of VLA4-Fc

### A. Construction of Human α4-Fc Plasmid

Human α4 integrin cDNA was PCR-amplified from a leukocyte cDNA library (Human Leukocyte Quick-Clone cDNA, Clontech Catalog No. 637240) as three fragments; The first fragment contained the first 317 nucleotides of mature human α4 integrin and used primers TBO358 and TBO357 (SEQ ID NOs:57 and 58, respectively). The second fragment was generated using primers TBO365 and TBO367 (SEQ ID NOs:59 and 60). The third PCR reaction using the primers TBO366 and TBO352 (SEQ ID NOs:61 and 62, respectively) resulted in an 1155 bp amplicon containing the sequence of the human α4 integrin. The three PCR products were purified using standard procedures and served as templates for PCR reactions aimed at generating DNA encoding the full human α4-integrin extracellular domain with the following changes a mouse κ IgG light chain signal peptide instead of the endogenous signal, removal of an internal BamHI restriction site, introduction of the mutation R558L (to remove a potential protease cleavage site), and introduction of restriction sites for subsequent subcloning.

The first PCR fragment was reamplified with primers TBO359 and TBO355 (SEQ ID NOs:63 and 64, respectively). Primer TBO359 contained a BamHI restriction site followed by a Kozak consensus translation initiation signal sequence encoding mouse IgGκ signal peptide (SEQ NO:65), and sequence hybridizing to the 5′ of the first fragment. TBO355 contained a silent mutation that disrupted the BamHI site toward the 3′ end of the first fragment. The resulting product was 412 bp long and contained a BamHI site, followed by the Kozak sequence, and DNA encoding a fusion of the mouse IgGκ signal to the mature human α4-integrin gene.

The second PCR product was amplified with primers TBO354 and TBO369 (SEQ ID NOs:66 and 67, respectively), to create a 33 bp overlapping 5′ end matching that of the first PCR fragment and containing a mutation R558L in the human α4 integrin.

The third PCR product was amplified with oligonucleotides TBO365 and TBO353 (SEQ ID NOs:68 and 69, respectively) to create a 36 bp overlapping 5′ end matching that of the second PCR product and containing the restriction site SalI for subsequent subcloning.

The three PCR products were purified using standard procedures and subsequently assembled and amplified by PCR using the flanking primers TBO359 and TBO353. The final PCR fragment encoded a translational fusion between the mouse IgGκ signal peptide and the human α4 integrin containing the mutation R558L. The 2907 bp amplicon was cloned into the pCR4-TOPO vector from the TOPO-TA cloning kit (Invitrogen, Catalog No. 45-0030) to create plasmid pVM017.

The human α4 integrin extracellular domain was fused to human IgG1 Fc. The human IgG1 Fc nucleic acid sequence was amplified from human leukocyte cDNA library (Human. Leukocyte Quick-Clone cDNA, Clontech Catalog No. 637240) by PCR using oligonucleotides TBO351 and TBO305 (SEQ ID NOs:70 and 71, respectively). A unique XhoI restriction site was introduced at the beginning of the IgG1 CH3 domain by a SOE (splicing by overlap extension) strategy (see, for example, Horton, et al., “Gene Splicing by overlap extension: tailor-made genes using the polymerase chain reaction”, Biotechniques, 8(5):528-35 (1990), which is incorporated herein by reference). The IgG1-Fc template was amplified in two independent PCR reactions using the oligonucleotide combinations TBO351 (SEQ ID NO:70) and TBO377 (SEQ ID NO:72) winch resulted in a 409 bp fragment and TBO376 (SEQ ID NO:73) and TBO305 (SEQ ID NO:71) which generated a 326 bp fragment. The two PCR fragments were assembled and amplified by PCR using oligonucleotides TBO351 and TBO305.

The resulting IgG1-Fc product served as template to introduce the mutation Y407T in the IgG1 Fc protein coding sequence. Two PCR reactions were performed using oligonucleotide combinations TBO361 (SEQ ID NO:74) and TBO365 (SEQ ID NO:59) to generate a 626 bp fragment and TBO364 and TBO360 (SEQ ID NOs:75 and 76, respectively) to generate a 159 bp fragment. The two PCR products TBO361-TBO365 and TBO364-TBO360 were assembled by PCR using the oligonucleotides TBO361 and TBO360 giving rise to a 753 bp amplicon containing the human IgG1 Fc sequence with the Y407T mutation and BamHI, SalI, XhoI and PmeI restriction sites that were introduced for subcloning purpose. The TBO361-TBO360 PCR product was cloned into the pCR4-TOPO vector from the TOPO-TA cloning kit (Invitrogen, Catalog No. 45-0030) generating plasmid PVM001.

A BamHI-SalI fragment containing the muIgG1κ signal and α4 integrin R558L sequence was excised from pVM017 and subcloned into the corresponding sites in pVM001 to generate plasmid pVM003, which contained a functional fusion of the muIgG1κ signal, α4 integrin R558L mutant ECD, and Fc^{Y407T} sequences. This fusion was subcloned into pCDNA3.1 Hyg (Invitrogen) using BamHI and PmeI enzymes to obtain plasmid pVM004 which contained a hygromycin resistance marker and a CMV-promoter, which drove expression of the soluble human α4 integrin-PcY407T (referred to herein as “α4-Fc”; the nucleic acid sequence provided herein as SEQ ID NO:77 and amino acid sequence SEQ ID NO:78).

To generate cell lines that produce the highest possible levels of soluble α4-Fc the plasmid vector pVM007 based on Regulated Readthrough technology (as described in WO 2005/073375, which is incorporated herein by reference) was constructed by introducing a UGA stop codon, the V5 epitope and the GPI anchor (referred to herein as UGA-V5-GPI) downstream of the α4-Fc translational fusion in plasmid pVM004. The UGA-V5-GPI cassette was generated as follows: plasmid pVM001 served as template in a PCR reaction using oligonucleotides TBO376 and TBO379 (SEQ ID NO:79) to produce a 347 bp amplicon containing the CH3 region of Fc^{Y407T}, a UGA stop codon and the upstream part of the V5 epitope. A second PCR reaction was performed using plasmid pB183 as template and oligonucleotides TBO378 and TBO104 (SEQ ID NOs:80 and 81, respectively) to generate a 235 bp amplicon containing a 37 bp overlap with the 3′ end of the PCR product TBO376-TBO379, the remaining V5 epitope, a GPI sequence, and the restriction site PmeI for subcloning. The two PCR products TBO376-TBO379 and TBO378-TBO104 were assembled and amplified by PCR using oligonucleotides TBO376 and TBO104 and the resulting 545 bp amplicon was digested with XhoI and PmeI and used to replace the XhoI-PmeI fragment in pVM004, generating pVM007. Plasmid pVM007 allowed alternative production of soluble or membrane-anchored α4-Fc through the Regulated Readthrough approach, as described in WO 2005/073375, which is incorporated herein by reference.

A second set of α4-Fc and β1-Fc expression plasmids was produced using ubiquitous chromatin-opening element technology (UCOE) (Millipore, Inc.). Vectors CET1019AS-puro and CET1019AS-hygro contain a DNA element that induces local chromatin unwinding to improve expression after plasmid integration in the genomic DNA. Both vectors have a promoter A (from guinea pig CMV) and SV40 polyA terminator. The α4-Fc from pVM007 was excised using NheI and PmeI restriction sites and cloned in CET1019AS-puro digested with NheI and SnaBI restriction enzymes to obtain plasmid pVM162. The α1-Pc ORF from pVM008 was excisted using NheI and PmeI restriction enzymes and cloned into CET1019AS-hygro digested with the same enzymes to obtain plasmid pVM168.

### B. Construction of Human β1-Fc Plasmid

Human α1 integrin was cloned by PCR using a leukocyte cDNA library as template (Human Leukocyte Quick-Clone cDNA; Clontech, Catalog No. 637240) and oligonucleotides TBO370 and TBO371 (SEQ ID NOs:82 and 83, respectively) to obtain a PCR product containing the first 2187 nucleotides of human β1 integrin gene, which included the secretory signal sequence and the extracellular domain. The PCR product was reamplified using oligonucleotides TBO372 and TBO373 (SEQ ID NOs:84 and 85, respectively) to introducing flanking BamHI and PmeI restriction sites. The fragment was cloned into pCR4-TOPO vector from the TOPO-TA cloning kit, (Invitrogen, Catalog No. 45-0030), resulting in the plasmid pVM016.

The human β1 signal sequence and β1 integrin was then fused to human IgG1-Fc sequence. First, the human IgG1-Fc sequence obtained in PCR product TBO351-TBO305 was mutated to contain the T366Y mutation by two PCR reactions: the first reaction with oligonucleotides TBO361 (SEQ ID NO:74) and TBO363 (SEQ ID NO:86) which gave a 505 bp fragment and the second with TBO362 (SEQ ID NO:87) and TBO360 to give a 277 bp fragment. The two PCR products TBO361-TBO363 and TBO362-TBO360 were assembled and amplified b PCR using oligonucleotides TBO361 and TBO360, giving rise to a 753 bp amplicon containing the human IgG1-Fc sequence with the T366Y mutation and BamHI, SalI, XhoI and PmeI restriction sites. The assembled product was then cloned into pCR4-TOPO vector from the TOPO-TA cloning kit resulting in the plasmid pVM002. The human β1 integrin sequence was excised using enzymes BamHI and SalI from plasmid pVM016 and subcloned into the corresponding sites in plasmid pVM002 to obtain pVM005 which contained a translation fusion between the human β1 integrin and the human IgG1 Fc^{T366Y} (referred to herein as “β-Fc”; the nucleic acid sequence provided as SEQ ID NO:88 and amino acid sequence SEQ ID NO:89).

The β1-Fc fusion was subcloned into an expression vector derived from pCDNA6-myc-His-A (Invitrogen, Catalog No. V22120). The 5′ untranslated region (5′ UTR) and β-globin intron sequences were PCR amplified from plasmid pCDNA3.1-Hyg (pF377) (Invitrogen) using oligonucleotides TBO374 and TBO375 (SEQ ID NOs:90 and 91, respectively) to generate a 406 bp amplicon flanked by the HindIII and BamHI restriction sites, which was cloned using HindIII and BamHI into pCDNA6-myc-His-A to obtain plasmid pCDNA6-myc-His-A-intron. The β1-Fc cassette was excised from plasmid pVM005 using enzymes BamHI and PmeI and subcloned into plasmid pCDNA6-mye-His-A-intron to obtain plasmid pVM006. Plasmid pVM006 contained a blasticidin resistance marker and a CMV promoter which drove expression of soluble β1-Fc.

To generate cell lines producing the highest possible levels of soluble β1-Fc, plasmid vector pVM008 based on the Regulated Readthrough technology was constructed by introducing a UGA stop codon, the V5 epitope and the GPI anchor (referred herein as UGA-V5-GPI) downstream of the β1-Fc translational fusion of the pVM006 plasmid. Plasmid pVM002 served as template in a PCR reaction using oligonucleotides TBO376 and TBO379 to generate a 347 bp amplicon containing the 3′ end of the Fc domain, a UGA stop codon and the upstream part of the V5 epitope. A second PCR reaction was performed using plasmid pB183 as template and using oligonucleotides TBO378 and TBO104. The resulting 235 bp amplicon contained a 37 bp overlap with the 3′ end of PCR product TBO376-TBO379, the remaining V5 epitope, a GPI sequence and a PmeI restriction site for subcloning. The two PCR products TBO376-TBO379 and TBO378-TBO104 were assembled and amplified by PCR using oligonucleotides TBO376 and TBO104 to obtain a 545 bp amplicon that was digested with XhoI and PmeI restriction enzymes and cloned at the corresponding sites in vector pVM006, giving plasmid pVM008, Plasmid pVM008 allowed alternative production of soluble or membrane-anchored □1-Fc through the Regulated Readthrough approach, as described in WO 2005/073375, which is incorporated herein by reference.

A second set of α-4-Fc and β1-Fc expression plasmids was produced using ubiquitous chromatin-opening element (UCOE) technology (Millipore, Inc.). Vectors CET1019AS-puro and CET1019AS-hygro contain a DNA element that induces local chromatin unwinding to improve expression after plasmid integration in the genomic DNA. Both vectors have a promoter A (from guinea pig CMV) and SV40 polyA terminator. The αf-Fc from pVM007 was excised using NheI and PmeI restriction sites and cloned in CET1019AS-puro digested with NheI and SnaBI enzymes to obtain plasmid pVM162. The β1-Fc ORF from pVM008 was excised using NheI and PmeI restriction enzymes and cloned into CET1019AS-hygro digested with the same enzymes to obtain plasmid pVM168.

### C. Construction of α4-Fc/β1-Fc Producing Cell Lines:

To produce α4-Fc/β1-Fc (also referred to herein as “VLA4-Fc”) cell lines through the Regulated Readthrough method, we used a sequential approach where we first transfected CHO-K1 with the α4-Fc-based plasmid (pVM007) and generated and selected clones displaying high levels of α4-Fc. These clones were subsequently used as recipient cells for transfection with the β1-Fc-based plasmid (pVM008). The resulting transgenic cell lines were screened for heterodimer secretion by means of the sandwich ELISA (described in Example 9, below).

CHO-K1 cells were transfected with the pVM007 plasmid using Lipofectamin™ 2000 (invitrogen) according to the manufacturer's recommendations and selected for Hygromycin resistance. After generation of a stable cell pool, the cells were submitted to two rounds of FACS-based enrichment for human α4-Fc display via GPI anchoring. The percentages of sorted cells were respectively 0.8% and 0.7% for each enrichment round. To assess the success of the different FACS-based enrichment rounds, supernatants from cell pools corresponding to the original transgenic cells (round R0) or cells submitted to one (round R1) or two (round R2) rounds of FACS were assayed for α4-Fc production (as described in Example 9 below). There was a dramatic improvement in α4-Fc secretion between the different enrichment rounds, indicating that the FACS steps had been very successful to enrich for high expresser cells. Indeed, the α4-Fc secretion level in the cell pool that had been subjected to two FACS rounds (pVM007-R2) was 87 folds that of the original transgenic cell pool (pVM007-R0).

The pVM007-R2 cell pool was subsequently submitted to limited dilution cloning in five 96-well culture plates. After 2 weeks of growth, approximately 300 clones were visually identified and ranked for α4-Fc production by means of ELISA (see Example 9). Twenty-four clones exhibiting the highest α4-Fc recombinant protein production were pooled, amplified and transfected with plasmid pVM008 using LIPTOFECTAMINE 2000 according to the manufacturer's recommendations. Transgenic cell lines were obtained after approximately two weeks of growth in the presence of blasticidin antibiotic. Cells co-transfected with PVM007 and PVM008 were designated CHO-K1-007-008. Each cell pool was submitted to limited dilution cloning in five 96-well culture plates. After 2 weeks of growth, the CHO-K1-007-008 clones were ranked for α4-Fc and β1-Fc production by means of the sandwich ELISA (Example 9). Eight CHO-K1-007-008 clones were selected, transferred to T-flasks for further growth, and assessed for secreted heterodimer levels one week later using the sandwich ELISA. One clone was chose for further recombinant protein production in roller bottles.

A second VLA4-Fc cell-line was generated from CHO-S cells using plasmids pVM162 and pVM168 through sequential transfection process similar to the one described above.

### D. Expression and Purification of VLA4-Fc

The chosen cell line was transferred to roller bottles and adapted to serum-free medium. The supernatants were harvested daily and frozen at −80° C. At the end of the campaign, the supernatant was thawed, pooled, and purified by standard Protein A chromatography using a rProteinA-sepharose column (GE Healthcare) with glycine pH 3.2 as the elution buffer. Eluted fractions were collected and analyzed on an SDS-PAGE gel under reduced conditions. After electroblotting onto a PVDF membrane, the two main bands were sequenced. No sequence was obtained for the band with the lowest Mr (β1-Fc). The fractions were tested separately and by the functional cell binding assay (below). The fractions exhibiting equivalent activity were pooled and stored at −80° C. in the neutralized elution buffer. This was used subsequently as the standard for the functional ELISA assay (below). Active fractions can also be identified and pooled using BIACORE (below).

### E. Characterization of VLA4-Fc

1. MALDI-TOF mass spectrometry: MALDI-TOF mass spectrometry was carried out on the VLA4 Fc fusion protein. The results showed that the fusion protein had a heterogeneous mass around 280 kDa. The heterogeneity was likely caused by the attachment of N-glycans on a large number of N-glycosylation sites in both the alpha- and beta-chains. The mass of the attached carbohydrate was estimated to be around 45 kDa (approximately 18 N-glycans assuming an average mass of each N-glycan of 2500 Da).

2. N-terminal sequencing: N-terminal amino acid sequence determinations of the main two bands observed from the gel analysis described in part D above were carried out. For the band with the highest Mr, the N-terminal amino acid sequence Tyr-Asn-Val-Asp-Thr-Glu-Ser-Ala-Leu-Leu-Tyr-Gln-Gly-Pro- (SEQ ID NO:92) was found. This amino acid sequence is identical to the expected N-terminal amino acid sequence of the α4 chain. For the band with the lowest Mr, no N-terminal amino acid sequence was found. This result is not surprising as the expected N-terminal amino acid residue in the β1 chain is a Gln-residue that is highly likely to cyclize, rendering the N-terminus inaccessible to amino acid sequence determination.

3. Functional cell-binding assay: To assess functionality of purified soluble VLA4-Fc receptor, transgenic CHO-K1 cells displaying 2D-VCAM-1-Cκ-V5 (either wildtype, Q38L, L80A or D40A) were incubated with a series of VLA4-Fc dilutions. The binding of soluble VLA4-Fc receptor to 2D-VCAM-1 was detected by incubating the cells with PE-conjugated anti-Human IgG Fc chain antibody. These results indicate that the soluble VLA4-Fc bound to the 2D-VCAM-1 protein displayed on the surface of CHO-K1 cells.

4. Functional ELISA assay: To compare functionality of later purification hatches of VLA4-Fc with a standard batch, we used the ELISA assay described in Example 10. The VLA4-Fc protein fractions and the standard. VLA4-Fc preparation were coated on the same ELISA plate at 2 ug/ml in TBS-M and the ELISA was performed using two different 2D-VCAM-1 variants, one of which was Q38L. Only fractions that were identical to the standard batch with both 2D-VCAM-1 variants were considered acceptable and pooled to generate a new batch of VLA4-Fc protein.

### Example 4

### Cloning, Expression, Purification, and Characterization of LPAM-1-Fc

### A. Construction of Human β7-Fc Plasmid

Human β7 integrin was cloned by PCR using a leukocyte cDNA library as template (Human Leukocyte Quick-Clone cDNA, Clontech, Catalog No. 637240) and oligonucleotides TBO395 and TBO396 (SEQ ID NOs:93 and 94 respectively) to obtain a PCR product containing the first 2169 nucleotides of human β7 integrin gene, which included the secretory signal sequence and the extracellular domain. The PCR product was reamplified using oligonucleotides TBO397 and TBO398 (SEQ ID NOs:95 and 96, respectively) to introduce flanking BamHI and SalI restriction sites. The BamHI and SalI sites were used to subclone the β7 fragment in place of the β1 fragment in plasmid pVM006 to obtain plasmid pVM012, which contained the β7 signal sequence and extracellular domain fused to IgG1-FcT366Y sequence. The sequence encoding the fusion was subsequently excised using BamHI and PmeI restriction enzymes and cloned in between the corresponding sites in plasmid pCDNA6-myc-His-A (Invitrogen, Catalog No. V22120) to obtain plasmid pVM013. Plasmid pVM013 contained a blasticidin resistance marker, and a CMV promoter which drove expression of soluble human β7 integrin-Fc^{T366Y} (referred to herein as “β7-Fc”; the nucleic acid sequence provided as SEQ ID NO:97 and amino acid sequence SEQ ID NO:98)

A UCOE-technology-based β7-Fc plasmid was also generated by subcloning the NheI-PmeI fragment from pVM013 into CET1019AS-hygro to digested with the same enzymes. The resulting plasmid was named pVM172.

### B. Construction of α4-Fc/β7-Fc CHO Cell Line

A cell line expressing α4-Fc/β7-Fc (also referred to herein as “LPAM-1-Fc” or “LPAM-1-Fc) were produced using a sequential approach essentially as described above for production of the α4-Fc/β1-Fc cell line. As described above, twenty-four clones exhibiting the highest α4-Fc recombinant protein production were pooled, amplified, and transfected with plasmid pVM013 using LIPTOFECTAMINE 2000 according to the manufacturers recommendations. Transgenic cell lines were obtained after approximately two weeks of growth in the presence of blasticidin antibiotic. Cells co-transfected with pVM007 and pVM013 were designated CHO-K1-007-013. Each cell pool was submitted to limited dilution cloning in five 96-well culture plates. After 2 weeks of growth, CHO-K1-007-013 clones were ranked for α4-Fc and β7-Fc production by means of the sandwich ELISA described in Example 9. Eight CHO-K1-007-013 clones were selected, transferred to T-flasks for further growth, and assessed for secreted heterodimer levels one week later using the sandwich ELISA. One clone was chosen for further recombinant protein production in roller bottles.

A second LPAM-1-Fc-expressing cell-line was generated from CHO-S cells using plasmids pVM162 and pVM172 through a sequential transfection process similar to the one described above.

### C. Expression and Purification of LPAM-1-Fc:

The chosen cell-line was transferred to roller bottles and adapted to serum-free medium. The supernatants were harvested daily and frozen at −80 C. At the end of the campaign, the supernatant was thawed, pooled, and purified by standard Protein A chromatography using a rProteinA-sepharose column (GE Healthcare) with glycine pH 3.2 as the elution buffer. The fractions were tested separately and functional fractions were pooled and stored at −80 C in the neutralized elution buffer.

### D. Characterization of LPAM-1-Fc:

Functional ELISA assay: To compare functionality of later purification batches of LPAM-1-Pc with a standard batch, we used the ELISA a4ssay described in Example 11. The LPAM-1-Fc protein fractions and a standard LPAM-1-Fc preparation were coated on the same ELISA plate at 2 ug/ml in TBS-M and the ELISA was performed using two different 2D-VCAM-1 variants, one of which was Q38L. Only fractions that were identical to the standard batch with both 2D-VCAM-1 variants were considered acceptable and pooled to generate a new batch of LPAM-1-Fc protein.

### Example 5

### Preparing CHO Cell-Surface Display and Phage Display Libraries

Regions in 2D-VCAM-1 responsible for binding to VLA4 were identified based on manual docking of available VCAM-1 structures into structures of integrins homologous to VLA4 (Newham, P. et al. (1997) J. Biol. Chem. 272:19429-19440; Jin, M. et al. (2006) Proc. Natl. Acad. Sci. USA 103: 5758-5763; Song, G. et al. (2006) J. Biol. Chem. 281:5042-5049). Residues on the surface of these regions predicted to be directly involved in VLA4 binding were included in the libraries. The following residues were targeted for mutagenesis: R36, T37, Q38, I39, S41, P42 (Library 1); F32, S34, T72, T74, K79, L80, E81 (Library 2); R123, L141, E142, D143, D145, R146, and K147 (Library 3).

A library design strategy based on the use of custom designed wobbles known as “doped” or “spiked” oligonucleotides was used. The wobbles were optimized to give an average mutation frequency of 2-3 mutations per variant. The design strategy was optimized to allow for all amino acids except cysteine, while minimizing the amount of stop codons and at the same time, minimizing the number of different wobbles for oligonucleotide synthesis.

For the CHO-cell-surface-displayed libraries 1 and 3, 2D-VCAM-1 in vector pVM036 was used as a template for PCR mutagenesis. The template was amplified with mutagenic primers in the targeted region and the flanking regions were amplified with non-mutagenic primers. The PCR fragments were assembled and amplified in a final PCR reaction with flanking primers that also introduced the BamHI and PstI cloning sites. The resulting library of variant sequences was digested with BamHI and PstI enzymes and used to replace the human 2D-VCAM-1 insert sequence in vector pVM036. The ligation was used to make the retroviral library as described in Example 6 below.

For library 2, human wildtype 2D-VCAM-1. (SEQ ID NO:2) was used as a template. Mutagenic primers spanning residues on the surface of domain-1 of 2D-VCAM-1 predicted to be directly involved in VLA4 binding were used to incorporate mutations at various positions. Several overlapping fragments were amplified using these primers and assembled by PCR. The resulting library of 2D-VCAM-1 variant sequences was amplified using primers to insert SfiI and NotI cloning sites at the 5′ and 3′ end of the inserts, respectively. The library products were digested with SfiI and NotI restriction enzymes, agarose gel-purified, ligated into the corresponding sites in pS0124 vector and transformed into E. coli TOP10 cells (Invitrogen) by electroporation. After 1 h of recovery, the transformants were grown in 2×YT media with 50 ug/mL carbenicillin overnight at 37° C. The resulting library culture used to generate library DNA using a maxiprep kit (Quiagen). A typical library contained approximately 9×108 independent transformants.

### Example 6

### Expression and Screening of 2D-VCAM-1 Variant Polypeptides Using CEO Cell-Surface Display

A retroviral library derived from pLenti6/V5-D-TOPO® was produced in 293FT cells following recommendations included in the ViraPower™ lentiviral kit (Invitrogen, Catalog No. K4950-00). Briefly, 293FT cells were co-transfected with the pLenti6/V5-D-TOP®-based library and the packaging plasmids (included in the ViraPower™ lentiviral kit) to produce retrovirus particles. After 48 hours, supernatants containing retroviral particles were harvested, filter-sterilized to remove cell debris, and subsequently used to infect CHO-K1 cells. Retrovirus titers were assessed by infecting CHO K1 cells with a series of virus dilutions and following manufacturer's recommendations, Blasticidin selection (5 mg/L) was started 3 days post-infection and the selection medium was replenished every 2-3 days until generation of a stable cell pool (12 days).

The CHO cell-surface displayed 2D-VCAM-1 variant library was submitted to several rounds of FACS-based enrichment. The first round aimed at enriching cells that displayed detectable levels of 2D-VCAM-1-Cκ-V5 recombinant protein, which were labeled with anti-V5-FITC mAb. Cells positive for the anti-V5-FITC mAb were collected (22% of the population) and amplified in cell culture. The enriched library cells were subjected to subsequent rounds of FACS aimed at sorting cells that displayed 2D-VCAM-1 variants that bound soluble VLA4-Fc. Cells were incubated with 200 nM VLA4-Fc at 21° C. for 1 h and bound VLA4-Fc was quantified by means of an anti-IgG1-PE monoclonal antibody. Recombinant protein display levels were assessed by co-staining the cells with an anti-V5-FITC monoclonal antibody. Cells that showed higher VLA4-Fc/V5 staining as compared with cells that displayed human 2DVCAM-1 were sorted and amplified in cell culture. The sorted populations represented 6% and 14.5% of the total cells in the subsequent rounds.

Finally, the library was submitted to two consecutive rounds of off-rate-based competition during the FACS analysis. Cells displaying the 2D-VCAM-1 variant library were allowed to bind VLA4-Fc in the presence of 5 μM Q38L-2D-VCAM-1 polypeptide (SEQ ID NO:10) prior to staining with anti-IgG-PE monoclonal antibody. Thus, only those cells that displayed 2D-VCAM-1 variants that bound VLA4-Fc better than the Q38L-2D-VCAM-1 variant were expected to retain substantial PE signal. Approximately 5% and 14% of the cells were sorted in the two consecutive off-rate-competition rounds.

Cells from rounds 4 and 5 of enrichment were seeded as single cells into a 96-well culture plate using limited dilution cloning and amplified to obtain clonal cell lines. These cell lines were subjected to FACS staining for VLA4-Fc and V5 binding and compared to control cells that expressed either the human 2D-VCAM-1 or the Q38L-2D-VCAM-1 polypeptides. In parallel, the 2D-VCAM-1 insert DNA was amplified by PCR from cell line and subjected to sequencing. The sequence data was correlated with the FACS binding data to identify 2D-VCAM-1 variants with improved VLA4-Fc binding.

### Example 7

### Expression, Panning, and Screening of 2D-VCAM-1 Variant Polypeptides Using Phage Display

Library DNA was transformed into E. coli TG1 cells (Stratagene) by electroporation. The library culture was grown overnight with the addition of M13K07 (˜1 E+10 cfu/ml) helper phage to support product of phage particles that displayed a library of 2D-VCAM-1 variants. The overnight culture was centrifuged for 10 min at 6,000 rpm and library phage particles in the supernatant were precipitated by incubation with one-fourth volume of 5×PEG/NaCl (20% w/v polyethylene glycol (PEG) 8000, 2.5M NaCl) for 30 min on ice and centrifugation for 40 min at 10,000 rpm. The phage pellet was resuspended in 1-2 ml TBS+1% BSA, clarified by centrifugation in a microfuge for 5 min. at Maximum speed and stored on ice until use.

NUNC Maxisorb™ immunotubes Were coated overnight at 4° C. with 1 mL of 2 ug/mL VLA4-Fc protein in TBS-M (25 mM Tris pH 7.5; 150 mM NaCl; 1 mM MnCl₂). An additional tube was coated with 1 mL of BSA as a control. The immunotubes were washed ire TBS-MT (TBS-M+0.05% TWEEN-20) and blocked at room temperature for 1 hr with 3% milk in TBS-MT. The library phage preparation from above was added to the immunotubes and allowed to bind for 2 h. The tubes were rigorously washed with TBS-T (TBS with 0.05% TWEEN-20) and bound phage particles were eluted with 0.2M Glycine, pH 2.2 in 1% BSA, neutralized with 1/10 volume 1 M Tris pH 9.0. The eluate was used to infect naïve TG1 cells and a sample the infection culture from both. VLA4- and BSA-coated tubes was plated on selective media to estimate the number of phage particles and to obtain individual colonies. The remaining culture from the VLA4 coated tube was used to prepare phage as described above. The panning process was repeated for five rounds.

A sample of the individual phagemid clones obtained after each round of enrichment was screened for VLA4-Fc binding using a phage ELISA format. Individual phagemid clones were inoculated into a 96-well plate containing 2×YT+Carb50 and used to make a high-throughput (HTP) phage preps as follows. The clones were first inoculated into 1 ml 2×YT+Carb50/well in a 96-well deepwell block and grown overnight in the presence of M13KO7 helper phage (Invitrogen). The blocks were centrifuged and the supernatants were recovered and screened for binding to VLA4-Fc using an ELISA assay. NUNC MAXISORP plates were coated overnight at 4° C. with 5 ug/ml of VLA4-Fc in TBS-M. After washing with TBS-MT, the phage supernatants were incubated for 1 h, washed, and the bound phage was detected by incubation with 1:5000 dilution of anti-M13-HRP antibody conjugate (GE Health Care). After washing with TBS-MT, the bound antibody signal was estimated by incubation with TMB-H₂O₂ (Pierce) and the reaction was stopped with 2M H₂SO₄ after color development. The plates were read for absorbance at 450 nm. The phagemid clones were also used to prepare phagemid DNA using a QIAGEN miniprep kit and sequenced. The sequence data was combined with the ELISA data to identify clones that appeared at the highest frequency and/or demonstrated good VLA4-Fc binding, which were purified as described in Example 8.

### Example 8

### Expression, Refolding, and Purification of 2-D VCAM-1 Variant Polypeptides

A. Subcloning of 2D-VCAM-1 Sequences for Expression in E. coli:

The 2D-VCAM-1 sequences to be expressed were subcloned into a bacterial expression vector, pVM-EcVec. Plasmid pVM-EcVec was derived from plasmid pQE81 (Clontech) and contains a colE1 origin and kanamycin resistance marker for propagation and selection in E. coli, an IPTG-inducible promoter consisting of the lac operator and T5 promoter sequences that drives the expression of the target gene and the lad repressor gene whose product controls the inducible promoter. The 2D-VCAM-1 gene to be subcloned was PCR amplified from the source vector (either the CHO-surface-display vector or phagemid vector obtained from the library screening) using primers which added a His-tag sequence to the 5′ end of the 2D-VCAM-1 sequence and a HindIII restriction site to the 3′ end. The PCR product was reamplified to add an EcoRI restriction site, a ribosome binding site, and an initiator ATG codon to the 5′ end of the 2D-VCAM-1 sequence. The PCR product was digested with EcoRI and HindIII and subcloned into the pVM-EcVec digested with the same enzymes. The resulting plasmid was sequenced to confirm the 2D-VCAM-1 sequence and then transformed into the W3110 E. coli strain for expression and purification of the 2D-VCAM-1 polypeptide.

Plasmids for expressing untagged versions of 2D-VCAM-1 variants were constructed as follows: plasmids containing the His-tagged versions of 2D-VCAM-1 variants (as described above) were used as template for PCR amplification using a forward primer that introduced an EcoRI restriction site, a ribosome binding site, and an initiator ATG codon immediately 5′ of the 2D-VCAM-1 variant ORF (open-reading frame) and a reverse primer that introduced a HindIII restriction site after the terminator codon of the 2D-VCAM-1 ORF. The PCR products were subcloned into pVM-EcVec using EcoRI and HindIII restriction enzymes as described above and then transformed into W3110 E. coil for expression and purification.

### B. Preparation of Bacterial Cultures:

Transgenic E. coli strains expressing the translational fusions of 2D-VCAM-1 variant polypeptide and His tag peptide were grown in 3 ml 2×YT liquid culture medium supplemented with 50 μg/mL Kanamycin at 37° C. for 4-6 hours in a shaker incubator. After 4-6 hours, the culture was transferred into a 500 ml shaker flask containing 100 ml 2×YT culture medium supplemented with 50 μg/mL Kanamycin and 0.5% glucose. The culture was allowed to grow overnight at 37° C. in a shaker incubator. The following day, the culture was transferred into a 1 L shaker flask containing 250 ml 2×YT liquid culture medium supplemented with 50 μg/mL Kanamycin. IPTG was added to 1.5 mM to the culture flask to promote expression of the recombinant protein. The culture was induced for 4 hours at 37° C. in a shaker incubator.

C. Isolation of Inclusion Bodies from E. coli:

Following IPTG treatment of the bacterial cultures, E. coli cells were isolated by centrifugation at 5000 g for 15 minutes. The supernatants were discarded. The bacterial pellets were stored at −20° C. until further use. The bacterial pellets were thawed for 1 hour before extraction. The thawed pellets were resuspended and lysed with BPER (Pierce, Catalog No. 78248) according to the manufacturer's instructions. After 20 minutes on ice, inclusion bodies were recovered by centrifugation at 15,000 g for 15 minutes. Supernatants were discarded. Pellets were then processed for purification.

### D. Purification:

1. Capture step for His-tagged 2D-VCAM variants: The isolated inclusion bodies from E. coli were dissolved in 30 ml of equilibration buffer for IMAC purification. Large debris was removed by centrifugation at 15,000 g for 15 minutes. The supernatant was decanted and equilibration buffer was added in a quantity sufficient to bring to the total volume of the supernatant to 40 mL The lysate was filtered over a 0.22 μm filter and subsequently applied onto a Ni-Sepharose column.

The Ni-Sepharose column was equilibrated with ten column volumes of 50 mM TRIS, 200 mM NaCl, 6 M Urea, 5 mM DTT pH 8.0. After application of the lysate, the column was washed with 20 column volumes of the equilibration buffer. The protein was eluted in 50 mM TRIS, 300 mM Imidazole, 6 M Urea, 2 mM DTT pH 8.0.

2. Refolding for His-tagged 2D-VCAM-1 variants: Refolding was accomplished by dilution into 50 mM, TRIS, 200 mM NaCl pH 8 containing 1 mM reduced glutathione and 1 mM oxidized glutathione. Protein concentration in the refold was kept to 0.1 mg/ml or less. A minimal dilution of 1/40 was used to negate urea and DTT effects. Refolding was achieved overnight at 4° C.

3. SEC Purification: In order to further purify the variant on a Superdex 200 column the refolded protein was first concentrated to approximately 1 ml using Centricon Plus 70 centrifugal filter devices. Approximately half of the concentrated protein was applied onto a Superdex 200 size exclusion column. The column was eluted in 50 mM TRIS, 200 mM NaCl pH 8.0. The purification was repeated with the second half of the concentrated protein. Proteins were analyzed by SDS-PAGE.

### Example 9

### ELISA Assays for α4-Fc, VLA4-Fc, and LPAM-1-Fc Expression

### A. ELISA Assay for α4-Fc:

A mouse anti-human IgG1 mAb specific for the Fc domain (Serotec, Catalog No. MCA514G) was adsorbed on a black MaxySorb 96-well plate (Nuns, Catalog No. 43711), using 100 μL PBS (Invitrogen, Catalog No. BE17-512F) supplemented with 2 μg/mL mAb. After approximately 16 hours incubation at 4° C., the wells were washed twice with 200 μL wash buffer (PBS-T) and subsequently blocked with 200 μL wash buffer containing 25 mg/mL casein (Fluka, Catalog No. 22080) for 90 min at 21° C. The plate was washed three times with wash buffer, incubated for 2 hours with culture medium samples (100 μL/well), and washed three times with wash buffer. Detection of α4-Fc recombinant protein was performed by incubating the plate with 100 μL/well PBS supplemented with 2.5 mg/mL Casein and 0.4 μg/mL biotinylated anti-human integrin α4 (Serotec, Catalog No. MCA1949B). After incubation at 21° C. for 90 minutes, the plate was washed three times and incubated for 30 minutes with 100 μL/well Horse Radish Peroxidase (HRP)-conjugated streptavidin (R&D Systems, Catalog No. 890803) diluted 200× in washing buffer supplemented with 2.5 mg/mL casein, HRP was detected by incubating the plate on an orbital shaker for 20 minutes with 100 μL/well. SuperSignal chemiluminescent substrate, (Pierce, catalog no. 37069). Luminescence signals were measured in a Microplate Scintillation and Luminescence Counter (Packard TopCount™ Liquid Scintillation Counter LSC).

### B. Sandwich ELISA for VLA4-Fc (α4-Fc/β1-Fc) Estimation:

NUNC MAXISORP plates were coated with anti-CD49d (i.e., anti-α4) antibody at 2 ug/ml in PBS overnight at 4° C. The plates were washed four times with PBS-T and blocked with 3% milk in PBS for 1 h at room temperature. After washing, the supernatant samples were titrated in a two-fold dilution. Each plate also included a standard VLA4-Fc two-fold titration series starting at 10 ug/ml. The binding was performed in the presence of 1% milk in PBS-T. After incubation for 1 h at room temperature, the plates were washed four times with PBS-T and incubated with 50 ul/well of biotinylated anti-CD29 (i.e., anti-β1) antibody at 0.4 ug/ml in PBS-T with 1% milk for 1 h at room temperature. The plates were washed four times with PBS-T and incubated with streptavidin-HRP at 1:2000 dilution in PBS-T and once again washed four times with PBS-T. HRP was detected by adding 50 ul/well TMBPlus substrate, incubating until color development at room temperature after which the reaction was stopped using 2M H₂SO₄ and the plates were read on an plate reader using SoftMaxPlus software. The data was analyzed using GraphPad Prism (GraphPad Software, Inc., La Jolla, Calif.) to obtain a standard curve, which was used to calculate the concentration of the heterodimer in the supernatant samples.

### C. Sandwich ELISA for LPAM-1-Fc (α4-Fc/β1-Fc) Estimation:

The sandwich ELISA used to estimate α4-Fc/β7-Fc heterodimers in supernatants was similar to the procedure used above with the following changes. The plates were coated with anti-β7 antibody at 2 ug/ml in PBS and the heterodimers were detected using biotinylated anti-α4 antibody at 0.4 ug/ml. A standard LPAM1-Fc titration was included on each plate to plot the standard curve.

### Example 10

### ELISA Assay for 2D-VCAM-1:VLA4-Fc binding

The relative binding affinities of various 2D-VCAM-1 variants to immobilized VLA4-Fc (α4-β1-Fc) heterodimer, relative to a control protein (e.g., Q38L-2D-VCAM-1 or to human 2D-VCAM-1), were deter using a solid-phase ELISA assay. MAXISORP™ ELISA plates (NUNC) were coated overnight at 4° C. with 50 ul VLA4-Fc heterodimer, 2 ug/ml in TBS-M (TBS (25 mM Tris-HCl pH 7.5, 150 mM NaCl) with 1 mM MnCl2), washed with TBS-T (TBS with 0.05% TWEEN-20), blocked with 200 ul 3% milk in TBS-M for 1 h at room temperature, and washed again with TBS-T. A four-fold dilution series of the purified 2D-VCAM variants to be assayed was made in COSTAR U-bottom plates in TBS-TMC (TBS with 0.05% Tween-20, 1 mM MnCl2, and 1% casein; Sigma) with a starting concentration of 8.2 uM (200 ug/ml). The dilution series was transferred into the ELISA plate which was incubated at room temperature for 2 h and subsequently washed with TBS-T. Bound 2D-VCAM protein was detected by incubation with 0.5 ug/ml of biotinylated anti-human VCAM antibody (R&D Systems, Catalog No. BAF809) in TBS-TMC for 1 h at room temperature followed by a TBS-MT (TBS plus 1 mM MnCl2 and 0.02% TWEEN-20) wash and subsequent incubation with 1:2000 dilution of streptavidin-HRP (BD Biosciences, Catalog No. 554066) in TBS-TMC for 30 min at room temperature. After a final wash with TBS-MT, 50 ul of IMBPlus substrate (Kern Diagnostics, Catalog No. 4390A) equilibrated to room temperature in the dark was added to each well and color development was followed visually. Typical development times ranged from 5-15 mM and development was stopped by addition of 50 ul 2M H₂SO₄ (EM, Catalog No. SX1244-75). Absorbance at 450 nM was measured promptly using a SpectraMax SoRmax Pro spectrophotometer (Molecular Devices, Sunnyvale, Calif.) and EC50 values were calculated by plotting absorbance against protein concentration using GraphPad Prism™ software (GraphPad Software, Inc., La Jolla, Calif.). Fold-improvement in binding was calculated relative to a control protein (e.g., Q38L-2D-VCAM-1 or human 2D-VCAM-1) assayed on the same plate. Table 3A shows the fold improvement in binding to VLA4 exhibited by representative 2D-VCAM-1 variant polypeptides of the invention, relative to either Q38L-2D-VCAM-1 (SEQ ID NO:10) or to human 2D-VCAM-1 (SEQ ID NO:2).

TABLE 3A
Fold-improvement in binding to VLA4-Fc in the ELISA assay

| | | Relative to | Relative to |
|---|---|---|---|
| 2D-VCAM-1 | SEQ | Q38L- | human |
| polypeptide | ID NO: | 2D-VCAM-1 | 2D-VCAM-1 |
| Q38L | 10 | 1 | 8 |
| Clone 47 | 14 | 16 | 128 |
| Clone 146 | 18 | 37 | 298 |
| Clone 46 | 12 | 21 | 172 |
| Clone 150 | 22 | 4 | 32 |
| Clone 147 | 20 | 11 | 88 |
| Clone 151 | 24 | 2 | 16 |
| Clone 59 | 16 | 34 | 272 |

Table 3B provides a Sequence Substitution Table

| | | Substitutions | | |
|---|---|---|---|---|
| 2D | | Relative to Wild- | Substitutions | Substitutions |
| VCAM-1 | SEQ | type 2D-VCAM-1 | Relative to | Relative to |
| polypeptide | ID NO: | (SEQ ID NO: 2) | SEQ ID NO: 12 | SEQ ID NO: 18 |
| Q38L | 10 | Q38L | P37T + L39I + | L32F + F34S + |
| | | | A41S | P37T + L39I + |
| | | | | R74T + S79K |
| Clone 47 | 14 | T37P + Q38L + | Q381 + A41S | L32F + F34S + |
| | | I39L | | R74T + S79K |
| Clone 146 | 18 | F32L + S34F + | F32L + S34F + | |
| | | T37P + Q38L + | Q38L + A41S + | |
| | | I39L + T74R + | T74R + K79S | |
| | | K79S | | |
| Clone 46 | 12 | T37P + I39L + | | L32F + F34S + |
| | | S41A | | L38Q + S41A + |
| | | | | R74T + S79K |
| Clone 150 | 22 | F32L + S34F + | F32L + S34F + | Q38L + S41A |
| | | T37P + I39L + | T74R + K79S | |
| | | S41A + T74R + | | |
| | | K79S | | |
| Clone 147 | 20 | F32S + S34Y + | F32S + S34Y + | L32Y + R74A + |
| | | T37P + Q38L + | Q38L + A41S + | S79R |
| | | I39L + T74A + | T74A + K79R | |
| | | K79R | | |
| Clone 151 | 24 | F32S + S34Y + | F32S + S34Y + | L32S + F34Y |
| | | T37P + I39L + | T74A + K79R | S41A + R74A + |
| | | S41A + T74A + | | S79R |
| | | K79R | | |
| Clone 59 | 16 | T37P + A38L + | Q38L + A41S + | L32F + F34S + |
| | | I39L + L141F + | L141F + D145A + | R74T + S79K + |
| | | D145A + R146W | R146W | L141F + D145A + |
| | | | | R146W |

### Example 11

### ELISA Assay for 2D-VCAM-1:LPAM-1-Fc Binding

The relative binding affinities of various 2D-VCAM variant proteins to immobilized LPAM-1-Fc (α4-Fc/β-7-Fc) heterodimer, relative to a control protein (e.g., Q38L-2D-VCAM-1 or human 2D-VCAM-1), were determined using a solid-phase ELISA assay. MAXISORP™ ELISA plates (NUNC) were coated overnight at 4° C. with 50 ul LPAM-1-Fc heterodimer (2 ug/ml in TBS-M, TBS (25 mM TRIS-HCl pH 7.5, 150 mM NaCl with 1 mM MnCl2), washed with TBS-T (TBS with 0.05% Tween-20), blocked with 200 ul 3% milk in TBS-M for 1 hr at room temperature, and washed again with TBS-T. A four-fold dilution series of the purified 2D-VCAM-1 variants to he assayed was made in COSTAR U-bottom plates in TBS-TMC (TBS with 0.05% Tween-20, 1 mM MnCl2 and 1% casein; Sigma) with a starting concentration of 8.2 uM (200 ug/ml). The dilution, series were transferred into the ELISA plate which was incubated at room temperature for 2 h and subsequently washed with TBS-T. Bound 2D-VCAM-1 protein was detected by incubation with 0.5 ug/ml of biotinylated anti-human VCAM antibody (R&D Systems, Catalog No. BAF809) in TBS-TMC for 1 h at room temperature followed by a TBS-T wash and subsequent incubation with 1:2000 dilution of streptavidin-HRP (BD Biosciences, Catalog No. 554066) in TBS-TMC for 30 min at room temperature. After a final wash with TBS-T, 50 ul of TMB Plus substrate (Kern Diagnostics, Catalog No. 4390A) equilibrated to room temperature in the dark was added to each well and color development was followed visually. Typical development times ranged from 5-15 min and development was stopped by addition of 50 ul 2M H₂SO₄ (EM, Catalog No. SX1244-75). Absorbance at 450 nM was measured promptly using a SpectraMax Sofimax Pro spectrophotometer (Molecular Devices, Sunnyvale, Calif.) and EC50 values were calculated by plotting absorbance against protein concentration using GraphPad Prism software (GraphPad Software, Inc., La Jolla, Calif.). Fold-improvement in binding was calculated relative to a control protein (Q38L-2D-VCAM-1) assayed on the same plate.

Table 4 shows the fold improvement in binding to LPAM-1 exhibited by representative 2D-VCAM-1 variant polypeptides of the invention, relative to Q38L-2D-VCAM-1.

TABLE 4
Fold improvement in binding to LPAM-1-Fc in the ELISA assay

| 2D-VCAM-1 | SEQ | Relative to |
|---|---|---|
| polypeptide | ID NO: | Q38L-2D-VCAM-1 |
| Q38L | 10 | 1 |
| Clone 47 | 14 | 17 |
| Clone 146 | 18 | 74 |
| Clone 46 | 12 | 1 |
| Clone 150 | 22 | 5 |
| Clone 147 | 20 | 23 |
| Clone 151 | 24 | n.d. |
| Clone 59 | 16 | 80 |

### Example 12

### Measuring Binding Affinities of 2D-VCAM-1 Variant Polypeptides to VLA4-Fc and LPAM-1-Fc Fusion Proteins Using Surface Plasmon Resonance (SPR: BIACORE Analysis)

This example describes a procedure for screening 2D-VCAM-1 variant polypeptides for improved binding affinity to VLA4-Fc and/or LPAM-1-Fc ligands using a Biacore interaction analysis. In the nomenclature used to describe this type of analysis, the immobilized binding partner is referred to as the “ligand”, and the binding partner in the mobile phase is referred to as the “analyte”. The strength of binding affinity is typically described in terms of the equilibrium dissociation constant (K_{D}), which describes the molar concentration of analyte at which 50% of available ligand is bound at equilibrium.

In this screening method, Biacore sensor chips were derivatized with anti-human IgG. After capture of VLA4-Fc or LPAM-1-Fc ligands on these surfaces, 2D-VCAM-1 variant polypeptides in buffer were allowed to flow over the ligand-coated sensor chips. The ability of a 2D-VCAM variant polypeptide to bind to a specific binding partner (i.e., VLA4-Fc or LPAM-1-Fc) was evaluated. Control protein (i.e., human 2D-VCAM-1 polypeptide (SEQ ID NO:2) or Q38L-2D-VCAM-1 polypeptide (SEQ ID NO:10)), was also allowed to flow over the ligand-coated sensor chips and the abilities of these molecules to bind to VLA4-Fc or LPAM-1-Fc were similarly evaluated for comparison. Using the Biacore system, the association (kₒₙ) and dissociation (k_{off}) rate constant of the protein of interest binding to VLA4-Fc or LPAM-1-Fc ligands can be evaluated and used to calculate the equilibrium dissociation constant, K_{D}. Variant 2D-VCAM-1 polypeptides having increased binding affinities for VLA4-Fc and/or LPAM-1-Fc, compared to human 2D-VCAM-1 and/or Q38L-2D-VCAM-1, were identified.

All BIACORE analyses were performed on a BIACORE 2000 system or BIACORE 3000 system (GE Healthcare) at room temperature (RT, 25° C.), HBS-P buffer (10 mM Hepes (pH 7.4), 150 mM NaCl, 0.005% surfactant P20) supplemented with 1 mM MnCl₂ (HBS-P-Mn) was used as the flow buffer for all experiments except where indicated.

### A. Kinetic 2D-VCAM-1 Binding Assay

The kinetic 2D-VCAM-1 assay measures binding kinetics of a dimeric ligand (e.g., VLA-Fc fusion protein or LPAM-1-Fc fusion protein) coated to sensor chips and monomeric analyte (e.g., 2D-VCAM-1 variant polypeptides) in the mobile phase. Goat anti-human IgG antibody (Jackson ImmunoResearch, Catalog No. 105-005-098) was immobilized on CM-5 sensor chips (GE Healthcare, Catalog No. BR-1000-14) according to the manufacturer's protocol. Antibody was diluted to 30 μg/ml in immobilization buffer (10 mM sodium acetate, pH 5.0 (GE Healthcare, Catalog No. BR-1003-51)). At a flow rate of 5 μl/minute, sensor chip CM-5 was activated with a 35 μl injection of an EDC/NHS mixture (made by mixing equal volumes of 11.5 mg/ml EDC and 75 mg/ml NHS (GE Healthcare, Catalog No. BM 000-50)), followed by a 35 μl injection of diluted antibody. Un-reacted sites were quenched with 35 μl of 1 M ethanolamine-HCl pH 8.5 (GE Healthcare, Catalog No. BR-1000-50). This procedure typically yielded 15,000 response units (RU) of coupled antibody. VLA4-Fc or LPAM-1-Fc, prepared as described in Examples 3 and 4, respectively, were bound to antibody-coated sensor chips by injection of 20 μl of ligand solution (20 μg/ml protein in HBS-P-Mn buffer at a flow rate of 10 μl/min). Ligand capture levels were typically 200-300 RU. 2D-VCAM-1 variant polypeptides were diluted in in HBS-P-Mn and flowed over ligand-coated sensor chips for 2 min at 30 μl/min, followed by 5 min incubation with in HBS-P-Mn containing no protein at the same flow rate. For 2D-VCAM-1 variant polypeptides having very slow dissociation rates from VLA4-Fc or LPAM-1-Fc, kinetic assays were also conducted using longer dissociation times (e.g., 20 min). Rmax signal levels for 2D-VCAM-1 variant polypeptides ranged from approximately 5-25 RU. Regeneration between cycles was performed by 200 sec incubation with 10 mM glycine buffer (pH 1.7) at 50 μl/min. New chips were subjected to 4-5 cycles of capture/binding/regeneration prior to use in actual experiments. Data from a reference cell containing goat anti-human IgG capture antibody alone was subtracted from data obtained from flow cells containing captured VLA4-Fc or LPAM-1-Fc. Typically, 8 dilutions of 2D-VCAM-1 variant polypeptides ranging from 150 nM to 0.069 nM were analyzed against a blank reference (HBS-P-Mn buffer alone). Each monomeric fusion protein comprises a mature 2D-VCAM-1 polypeptide, or a mature 2D-VCAM-1 variant polypeptide of the invention (such as, SEQ ID NO:12, 14, 16, 18, 20, 22, or 24), covalently fused at its C-terminus to a Histidine tag.

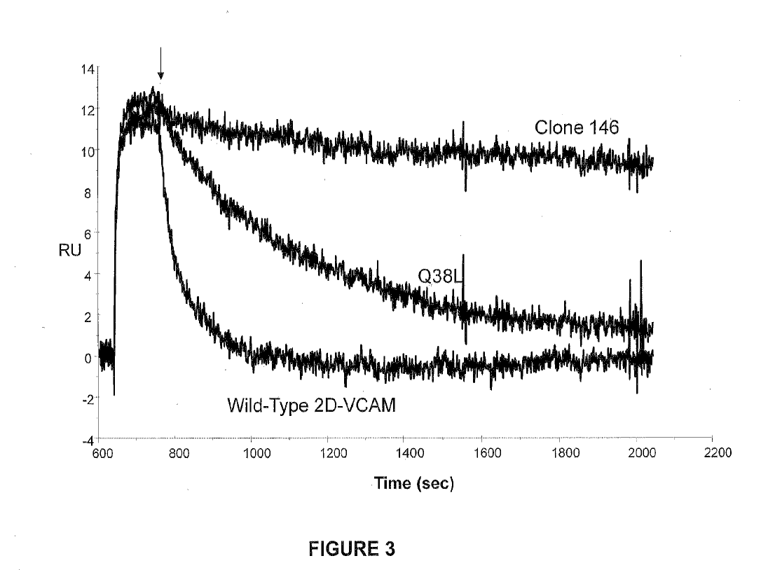

Sensorgram traces from a typical Biacore analysis are shown in FIG. 3. This figure shows the response (RU) over time (in seconds (s)) generated by the binding of the following 2D-VCAM-1 proteins to the VLA4-Fc fusion protein: (1) human 2D-VCAM-1 (SEQ ID NO:2, serving as a control and for comparison); (2) Q38L-2D-VCAM-1 (“Q38L,”, SEQ ID NO:10); and (3) an exemplary 2D-VCAM-1 variant polypeptide of the invention designated Clone 146 (SEQ ID NO:18).

The association phase reflects the binding between the analyte of interest and the ligand of interest. In FIG. 3, the association phase for each analyte is represented by the curve at times prior to the time marked by the arrow and is characterized by the binding of analyte (i.e., human 2D-VCAM-1 (SEQ ID NO:2) Q38L-2D-VCAM-1 (“Q38L”, SEQ ID NO:10) or 2D-VCAM-1 Clone 146 (SEQ ID NO:18)) to the VLA4-Fc ligand. The rate at which an analyte associates with the VLA4-Fc ligand is reflected in the curve—see, e.g., the sharp rate of increase in the response units beginning at about 620 seconds.

The dissociation phase of the analysis begins at the time marked by the arrow in FIG. 3. During the dissociation phase, the analyte and ligand dissociate from their bound conformation. In FIG. 3, the rate at which an analyte dissociates from the VLA4-Fc ligand is represented by the decrease in response units (rate of decrease of the response units over time). Based on these data, the relative dissociation rate constant (“off” rates, k_{off} or k_{d}) and association rate constants (“on” rates, kₒₙ or kₐ) can be determined. The total affinity of the interaction can be described by the K_{D}, (k_{off})/(kₒₙ). Increased binding affinities are often manifested in slower dissociation rates. If the slower dissociation rate is accompanied by an equal, higher, or marginally slower association rate, such that the calculated equilibrium dissociation constant K_{D} is lower, the affinity will be greater. In this case, a 2D-VCAM-1 variant polypeptide that has a binding affinity for the VLA4-Fc ligand that is greater than the binding affinity of the wild type 2D-VCAM-1 for the same ligand will also have a slower dissociation rate from the ligand than the wild type 2D-VCAM-1.

A 2D-VCAM-1 variant polypeptide that has a binding affinity for the VLA4-Fc ligand that is greater than the binding affinity of the Q38L-2D-VCAM-1 polypeptide for the same ligand may also have a slower dissociation rate from the ligand than the Q38L-2D-VCAM-1 polypeptide.

FIG. 3 shows that the dissociation rate of the Q38L-2D-VCAM-1 polypeptide from the VLA4-Fc ligand is significantly slower than the dissociation rate of the wild type 2D-VCAM-1 polypeptide from the same ligand. The Q38L-2D-VCAM-4 polypeptide also has an association rate for binding to VLA4-Fc ligand similar to that observed for wild type 2D-VCAM-1. Thus, the Q38L-2D-VCAM-1 polypeptide has a higher affinity for VLA4-Fc ligand as compared to wild type 2D-VCAM-1. FIG. 3 also shows that the exemplary 2D-VCAM-1 variant polypeptide of the present invention (Clone 146; SEQ ID NO:18) has a slower dissociation rate from the VLA4-Fc ligand than either wild type 2D-VCAM-1 or the Q38L-2D-VCAM-1 polypeptide. The exemplary 2D-VCAM-1 variant polypeptide also has a similar association rate for binding VLA4-Fc ligand compared to the association rates of either wild type 2D-VCAM-1 or the Q38L, 2D-VCAM-1 polypeptide for VLA4-Fc ligand. Thus, the exemplary 2D-VCAM-1 variant polypeptide of the present invention exhibits a higher binding affinity for the VLA4-Fc ligand than either wild type 2D-VCAM-1 or the Q38L-2D-VCAM-1 polypeptide. Therefore, the exemplary 2D-VCAM-1 variant polypeptide of the present invention would be expected to bind native VLA4, including, e.g., activated VLA4 as expressed in vivo on rolling lymphocytes in mammals, such as humans, with higher binding affinity. This conclusion is further supported by the functional cell-based assays discussed in the Examples below.

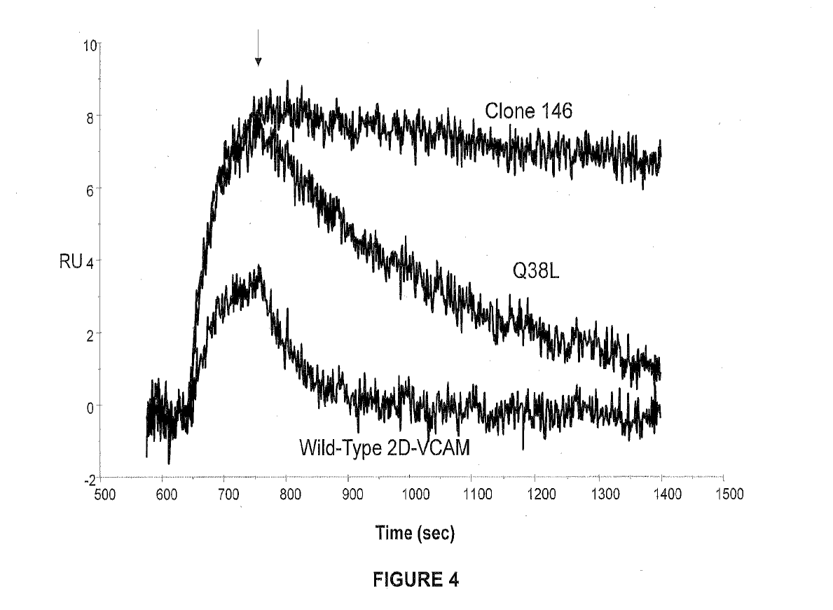

Biacore analyses were performed as described above using other 2D-VCAM-1 variant polypeptides of the present invention. In addition, Biacore analyses were performed with LPAM-1-Fc -coated sensor chips and 2D-VCAM-1 variant polypeptides of the present invention, FIG. 4 shows sensorgram traces of (RU) over time (in seconds (s)) generated by the binding of the following 2D-VCAM-1 proteins to the LPAM-1-Fc fusion protein: (1) human 2D-VCAM-1 (SEQ ID NO:2, serving as a control and for comparison); (2) Q38L-2D-VCAM-1 (“Q38L”, SEQ ID NO:10); and (3) an exemplary 2D-VCAM-1 variant polypeptide of the invention designated Clone 146 (SEQ ID NO:18).

The dissociation rates and binding affinities of these 2D-VCAM-1 variant polypeptides were determined and compared to the dissociation rates and binding affinities of wild type 2D-VCAM-1 and Q38L-2D-VCAM-1 polypeptides. Representative results are shown below in Tables 5 and 6.

### B. Standard BIACORE Data Analyses

After deletion of the regeneration and capture portions of the sensorgrams, curves were zeroed to a 5 s average of all curves approximately 10 s prior to sample injection. A blank curve was subtracted from each test curve. Data were analyzed by BIAevaluation software (v 4.1, available from GE Healthcare) using the “Fit kinetics, Simultaneous kₐ/k_{d}” function. The injection start time was defined as a time prior to the association phase where all curves were close to zero. Data selection for the association phase began approximately 10 s after the injection start time and ended approximately 10 a prior to the injection stop time. The injection stop time was defined as a time prior to the appearance of any signal spikes associated with the dissociation phase. The dissociation phase was selected to start 10 s after the injection stop time and included 280-295 s of the 5 min dissociation phase. The 1:1 Langmuir model describes the reaction A+B<=>AB. This model represents a single ligand binding to a single protein of interest (e.g., receptor). The 1:1 Langmuir model from the BIAevaluation software was used to determine the association rate constant (ka) and the dissociation rate constant (kd) and to calculate the equilibrium dissociation constant, K_{D}. K_{D}=k_{d}/kₐ. K_{D}=([A].[B])/[AB]. The equilibrium dissociation constant, K_{D}, is equal to the inverse of the equilibrium association constant, K_{A}. K_{D}=1/K_{A}. The rate equations for the reaction (analyte A plus ligand B yielding complex AB), where A=analyte injected, B=free ligand, and t=time, are: d[B]/dt=−(kₐ[A][B ]−k_{d}[AB]) and d[AB]/dt=kₐ[A][B]−kd[AB], Substituting R, Biacore response units (RU) at a given time, for [AB], Rmax-R for [B], and C (analyte concentration) for [A], the net rate expression in Biacore units is dR/dt=kₐC(Rmax-R)−k_{d}R, where R at t₀=0, B[0]=Rmax, and AB[0]=0 RU, with the total response=[AB]+RI. The bulk shift (RI) was set to zero, and Rmax, ka and led were fit globally for all curves.

The standard kinetic assays and data analyses described above were performed on preparations of representative 2D-VCAM-1 variant polypeptides of the invention, as well as the Q38L 2D-VCAM-1 polypeptide and the human 2D-VCAM-1 polypeptide. Tables 5 and 6 summarize the binding data for representative 2D-VCAM-1 variant polypeptides of the invention.

TABLE 5A

| | | | Binding Affinity for |
|---|---|---|---|
| | | | VLA4-Fc |
| 2D-VCAM-1 | SEQ | VLA4-Fc K_{D} | (relative to human |
| Polypeptide | ID NO | (M) | 2D-VCAM-1) |
| human 2D-VCAM-1 | 2 | 1.52 × 10⁻⁸ | 1 |
| Q38L | 10 | 1.87 × 10⁻⁹ | 8 |
| Clone 150 | 22 | 4.17 × 10⁻¹⁰ | 36 |
| Clone 47 | 14 | 1.48 × 10⁻¹⁰ | 103 |
| Clone 46 | 12 | 1.23 × 10⁻¹⁰ | 124 |
| Clone 59 | 16 | 5.01 × 10⁻¹¹ | 303 |
| Clone 146 | 18 | 4.97 × 10⁻¹¹ | 306 |

Table 5A presents binding affinities of representative 2D-VCAM-1 variant polypeptides of the invention to VLA4-Fc, as measured by the standard BIACORE assay described above. Specifically, Table 5A shows the designation of each 2D-VCAM-1 variant polypeptide of the invention; the sequence identification number (SEQ ID NO) corresponding to the polypeptide sequence of the 2D-VCAM-1 polypeptide or polypeptide variant; the equilibrium dissociation constant (K_{D} (Molar (M)) determined based on the binding of the 2D-VCAM-1 variant polypeptide to VLA4-Fc; and the binding affinity of each 2D-VCAM-1 variant polypeptide to VLA4-Fc relative to the binding affinity of wild type 2D-VCAM-1 to VLA4-Fc. This relative binding affinity (shown in the far-right column) is shown as a fold improvement in binding affinity of the 2D-VCAM-1 variant polypeptide to VLA4-Fc as compared to the binding affinity of wild type 2D-VCAM-1 polypeptide to VLA4-Fc. In addition, the fold improvement in VLA4-Fc binding affinity of the Q38L, 2D-VCAM-1 polypeptide as compared to VLA-4-Fc binding affinity of human 2D-VCAM is also shown.

As shown in Table 5A, representative 2D-VCAM-1 variant polypeptides of the invention have VLA4-Fc binding affinities that are: (1) at least about equal to or greater than the binding affinity of wild type 2D-VCAM-1 to the VLA4-Fc ligand; and/or (2) at least about equal to or greater than the binding affinity of the O38L, 2D-VCAM-1 polypeptide to the VLA4-Fc ligand. The fold improvement in binding affinity to the VLA4-1-Fc ligand relative to the binding affinity of the wild type 2D-VCAM-1 to VLA4-Fc is indicated for each representative 2D-VCAM-1 mutant (See 4th column in Table 5A).

Many of the 2D-VCAM-1 variant polypeptides of the invention were found to have a rate of dissociation from VLA4-Fc fusion protein that is slower than the rate of dissociation of wild type 2D-VCAM-1 from VLA4-Fc.

All of the representative 2D-VCAM-1 variant polypeptides shown in Table 5A had VLA4-Fc equilibrium dissociation constants (K_{D}) that were lower than the VLA4-Fc equilibrium dissociation constant for Wild type 2D-VCAM-1. Furthermore, most of the 2D-VCAM-1 variant polypeptides shown in Table 5A had VLA4-Fc equilibrium dissociation constants that were lower than the VLA4-Fc equilibrium constant for the Q38L, 2D-VCAM-1 polypeptide.

All of the representative 2D-VCAM-1 variant polypeptides shown in Table 5A had binding affinities for VLA4-Fc that are greater than the binding affinities of the human wild type 2D-VCAM-1 for VLA4-Fc (the calculated fold improvement in VLA4-Fc binding affinity relative to wild type 2D-VCAM-1 is shown the 4th column of Table 5A). Moreover, many of the 2D-VCAM-1 variant polypeptides shown in Table 5A had binding affinities for VLA4-Fc that are greater than the binding affinity of the Q38L, 2D-VCAM-1 polypeptide for VLA4-Fc (see 3^{rd} column of Table 5A).

A 2D-VCAM-1 variant polypeptide of the present invention which has a higher binding affinity to the VLA4-Fc ligand, as compared to VLA4-Fc binding affinity for human 2D-VCAM-1 or the Q38L, 2D-VCAM1 polypeptide, will likely have improved efficacy in suppression of inflammation in vivo compared to the human 2D-VCAM-1 or the Q38L 2D-VCAM-1 polypeptide, respectively. Such a 2D-VCAM-1 variant polypeptide of the present invention may be highly effective in inhibiting the recruitment of lymphocytes and monocytes to sites of inflammation, and thus be an effective treatment for a variety of inflammatory diseases and autoimmunity-related disorders.

Table 5B presents binding affinities of representative 2D-VCAM-1 variant polypeptides of the invention to VLA4-Fc when retested with new polypeptide preparations and new VLA-Fc ligand and measured by the standard BIACORE assay described above. The controls human 2D-VCAM-1 or the Q38L 2D-VCAM-1 polypeptide was not reassayed.

TABLE 5B

| 2D-VCAM-1 | SEQ | VLA4-Fc K_{D} |
|---|---|---|
| Polypeptide | ID NO | (M) |
| Clone 46 | 12 | 1.95 × 10⁻⁹ |
| Clone 47 | 14 | 6.8 × 10⁻¹⁰ |
| Clone 59 | 16 | 9.5 × 10⁻¹¹ |
| Clone 146 | 18 | 1.8 × 10⁻¹¹ |

Table 6A presents binding affinities of representative 2D-VCAM-1 variant polypeptides of the invention to LPAM-1-Fc, as measured by the standard BIACORE assay.

TABLE 6A

| | | | Binding Affinity |
|---|---|---|---|
| | | | for LPAM-1-Fc |
| 2D-VCAM-1 | SEQ | LPAM-1-Fc K_{D} | (relative to human |
| Polypeptide | ID NO | (M) | 2D-VCAM-1) |
| human 2D-VCAM-1 | 2 | 2.48 × 10⁻⁷ | 1 |
| Clone 150 | 22 | 3.12 × 10⁻⁸ | 8 |
| Clone 46 | 12 | 2.72 × 10⁻⁸ | 9 |
| Q38L | 10 | 2.05 × 10⁻⁸ | 12 |
| Clone 47 | 14 | 6.54 × 10⁻⁹ | 38 |
| Clone 146 | 18 | 4.39 × 10⁻¹⁰ | 565 |
| Clone 59 | 16 | 4.05 × 10⁻¹⁰ | 612 |

Specifically, for each representative 2D-VCAM-1 variant polypeptide of the invention, Table 6A provides equilibrium dissociation constants (K_{D} (Molar (M)) for LPAM-1-Fc ligand binding, and the binding affinity to LPAM-1-Fc relative to binding affinity of wild type 2D-VCAM-1 fusion protein to the same ligand. For each 2D-VCAM-1 variant polypeptide, the fold improvement in binding affinity to the LPAM-1-Fc ligand compared to the binding affinity of wild type 2D-VCAM4 fusion protein to the LPAM-1,-Fc ligand is shown (see 4th column in Table 6A). Wild type human 2D-VCAM-1 serves as the reference, i.e., with the binding affinity to LPAM-1-Fc set to 1. As shown in Table 6A, the representative 2D-VCAM-1 variant polypeptides of the invention have binding affinities to the LPAM-1-Fc ligand that are (1) at least about equal to or greater than the binding affinity of wild type 2D-VCAM-1 to the LPAM-1-Fc ligand; and/or (2) at least about equal to or greater than the binding affinity of the Q38L 2D-VCAM-1 polypeptide to the LPAM-1-Fc ligand.

A number of 2D-VCAM-1 variant polypeptides were found to have a rate of dissociation from LPAM-1-Fc ligand that is about equal to or greater than the rate of dissociation of the wild type 2D-VCAM-1 from the same ligand. Some 2D-VCAM-1 variant polypeptide were found to have a rate of association to LPAM-1-Fc about equal to or greater than the rate of association of wild type 2D-VCAM-1 fusion protein to the same ligand.

Many representative 2D-VCAM-1 variant polypeptides shown in Table 6 had LPAM-1-Fc equilibrium dissociation constants (K_{D}) that were lower than the LPAM-1-Fc equilibrium dissociation constant of wild type 2D-VCAM-1. Furthermore, several representative 2D-VCAM-1 variant polypeptides shown in Table 6A had LPAM-1-Fc equilibrium dissociation constants that were at least about equal to the LPAM-1-Fc equilibrium constant of the Q38L, 2D-VCAM-1 polypeptide.

Many 2D-VCAM-1 variant polypeptides shown in Table 6A had binding affinities for LPAM-1-Fc that are greater than the binding affinity of human wild type 2D-VCAM-1 for the same ligand (fold improvement in LPAM-1-Fc binding affinity relative to wild type 2D-VCAM-1 is shown in the 4th column of Table 6A). Additionally, several 2D-VCAM-1 variant polypeptides shown in Table 6A had binding affinities for LPAM-1-Fc that are about equal to the binding affinity of the Q38L-2D-VCAM-1 polypeptide for the same ligand.

Table 6B presents binding affinities of representative 2D-VCAM-1 variant polypeptides of the invention to LPAM-1-Fc when retested with new polypeptide preparations and new LPAM-1-Fc ligand and measured by the standard BICORE assay described above. The controls human 2D-VCAM-1 or the Q38L 2D-VCAM-1 polypeptide was not reassayed.

TABLE 6B

| 2D-VCAM-1 | SEQ | LPAM-1-Fc K_{D} |
|---|---|---|
| Polypeptide | ID NO | (M) |
| Clone 46 | 12 | 7.7 × 10⁻⁸ |
| Clone 47 | 14 | 4.0 × 10⁻⁸ |
| Clone 59 | 16 | 2.6 × 10⁻⁹ |
| Clone 146 | 18 | 1.2 × 10⁻⁹ |

Comparing the binding affinities for VLA4 (Table 5A) and LPAM-1 (Table 6A), it is apparent that many of the exemplary 2D-VCAM-1 variant polypeptides of the invention exhibit a ratio of VLA4/LPAM-1 binding affinity which is greater than the ratio of VLA4/LPAM-1 binding affinities exhibited by either human 2D-VCAM-1 or Q38L-2D-VCAM-1. In other words, some 2D-VCAM-1 variants of the invention exhibit a greater improvement in binding affinity to VLA4 (integrin α4β1) than in binding affinity to LPAM-1 (integrin α4β7), in comparison to the relative affinities of either human 2D-VCAM-1 or Q38L-2D-VCAM-1 for VLA4 versus LPAM-1.

Table 7 provides ratios of the binding affinities for VLA4-Fc versus LPAM4-Fc for the Q38L-2D-VCAM-1 polypeptide and for representative 2D-VCAM-1 variant polypeptides of the invention, relative to that of human 2D-VCAM-1. The ratios are derived from experimental data shown in Tables 5A and 6A, which is based on experiments in which both controls and variants were assayed.

TABLE 7

| | | Binding Affinity | Binding Affinity | VLA4/LPAM-1 | VLA4/LPAM-1 |
|---|---|---|---|---|---|
| | | for VLA4-Fc | for LPAM-1-Fc | Binding Affinity | Binding Affinity |
| | | (relative to | (relative to | Ratio (relative | Ratio (relative |
| | SEQ | human | human | to human | to Q38L- |
| 2D-VCAM-1 | ID NO | 2D-VCAM-1) | 2D-VCAM-1) | 2D-VCAM-1) | 2D-VCAM-1) |
| human 2D-VCAM-1 | 2 | 1 | 1 | 1 | 1.4 |
| Q38L | 10 | 8 | 12 | 0.7 | 1 |
| Clone 150 | 22 | 36 | 8 | 4.5 | 6.4 |
| Clone 47 | 14 | 103 | 38 | 2.7 | 3.8 |
| Clone 46 | 12 | 124 | 9 | 14 | 20 |
| Clone 59 | 16 | 303 | 612 | 0.5 | 0.7 |
| Clone 146 | 18 | 306 | 565 | 0.5 | 0.7 |

A 2D-VCAM-1 variant polypeptide having a high VLA4/LPAM-1 binding affinity ratio, relative to that of human 2D-VCAM-1 or Q38L-2D-VCAM-1 (e.g., a VLA4/LPAM-1 binding affinity ratio of greater than 2, such as at least 4, at least 5, at least 6, or at least 10), may be advantageous if LPAM-1 binding is undesirable, for instance if LPAM-1 binding is correlated with adverse side effects, such as progressive multifocal leukoencephalopathy (PML).

### Example 13

### Static Cell-Adhesion Assay for α4 Integrin Blockade

The ability of the 2D-VCAM-1 variants to compete with immobilized VCAM-1 protein for binding to cell-surface human VLA4 was quantitated using a U937-VCAM-1 cell adhesion assay. U937 cells (ATCC) hearing VLA4 on the cell surface were labeled with fluorescent dye Calcein AM (Invitrogen) and allowed to bind 7D-VCAM-1 in the presence or absence of 2D-VCAM-1 variant polypeptides and control. A reduction in fluorescence intensity associated with adherent cells reflected competitive inhibition of VLA4 mediated cell adhesion by the 2D-VCAM-1 variant polypeptides and controls.

Initially, this entailed coating 96 well plates (MICROLON 600 High Binding; Greiner Bio-One) with recombinant human 7D-VCAM-1 (SEQ ID NO:6, 7 domain form of VCAM-1, R&D Systems) at 5 μg/ml in 100 μl coating buffer (HEPES CaMg buffer: 50 mM HEPES, 150mM NaCl, 1 mM CaCl₂, 1 mM MgCl₂, pH 7.4), sealing each plate and allowing the plates to incubate for 2 hours at 37° C. The coating solution was decanted by inversion and 200 μl blocking buffer (HEPES CaMg buffer with 2% BSA) was added and incubated at room temperature for another hour. Just prior to assaying, the VCAM-1-coated plates were washed three times with 0.05% TWEEN-20 in PBS by hand or by using an automatic 96 well plate washer (Molecular Devices, Sunnyvale, Calif.), then the plates were inverted and blotted,

The U937 cells were maintained in RPMI 1640 culture media (RPMI media 1640 with phenol red, Invitrogen) containing 10% PBS, 1% Pen/Strep (Invitrogen). Prior to assaying, the U937 cells were re-suspended in labeling buffer (RPMI 1640 culture media without phenol red, 10% FBS; Invitrogen), then labeled with 0.5 μg/ml Calcein-AM at 37° C. for 30 minutes on a rotator. Subsequently, the cells were washed three times with labeling buffer then resuspended at 5×106 cells/ml in assay buffer (HEPES CaMgMn buffer: 50 mM HEPES, 150 mM NaCl, 1 mM CaCl₂, 1 mM MgCl₂, 1 mM MnCl2 pH 7.4).

The test compounds (i.e., the 2D-VCAM-1 variant polypeptides) were diluted in dilution buffer and added to VCAM-1-coated plates. VCAM-1 coated wells filled with 100 μl of dilution buffer (assay buffer above, with 0.1% BSA) were used as a control to show maximal cell adherence to the immobilized VCAM-1. 100 μl of 10 μg/ml anti-VLA-4 antibody (R&D Systems, Clone 2B4) was used as positive control showing maximal inhibition of cell adherence. Wells which were coated with non-VCAM protein (i.e., blocked with BSA) were used as the “Blank”. 100 μl of Calcein-AM labeled cells were added to each well. Plates were incubated at room temperature for 20 minutes and washed with assay buffer in a static cell adhesion wash chamber (GlycoTeeh). Plates were spun down at 280×g for 5 minutes with assay buffer decanted by inversion before measuring fluorescence intensity (excitation=485 nm, emission=530 nm) with LJL Analyst HT 96.384 (Molecular Devices, Sunnyvale, Calif.).

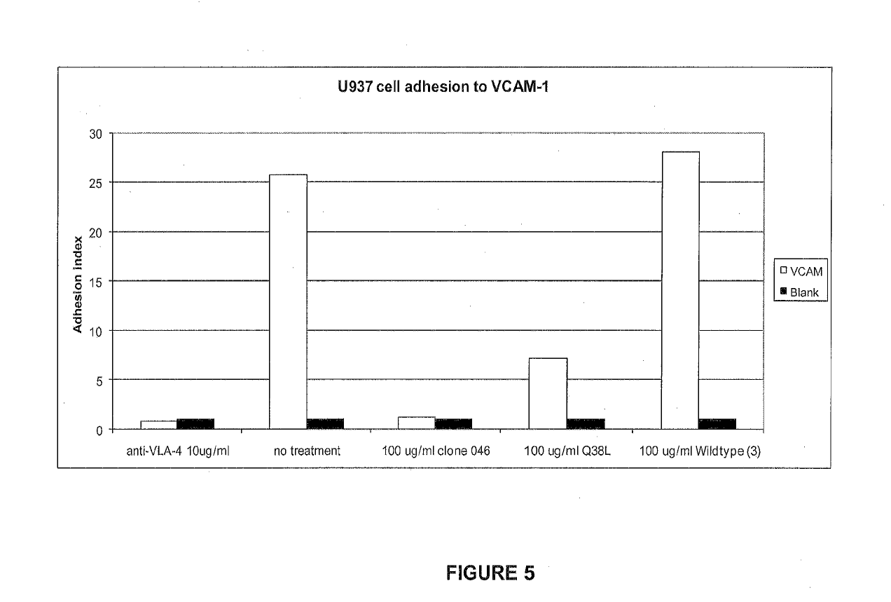

FIG. 5 is a graph showing the cell adhesion index for anti-VLA4-antibody (used as a positive control for maximal inhibition of cell adherence), dilution buffer alone (“no treatment”, a control to show maximal cell adherence to the immobilized VCAM-1), 2D-VCAM-1 valiant Clone 46 (SEQ ID NO:12), Q38L-2D-VCAM-1 polypeptide (SEQ ID NO:10), and human 2D-VCAM-1 (SEQ ID NO:2). The cell adhesion index was calculated as (fluorescence intensity, VCAM-coated well)/(fluorescence intensity, non-CAM-1-coated well). The non-VCAM-1-coated wells (“blank”) are given a cell adhesion index=1. FIG. 5 shows that 100 μg/ml of 2D-VCAM-1 variant Clone 46 inhibited U937 cell adhesion essentially as well as did the anti-VLA4 antibody. On the other hand, 100 μg/ml Q38L-2D-VCAM-1 showed only partial inhibition of U937 cell adhesion, while 100 μg/ml human 2D-VCAM-1 showed essentially no inhibition of U937 cell adhesion.

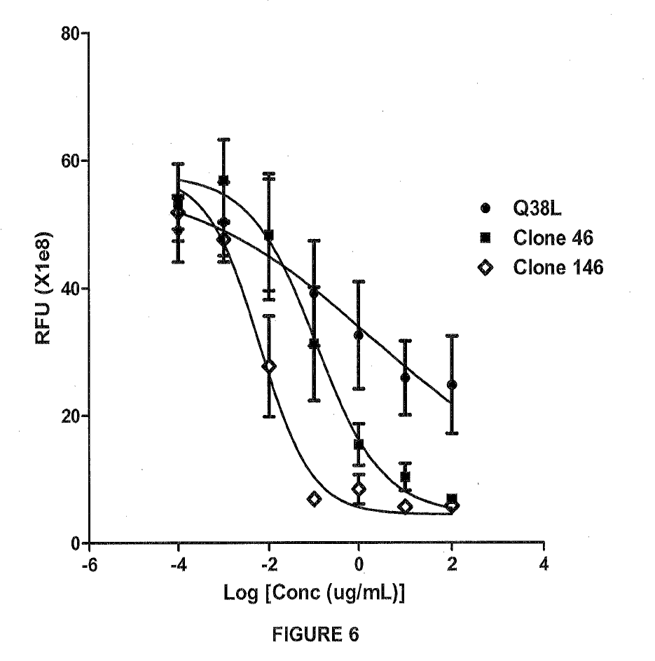

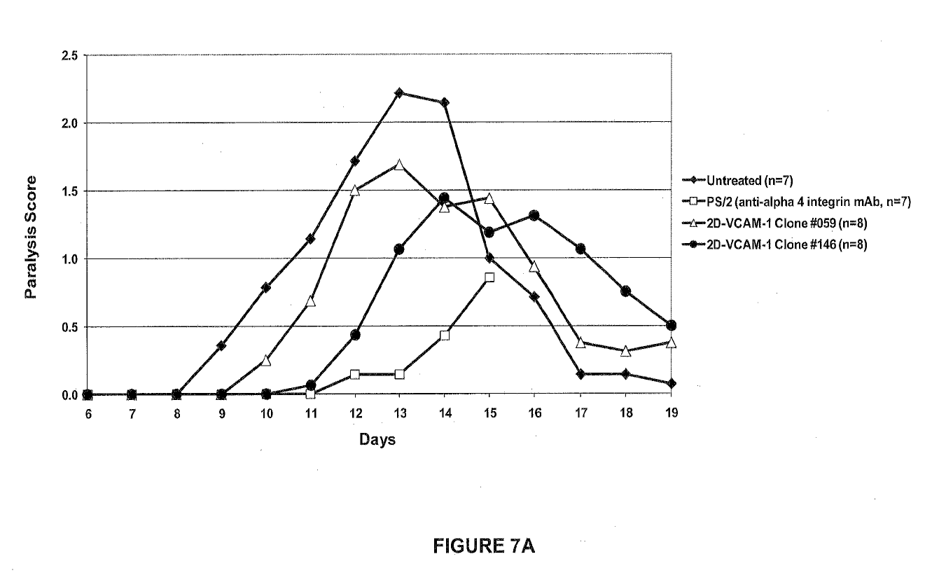

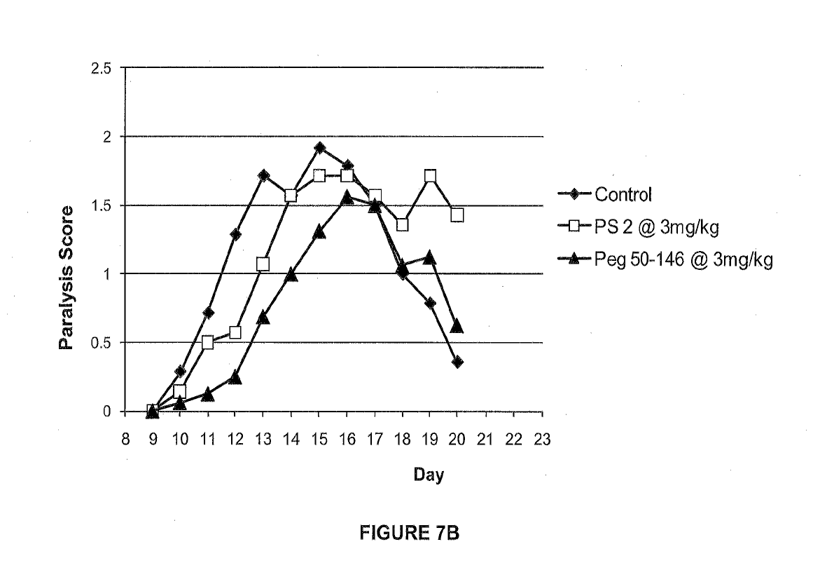

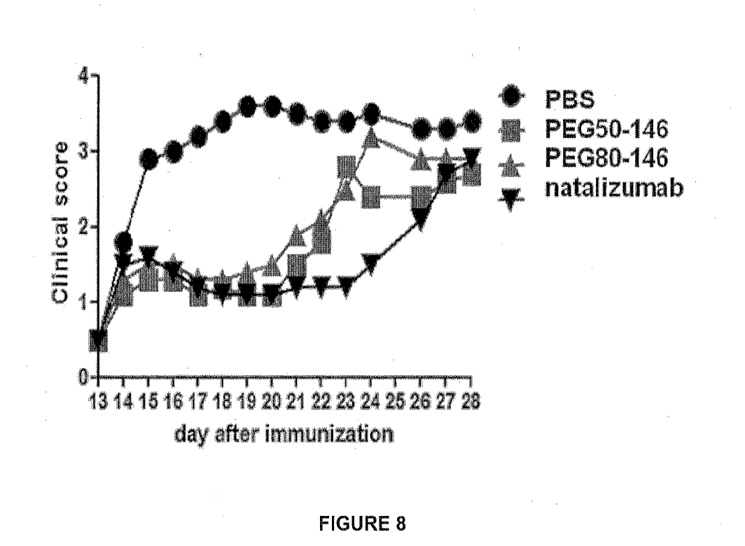

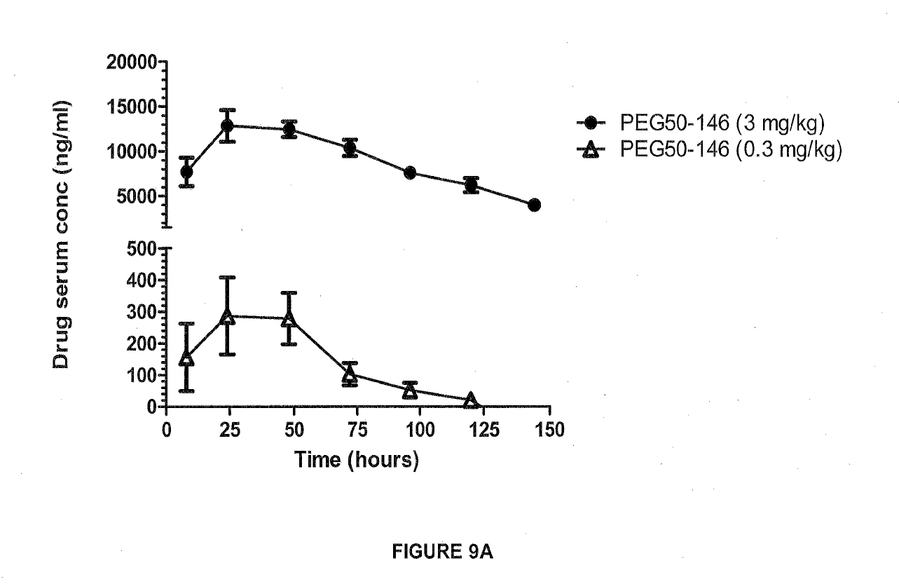

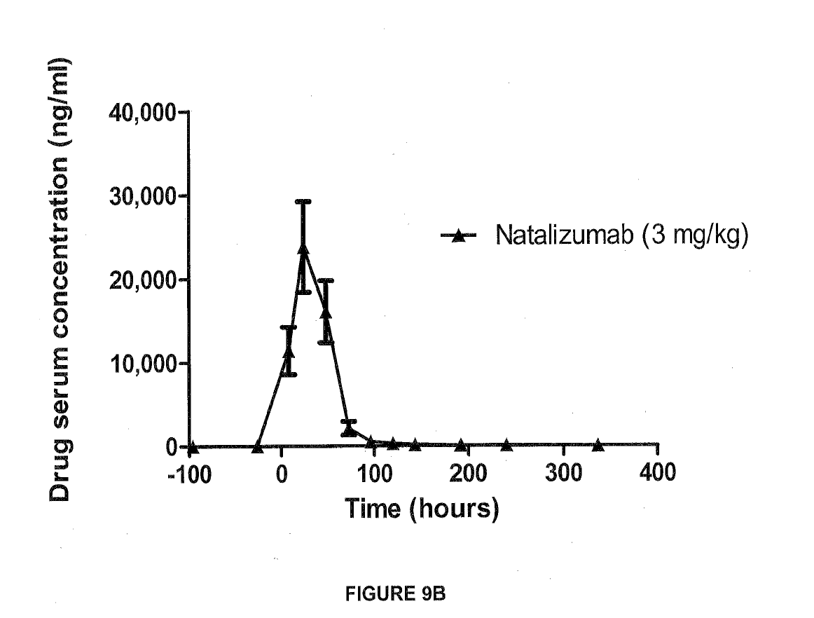

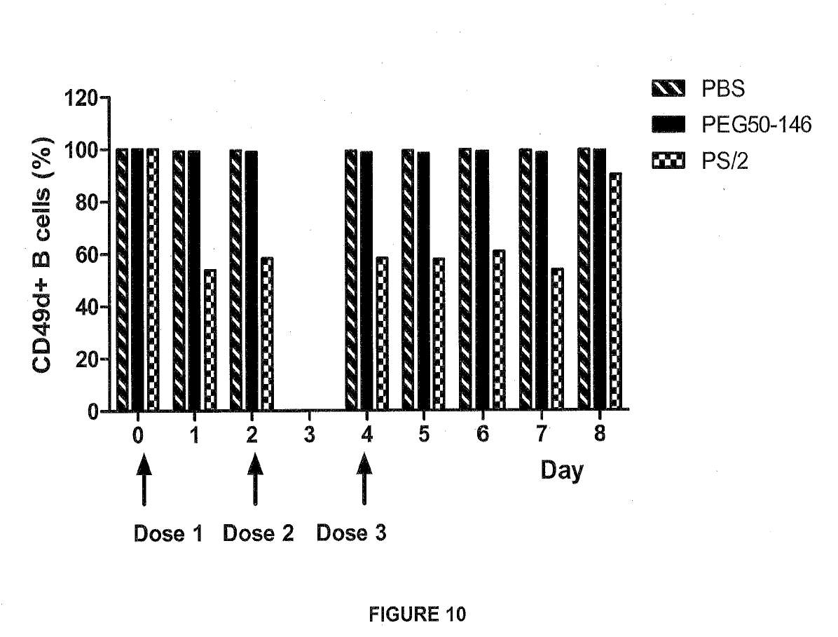

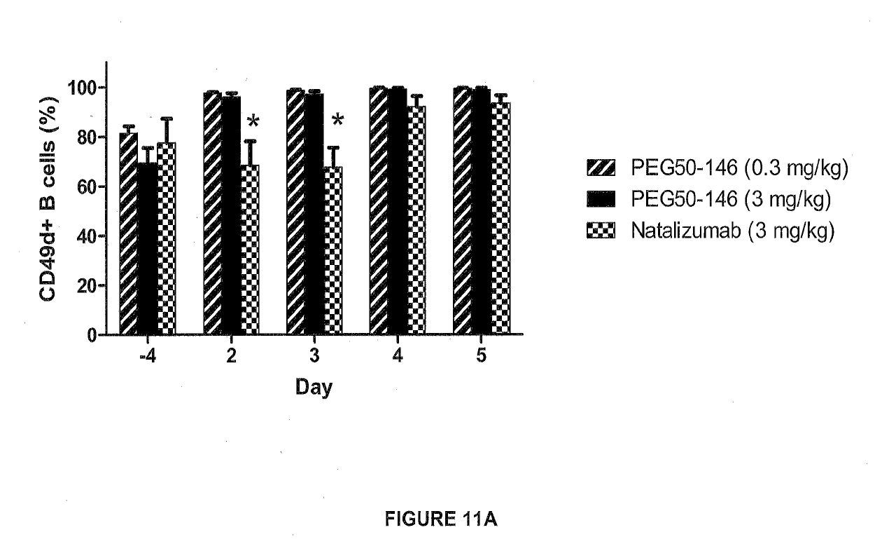

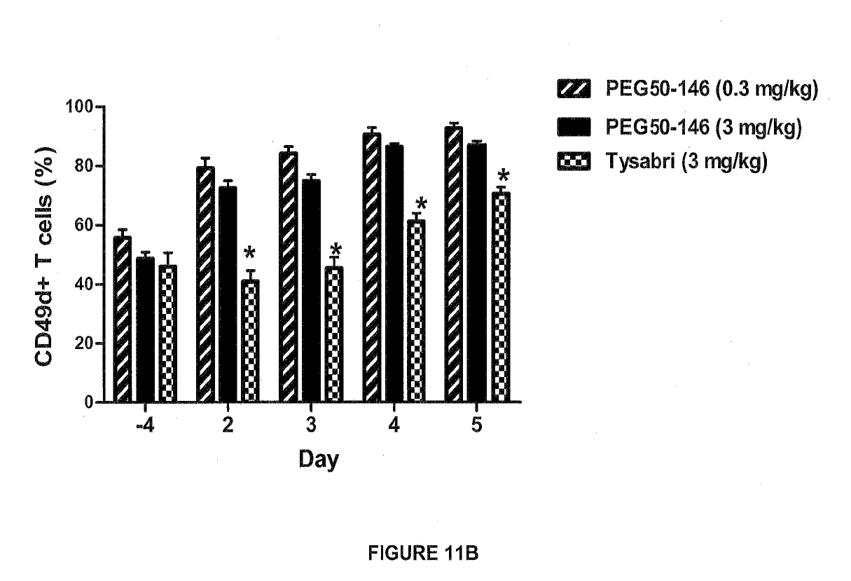

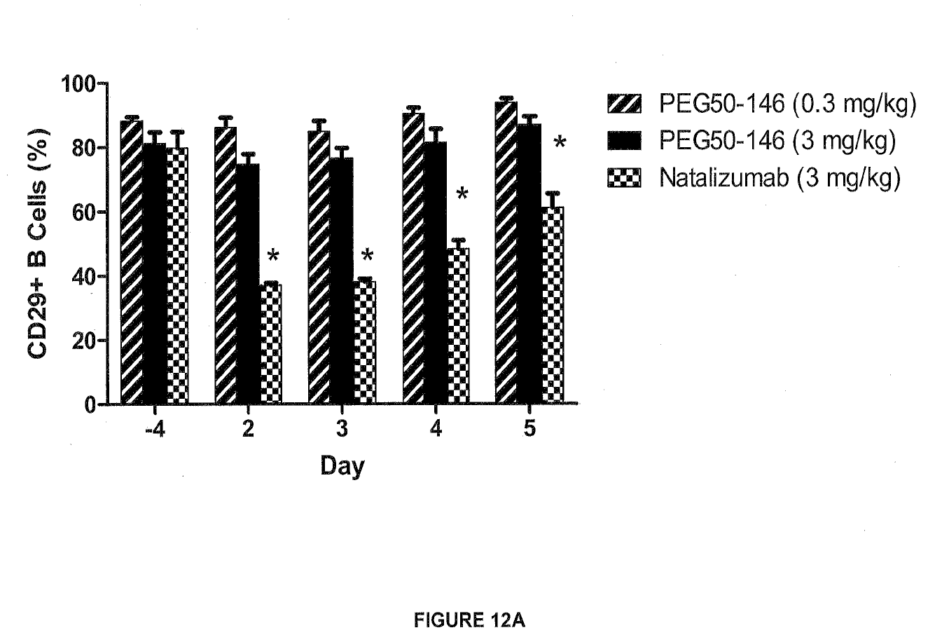

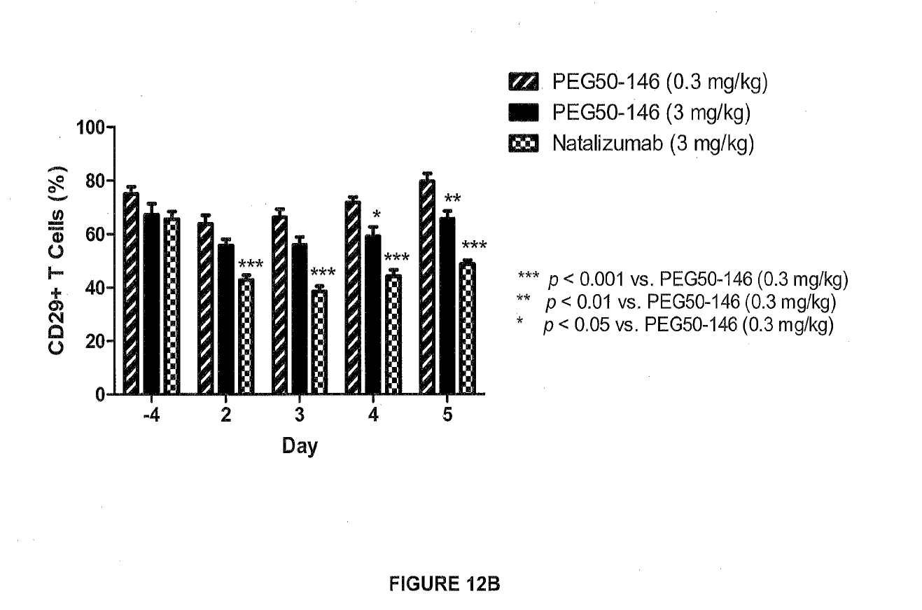

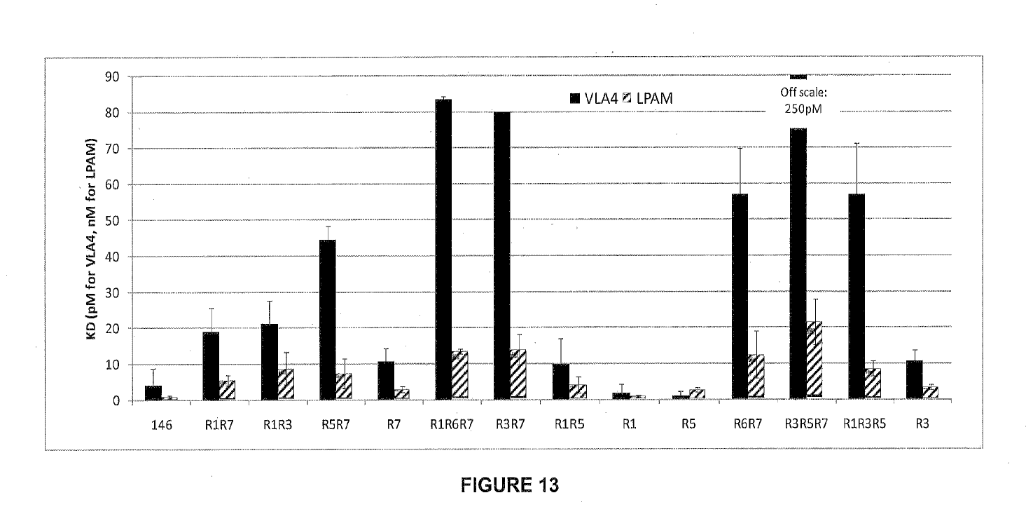

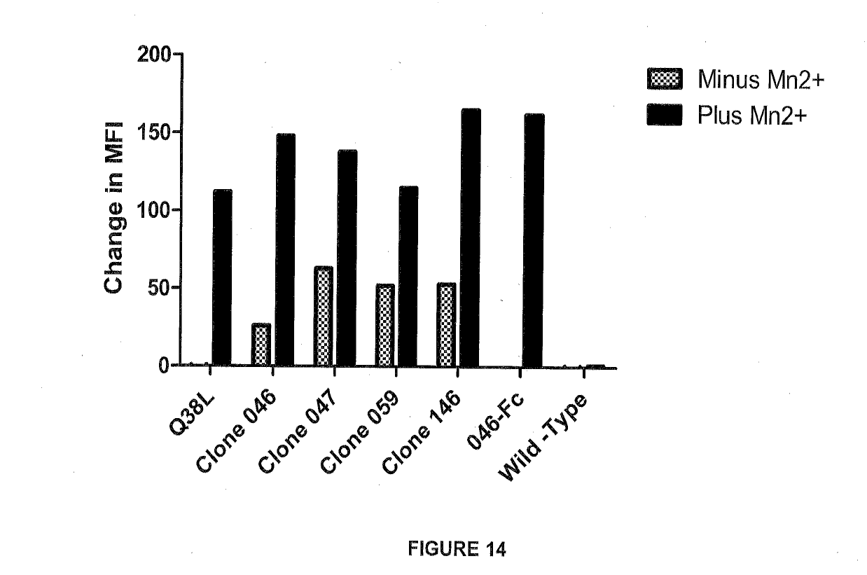

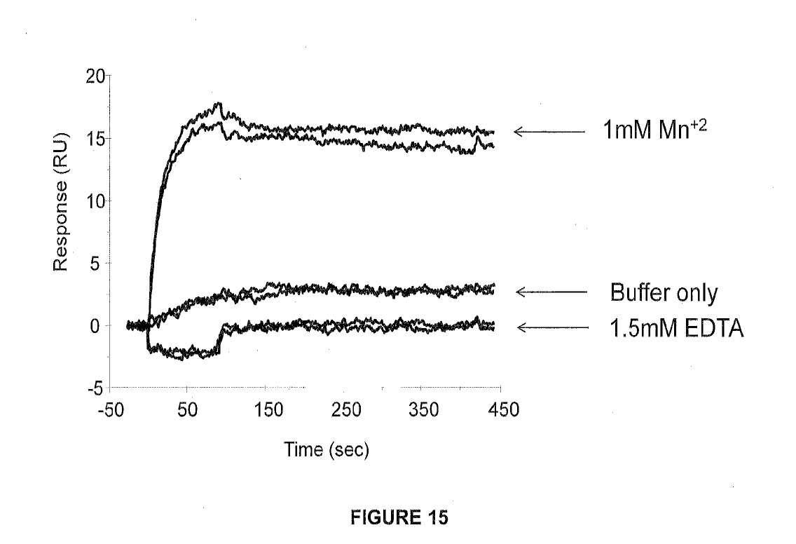

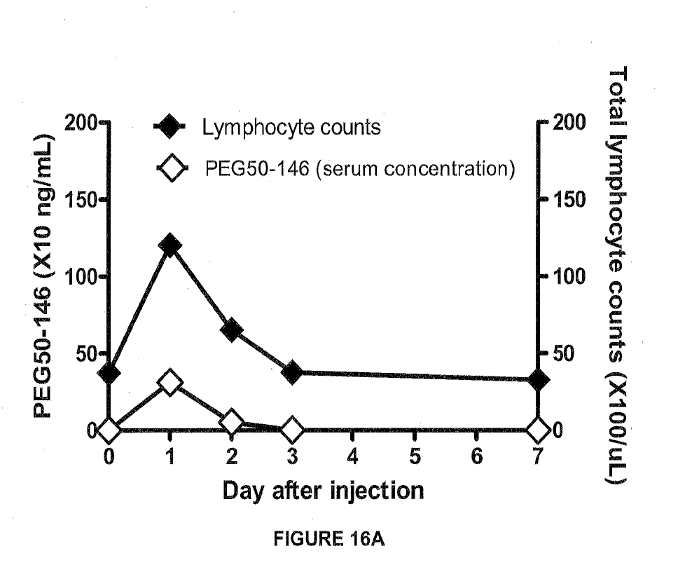

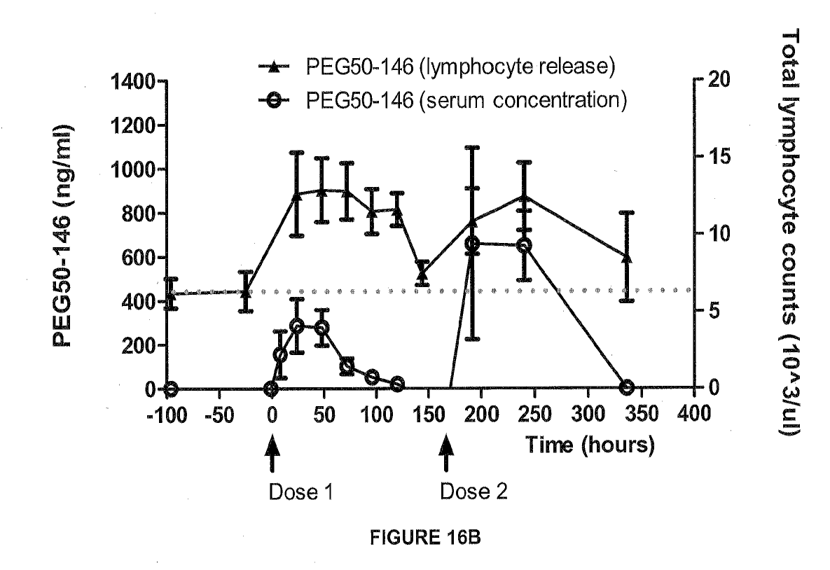

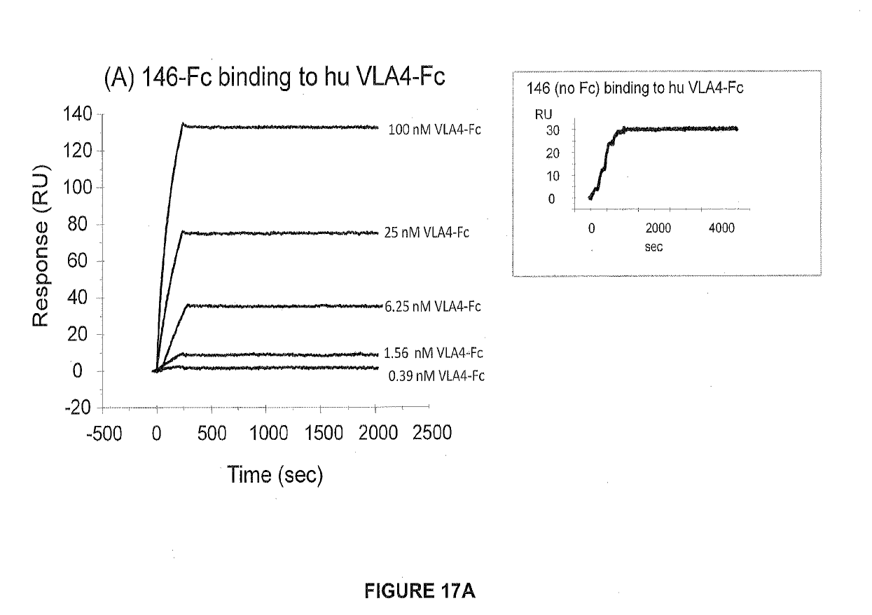

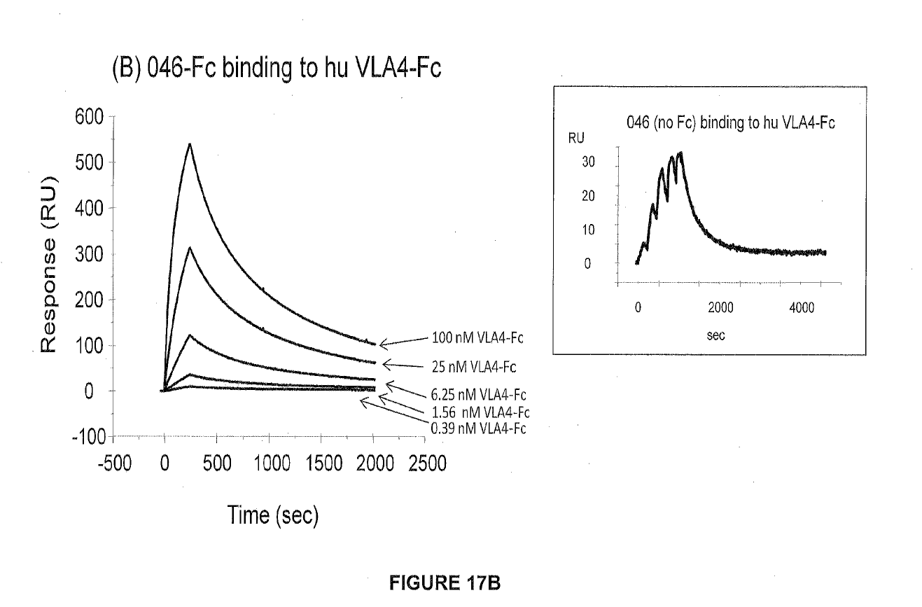

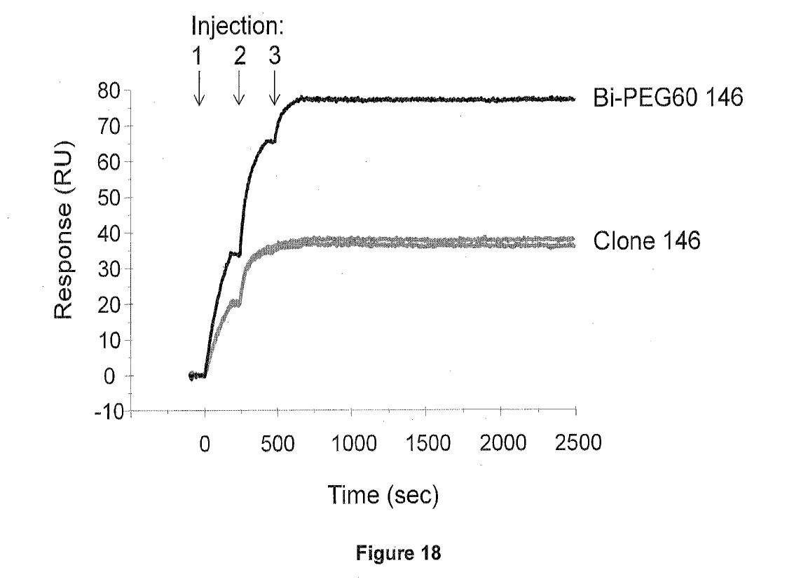

FIG. 6 depicts titration curves of U937 cell adhesion to immobilized VCAM-1 in the presence of varying concentrations of soluble 2D-VCAM-1 polypeptides. From the curves, the IC50 of variant Clone 46 (SEQ ID NO:12) and variant Clone 146 (SEQ ID NO:18) were estimated to be 6.4 nM (0.16 mg/ml) and 0.8 nM (0.02 mg/ml), respectively. The Q38L-2D-VCAM-1 polypeptide (SEQ ID NO:10) did not show complete inhibition and the IC50 was not estimated. The cell binding inhibition curves depicted in FIG. 6 were generated in Graphpad Prism software using a non-linear regression model (sigmoidal dose-response, variable slope, shared top and bottom, Y=Bottom+(Top-Bottom)/(1+10̂((LogIC50-X)*HillSlope) to estimate IC50 values.

### Example 14

### 2D-VCAM-1 Variant Polypeptides are Effective in a Murine Experimental Autoimmune Encephalomyelitis (EAE) Model

The following example demonstrates that 2D-VCAM-1 variant polypeptides of the present invention are effective in vivo in delaying the onset of paralysis and reducing the severity of disease symptoms in a murine experimental autoimmune encephalomyelitis (EAE) model, a recopized animal model of human multiple sclerosis (MS).

EAE is an acute or chronic-relapsing, acquired, inflammatory and demyelinating autoimmune disease. EAE is generally induced by injecting an animal with proteins which are found in myelin, the insulating sheath that surrounds neurons, such as myelin basic protein (MBP) or proteolipid protein (PLP). Alternatively, synthetic peptides with sequences corresponding to known epitopes of these proteins are injected. The injected proteins or immunogenic peptides induce an autoimmune response in the animal, such that the animal's immune system mounts an attack on its own myelin. The animal undergoes a disease process that closely resembles MS in humans.

Female SJL mice 6-7 weeks old (Charles River Labs) were weighed and injected subcutaneously across the flank with a total of 100 ul of emulsion 1000 ug/ml PLP (PLP Peptide 139-151. HSLGKWLGHPDKF (SEQ ID NO:99), Anaspec) and 2 mg/ml Mycobacterium tuberculosis H37Ra (Fisher) in Incomplete Freund's Adjuvant (IFA; Sigma). Mice were placed in four groups of 7-8 annuals per group. Starting on day 7 until day 15, mice were either left untreated or injected intravenously with 100 ug (in 100 ul PBS) of either rat anti-murine α4 integrin monoclonal antibody PS/2 (AbDSerotec) every other day (control group), 2D-VCAM-1 variant Clone 59 (SEQ ID NO:16) every day, or 2D-VCAM-1 variant Clone 146 (SEQ ID NO:18) every day. Mice were observed daily post-immunization, and checked for injection site complications such as abscesses. Starting on day 10 post-injection, body weights were determined every other day and the animals were inspected daily and disease progression was graded. The standard grading system for disease progression was used, as follows:

TABLE 8

| | |
|---|---|
| 0.5 | Half of tail limp |
| 1.0 | Whole tail limp |
| 1.5 | Whole tail limp with a small amount of gait disruption |
| 2.0 | Hind limb weakness (waddling gait or one dragging leg) |
| 2.5 | Hind limb weakness (one dragging leg with a small amount of loss |
| | of motile function in opposite leg) |
| 3.0 | Hind limb paralysis (both legs dragging; some slight hind limb |
| | movement) |
| 3.5 | Hind limb paralysis (both legs dragging with a small amount of |
| | forelimb weakness) |
| 4.0 | Hind limb and front limb paralysis (sufficient to prevent movement) |
| 5.0 | Moribundity or death |

If an animal received a score of 4.0 or higher, it would have been humanely euthanized. Observations were discontinued in the control group, past day 15.

As shown in FIG. 7, the onset of EAE symptoms was delayed and the overall severity of disease was reduced in the animals treated with the 2D-VCAM variant polypeptides, compared to the untreated animals. 2D-VCAM-1 variant Clone 146 appeared to be more effective than clone 2D-VCAM-1 variant Clone 59, which may correlate with the difference in VLA-4 integrin binding affinity of these two molecules. The average days of onset of disease for the untreated group, the Clone 59 treated group, and the Clone 146 treated group are 9.6±0.8 days, 11.1±1.4 days, and 12.8±1.1 days, respectively. These are statistically significant improvements (p=0.016 for Clone 59 and p=0.0016 for Clone 146). Further improvements in protecting the animals from disease in the mouse EAE model may he achieved by optimizing the dosage levels and dosing frequency of the 2D-VCAM-1 varians and using 2D-VCAM-1 variants with extended half lives such as those modified with a PEG reagent.

These results show that 2D-VCAM-1 variant polypeptides of the invention exhibiting VLA-4 integrin binding affinity are efficacious in a rodent EAE model, and suggests that 2D-VCAM-1 variant polypeptides of the present invention will be efficatious in the treatment of multiple sclerosis and related disorders.

### Example 15

### Refolding, PEGylation, and Purification of “Tagless” 2-D VCAM-1 Variant Polypeptides

The following example provides exemplary procedures for refolding, PEGylating, and purifying 2D-VCAM-1 variant polypeptides of the invention which are expressed without a histidine tag used to the N-terminus of the polypeptide (in other words, “tagless” 2D-VCAM-1 variants). This procedure was used to refold, PEGylate, and purify tagless Clone 146 (SEQ ID NO:18) and other variants of the present invention.

1. Capture step: Inclusion bodies (IB) from E. coli were adjusted to 25 mM sodium acetate, 25 mM NaH₂PO₄, 8M urea, pH 4 (0.1 g IB/ml). The material was incubated overnight at room temperature, then centrifuged at 20,000 RPM for 1 hour to remove insoluble components. The supernatant was filtered with a 0.22 μm filter, then applied to a Sartobind Q Mini filter in flow-through mode to remove DNA. The Sartobind filter was equilibrated with 25 mM sodium acetate, 25 mM NaH₂PO₄, 8M urea, pH 4 before solubilized protein application. The Sarobind Q flow through material was readjusted to pH 4 with 0.5 M HCl before application onto an SP Sepharose FF column equilibrated with 25 mM sodium acetate, 25 mM NaH₂PO₄, 8 M urea, pH 4. After an equilibration buffer wash, the protein was eluted into 25 mM sodium acetate, 25 mM NaH₂PO₄, 300 mM NaCl, 8 M urea, pH 4.

2. Refolding of the tagless 2D-VCAM-1 variant: Refolding was accomplished by dilution into 50 mM Tris, 2 mM oxidized glutathione, 1 mM reduced glutathione, pH 8, with urea supplementation to 0.3 M final concentration. The protein concentration of the refold was kept at 0.2 mg/ml with a minimum dilution of 1/26.7 to prevent the urea concentration from exceeding 0.3 M. Refolding was achieved over a minimum of 40 hours at 4° C.

3. Q Sepharose HP Purification: Purification of the refolded material was achieved by loading the refold onto a Q HP column equilibrated with 25 mM Tris, pH 6. After an equilibration buffer wash, the protein was eluted with a gradient to 25 mM Tris, 100 mM NaCl, pH 6.

4. Conditioning for PEGylation: One of the two methods below was used to condition the 2D-VCAM-1 variant for PEGylation.
A) The protein was dialyzed into 100 mM NaH2PO4 using a 3.5K molecular weight cut off (MWCO) dialysis cassette, then concentrated to between 1 and 5 mg/ml using a 3K MWCO centrifugal concentrator; or
B) The protein was loaded onto an SP Sepharose FF column equilibrated with 50 mM KH2PO4, 0.01% Tween-80, pH 4.8. The column was washed with equilibration buffer, then eluted with 50 mM KH₂PO₄, 200 mM KCl, 0.01% Tween-80, pH 5. The eluate was adjusted to pH 4 with 0.5M HCl.

5. N-terminal PEGylation of tagless 2D-VCAM-1 variant: 1 to 4 mg/ml protein was reacted with a 2 to 1-fold molar excess (depending on protein concentration) of 50K branched PEG-aldehyde (SUNBRIGHT GL3-400AL1001U; NOF Corporation, Tokyo, Japan) to protein and 0.63 mg/ml NaCNBH₃. The reaction was incubated for 3 hours at room temperature.

6. Isolation of the monoPEGylated 2D-VCAM-1 variant on SP Sepharose HP: The PEGylated material was diluted with water to a conductivity <8 mS/cm and loaded onto an SP Sepharose HP column equilibrated with 20 mM each of malic acid, citric acid, succinic acid, NaH₂PO₄, and HEPES (MCSPH), 0.01% Tween-80, pH 4. Ater an equilibration buffer wash, the protein was eluted with a gradient to 20 mM each MCSPH, 100 mM NaCl, 0.01% Tween-80, pH 5.5. The monoPEGylated fractions were pooled, concentrated with a 3K MWCO centrifugal concentrator and buffer-exchanged into PBS using a 3.5K MWCO dialysis cassette. Proteins were analyzed by SDS-PAGE and size exclusion chromatography.

Alternatively, steps 3 and 4 above may be replaced with step 3′ below:

3′. Ceramic Hydroxyapatite (CHT) Type I Purification: The refolded protein was adjusted to 1 mM NaH₂PO₄, 50 mM MES, pH 6 and loaded onto a CHT column equilibrated with 1 mM NaH₂PO₄, 50 mM MES, 150 mM NaCl, pH 6. The column was washed with equilibration buffer and then with 5 mM NaH₂PO₄, 150 mM NaCl, pH 6. The protein was then eluted with 19 mM NaH₂PO₄, 243 mM NaCl, pH 6.5. The eluate was adjusted to pH 4 with 0.5 M HCl.

Step 3′ may also be replaced by a Capto mixed mode chromatography (MMC).

### Example 16

### Extended Half-Life and In Vivo Activity Efficacy of an N-Terminally PECylated 2D-VCAM-1 Variant

Exemplary 2D-VCAM-1 variant Clone 146 (SEQ ID NO:1.8) was produced in E. coli and N-terminally PEGylated with a 50K branched PEG-aldehyde reagent as described above. The PEGylated Clone 146 molecule (“PEG50-146”) exhibited a significantly longer half-life in mouse sera than the non-PEGylated Clone 146 polypeptide (Table 9).

TABLE 9
Drug serum conc (ug/ml)- IV Administration

| | | | | | | | Standard |
|---|---|---|---|---|---|---|---|
| | | | Mouse 1 | Mouse 2 | Mouse 3 | Average | deviation |
| Clone 146 | 5 | min | 32.5 | 34.4 | 28.0 | 31.6 | 3.3 |
| Clone 146 | 24 | h | Below limit | Below limit | Below limit | — | — |
| | | | of detection | of detection | of detection | | |
| PEG50-146 | 5 | min | 33.9 | 33.0 | 27.1 | 31.3 | 3.7 |
| PEG50-146 | 24 | h | 35.3 | 19.5 | 28.3 | 27.7 | 7.9 |

In mice, the anti-α4 integrin antibody PS/2 inhibits the interaction between VLA-4 expressed on immature lymphocytes and VCAM-1 expressed on bone marrow cells, thus causing the immature lymphocytes to egress from bone marrow to peripheral blood in a process termed peripheral blood leukocytosis. The PEG50-146 molecule likewise induced an increase in peripheral blood leukocytes in mice (Table 10) and in cynomolgus monkeys (described in Example 22), thereby indicating that the 2D-VCAM-1 variant conjugate inhibits the interaction of VLA-4 and VCAM-1 in vivo. The PEG50-146 molecule also showed in vivo activity by delaying the onset of EAE symptoms in female SJL mice (as described in Example 14) compared to untreated controls when administered at 3 mg/kg on days 6, 8, 10 and 13 after PLP administration (FIG. 7B).

TABLE 10

| | PBS Treated Mice | PEG50-146 (0.4 mg/kg) | |
|---|---|---|---|
| | (n = 5-6) | Treated Mice (n = 5-6) | Fold Increase |
| | Lymphocyte counts/ul | Lymphocyte counts/ul | Over PBS |
| 24 h | 10453 | 13839 | 1.3 |
| 48 h | 7738 | 1590 | 2.1 |

### Example 17

### N-Terminally PEGylated 2D-VCAM-1 Variant Polypeptides are Effective in a Guinea Pig Experimental Autoimmune Encephalomyelitis (EAE) model

The following example demonstrates that N-terminally PEGylated 2D-VCAM-1 variant polypeptides of the present invention are effective in vivo in delaying the onset of paralysis in a guinea pig experimental autoimmune encephalomyelitis (EAE) model, a recognized animal model of human multiple sclerosis (MS).

Female Hartley guinea pigs were immunized by injection of whole guinea pig brain homogenates plus Mycobacterium tuberculosis in complete Freund adjuvant on day 0. Guinea pigs were placed into groups of 9 animals per group. Starting on day 13, guinea pigs were either left untreated or injected subcutaneously on days 13, 15 and 17 with 0.3 mg/kg of 2D-VCAM-1 variant Clone 146 N-terminally PEGylated with a 50K branched PEG-aldehyde reagent (PEG50-146) or with 0.3 mg/kg of 2D-VCAM-1 variant Clone 146 N-terminally PEGylated with a 80K branched PEG-aldehyde reagent (FEG80-146). The humanized anti-α4 integrin monoclonal antibody natalizumab (Biogen Idec) was administered on day 13 at 3 mg/kg (control). Guinea pigs were observed daily post-immunization and disease progression was graded. The standard grading system for disease progression was used as follows:

TABLE 11

| | |
|---|---|
| 0.5 | Weight loss |
| 1.0 | Limb weakness |
| 2.0 | Limb weakness, incontinence |
| 3.0 | Paralysis |
| 4.0 | Sever paralysis |
| 5.0 | Moribundity or death |

As shown in FIG. 8, the onset of EAE symptoms was delayed in the animals treated with PEG50-146 and PEG80-146 polypeptides compared to the untreated controls. The animals treated with PEG50-146 and PEG80-146 protected the guinea pigs from disease until approximately days 19 and 21 after immunization or 2 and 4 days after the last polypeptide administration. The animals treated with natalizumab were also protected from disease and began to show symptoms of disease on day 24 or 11 days after the last drug administration. These differences in the amount of time that it took for the animals to develop disease following the last administration of drug may reflect differences in the pharmacokinetics and/or duration of VLA4 receptor occupancy between the PEGylated 2D-VCAM-1 variant polypeptides and the control antibody.

These results show that N-terminally PEGylated 2D-VCAM-1 variant polypeptides of the present invention exhibit VLA4 integrin binding affinity and are efficacious in a rodent EAE model. The data also suggests that 2D-VCAM-1 variant polypeptides of the present invention will be efficacious in the treatment of multiple sclerosis and related disorders.

### Example 18

### ELISA Assay for Detecting Serum Concentrations of PEG50-146 in Cynomolgus Monkeys

The amount of functional PEG50-146 in the serum of animals at various times after subcutaneous administration of PEG50-146 polypeptide in cynomolgus monkeys was determined using a solid-phase ELISA assay. MAXISORP™ ELISA plates (NUNC) were coated overnight at 4° C. with 50 ul VLA4-Fc heterodimer, 1 ug/ml in TBS-KCl (TBS minus KCl; 24.7 mM Tris-HCl pH 7.4, 137 mM NaCl), washed with TBS-T (TBS with 0.05% TWEEN-20), blocked with 200 ul 11% bovine serum albumin (BSA) in TBS-KCl for 1 h at room temperature. A standard curve of PEG50-146 5-fold dilutions starting at 5 ug/ml was prepared in TBS-KCl with 1% BSA containing epomolgus monkey serum (the final concentration of cynomolgus monkey serum was 20%). The experimental unknown samples were prepared by preparing a 2-fold serial dilution in TBS-KCL with 1% BSA and 20% monkey serum. Both the PEG50-146 and experimental unknown samples were transferred to the ELISA plate which was incubated at room temperature for 1 h with shaking and subsequently washed with TBS-T. Bound PEG50-146 protein was detected by incubation with 0.25 ug/ml of biotinylated anti-human VCAM antibody (R&D Systems, Catalog No. BAF809) in TBS-KCl with 1% BSA for 1 h at room temperature with shaking followed by washing and subsequent incubation with 1:100,000 dilution of streptavidin-HRP (BD Biosciences, Catalog No. 554066) in TBS-KCl with 1% BSA for 1 hour at room temperature. After a final wash with TBS-T, 100 ul of TMB Plus substrate buffer (Kem Diagnostics, Catalog No. 4390A) equilibrated to room temperature in the dark was added to each well and color development was followed visually. Typical development times ranged from 15 minutes plus or minus 2 minutes and development was stopped by addition of 50 ul of Stop Solution (R&D Systems, Catalog No DY994). Absorbance at 450 nM was measured promptly using a SpectraMax Softmax Pro spectrophotometer (Molecular Devices, Sunnyvale, Calif.).

Cynomolgus monkeys were injected subcutaneously on day 0 (0 hours) with 0.3 mg/kg of 2D-VCAM-1 variant Clone 146 N-terminally PEGylated with a 50K branched PEG-aldehyde reagent (PEG50-146) or 3 mg/kg of PEG50-146. At 8, 24, 48, 72, 96, 120 and 144 hours post-administration, serum samples were collected. There were 4 animals per group. Serum samples were analyzed for the quantity of functional PEG50-146 polypeptide by the ELISA method described above.

As shown in FIG. 9A, following subcutaneous administration of PEG50-146 at 0.3 mg/kg and 3 mg/kg, the amount of PEG50-146 in the serum is approximately 300 ng/ml and 1300 ng/ml, respectively, at 24 hours. The amount of PEG50-146 polypeptide in the serum then decreases with an approximate half-life in the range of 20-60 hours. The amount of PEG50-146 capable of binding to VLA4-Fc is below the limit of the assay at 144 hours post-administration in the low dose group (0.3 mg/kg).

### Example 19

### 2D-VCAM-1 Variant Polypeptides do not Decrease Surface Expression of α4 and β1 Integrins on Peripheral Blood Lymphocytes

The impact of 2D-VCAM-1 polypeptide binding to the surface expression of α4 and β1 integrin on peripheral blood lymphocytes was examined in both mice and cynomolgus monkeys using a fluorescent activated cell sorting (FACS) assay.
A. Evaluation of Cell Surface Expression of α4 and β1 Integrin on Murine Peripheral Blood Lymphocytes

Whole mouse blood collected in EDTA was diluted 1:10 with 1× Lysing Buffer (BD Biosciences, Catalog No 555899), gently vortexed, incubated at room temperature for 5 minutes, and washed in PBS plus 1% heat-inactivated fetal bovine serum (PBS). The cell suspension was then incubated with mouse Fc Block (BD Biosciences, Catalog No 553142) for 5 minutes (2 ul of Fc Block per 1×106 cells). Cells were then double stained for α4 integrin or β1 integrin cell surface levels on peripheral B cells with a rat anti-murine B220 antibody conjugated to FITC (Clone RA3-6B2, BD Sciences, Catalog No. 553088) and a non-blocking rat anti-mouse CD49d (α4 integrin) monoclonal antibody conjugated to PE (clone 9C10-PE; BD Sciences, Catalog No. 557420) or a non-blocking hamster anti-mouse CD29 (β1 integrin) biotinylated antibody (clone 6A267, USBiological, Catalog No. C2381-10N)). Biotinylated anti-mouse CD29 was detected with streptavidin-PE (SouthernBiotech, Catalog No. 9190-09) diluted 1:200. Isotype antibodies were used as negative controls. Stained cells were resuspended in PBS plus 2% FBS and analyzed on a FACS Calibur. Flow cytograms were generated to establish the fraction of cells positive for each cell surface marker. Live lymphocytes were selected based on exclusion of propidium iodide (PI) (Roche, Ref. 11 348 639 001). An electronic gate was drawn within the Forward vs. Side Scatter cytogram to include the lymphocytes which were used as denominator for percent calculations of antibody positive cells.

Female Balb/c mice were injected subcutaneously on day 0, day 2 and day 4 with 0.4 mg/kg of PEG50-146 or 3 mg/kg of rat anti-murine α4 integrin monoclonal antibody PS/2 (AbDSerotec) or PBS. Mice were placed in three groups of 5 animals per group. On days 1, 2, 4, 5, 6, 7, and 8, whole blood was collected for each group, pooled and analyzed for α4 and β1 integrin levels on B220+ peripheral B cells by flow cytometry using the assay described above. No samples were taken on day 3.

As shown in FIG. 10, there was no change in the percentage of peripheral B cells that were CD49d+ (α4 integrin) in the mice treated with PEG50-146 compared to the PBS treated controls. In contrast, PS/2 decreased the percentage of CD49+ B cells as early as 1 day post-treatment. This reduction in the percentage of CD49+ B cells with PS/2 treatment was sustained at approximately 55% through day 7. On day 8, the percentage of CD49+ B cells began to normalize reaching 90% positivity 4 days after the last administration of PS/2 antibody. The cell surface levels of β1 integrin were also unchanged in the PEG50-146 treated mice whereas there was an observable decrease in β1 integrin positive B cells in PS/2-treated mice following a single subcutaneous dose (Table 12).

TABLE 12

| | | | | | % Reduction |
|---|---|---|---|---|---|
| Day | | % CD29+ | % Reduction | % CD29+ | of CD29+ B |
| after | | B cells in | of CD29+ B | B cells in | cells in |
| injec- | | PS/2 | cells in PS/2 | PEG50-146 | PEG50-146 |
| tion | PBS | Treated Mice | Treated Mice | Treated Mice | Treated Mice |
| DAY 1 | 99.9 | 89.9 | 10.0 | 99.9 | 0 |
| DAY 2 | 99.9 | 75.0 | 25.0 | 99.7 | 0.2 |
| DAY 3 | 99.9 | 88.7 | 11.3 | 97.4 | 2.6 |
| DAY 4 | 100 | 94.2 | 5.8 | 100 | 0 |
| DAY 7 | 100 | 100 | 0 | 100 | 0 |

As shown in Table13, the treatment of mice with rat anti-murine α4 integrin monoclonal antibody R1-2 (Biolegend, Catalog No. 103610) also reduced the number of CD49d+ B cells and CD29+ B cells in the spleen of Balb/c mice treated with 3 mg/kg of R1-2. R1-2 binds to a different epitope on u4 integrin compared to that of PS/2.

TABLE 13

| Percentage of CD49d+ B Cells | | | Percentage CD29+ B Cells | | |
|---|---|---|---|---|---|
| PBS Treated | R1-2 Treated | Percent Reduction | PBS Treated | R1-2 Treated | Percent Reduction |
| Mice (%) | Mice (%) | from PBS control | Mice (%) | Mice (%) | from PBS control |
| 98.9 | 60.3 | 39.0 | 98.6 | 86.4 | 12.4 |
| 99.2 | 67.9 | 31.5 | 84.2 | 54.5 | 35.2 |

These results show that 2D-VCAM-1 variant polypeptides of the invention do not decrease cell surface expression of α4 or β1 integrin on peripheral B cells unlike the anti-α4 monoclonal antibodies PS/2 and R1-2. These results also suggest that a decrease in α4 and β1 integrin on B cells resent in either the peripheral blood or the spleen) may be a general property of monoclonal antibodies that bivalently bind and cross-link α4 integrin on the cell surface.

### A. Evaluation of Cell Surface Expression of α4 and β1 Integrins in Cynomolgus Monkeys Peripheral Blood Lymphocytes:

Whole blood samples for flow cytometry analysis (approximately 1.0 mL) were collected into tubes treated with K3EDTA. Isolated leukocytes were stained with antibodies to CD3 (SP-34(2)-APC-Cy, BD Biosciences, Catalog No. 557757), CD20 (2H7-PE-Cy7, BD Biosciences, Catalog No. 560734), CD29 (MAR4-APC, BD Sciences, Catalog No. 557332), and CD49d (9F10-PE, BD Sciences, Catalog No. 556635) using a lyse/wash method. Flow cytometric data was captured using FACSCanto II (Becton Dickinson BD) and FACSDiva analysis software. Acquired data was analyzed on FACSDiva analysis software. Flow cytograms were generated to establish the fraction of cells positive for each cell surface marker. An electronic gate was drawn within the Forward vs. Side Scatter cytogram to include the lymphocytes which were used as denominator for percent calculations of antibody positive cells. The absolute counts of the individual lymphocyte subpopulations will be calculated from their relative percentages as derived from the lymphocyte gate and the absolute lymphocyte count from the Advia 120 Hematology Analyzer according to the formula: Absolute lymphocyte count (×103/μL)=(lymphocyte subpopulation relative %×total lymphocyte absolute count)/100.

Male cynomolgus monkeys were injected subcutaneously on day 1 with 0.3 mg/kg of PEG50-146 or 3 mg/kg of PEG50-146 or 3 mg/kg of humanized anti-α4 integrin monoclonal antibody natalizumab (Biogen Idec) (control). The animals were divided into three groups of 4 animals each. On days −4, 2, 3 4 and 5 post-administration, whole blood samples were collected and analyzed for α4 integrin and β1 integrin cell surface levels on CD20+ peripheral B cells and CD3+ peripheral T cells by flow cytometry using the assay described above.

As shown in FIG. 11A, the baseline (day −4) percentage of CD49d+ B cells in the peripheral blood of all cynomolgus monkeys was approximately 70-80% for all 3 groups of animals on day −4. On days 2-5 after drug administration, the percentage of CD49d+ B cells in the peripheral blood of the PEG50-146 treated monkeys was 96-97%. The increase in the percentage of CD49d+ B cells from predose (day −4) to day 2 post-administration for the PEG50-146 groups most likely reflects the overall increase in peripheral lymphocytes (leukocytosis) in the blood that occurs as a result of blocking the interaction of VCAM-1 with VLA4 as described in Example 16. Despite leukocytosis in the natalizumab treated animals to a similar to higher extent than that observed with a single dose of PEG50-146 (Table 14), the percentage of CD49d4d+ B cells in the periphery remained at approximately 68% for days 2 and 3 after which it increased to 93-94% positive. The difference in percentage of CD49d+ B cells in the natalizumab treated animals compared to the PEG50-146 groups at days 2 and 3 was statistically significant (*p<0.001; Two Way ANOVA with Bonferroni post-hoc test). A similar reduction in the percentage of T cells that were CD49d+ was also observed with the natalizumab treated monkeys, whereas both PEG50-146 treatment groups had no effect on the percentage of CD49d+ Tcells (FIG. 11B) (*p<0.001; Two Way ANOVA with Bonferroni post-hoc test).

TABLE 14

| | PEG50-146 (0.3 mg/kg) | | PEG50-146 (3 mg/kg) | | Natalizumab (3 mg/kg) | |
|---|---|---|---|---|---|---|
| | | Fold | | Fold | | Fold |
| | | change | | change | | change |
| | Lymphocyte | from | Lymphocyte | from | Lymphocyte | from |
| | counts | average | counts | average | counts | average |
| Day | (10\hat{ }3/ul) | baseline) | (10\hat{ }3/ul) | baseline) | (10\hat{ }3/ul) | baseline) |
| −4 | 6.20 | 1.0 | 5.5 | 1.0 | 4.49 | 1.0 |
| (baseline) | | | | | | |
| −1 h | 6.34 | 1.0 | 5.6 | 1.0 | 4.84 | 1.0 |
| (baseline) | | | | | | |
| 2 | 12.64 | 2.0 | 12.6 | 2.3 | 11.24 | 2.4 |
| 3 | 12.90 | 2.1 | 14.8 | 2.7 | 13.90 | 3.0 |
| 4 | 12.82 | 2.0 | 15.5 | 2.8 | 14.77 | 3.2 |
| 5 | 11.51 | 1.8 | 15.0 | 2.7 | 16.35 | 3.5 |
| 6 | 11.64 | 1.9 | 16.7 | 3.0 | 15.51 | 3.3 |
| 7 | 7.48 | 1.2 | 12.7 | 2.3 | 13.71 | 2.9 |

As shown in FIG. 12A, there was no significant change in the percentage of peripheral B cells that were CD29+ in monkeys treated with either dose of PEG50-146 compared to the pretreatment sample (day −4). In contrast, there was an approximately 2-fold reduction in the percentage of CD29+ B cells in the natalizumab treated monkeys between day −4 and day 2 (from 80% positive on day −4 to 37% positive on day 2). The reduction in the percentage of CD29+ B cells in the natalizumab treated monkeys was statistically significant from that of the PEG50-146 dose groups at day 2, 3, 4 and 5 (*p<0.001; Two Way ANOVA with Bonferroni post-hoc test). A similar reduction in the percentage of T cells that were CD29+ was observed with the natalizumab treated monkeys whereas both PEG50-146 treatment groups had no effect on the percentage of CD29+ T cells (FIG. 12B) (*p<0.001; Two Way ANOVA with Bonferroni post-hoc test).

The results in FIGS. 11 and 12 show that PEG50-146 does not decrease the cell surface expression of α4 or β1 heterodimeric integrin adhesion molecules on peripheral B or T cells in cynomolgus monkeys at either dose tested. These results are in contrast to that observed in natalizumab-treated monkeys where both α4 and β1 integrin levels were decreased on the surface of peripheral lymphocytes. The transient downregulation of α4 levels with natalizumab treatment on days 2-3 (FIG. 11) most likely reflects the conversion of bivalent, intact natalizumab antibody found in the serum of these animals to natalizumab that is essentially monovalent for VLA4 binding due to chain swapping with endogenous monkey IgG4 antibodies. This was supported by ELISA data showing the majority of bivalent, functional natalizumab was cleared from the monkeys between days 3 and 4 after drug administration (FIG. 9B). The functional natalizumab concentration in monkey serum was determined by binding to human VLA4-Fc in an ELISA assay similar to that described in Example 18 except that bound Tysabri was detected with a mouse monoclonal secondary antibody to human IgG4 conjugated to horse radish peroxidase (HRP) (Abeam, Catalog No. ab99823). IgG4 chain swapping of natalizumab with endogenous human IgG4 has been observed in patients and in animals (Labrijn, A. F., et al., Nat. Biotechnol. 27(8):767-771 (2009); Shapiro, R. I. et al., J. PHarm. Biomed. Anal. 55(1):168-175 (2011)). Multiple doses of natalizumab in the monkeys most likely would have maintained a steady state level of bivalent natalizumab in the animals and sustained down regulation of cell surface VLA4 on peripheral lymphocytes as was observed for anti-4 monoclonal antibody in mice (FIG. 10) and humans (Putzki, N., et al., eur. Neurology, 63:311-317 (2010); Wipfler, et al., Mult. Schier, 17:16-23 (2011); Harrer, et al., J. Neuroimmunol. “Natalizumab therapy decreases surface expression of both VLA-heterodimer subunits on peripheral blood mononuclear cells,” Epub (2011).

A single dose of natalizumab had a sustained impact on β1 containing integrin receptors with receptor down regulation observed through day 5 in monkeys (FIG. 12). This may reflect a broader impact of natalizumab on cell adhesion molecules other than VLA4 and LPAM-1 as beta-1 non-covalently associates with at least nine different chains. This impact of natalizumab on beta-1 containing adhesion molecules could have profound effects on immunological activities dependent upon these adhesion molecules such as immune surveillance and migration of immune cells to sites of inflammation. As PEG50-146 does not impact adhesion molecule cell surface expression, it may have a reduced effect on immune surveillance, immune cell trafficking, immune homeostasis, and risk of opportunistic infections, such as JC virus, relative to an α4 monoclonal antibody.

### Example 20

### Sequence Optimization of Clone 146 (SEQ ID NO:18)

A first set of twelve sequence optimized variants (R1-R12) of 2D-VCAM-1 variant Clone 146 (SEQ ID NO: 18) were designed and synthesized at GeneArt (Invitrogen, Inc). The sequence optimized variants contained various combinations of the seven mutations present in 2D-VCAM-1 variant Clone 146 (SEQ ID NO:18) (F32L S34F T37P Q38L, I39L T74R K79S relative to human 2D-VCAM-1, SEQ ID NO:2) but with lower total mutation loads (Table 15). Each synthetic gene contained a 5′ NheI site, a Kozak sequence, the variant ORF (consisting of the endogenous VCAM-1 signal sequence, the 2D-VCAM-1 variant ORF and three consecutive spacer-FLAG tags (GGGS-DYKDDDDK)), and a terminator codon followed by a 3′ PmeI site. Unique restriction sites (HindIII, NotI, and BamHI) were engineered at different locations in the ORF by appropriate codon usage to allow us to make new clones by exchanging fragments amongst the first set of clones. The synthetic genes were subcloned into the pCDNA3.1(+) (Invitrogen, Inc.) mammalian expression vector using NheI and PmeI restriction sites and the plasmids were transiently transfected into CHO-S cells. The cell-culture supernatants were concentrated and filtered and assayed for binding to VLA4-Fc by BIACORE. Plasmids for 2D-VCAM-1 variant Clone 146 expression were also generated in this fashion and transfected and assayed to obtain control data. The initial set of single revertant clones (R1-R7) suggested that F32L and K79S made a small contribution, if any, to the activity improvement of Clone 146, T37P and I39L make modest contributions to the activity improvement of Clone 146 and S34F, Q38L, and T74R make the greatest contributions to the activity improvement of Clone 146 compared to wt-2D-VCAM-1. Based on the initial results from 146R1-146R12, a second set of sequence optimized clones (Table 15) were derived from the first set through subcloning using the unique restriction sites in the ORFs or through the SOB (Splicing by Overlap Extension) technology. The plasmids were transfected and the crude supernatants were assayed as described above (Table 15). Table 15 shows the various combinations of seven mutations present in each sequence optimized variant, the total number of mutations in the variant (Mutation Load) and the reduction in binding affinity relative to 2D-VCAM-1 variant Clone 146 (146R0) as measured by BIACORE(=K_{D}(variant)/K_{D}(146R0)).

TABLE 15
Sequence Optimized Variants of 2D-VCAM-1 variant
Clone 146 (“146R0”, SEQ ID NO: 18).

| | | | | | | | | | Fold Weaker |
|---|---|---|---|---|---|---|---|---|---|
| | | | | | | | | | Binding |
| | Mutations | | | | | | | Mutation | Relative To |
| Variants | F32L | S34F | T37P | Q38L | I39L | T74R | K79S | Load | 146R0 |
| 146R0 | + | + | + | + | + | + | + | 7 | 1.0 |
| (Clone | | | | | | | | | |
| 146) | | | | | | | | | |
| SEQ ID | | | | | | | | | |
| NO: 18 | | | | | | | | | |
| 146R1 | | + | + | + | + | + | + | 6 | 1.1 |
| SEQ ID | | | | | | | | | |
| NO: 101 | | | | | | | | | |
| 146R2 | + | | + | + | + | + | + | 6 | 2.7 |
| SEQ ID | | | | | | | | | |
| NO: 103 | | | | | | | | | |
| 146R3 | + | + | | + | + | + | + | 6 | 1.3 |
| SEQ ID | | | | | | | | | |
| NO: 105 | | | | | | | | | |
| 146R4 | + | + | + | | + | + | + | 6 | 2.6 |
| SEQ ID | | | | | | | | | |
| NO: 107 | | | | | | | | | |
| 146R5 | + | + | + | + | | + | + | 6 | 1.2 |
| SEQ ID | | | | | | | | | |
| NO: 109 | | | | | | | | | |
| 146R6 | + | + | + | + | + | | + | 6 | 1.8 |
| SEQ ID | | | | | | | | | |
| NO: 111 | | | | | | | | | |
| 146R7 | + | + | + | + | + | + | | 6 | 1.0 |
| SEQ ID | | | | | | | | | |
| NO: 113 | | | | | | | | | |
| 146R8 | + | + | + | + | + | | | 5 | 1.7 |
| SEQ ID | | | | | | | | | |
| NO: 115 | | | | | | | | | |
| 146R9 | | + | + | | + | | | 5 | 2.0 |
| SEQ ID | | | | | | | | | |
| NO: 117 | | | | | | | | | |
| 146R10 | | + | + | + | + | | | 4 | 2.7 |
| SEQ ID | | | | | | | | | |
| NO: 119 | | | | | | | | | |
| 146R11 | + | + | + | | + | | | 4 | 25.9 |
| SEQ ID | | | | | | | | | |
| NO: 121 | | | | | | | | | |
| 146R12 | | | + | + | + | | | 3 | 21.7 |
| SEQ ID | | | | | | | | | |
| NO: 123 | | | | | | | | | |
| 146R13 | | + | + | + | + | + | | 5 | 2.3 |
| SEQ ID | | | | | | | | | |
| NO: 125 | | | | | | | | | |
| 146R14 | + | + | + | + | | + | | 5 | 1.5 |
| SEQ ID | | | | | | | | | |
| NO: 127 | | | | | | | | | |
| 146R15 | | + | + | | | + | | 4 | 5.3 |
| SEQ ID | | | | | | | | | |
| NO: 129 | | | | | | | | | |
| 146R16 | | + | + | + | | + | + | 5 | 1.1 |
| SEQ ID | | | | | | | | | |
| N: 131 | | | | | | | | | |
| 146R17 | | + | + | + | | + | | 4 | 2.8 |
| SEQ ID | | | | | | | | | |
| NO: 133 | | | | | | | | | |
| 146R18 | | + | + | + | | | | 3 | 6.3 |
| SEQ ID | | | | | | | | | |
| NO: 135 | | | | | | | | | |
| 146R19 | | + | + | | | + | | 3 | 40.0 |
| SEQ ID | | | | | | | | | |
| NO: 137 | | | | | | | | | |
| 146R20 | | + | + | | | + | + | 4 | 9.3 |
| SEQ ID | | | | | | | | | |
| NO: 139 | | | | | | | | | |
| I46R21 | | + | | + | + | + | + | 5 | 1.0 |
| SEQ ID | | | | | | | | | |
| NO: 141 | | | | | | | | | |
| 146R22 | + | + | | + | + | + | | 5 | 1.6 |
| SEQ ID | | | | | | | | | |
| NO: 143 | | | | | | | | | |
| 146R23 | | + | | + | + | + | | 4 | 6.1 |
| SEQ ID | | | | | | | | | |
| NO: 145 | | | | | | | | | |

Based on the results from the crude supernatant assay, thirteen sequence optimized variants were chosen for further study as purified proteins. The variants were derived from the original plasmid PVM146 (VCAM146 in pVM-EcVec) by reversion of the appropriate mutations using the technique of splicing by SOB technology. Sequence information for the variants is provided in Table 16. The plasmids were transformed into W3110 E. coli strain for production and purified and assayed for VLA-4 and LPAM-1 affinity as described above in Examples 8 and 12.

TABLE 16
Sequence optimized variants of 2D-VCAM-1 variant Clone
146 that were cloned into E. coli expression system.

| | | | | | | | | Muta- |
|---|---|---|---|---|---|---|---|---|
| | Mutations | | | | | | | tion |
| Variants | F32L | S34F | T37P | Q38L | I39L | T74R | K79S | Load |
| Clone 146 | + | + | + | + | + | + | + | 7 |
| SEQ ID | | | | | | | | |
| NO: 18 | | | | | | | | |
| 146R1R7 | | + | + | + | + | + | | 5 |
| SEQ ID | | | | | | | | |
| NO: 147 | | | | | | | | |
| 146R1R3 | | + | | + | + | + | + | 5 |
| SEQ ID | | | | | | | | |
| NO: 149 | | | | | | | | |
| 146R5R7 | + | + | + | + | | + | | 5 |
| SEQ ID | | | | | | | | |
| NO: 151 | | | | | | | | |
| 146R7 | + | + | + | + | + | + | | 6 |
| SEQ ID | | | | | | | | |
| NO: 113 | | | | | | | | |
| 146R1R6R7 | | + | + | + | + | | | 4 |
| SEQ ID | | | | | | | | |
| NO: 153 | | | | | | | | |
| 146R3R7 | + | + | | + | + | + | | 6 |
| SEQ ID | | | | | | | | |
| NO: 155 | | | | | | | | |
| 146R1R5 | | + | + | + | | + | + | 5 |
| SEQ ID | | | | | | | | |
| NO: 157 | | | | | | | | |
| 146R1 | | + | + | + | + | + | + | 6 |
| SEQ ID | | | | | | | | |
| NO: 101 | | | | | | | | |
| 146R5 | + | + | + | + | | + | + | 6 |
| SEQ ID | | | | | | | | |
| NO: 109 | | | | | | | | |
| 146R6R7 | + | + | + | + | + | | | 5 |
| SEQ ID | | | | | | | | |
| NO: 159 | | | | | | | | |
| 146R3R5R7 | + | + | | + | | + | | 4 |
| SEQ ID | | | | | | | | |
| NO: 161 | | | | | | | | |
| 146R1R3R5 | | + | | + | | + | + | 4 |
| SEQ ID | | | | | | | | |
| NO: 163 | | | | | | | | |
| 146R3 | + | + | | + | + | + | + | 6 |
| SEQ ID | | | | | | | | |
| NO: 105 | | | | | | | | |

The results from the Biacore measurement are shown in FIG. 13 and suggest that most of the double revertants with less than or equal to 5 mutations show a decrease in VLA-4 and LPAM-1 binding. Sequence optimized variants (Table 16) were expressed and purified from E. coli expression system and assayed for VLA-4 and LPAM-1 binding by BIACORE. The KDs for VLA-4 in pM (black bars) and LPAM-1 in nM (gray bars) are shown.

### Example 21

### 2D-VCAM-1 Variant Polypeptides Preferentially Bind to Integrin in a High Affinity (or Activated) State

The following example demonstrates that 2D-VCAM-1 variant polypeptides of the present invention bind preferentially to integrin receptors in the high affinity or activated state. In this experiment, conversion of VLA4 to the activated format was induced by adding 1 mM MnCl₂ to the assay buffer.

### A. Binding of 2D-VCAM-1 Polypeptides to Activated Guinea Pig VLA4 Using a Flow Cytometry Assay:

Guinea pig splenocytes were resuspended in TBS (25 mM TRIS-HCl pH 7.5, 150 mM NaCl) and incubated with the purified 2D-VCAM-1 polypeptides at a final concentration of 50 ug/ml for 15 minutes on ice. The cells were then washed with TBS plus 2% fetal bovine serum (FBS). A duplicate set of samples was prepared in which the guinea pig splenocytes were resuspended in TBS containing 1 mM MnCl2 (TBS-M) prior to addition of the 2D-VCAM-1 polypeptides on ice for 15 minutes and washing with TBS-M plus 2% FBS. Both sets of samples (referred to in FIG. 14 as “plus Mu²⁺” and “minus Mn²⁺”) were blocked in TBS plus 1% normal sheep serum for 10 minutes on ice. The bound 2D-VCAM-1 polypeptides were then detected with a biotinylated sheep polyclonal anti-human VCAM-1/CD106 antibody (R & D Systems, Catalog No BAF809) at 1 ug/ml in TBS plus 1% normal sheep serum for 15 minutes on ice. The cells were then washed and incubated with streptavidin-PE (SA-PE) (Southern Biotech, Catalog No 9190-09) diluted 1:200 on ice for 15 minutes. Stained cells were washed and resuspended in TBS plus 2% FBS and analyzed on a FACS Calibur. An electronic gate was drawn within the Forward vs. Side Scatter cytogram to include the lymphocytes. Unstained cells and cells treated only with SA-PE were used as assay controls. The data was plotted as change in mean fluorescent intensity (MFI) of the cells incubated with the 2D-VCAM-1 polypeptides plus biotinylated anti-VCAM and SA-PE minus the SA-PE only background control samples.

As seen in FIG. 14, binding to the guinea pig splenocytes was detected for Q38L (SEQ ID NO:10), Clone 046 (SEQ ID NO:12), Clone 047 (SEQ ID NO:14), Clone 059 (SEQ ID NO:16), and Clone 146 (SEQ ID NO:18) 2D-VCAM-4 polypeptides when the polypeptides were incubated with the cells in buffer containing 1 mM MnCl2 (“plus Mn2+”) representing the activated VLA4 state. Binding was also detected for a dimeric Clone 046 fusion protein (046-Fc) in the presence of MnCl₂. For all of these polypeptides, binding to the cell surface integrin on the guinea pig splenocytes was greater in the presence of 1 mM MnCl2 than in the absence suggesting that these proteins preferentially bind to integrins in the high affinity or active state. No binding of Q38L or Clone 046-Fc was observed in the absence of Mn2+. As a chelating agent such as EDTA was not added to the “minus Mn2+” samples to remove any bivalent cations introduced in the assay buffers, it is possible that a portion of the integrin receptors in the “minus Mn²⁺” samples were in the activated state and that the lower amount of binding of the polypeptides in the “minus Mn²⁺” samples was due to binding to residual high affinity receptors and not to the low affinity receptors. In comparison to Q38L or the 2D-VCAM-1 variants, no significant binding of wild-type 2D-VCAM-1 was detected in this assay either in the presence or absence of 1 mM MnCl2 which may due to the low binding affinity of wild-type 2D-VCAM-1 to purified VLA-4 as was demonstrated by BIACORE (Example 12). As a the binding of the wild-type and variant 2D-VCAM-1 polypeptides to the guinea pig splenocytes in this assay was detected indirectly with a polyclonal antibody directed against human VCAM-1, the different amount of binding of the variant polypeptides to the guinea pig splenocytes may not reflect their relative affinities to purified VLA4, but rather may be a reflection of their affinity to the anti-VCAM polyclonal antibody. The binding affinity of the wild-type and variant 2D-VCAM-1 polypeptides to the anti-VCAM polyclonal antibody was not determined.

### B. Binding of 2D-VCAM-1 Clone 146 to Activated Human VLA4-Fc in a BIACORE Assay:

The effect of the VLA4 activation state on Clone 146 (SEQ ID NO:18) binding was measured by BIACORE using the same methods described above in Example 12 with several exceptions (described below). Goat anti-human IgG antibody was immobilized on a CM5 chip and human VLA4-Fc in HBS-P was captured as described in Example 12. A single injection of Clone 146 was performed in each binding cycle, always at 150 nM. Clone 146 was diluted in HBS-P buffer, HBS-P buffer supplemented with 1 mM Mn2+ or HBS-P buffer supplemented with 1.5 mM EDTA (ethylenediaminetetraacetic acid). Clone 146 was injected for 90 seconds at a flowrate of 30 ul/min. The dissociation was monitored for 5 minutes. Each buffer condition was tested twice. The chip was regenerated with a three minute injection of Glycine, pH 1.7 after each Clone 146 injection.

Clone 146 in Mn²⁺ containing buffer bound to human VLA4-Fc (FIG. 15), confirming previous data indicating that the 2D-VCAM-1 polypeptide binds to activated VLA4. Clone 146 in EDTA containing buffer did not bind to VLA4 indicating that Clone 146 does not bind tightly to unactivated VLA4. The small binding signal observed when Clone 146 in HBS-P without added EDTA or Mn²⁺ most likely indicates that VLA4 in HBS-P is a mixture of activated and unactivated states. No significant dissociation was observed.

Taken together, the data shown in FIGS. 14 and 15 demonstrate that the 2D-VCAM-1 variant polypeptides of the present invention exhibit a higher binding affinity for VLA4 that is in the activated or high affinity state than VLA4 that is in the inactive or low affinity state using both a cell-based flow cytometry and an in vitro BIACORE assay. Therefore, the 2D-VCAM-1 variant polypeptides would be expected to bind preferentially to activated VLA4 in vivo, such as that expressed on rolling lymphocytes in mammals, such as humans, with higher binding affinity than to VLA4 in the inactive state. This preference of 2D-VCAM polypeptides to activated VLA4 may lead to selective blocking of the activated receptors relevant to disease and reducing the potential pool of polypeptide bound to irrelevant integrin receptors. Selectively targeting the relevant VLA4 receptors may also allow optimal 2D-VCAM-1 variant polypeptide dose levels that enable prevention of the disease without excessive immune suppression, thereby reducing the risk of opportunistic infections such as that caused by JC virus.

### Example 22

### N-terminally PEGylated 2D-VCAM-1 Variant Pharmacokinetics is Linked to Lymphocyte Release In Vivo

The following example demonstrates that the pharmacokinetics (PK) or serum concentration of N-terminally PEGylated 2D-VCAM-1 variant polypeptide of the present invention in animals is tightly linked to an elevation of peripheral blood lymphocytes, a process termed lymphocytosis. Lymphocytosis is due to inhibition of the interaction of VCAM-1 with VLA4 on either peripheral blood lymphocytes, thereby preventing their transmigration into peripheral compartments, and/or on immature lymphocytes in the bone marrow, leading to their release into the peripheral blood. In both cases, lymphocytosis in polypeptide treated animals is an indication that the 2D-VCAM-1 variant conjugate inhibits the interaction of VLA4 and VCAM-1 in vivo as described in Example 16.

In a second experiment, four cynomolgus monkeys were injected subcutaneously on day 0 (0 hours) and day 8 (168 hours) with 0.3 mg/kg of 2D-VCAM-1 variant Clone 146 N-terminally PEGylated with a 50K branched PEG-aldehyde reagent (PEG50-146). Serum samples were collected at −96, −1, 8, 24, 48, 72, 96, 120, 144, 192, 240 and 336 hours and the amount of PEG50-146 in the serum samples was determined using the ELISA assay described in Example 18. Whole blood samples were collected at −96, −1, 24, 48, 72, 96, 120, 144, 192, 240 and 336 hours and absolute lymphocyte counts were obtained using a Bayer Advia 120 automated analyzer.

As shown in FIG. 16B, treatment of cynomolgus monkeys with PEG50-146 induced peripheral blood lymphocytosis, Lymphocytosis was induced by 24 hours after the first drug administration, remained elevated from approximately 24-72 hours post-administration and began to return to baseline lymphocyte levels (indicated by the dotted line in FIG. 16) at 72-144 hours post-administration. The lymphocyte counts in the peripheral blood increased again after the second administration of PEG50-146 at 168 hours, reached a peak at 240 hours and then began to return to base line levels at 336 hours. As seen in FIG. 16B, the pattern of lymphocytosis observed in the PEG50-146 treated monkeys reflected the serum concentration of PEG50-146 in the animals. In other words, as the PEG50-146 serum levels increased after drug administration, the lymphocyte counts increased, and as the PEG50-146 serum levels decreased after drug administration, the lymphocyte counts decreased.

Taken together, the results shown in FIGS. 16A and 16B for PEG50-146 treated guinea pigs and cynomolgus monkeys, respectively, suggest that the changes in lymphocyte counts in the peripheral blood are linked to the drug serum concentrations (or PK) of PEG50-146 in vivo. As lymphocyte release is a pharmacodynamic maker for in vivo inhibition of the VLA4 and VCAM-1 interaction, the results presented in this example, suggest that PEG50-146 has a tight PK/PD linkage in both cynomolgus monkeys and guinea pigs. The tight PK/PD linkage of PEG50-146 may be beneficial in treating autoimmune diseases in that it allows a direct relationship between drug serum levels and in vivo activity as well as the potential for rapid restoration of normal lymphocyte function in patients as the polypeptide is cleared naturally. This, in turn, may obviate the need for PLEX or immunoabsorption in patients in the event of an opportunistic infection such as that caused by the JC virus, as may be required for other VLA4 antagonists with long pharmacokinetics and a discordance between PK and PD.

### Example 23

### Expression, Purification and Activity Confirmation of 2-D VCAM-1 Variant Polypeptide Fc Fusion Proteins

### A. Subcloning of 2D-VCAM-1 Fc Fusion Sequences for Expression in Mammalian Cells:

The 2D-VCAM-1 Clone 046 (SEQ NO:12) and Clone 146 (SEQ ID NO:18) polypeptides were fused to human IgG2 Fc domains with three of the four cysteines mutated to serine, or to a human IgG1 Fc domain mutated to eliminate effector function to produce 046 and 146 PIg17 (IgG2 SSSC), PIg18 (IgG2 SSCS), and ZIg, respectively, using the SOE (Splicing by Overlap Extension) PCR technology.

The 2D-VCAM-1 clone 046 and 146 genes were PCR amplified from the source vector pVM046 and Cet1019-2DVCAM 146, respectively, using a 5′ primer complementary to the domain 1 region and a 3′ primer complementary to the domain 2 region containing an extension that overlaps with the modified IgG2 or IgG1 Fc domain. The modified IgG2 Fc PIg17 and PIg18 fragments were PCR amplified from source vectors containing these Fc domains using 5′ primers containing extensions that overlap with the VCAM clone and an internal 3′ primer. The modified IgG1 fragment was PCR amplified from a source vector containing this Fc domain using a 5′ primer containing an extension that overlaps with the VCAM clone and a 3′ primer complementary to the polyA sequence downstream of the IgG1 Fc domain. The endogenous VCAM signal peptide sequence was PCR amplified from the source vector pcDNA-2D-VCAM-146-IgG2FcSS using a 5′ primer complementary to the signal sequence with an extension containing an AgeI site and a 3′ primer complementary to the VCAM domain 1 region. The amplified signal sequence, VCAM, and PIg17, PIg18, or ZIg Fc products were mixed for the SOEing reaction and amplified with the signal sequence 5′ primer and the respective IgG Fc 3′ primers to generate the signal sequence-VCAM-Fc products. For the Clone 046 Fc fusion constructs, the PCR products were digested with AgeI and PmlI (PIg17 and PIg18,) or AgeI and SalI (ZIg), and inserted into CET vectors digested with the same enzymes. For the Clone 046 PIg17 and PIg18 constructs, the vector contained the IgG2 sequences 3′ of the PmlI site. For the ZIg construct, an empty vector was used. For the Clone 146 Fc fusion constructs, the PCR products were digested with AgeI and SalI (PIg17, PIg18, and ZIg) and inserted into CET vectors digested with the same enzymes. The resulting plasmids were sequenced to confirm the signal peptide, 2D-VCAM-1, and IgG Fc sequences.

### B. Purification of 2D-VCAM-1 Fc Fusion Proteins

The DNA plasmids encoding the 046-Fc and 146-Fc fusion proteins were transfected into suspension Opti-CHOK1 cells by electroporation with GenePulser (Bio-Rad). After the cells were grown for 48 hours, the cells were selected for expression of the puromycin resistance cassette in medium containing 9 ug/mL of puromycin. The stable pools developed around day 7-8 post selection. The pools were expanded from a T25 flask to a 125 mL shaker flask. Protein A affinity high-performance liquid chromatography (ProA HPLC) was performed to check the expression level of the Fc fusion proteins in the stable pools. Based on the expression titer, cultures of cells were expanded and the supernatants were harvested for purification. The proteins were purified by standard Protein A chromatography using a MabSelect Sure column purchased from GE Healthcare, and with 100 mM Glycine pH 3.7 as the elution buffer. Fluted proteins were dialyzed against PBS overnight. The proteins were analyzed on SDS-PAGE gel under non-reduced and reduced conditions, and by SEC analysis for monomer content.

### C. BIACORE Binding of 2D-VCAM-1 Fc Fusion Proteins

The binding of 2D-VCAM-1 Fc fusions to human VLA4-Fc was performed essentially as described in Example 12 with the following exceptions. Clone 146-Fc and Clone 046-Fc were immobilized on a sensor CM-3 chip according to the manufacturer's protocol. At a flow rate of 5 μl/minute, sensor chip CM-3 was activated with a 35 μl injection of an EDC/NHS mixture (made by mixing equal volumes of 11.5 mg/ml EDC and 75 mg/ml NHS (GE Healthcare, Catalog No. BR1000-50). Activated surfaces were exposed for 3 to 5 minutes to Clone 146-Fc or Clone 046-Fc diluted to 30 ug/ml in immobilization buffer (10 mM sodium acetate, pH 5.0 (GE Healthcare, Catalog No. BR-1003-51)). Un-reacted sites were quenched with 35 μl of 1 M ethanolamine-HCl pH 8.5 (GE Healthcare, Catalog No. BR-1000-50). Approximately 1900 RU of Clone 146-Fc and 2500 RU of Clone 046-Fc were immobilized. VLA4-Fc was diluted in HBS-P buffer plush 1 mM Mn+2 to concentrations of 0.39, 1.56, 6.25, 25 and 100 mM. These were injected over the Clone 146-Fc and Clone 046-Fc for 4 minutes at 30 ul/min. Dissociation was monitored for 30 minutes. After each injection of VLA4-Fc the chip was regenerated by two 30 second injections of 10 mM Glycine, pH 1.7. Dissociation of Clone 146-Fc and Clone 046-Fc were compared qualitatively to historical dissociation curves of their non-Fc, monomeric parental polypeptides (Clone 146 and Clone 046, respectively) binding to human VLA4-Fc wherein the VLA4-Fc was immobilized with goat anti-human IgG as described in Example 12 and the 2D-VCAM polypeptide was injected at 0.16, 0,8, 4.0, 20 and 100 nM in a single cycle. Use of a different assay format to assess the binding affinity for the 2D-VCAM-Fc fusion proteins to human VLA4-Fc was necessary as the anti-human IgG used to immobilize the non-Fc proteins onto the chip in the historical assay would cross-react to the Fc region of both the 2D-VCAM-Fc and VLA4-Fc proteins. Although the two different assay formats to measure 2D-VCAM binding to VLA4 give different RU scales, the dissociation curves and corresponding K_{D}s are comparable for both assay formats.

As shown in FIG. 17A, Clone 146-Fc bound tightly to human VLA4-Fc. Similar shaped dissociation curves were obtained with all 5 concentrations of human VLA4-Fc tested with 100 nM having the highest RU. The dissociation curve of Clone 146-Fc binding to human VLA4-Fc in this assay was qualitatively similar to that of the non-Fc version of Clone 146 (inset in FIG. 17A). Similarly, the data in FIG. 17B demonstrates binding of Clone 046-Fc to human VLA-Fc with the highest RU obtained with 100 nM VLA4-Fc. The dissociation curves for 046-Fc to human VLA4-Fc were qualitatively similar to that obtained for the non-Fc version of clone 046 (inset in FIG. 17B). Taken together, the data in FIGS. 17A and 17B demonstrate that Fc fusions are a functional drug format for 2D-VCAM-1 polypeptides. This assay was designed to measure monomeric binding affinity of one arm of the 2D-VCAM-1 Fc fusion protein to human VLA4 and does measure the enhanced antigen binding of the Fc fusions due to an avidity effect. In vivo, the 2D-VCAM-1 Fc fusions may have an advantage over monomeric 2D-VCAM-1 polypeptides with regards to binding affinity due to enhanced avidity for the VLA4 integrin receptor.

### Example 24

### PEGylation, Isolation and Activity Confirmation of Bifunctionally PEGylated 2D-VCAM-1 Variant Polypeptides

### A. PEGylation of Bifunctionally PEGylated 2D-VCAM-1 Clone 146 (SEQ ID NO:18)

N-terminal PEGylation of tagless 2D-VCAM-1 variant using bifunctional PEG (allows attachment of two protein molecules to one PEG molecule): 5 mg/ml protein (described in Example 15) was reacted with a 0.25 to 1-fold molar excess of 60K bifunctional FEG-aldehyde (SUNBRIGHT DE-600AL2; NOF Corporation, Tokyo, Japan) to protein and 1.26 mg/ml NaCNBH₃. The reaction was incubated for 3 hours at room temperature.

### B. Isolation of the 60K Bifunetionally PEGylated 2D-VCAM-1 Variant on SP Sepharose HP

The FEGylated material was diluted with 1 part water and 2 parts equilibration buffer to a conductivity <6 mS/cm. The material was loaded onto an SF Sepharose UP column equilibrated with 30 mM arginine, 20 mM malic acid, 500 mM glycine, 0.01% Tween-80, pH 4. After an equilibration buffer wash, the protein was elated with a gradient to 250 mM arginine, 20 mM malic acid, 500 mM glycine, 0.01% Tween-80, pH 4. The bifimetionally PEGylated fractions were pooled, concentrated with a 3K MWCO centrifugal concentrator and buffer-exchanged into PBS using a 3.5K MWCO dialysis cassette. Proteins were analyzed by SDS-PAGE and size exclusion chromatography.

C. BIACORE binding of bifunctionally PEGylated 2D-VCAM-1 Clone 146

Bivalent PEG60 Clone 146 was shown to bind captured human VLA4-Fc similarly to Clone 146. Goat anti-human IgG antibody (Jackson ImmunoResearch, Catalog No. 105-005-098) was immobilized on CM-5 sensor chips (GE Healthcare, Catalog No. BR-1000-14) as described above in Example 12. Human VLA4-Fc was diluted to 20 ug/ml in HBS-P and captured for 3 minutes at 15 ul/min. Capture was typically around 600 RU. Clone 146 (SEQ ID NO:18) was diluted to 6, 30 and 150 nM in HBS-P plus 1 mM Mn+2. These concentrations were injected sequentially, starting with the most dilute, for 3 minutes for each concentration at 30 ul/min. Bivalent PEG60 Clone 146 was diluted to 16, 80 and 400 nM in the same buffer and injected in the same manner. Dissociation was monitored for 30 minutes. Between binding cycles the chip was regenerated for 3 minutes with 10 mM glycine, pH 1.7 at 10 ul/min. Clone 146 binding was measured twice to confirm the reproducibility of the assay.

The data shown in FIG. 18 shows that bivalent PEG60 Clone 146 bound human VLA4-Fc. It took a somewhat higher concentration to saturate the human VLA4-Fc on the chip compared to the non-PEGylated Clone 146 polypeptide. Which is typical of all PEGylated 2D-VCAM-1 Clones such as PEG50-146 and PEG80-146. No dissociation was observed during the experiment, indicating very tight binding by both. Clone 146 and bivalent PEG60 Clone 146. Bivalent PEG60 Clone 146 is expected to have a higher binding affinity than monomeric PEGylated Clone 146 (such as PEG50-146 or PEG80-146) to human VLA4 in vivo due to enhanced avidity for the VLA4 integrin receptor. This data demonstrates that linking two identical 2D-VCAM-1 polypeptides with a single PEG moiety to form a bivalent molecule is a functional drug format for 2D-VCAM-1 polypeptides.

While preferred embodiments of the invention have been illustrated and described, it will be readily appreciated that various changes can be made therein without departing from the spirit and scope of the invention.

We claim:

1. A recombinant 2D-VCAM-1 variant polypeptide comprising a sequence which differs in 0-8 amino acid positions from SEQ ID NO:18, and contains at least two amino acid residues selected from the group consisting of: phenylalanine or tyrosine at position 34 relative to SEQ ID NO:18 (F34 or F34Y); praline at position 37 relative to SEQ ID NO:18 (P37); leucine at position 39 relative to SEQ ID NO:18 (L39); and arginine at position 74 relative to SEQ ID NO:18 (R74),
wherein the polypeptide has a binding affinity for a human VLA4 (integrin α4β1) protein that is greater than the binding affinity of Q38L-2D-VCAM-1 (SEQ ID NO:10) for the human VLA4 protein.

2. The 2D-VCAM-1 variant polypeptide of claim 1, wherein the sequence contains praline at position 37 relative to SEQ ID NO:18 (P37) and/or leucine at position 39 relative to SEQ ID NO:18 (139).

3. The 2D-VCAM-1 variant polypeptide of claim 2, wherein the sequence contains praline at position 37 relative to SEQ ID NO:18 (P37) and leucine at position 39 relative to SEQ ID NO:18 (L39).

4. The 2D-VCAM-1 variant polyopeptide of claim 1, wherein the sequence contains phenylalanine at position 34 relative to SEQ ID NO:18 (F34).

5. The 2D-VCAM-1 variant polypeptide of claim 1, wherein the sequence contains tyrosine at position 34 relative to SEQ ID NO: 18 (F34Y).

6. The 2D-VCAM-1 variant polypeptide of claim 1, wherein the sequence contains at least three amino acid residues selected from the group consisting of: phenylalanine or tyrosine at position 34 relative to SEQ ID NO:18 (F34 or F34Y); proline at position 37 relative to SEQ ID NO:18 (P37); leucine at position 39 relative to SEQ ID NO:18 (L39); and arginine at position 74 relative to SEQ ID NO:18 (R74).

7. The 2D-VCAM-1 variant polyopeptide of claim 1, wherein the sequence contains: phenylalanine at position 34 relative to SEQ ID NO:18 (F34); proline at position 37 relative to SEQ ID NO:18 (P37); leucine at position 39 relative to SEQ ID NO:18 (L39); and arginine at position 74 relative to SEQ NO:18 (R74).

8. The 2D-VCAM-1 variant polypeptide claim 1, wherein the sequence further comprises leucine at position 32 relative to SEQ ID NO:18 (L32).

9. The 2D-VCAM-1 variant polypeptide of any of claim 1, wherein the sequence further comprises leucine at position 38 relative to SEQ ID NO:18 (L38).

10. The 2D-VCAM-1 variant polypeptide of any of claim 1, wherein the sequence further comprises serine at position 79 relative to SEQ ID NO:18 (S79).

11. The 2D-VCAM-1 variant polypeptide of claim 1, wherein the sequence differs in 0-5 amino acid positions from SEQ ID NO:18.

12. The 2D4VCAM-1 variant polypeptide of claim 1, wherein the sequence differs in 0-3 amino acid positions from SEQ ID NO:18.

14. The recombinant 2D-VCAM-1 variant polypeptide of claim 1, wherein the polypeptide has a binding affinity for human VLA4 protein that is at least 4-fold greater than the binding affinity of Q38L-2D-VCAM-1 (SEQ ID NO:10) for human VLA4 protein in the assay of Example 10.

15. The recombinant 2D-VCAM-1 variant polypeptide of claim 1, wherein the polypeptide exhibits a ratio of VLA4/LPAM-1 binding affinity that is greater than 1.

16. The recombinant 2D-VCAM-1 variant polypeptide of claim 1, wherein the polypeptide comprises the sequence of SEQ ID NO:18.

17. The recombinant 2D-VCAM-1 variant polyeptide of claim 1, wherein the variant exhibits greater binding to activated VLA4 as compared inactive VI A4 in the assay of Example 21.

18. The recombinant 2D-VCAM-1 variant polypeptide of claim 1, wherein the variant exhibits greater binding to activated VLA4 as compared to the binding of Q38L-2D-VCAM-1 variant (SEQ ID NO:10) to activated VLA4 in the assay of Example 21.

19. The recombinant 2D-VCAM-1 variant polypeptide of claim 1, wherein the 2D-VCAM-1 variant polypeptides does not induce a decrease in the percentage of CD49d+ or CD29+ B cells or T cells in peripheral blood when administered to a mammal, as compared to an untreated control mammal using the assay of Example 19

20. A protein fusion comprising a first polypeptide wherein the first polypeptide is a 2D-VCAM-1 variant polypeptide of claim 1 and a second polypeptide or peptide fused to the N- or C-terminus of the 2D-VCAM-1 variant.

21. The protein fusion of claim 20, wherein the second polypeptide or peptide is selected from the group consisting of a poly-histidine tag, an Fc region of an immunoglobulin, and human serum albumin.

22. The protein fusion of claim 21, the second polypeptide or peptide is an Fc region of an immunoglobulin.

23. A 2D-VCAM-1 variant conjugate comprising a recombinant 2D-VCAM-1 variant polypeptide of claim 1 and at least one conjugation moiety covalently hound to the polypeptide, wherein the conjugation moiety is selected from the group consisting of a non-polypeptide polymer moiety, a sugar moiety, and a non-polypeptide lipophilic moiety.

24. The 2D-VCAM-1 variant conjugate of claim 23, wherein the at least one conjugation moiety is a non-polypeptide polymer moiety.

23. The conjugate of claim 22, wherein the non-polypeptide polymer moiety is a polyethylene glycol (PEG).

25. The conjugate of claim 24, wherein the PEG is a branched PEG of about 20-80 kdal molecular weight.

26. The conjugate of claim 24, wherein the PEG is covalently bound to a lysine or to the N-terminus of the polypeptide.

27. The conjugate of claim 26, wherein the PEG is covalently bound to the N-terminus of the polypeptide.

28. A 21D-VCAM-1 variant conjugate comprising two 2D-VCAM-1 variant polypeptides of claim 1 covalently bonded to one bi-functional PEG.

29. A composition comprising a 2D-VCAM-1 variant polypeptide of claim 1 or a protein fusion or conjugate thereof and a pharmaceutically acceptable carrier or excipient.

30. The composition of claim 29 comprising a conjugate of the 2D-VCAM-1 variant polypeptide, wherein the conjugate comprises a PEG moiety covalently bound to the 2D-VCAM-1 variant polypeptide.

31. The composition of claim 30 comprising a conjugate of the 2D-VCAM-1 variant polypeptide, wherein the conjugate comprises a bifunctional PEG moiety covalently bound to two 2D-VCAM-1 variant polypeptides.

32. A recombinant polynucleotide comprising a nucleic acid sequence that encodes the 2D-VCAM-1 variant polypeptide of claim 1.

33. A vector comprising the polynucleotide of claim 32 operably linked to a promoter.

34. A host cell transformed or transfected with the vector of claim 33.

35. A method of producing a recombinant 2D-VCAM-1 variant polypeptide, the method comprising:
(a) culturing a host cell transformed with the vector of claim 33 under conditions suitable for expression of the polypeptide; and
(b) recovering the polypeptide from the culture medium or from the host cell.

36. A method of producing a recombinant 2D-VCAM-1 variant polypeptide, the method comprising:
(a) culturing a host cell transformed with the vector of claim 33 under conditions suitable for expression of the polypeptide as inclusion bodies;
(b) recovering the inclusion bodies comprising the polypeptide;
(c) solubilizing the inclusion bodies to obtain solubilized polypeptide;
(d) incubating the solubilized polypeptide in a solution of urea and reduced/oxidized glutathione under conditions suitable for the solubilized polypeptide to refold, to obtain refolded polypeptide;
(e) purifying the refolded polypeptide; and
(f) recovering the polypeptide from step (e).

37. The method of claim 36, wherein step (e) comprises purifying the refolded polypeptide.

38. A method of producing a 2D-VCAM-1 variant conjugate comprising:
providing a 2D-VCAM-1 variant polypeptide of any of claims 1; and
covalently attaching at least one polyethyelene glycol (PEG) moiety to the recovered polypeptide.

39. The method of claim 38, wherein the step of covalently attaching at least one conjugation moiety to the polypeptide comprises incubating the polypeptide with a branched PEG-aldehyde of 20-80 kdal molecular weight under conditions effective to conjugate the PEG-aldehyde to the N-terminal-amino group of the polypeptide, thereby producing a 2D-VCAM-1 conjugate comprising a branched PEG moiety covalently attached to the N-terminus of the polypeptide.

40. A method for the therapeutic or prophylactic treatment of a subject for an inflammatory disease or disorder comprising administering to the subject a therapeutically effective amount of the pharmaceutical composition of claim 29.

41. The method of claim 40, wherein the inflammatory disease is multiple sclerosis.


SEQUENCE LISTING (164 sequences)

SEQ ID NO: 1 - DNA, 597 bp, Artificial Sequence
  Synthetic Construct - human 2D-VCAM-1
tttaaaatcgagaccaccccagaatctcgctatcttgctcagattggtgactccgtctcattgacttgcagcaccacaggctgtgagtccccatttttctcttggcgcacccagattgatagtccactgaatgggaaggtgacgaatgaggggaccacatctacgctgacaatgaatcctgttagttttgggaacgaacactcttacctgtgcacagcaacttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatatcaccc

SEQ ID NO: 2 - protein, 199 aa, Artificial Sequence
  Synthetic Construct
FKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 3 - DNA, 1701 bp, Artificial Sequence
  Synthetic Construct - 6D human VCAM-1 (mature form)
tttaaaatcgagaccaccccagaatctagatatcttgctcagattggtgactccgtctcattgacttgcagcaccacaggctgtgagtccccatttttctcttggagaacccagatagatagtccactgaatgggaaggtgacgaatgaggggaccacatctacgctgacaatgaatcctgttagttttgggaacgaacactcttacctgtgcacagcaacttgtgaatctaggaaattggaaaaaggaatccaggtggagatctactcttttcctaaggatccagagattcatttgagtggccctctggaggctgggaagccgatcacagtcaagtgttcagttgctgatgtatacccatttgacaggctggagatagacttactgaaaggagatcatctcatgaagagtcaggaatttctggaggatgcagacaggaagtccctggaaaccaagagtttggaagtaacctttactcctgtcattgaggatattggaaaagttcttgtttgccgagctaaattacacattgatgaaatggattctgtgcccacagtaaggcaggctgtaaaagaattgcaagtctacatatcacccaagaatacagttatttctgtgaatccatccacaaagctgcaagaaggtggctctgtgaccatgacctgttccagcgagggtctaccagctccagagattttctggagtaagaaattagataatgggaatctacagcacctttctggaaatgcaactctcaccttaattgctatgaggatggaagattctggaatttatgtgtgtgaaggagttaatttgattgggaaaaacagaaaagaggtggaattaattgttcaagcattccctagagatccagaaatcgagatgagtggtggcctcgtgaatgggagctctgtcactgtaagctgcaaggttcctagcgtgtacccccttgaccggctggagattgaattacttaagggggagactattctggagaatatagagtttttggaggatacggatatgaaatctctagagaacaaaagtttggaaatgaccttcatccctaccattgaagatactggaaaagctcttgtttgtcaggctaagttacatattgatgacatggaattcgaacccaaacaaaggcagagtacgcaaacactttatgtcaatgttgcccccagagatacaaccgtcttggtcagcccttcctccatcctggaggaaggcagttctgtgaatatgacatgcttgagccagggctttcctgctccgaaaatcctgtggagcaggcagctccctaacggggagctacagcctctttctgagaatgcaactctcaccttaatttctacaaaaatggaagattctggggtttatttatgtgaaggaattaaccaggctggaagaagcagaaaggaagtggaattaattatccaagttactccaaaagacataaaacttacagcttttccttctgagagtgtcaaagaaggagacactgtcatcatctcttgtacatgtggaaatgttccagaaacatggataatcctgaagaaaaaagcggagacaggagacacagtactaaaatctatagatggcgcctataccatccgaaaggcccagttgaaggatgcgggagtatatgaatgtgaatctaaaaacaaagttggctcacaattaagaagtttaacactt

SEQ ID NO: 4 - protein, 567 aa, Artificial Sequence
  Synthetic Construct
FKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQAFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTL

SEQ ID NO: 5 - DNA, 1977 bp, Artificial Sequence
  Synthetic Construct - 7D human VCAM-1 (mature form)
tttaaaatcgagaccaccccagaatctagatatcttgctcagattggtgactccgtctcattgacttgcagcaccacaggctgtgagtccccatttttctcttggagaacccagatagatagtccactgaatgggaaggtgacgaatgaggggaccacatctacgctgacaatgaatcctgttagttttgggaacgaacactcttacctgtgcacagcaacttgtgaatctaggaaattggaaaaaggaatccaggtggagatctactcttttcctaaggatccagagattcatttgagtggccctctggaggctgggaagccgatcacagtcaagtgttcagttgctgatgtatacccatttgacaggctggagatagacttactgaaaggagatcatctcatgaagagtcaggaatttctggaggatgcagacaggaagtccctggaaaccaagagtttggaagtaacctttactcctgtcattgaggatattggaaaagttcttgtttgccgagctaaattacacattgatgaaatggattctgtgcccacagtaaggcaggctgtaaaagaattgcaagtctacatatcacccaagaatacagttatttctgtgaatccatccacaaagctgcaagaaggtggctctgtgaccatgacctgttccagcgagggtctaccagctccagagattttctggagtaagaaattagataatgggaatctacagcacctttctggaaatgcaactctcaccttaattgctatgaggatggaagattctggaatttatgtgtgtgaaggagttaatttgattgggaaaaacagaaaagaggtggaattaattgttcaagagaaaccatttactgttgagatctcccctggaccccggattgctgctcagattggagactcagtcatgttgacatgtagtgtcatgggctgtgaatccccatctttctcctggagaacccagatagacagccctctgagcgggaaggtgaggagtgaggggaccaattccacgctgaccctgagccctgtgagttttgagaacgaacactcttatctgtgcacagtgacttgtggacataagaaactggaaaagggaatccaggtggagctctactcattccctagagatccagaaatcgagatgagtggtggcctcgtgaatgggagctctgtcactgtaagctgcaaggttcctagcgtgtacccccttgaccggctggagattgaattacttaagggggagactattctggagaatatagagtttttggaggatacggatatgaaatctctagagaacaaaagtttggaaatgaccttcatccctaccattgaagatactggaaaagctcttgtttgtcaggctaagttacatattgatgacatggaattcgaacccaaacaaaggcagagtacgcaaacactttatgtcaatgttgcccccagagatacaaccgtcttggtcagcccttcctccatcctggaggaaggcagttctgtgaatatgacatgcttgagccagggctttcctgctccgaaaatcctgtggagcaggcagctccctaacggggagctacagcctctttctgagaatgcaactctcaccttaatttctacaaaaatggaagattctggggtttatttatgtgaaggaattaaccaggctggaagaagcagaaaggaagtggaattaattatccaagttactccaaaagacataaaacttacagcttttccttctgagagtgtcaaagaaggagacactgtcatcatctcttgtacatgtggaaatgttccagaaacatggataatcctgaagaaaaaagcggagacaggagacacagtactaaaatctatagatggcgcctataccatccgaaaggcccagttgaaggatgcgggagtatatgaatgtgaatctaaaaacaaagttggctcacaattaagaagtttaacactt

SEQ ID NO: 6 - protein, 659 aa, Artificial Sequence
  Synthetic Construct
FKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPKNTVISVNPSTKLQEGGSVTMTCSSEGLPAPEIFWSKKLDNGNLQHLSGNATLTLIAMRMEDSGIYVCEGVNLIGKNRKEVELIVQEKPFTVEISPGPRIAAQIGDSVMLTCSVMGCESPSFSWRTQIDSPLSGKVRSEGTNSTLTLSPVSFENEHSYLCTVTCGHKKLEKGIQVELYSFPRDPEIEMSGGLVNGSSVTVSCKVPSVYPLDRLEIELLKGETILENIEFLEDTDMKSLENKSLEMTFIPTIEDTGKALVCQAKLHIDDMEFEPKQRQSTQTLYVNVAPRDTTVLVSPSSILEEGSSVNMTCLSQGFPAPKILWSRQLPNGELQPLSENATLTLISTKMEDSGVYLCEGINQAGRSRKEVELIIQVTPKDIKLTAFPSESVKEGDTVIISCTCGNVPETWIILKKKAETGDTVLKSIDGAYTIRKAQLKDAGVYECESKNKVGSQLRSLTL

SEQ ID NO: 7 - protein, 7 aa, Artificial Sequence
  Synthetic Construct - motif
RPQLDAP

SEQ ID NO: 8 - protein, 7 aa, Artificial Sequence
  Synthetic Construct - motif
RPLLDSP

SEQ ID NO: 9 - DNA, 597 bp, Artificial Sequence
  Synthetic Construct - Q38L-2D-VCAM-1
tttaaaatcgagaccaccccagaatctcgctatcttgctcagattggtgactccgtctcattgacttgcagcaccacaggctgtgagtccccatttttctcttggcgcaccctgattgatagtccactgaatgggaaggtgacgaatgaggggaccacatctacgctgacaatgaatcctgttagttttgggaacgaacactcttacctgtgcacagcaacttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcacca

SEQ ID NO: 10 - protein, 199 aa, Artificial Sequence
  Synthetic Construct
FKIETTPESRYLAQIGDSVSLTCSTTGCESPFFSWRTLIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 11 - DNA, 597 bp, Artificial Sequence
  Synthetic Construct - clone 46
tttaaaatcgagaccaccccagaatctcgctatcttgctcagattggtgactccgtctcattgacttgcagcaccacaggctgtgagtccccatttttctcttggcgtccgcagcttgatgctccgctgaatgggaaggtgacgaatgaggggaccacatctacgctgacaatgaatcctgttagttttgggaacgaacactcttacctgtgcacagcaacttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcacca

SEQ ID NO: 12 - protein, 199 aa, Artificial Sequence
  Synthetic Construct
FKIETTPESRYLAQIGDSVSLTCSTTGCESPFFSWRPQLDAPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 13 - DNA, 597 bp, Artificial Sequence
  Synthetic Construct - clone 47
tttaaaatcgagaccaccccagaatctcgctatcttgctcagattggtgactccgtctcattgacttgcagcaccacaggctgtgagtccccatttttctcttggcggccgctgcttgattcgcctctgaatgggaaggtgacgaatgaggggaccacatctacgctgacaatgaatcctgttagttttgggaacgaacactcttacctgtgcacagcaacttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcacca

SEQ ID NO: 14 - protein, 199 aa, Artificial Sequence
  Synthetic Construct
FKIETTPESRYLAQIGDSVSLTCSTTGCESPFFSWRPLLDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 15 - DNA, 597 bp, Artificial Sequence
  Synthetic Construct - clone 59
tttaaaatcgagaccaccccagaatctcgctatcttgctcagattggtgactccgtctcattgacttgcagcaccacaggctgtgagtccccatttttctcttggcggccgctgcttgattcgcctctgaatgggaaggtgacgaatgaggggaccacatctacgctgacaatgaatcctgttagttttgggaacgaacactcttacctgtgcacagcaacttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaattttttgaggatgcagcgtggaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcaccc

SEQ ID NO: 16 - protein, 199 aa, Artificial Sequence
  Synthetic Construct
FKIETTPESRYLAQIGDSVSLTCSTTGCESPFFSWRPLLDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFFEDAAWKSLETKSLEVTFTPVIEDIGKVLVCRAKLHIDEMDSVPTVRQAVKELQVYISP

SEQ ID NO: 17 - DNA, 597 bp, Artificial Sequence
  Synthetic Construct - clone 146
tttaaaatcgagaccaccccagaatctcgctatcttgctcagattggtgactccgtctcattgacttgcagcaccacaggctgtgagtccccattgttcttttggcgtccgctgcttgatagtccactgaatgggaaggtgacgaatgaggggaccacatctacgctgacaatgaatcctgttagttttgggaacgaacactcttacctgtgcacagcaaggtgtgaatctcgcagcttggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcacca

SEQ ID NO: 18 - protein, 199 aa, Artificial Sequence
  Synthetic Construct
FKIETTPESRYLAQIGDSVSLTCSTTGCESPLFFWRPLLDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 19 - DNA, 597 bp, Artificial Sequence
  Synthetic Construct - clone 147
tttaaaatcgagaccaccccagaatctcgctatcttgctcagattggtgactccgtctcattgacttgcagcaccacaggctgtgagtccccatctttctattggcgtccgctgcttgatagtccactgaatgggaaggtgacgaatgaggggaccacatctacgctgacaatgaatcctgttagttttgggaacgaacactcttacctgtgcacagcagcttgtgaatctcgcaggttggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcacca

SEQ ID NO: 20 - protein, 199 aa, Artificial Sequence
  Synthetic Construct
FKIETTPESRYLAQIGDSVSLTCSTTGCESPSFYWRPLLDSPLNGKVTNEGTTSTLTMNPVSFGNEHSYLCTAACESRRLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 21 - DNA, 597 bp, Artificial Sequence
  Synthetic Construct - clone 150
tttaaaatcgagaccaccccagaatctcgctatcttgctcagattggtgactccgtctcattgacttgcagcaccacaggctgtgagtccccattgttcttttggcgtccgcagcttgatgctccgctgaatgggaaggtgacgaatgaggggaccacatctacgctgacaatgaatcctgttagttttgggaacgaacactcttacctgtgcacagcaaggtgtgaatctcgcagcttggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcacca

SEQ ID NO: 22 - protein, 189 aa, Artificial Sequence
  Synthetic Construct
FKIETTPESRYLAQIGDSVSLTCSTTGCESPLFFWRPQLDAPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQA

SEQ ID NO: 23 - DNA, 597 bp, Artificial Sequence
  Synthetic Construct - clone 151
tttaaaatcgagaccaccccagaatctcgctatcttgctcagattggtgactccgtctcattgacttgcagcaccacaggctgtgagtccccatctttctattggcgtccgcagcttgatgctccgctgaatgggaaggtgacgaatgaggggaccacatctacgctgacaatgaatcctgttagttttgggaacgaacactcttacctgtgcacagcagcttgtgaatctcgcaggttggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcacca

SEQ ID NO: 24 - protein, 199 aa, Artificial Sequence
  Synthetic Construct
FKIETTPESRYLAQIGDSVSLTCSTTGCESPSFYWRPQLDAPLNGKVTNEGTTSTLTMNPVSFGNEHSYLCTAACESRRLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 25 - protein, 15 aa, Artificial Sequence
  Synthetic Construct - linker
GGGGSGGGGSGGGGS

SEQ ID NO: 26 - protein, 10 aa, Artificial Sequence
  Synthetic Construct - linker
GGXGGGXGGG

SEQ ID NO: 27 - protein, 6 aa, Artificial Sequence
  Synthetic Construct - tag
HHHHHH

SEQ ID NO: 28 - protein, 8 aa, Artificial Sequence
  Synthetic Construct - tag
MKHHHHHH

SEQ ID NO: 29 - protein, 10 aa, Artificial Sequence
  tag
MKHHAHHQHH

SEQ ID NO: 30 - protein, 14 aa, Artificial Sequence
  Synthetic Construct - tag
MKHQHQHQHQHQHQ

SEQ ID NO: 31 - protein, 10 aa, Artificial Sequence
  Synthetic Construct - tag
EQKLISEEDL

SEQ ID NO: 32 - DNA, 315 bp, Artificial Sequence
  Synthetic Construct - human IgG1 kappa light chain constant (CKappa) region
actgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgccggtcaccaagagcttcaacaggggagag

SEQ ID NO: 33 - protein, 105 aa, Artificial Sequence
  Synthetic Construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 34 - DNA, 42 bp, Artificial Sequence
  Synthetic Construct - SV5 V5 epitope
ggtaagcctatccctaaccctctcctcggtctcgattctacg

SEQ ID NO: 35 - protein, 14 aa, Artificial Sequence
  Synthetic Construct
GKPIPNPLLGLDST

SEQ ID NO: 36 - DNA, 135 bp, Artificial Sequence
  Synthetic Construct - GPI membrane anchor
ctggaacccacgtactgcgacctcgcccctcccgctggcacgaccgatgccgctcaccctggccggagcgtcgtgcctgccctcctgcctctgctcgccgggaccctcctgctcctggaaaccgctaccgctccc

SEQ ID NO: 37 - protein, 45 aa, Artificial Sequence
  Synthetic Construct
LEPTYCDLAPPAGTTDAAHPGRSVVPALLPLLAGTLLLLETATAP

SEQ ID NO: 38 - DNA, 21 bp, Artificial Sequence
  Synthetic Construct - TBO380
atgcctgggaagatggtcgtg

SEQ ID NO: 39 - DNA, 30 bp, Artificial Sequence
  Synthetic Construct - TBO381
actgtattcttgggtgatatgtagacttgc

SEQ ID NO: 40 - DNA, 40 bp, Artificial Sequence
  Synthetic Construct - TBO388
ttaaggatccgccgccaccatgcctgggaagatggtcgtg

SEQ ID NO: 41 - DNA, 46 bp, Artificial Sequence
  Synthetic Construct - TBO383
gccactcaaatgaatctctgggtccttaggaaaagagtagatctcc

SEQ ID NO: 42 - DNA, 46 bp, Artificial Sequence
  Synthetic Construct - TBO382
ggagatctactcttttcctaaggacccagagattcatttgagtggc

SEQ ID NO: 43 - DNA, 34 bp, Artificial Sequence
  Synthetic Construct - TBO385
gggtgatatgtacacttgcaattcttttacagcc

SEQ ID NO: 44 - DNA, 55 bp, Artificial Sequence
  Synthetic Construct - TBO392
gatggtgcagccacagtgggtgatatgtacacttgcaattcttttacagcctgcc

SEQ ID NO: 45 - DNA, 23 bp, Artificial Sequence
  Synthetic Construct - TBO389
caccatctgtcttcatcttcccg

SEQ ID NO: 46 - DNA, 23 bp, Artificial Sequence
  Synthetic Construct - TBO390
ctcaggccctgatgggtgacttc

SEQ ID NO: 47 - DNA, 52 bp, Artificial Sequence
  Synthetic Construct - TBO391
gcaagtgtacatatcacccactgtggctgcaccatctgtcttcatcttcccg

SEQ ID NO: 48 - DNA, 77 bp, Artificial Sequence
  Synthetic Construct - TBO393
ggcttaccgcctgcaggtcactctcccctgttgaagctcttggtgaccggcgagctcaggccctgatgggtgacttc

SEQ ID NO: 49 - DNA, 1173 bp, Artificial Sequence
  Synthetic Construct - 2D-VCAM-1-CKappa-UGA- V5-GPI
atgcctgggaagatggtcgtgatccttggagcctcaaatatactttggataatgtttgcagcttctcaagcttttaaaatcgagaccaccccagaatctagatatcttgctcagattggtgactccgtctcattgacttgcagcaccacaggctgtgagtccccatttttctcttggagaacccagatagatagtccactgaatgggaaggtgacgaatgaggggaccacatctacgctgacaatgaatcctgttagttttgggaacgaacactcttacctgtgcacagcaacttgtgaatctaggaaattggaaaaaggaatccaggtggagatctactcttttcctaaggacccagagattcatttgagtggccctctggaggctgggaagccgatcacagtcaagtgttcagttgctgatgtatacccatttgacaggctggagatagacttactgaaaggagatcatctcatgaagagtcaggaatttctggaggatgcagacaggaagtccctggaaaccaagagtttggaagtaacctttactcctgtcattgaggatattggaaaagttcttgtttgccgagctaaattacacattgatgaaatggattctgtgcccacagtaaggcaggctgtaaaagaattgcaagtgtacatatcacccactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgccggtcaccaagagcttcaacaggggagagtgacctgcaggcggtaagcctatccctaaccctctcctcggtctcgattctacgctggaacccacgtactgcgacctcgcccctcccgctggcacgaccgatgccgctcaccctggccggagcgtcgtgcctgccctcctgcctctgctcgccgggaccctcctgctcctggaaaccgctaccgctccc

SEQ ID NO: 50 - protein, 328 aa, Artificial Sequence
  Synthetic Construct - 2D-VCAM-1-CKappa-UGA- V5-GPI
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 51 - DNA, 60 bp, Artificial Sequence
  Synthetic Construct - TBO392
tgagctcgccggtcaccaagagcttcaacaggggagagcctgcaggcggtaagcctatcc

SEQ ID NO: 52 - DNA, 23 bp, Artificial Sequence
  TBO014
agaaggcacagtcgaggctgatc

SEQ ID NO: 53 - DNA, 1170 bp, Artificial Sequence
  Synthetic Construct - 2D-VCAM-1-CKappa-V5-GPI
atgcctgggaagatggtcgtgatccttggagcctcaaatatactttggataatgtttgcagcttctcaagcttttaaaatcgagaccaccccagaatctagatatcttgctcagattggtgactccgtctcattgacttgcagcaccacaggctgtgagtccccatttttctcttggagaacccagatagatagtccactgaatgggaaggtgacgaatgaggggaccacatctacgctgacaatgaatcctgttagttttgggaacgaacactcttacctgtgcacagcaacttgtgaatctaggaaattggaaaaaggaatccaggtggagatctactcttttcctaaggacccagagattcatttgagtggccctctggaggctgggaagccgatcacagtcaagtgttcagttgctgatgtatacccatttgacaggctggagatagacttactgaaaggagatcatctcatgaagagtcaggaatttctggaggatgcagacaggaagtccctggaaaccaagagtttggaagtaacctttactcctgtcattgaggatattggaaaagttcttgtttgccgagctaaattacacattgatgaaatggattctgtgcccacagtaaggcaggctgtaaaagaattgcaagtgtacatatcacccactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgccggtcaccaagagcttcaacaggggagagcctgcaggcggtaagcctatccctaaccctctcctcggtctcgattctacgctggaacccacgtactgcgacctcgcccctcccgctggcacgaccgatgccgctcaccctggccggagcgtcgtgcctgccctcctgcctctgctcgccgggaccctcctgctcctggaaaccgctaccgctccc

SEQ ID NO: 54 - protein, 390 aa, Artificial Sequence
  Synthetic Construct
MPGKMVVILGASNILWIMFAASQAFKIETTPESRYLAQIGDSVSLTCSTTGCESPFFSWRTQIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEPAGGKPIPNPLLGLDSTLEPTYCDLAPPAGTTDAAHPGRSVVPALLPLLAGTLLLLETATAP

SEQ ID NO: 55 - DNA, 597 bp, Artificial Sequence; identical to SEQ ID NO: 1
  Synthetic Construct - E. coli optimized human 2D-VCAM-1

SEQ ID NO: 56 - DNA, 1184 bp, Artificial Sequence
  Synthetic Construct - 2D-VCAM-1 phage display construct
atgaaaaagaatatcgcatttcttcttgcatctatgttcgttttttctattgctacggccgctatggcctttaaaatcgagaccaccccagaatctagatatcttgctcagattggtgactccgtctcattgacttgcagcaccacaggctgtgagtccccatttttctcttggagaacccagatagatagtccactgaatgggaaggtgacgaatgaggggaccacatctacgctgacaatgaatcctgttagttttgggaacgaacactcttacctgtgcacagcaacttgtgaatctaggaaattggaaaaaggaatccaggtggagatctactcttttcctaaggacccagagattcatttgagtggccctctggaggctgggaagccgatcacagtcaagtgttcagttgctgatgtatacccatttgacaggctggagatagacttactgaaaggagatcatctcatgaagagtcaggaatttctggaggatgcagacaggaagtccctggaaaccaagagtttggaagtaacctttactcctgtcattgaggatattggaaaagttcttgtttgccgagctaaattacacattgatgaaatggattctgtgcccacagtaaggcaggctgtaaaagaattgcaagtgtacatatcacccgcggccgcacaccatcaccatcaccactagcctagcaagatgggatccggtggcggctccggttccggagacttcgactacgagaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtc

SEQ ID NO: 57 - DNA, 22 bp, Artificial Sequence
  Synthetic Construct - TBO358
tacaacgtggacactgagagcg

SEQ ID NO: 58 - DNA, 22 bp, Artificial Sequence
  Synthetic Construct - TBO357
ccattttctcctggctgtctgg

SEQ ID NO: 59 - DNA, 41 bp, Artificial Sequence
  Synthetic Construct - TBO365
cggtgagcttgctggtgaggaagaaggagccgtcggagtcc

SEQ ID NO: 60 - DNA, 25 bp, Artificial Sequence
  Synthetic Construct - TBO367
tttactgatgacatgaggaccaagg

SEQ ID NO: 61 - DNA, 25 bp, Artificial Sequence
  Synthetic Construct - TBO366
agtacagaggaattcccaccacttc

SEQ ID NO: 62 - DNA, 27 bp, Artificial Sequence
  Synthetic Construct - TBO352
gaaataacgtttgggtctttgatgatg

SEQ ID NO: 63 - DNA, 100 bp, Artificial Sequence
  Synthetic Construct - TBO359
gcataggatccgccgccaccatgaagctgcctgtgaggctgctcgtgctgatgttctggattcctgccagctccagctacaacgtggacactgagagcgc

SEQ ID NO: 64 - DNA, 40 bp, Artificial Sequence
  Synthetic Construct - TBO355
ccacaagtcacgatggagccattttctcctggctgtctgg

SEQ ID NO: 65 - protein, 19 aa, Artificial Sequence
  Synthetic Construct - mouse IgG Kappa signal peptide
MKLPVRLLVLMFWIPASSS

SEQ ID NO: 66 - DNA, 39 bp, Artificial Sequence
  Synthetic Construct - TBO354
gccaggagaaaatggctccatcgtgacttgtgggcatag

SEQ ID NO: 67 - DNA, 43 bp, Artificial Sequence
  Synthetic Construct - TBO369
gaattcctctgtactcagtttactgatgacatgaggaccaagg

SEQ ID NO: 68 - DNA, 43 bp, Artificial Sequence
  Synthetic Construct - TBO368
cctcatgtcatcagtaaactgagtacagaggaattcccaccac

SEQ ID NO: 69 - DNA, 39 bp, Artificial Sequence
  Synthetic Construct - TBO353
agtcatgtcgacgaaataacgtttgggtctttgatgatg

SEQ ID NO: 70 - DNA, 26 bp, Artificial Sequence
  Synthetic Construct - TBO351
gacaagaaagttgagcccaaatcttg

SEQ ID NO: 71 - DNA, 27 bp, Artificial Sequence
  Synthetic Construct - TBO305
tttacccggagacagggagaggctctt

SEQ ID NO: 72 - DNA, 35 bp, Artificial Sequence
  Synthetic Construct - TBO377
cctgtggttctcgaggctgccctttggctttggag

SEQ ID NO: 73 - DNA, 36 bp, Artificial Sequence
  Synthetic Construct - TBO376
gccaaagggcagcctcgagaaccacaggtgtacacc

SEQ ID NO: 74 - DNA, 51 bp, Artificial Sequence
  Synthetic Construct - TBO361
gcataggatccaaagcagcaaagtcgacaagaaagttgagcccaaatcttg

SEQ ID NO: 75 - DNA, 43 bp, Artificial Sequence
  Synthetic Construct - TBO364
cggctccttcttcctcaccagcaagctcaccgtggacaagagc

SEQ ID NO: 76 - DNA, 43 bp, Artificial Sequence
  Synthetic Construct - TBO360
aaacttgtttaaacttactatttacccggagacagggagaggc

SEQ ID NO: 77 - DNA, 3540 bp, Artificial Sequence
  Synthetic Construct - alpha4-Fc
tacaacgtggacactgagagcgcgctgctttaccagggcccccacaacacgctgttcggctactcggtcgtgctgcacagccacggggcgaaccgatggctcctagtgggtgcgcccactgccaactggctcgccaacgcttcagtgatcaatcccggggcgatttacagatgcaggatcggaaagaatcccggccagacgtgcgaacagctccagctgggtagccctaatggagaaccttgtggaaagacttgtttggaagagagagacaatcagtggttgggggtcacactttccagacagccaggagaaaatggctccatcgtgacttgtgggcatagatggaaaaatatattttacataaagaatgaaaataagctccccactggtggttgctatggagtgccccctgatttacgaacagaactgagtaaaagaatagctccgtgttatcaagattatgtgaaaaaatttggagaaaattttgcatcatgtcaagctggaatatccagtttttacacaaaggatttaattgtgatgggggccccaggatcatcttactggactggctctctttttgtctacaatataactacaaataaatacaaggcttttttagacaaacaaaatcaagtaaaatttggaagttatttaggatattcagtcggagctggtcattttcggagccagcatactaccgaagtagtcggaggagctcctcaacatgagcagattggtaaggcatatatattcagcattgatgaaaaagaactaaatatcttacatgaaatgaaaggtaaaaagcttggatcgtactttggagcttctgtctgtgctgtggacctcaatgcagatggcttctcagatctgctcgtgggagcacccatgcagagcaccatcagagaggaaggaagagtgtttgtgtacatcaactctggctcgggagcagtaatgaatgcaatggaaacaaacctcgttggaagtgacaaatatgctgcaagatttggggaatctatagttaatcttggcgacattgacaatgatggctttgaagatgttgctatcggagctccacaagaagatgacttgcaaggtgctatttatatttacaatggccgtgcagatgggatctcgtcaaccttctcacagagaattgaaggacttcagatcagcaaatcgttaagtatgtttggacagtctatatcaggacaaattgatgcagataataatggctatgtagatgtagcagttggtgcttttcggtctgattctgctgtcttgctaaggacaagacctgtagtaattgttgacgcttctttaagccaccctgagtcagtaaatagaacgaaatttgactgtgttgaaaatggatggccttctgtgtgcatagatctaacactttgtttctcatataagggcaaggaagttccaggttacattgttttgttttataacatgagtttggatgtgaacagaaaggcagagtctccaccaagattctatttctcttctaatggaacttctgacgtgattacaggaagcatacaggtgtccagcagagaagctaactgtagaacacatcaagcatttatgcggaaagatgtgcgggacatcctcaccccaattcagattgaagctgcttaccaccttggtcctcatgtcatcagtaaactgagtacagaggaattcccaccacttcagccaattcttcagcagaagaaagaaaaagacataatgaaaaaaacgataaactttgcaaggttttgtgcccatgaaaattgttctgctgatttacaggtttctgcaaagattgggtttttgaagccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctggaagagaagcaaataaactgtgaagtcacagataactctggcgtggtacaacttgactgcagtattggctatatatatgtagatcatctctcaaggatagatattagctttctcctggatgtgagctcactcagcagagcggaagaggacctcagtatcacagtgcatgctacctgtgaaaatgaagaggaaatggacaatctaaagcacagcagagtgactgtagcaatacctttaaaatatgaggttaagctgactgttcatgggtttgtaaacccaacttcatttgtgtatggatcaaatgatgaaaatgagcctgaaacgtgcatggtggagaaaatgaacttaactttccatgttatcaacactggcaatagtatggctcccaatgttagtgtggaaataatggtaccaaattcttttagcccccaaactgataagctgttcaacattttggatgtccagactactactggagaatgccactttgaaaattatcaaagagtgtgtgcattagagcagcaaaagagtgcaatgcagaccttgaaaggcatagtccagttcttgtccaagactgataagaggctattgtactgcataaaagctgatccacattgtttaaatttcttgtgtaattttgggaaaatggaaagtggaaaagaagccagtgttcatatccaactggaaggccggccatccattttagaaatggatgagacttcagcactcaagtttgaaataagagcaacaggttttccagagccaaatccaagagtaattgaactaaacaaggatgagaatgttgcgcacgttctactggaaggactacatcatcaaagacccaaacgttatttcgtcgacaagaaagttgagcccaaatcttgtgacaaaactcacacatgcccaccgtgcccagcacctgaactcctggggggaccgtcggtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagcctcgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctcaccagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 78 - protein, 1180 aa, Artificial Sequence
  Synthetic Construct
YNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRPVVIVDASLSHPESVNRTKFDCVENGWPSVCIDLTLCFSYKGKEVPGYIVLFYNMSLDVNRKAESPPRFYFSSNGTSDVITGSIQVSSREANCRTHQAFMRKDVRDILTPIQIEAAYHLGPHVISKLSTEEFPPLQPILQQKKEKDIMKKTINFARFCAHENCSADLQVSAKIGFLKPHENKTYLAVGSMKTLMLNVSLFNAGDDAYETTLHVKLPVGLYFIKILELEEKQINCEVTDNSGVVQLDCSIGYIYVDHLSRIDISFLLDVSSLSRAEEDLSITVHATCENEEEMDNLKHSRVTVAIPLKYEVKLTVHGFVNPTSFVYGSNDENEPETCMVEKMNLTFHVINTGNSMAPNVSVEIMVPNSFSPQTDKLFNILDVQTTTGECHFENYQRVCALEQQKSAMQTLKGIVQFLSKTDKRLLYCIKADPHCLNFLCNFGKMESGKEASVHIQLEGRPSILEMDETSALKFEIRATGFPEPNPRVIELNKDENVAHVLLEGLHHQRPKRYFVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 79 - DNA, 43 bp, Artificial Sequence
  Synthetic Construct - TBO379
ggcttaccgcctgcaggtcatttacccggagacagggagaggc

SEQ ID NO: 80 - DNA, 48 bp, Artificial Sequence
  Synthetic Construct - TBO378
ccctgtctccgggtaaatgacctgcaggcggtaagcctatccctaacc

SEQ ID NO: 81 - DNA, 48 bp, Artificial Sequence
  Synthetic Construct - TBO104
atcagcggtttaaactttcactattactagggagcggtagcggtttcc

SEQ ID NO: 82 - DNA, 29 bp, Artificial Sequence
  Synthetic Construct - TBO370
atgaatttacaaccaattttctggattgg

SEQ ID NO: 83 - DNA, 24 bp, Artificial Sequence
  Synthetic Construct - TBO370
gatgtctggaccagtgggacactc

SEQ ID NO: 84 - DNA, 49 bp, Artificial Sequence
  Synthetic Construct - TBO372
gcataggatccgccgccaccatgaatttacaaccaattttctggattgg

SEQ ID NO: 85 - DNA, 36 bp, Artificial Sequence
  Synthetic Construct - TBO373
agtcatgtcgacgatgtctggaccagtgggacactc

SEQ ID NO: 86 - DNA, 38 bp, Artificial Sequence
  Synthetic Construct - TBO363
gcctttgaccaggcagtacaggctgacctggttcttgg

SEQ ID NO: 87 - DNA, 37 bp, Artificial Sequence
  Synthetic Construct - TBO362
aggtcagcctgtactgcctggtcaaaggcttctatcc

SEQ ID NO: 88 - DNA, 2838 bp, Artificial Sequence
  Synthetic Construct - beta1-Fc
caaacagatgaaaatagatgtttaaaagcaaatgccaaatcatgtggagaatgtatacaagcagggccaaattgtgggtggtgcacaaattcaacatttttacaggaaggaatgcctacttctgcacgatgtgatgatttagaagccttaaaaaagaagggttgccctccagatgacatagaaaatcccagaggctccaaagatataaagaaaaataaaaatgtaaccaaccgtagcaaaggaacagcagagaagctcaagccagaggatattactcagatccaaccacagcagttggttttgcgattaagatcaggggagccacagacatttacattaaaattcaagagagctgaagactatcccattgacctctactaccttatggacctgtcttactcaatgaaagacgatttggagaatgtaaaaagtcttggaacagatctgatgaatgaaatgaggaggattacttcggacttcagaattggatttggctcatttgtggaaaagactgtgatgccttacattagcacaacaccagctaagctcaggaacccttgcacaagtgaacagaactgcaccagcccatttagctacaaaaatgtgctcagtcttactaataaaggagaagtatttaatgaacttgttggaaaacagcgcatatctggaaatttggattctccagaaggtggtttcgatgccatcatgcaagttgcagtttgtggatcactgattggctggaggaatgttacacggctgctggtgttttccacagatgccgggtttcactttgctggagatgggaaacttggtggcattgttttaccaaatgatggacaatgtcacctggaaaataatatgtacacaatgagccattattatgattatccttctattgctcaccttgtccagaaactgagtgaaaataatattcagacaatttttgcagttactgaagaatttcagcctgtttacaaggagctgaaaaacttgatccctaagtcagcagtaggaacattatctgcaaattctagcaatgtaattcagttgatcattgatgcatacaattccctttcctcagaagtcattttggaaaacggcaaattgtcagaaggagtaacaataagttacaaatcttactgcaagaacggggtgaatggaacaggggaaaatggaagaaaatgttccaatatttccattggagatgaggttcaatttgaaattagcataacttcaaataagtgtccaaaaaaggattctgacagctttaaaattaggcctctgggctttacggaggaagtagaggttattcttcagtacatctgtgaatgtgaatgccaaagcgaaggcatccctgaaagtcccaagtgtcatgaaggaaatgggacatttgagtgtggcgcgtgcaggtgcaatgaagggcgtgttggtagacattgtgaatgcagcacagatgaagttaacagtgaagacatggatgcttactgcaggaaagaaaacagttcagaaatctgcagtaacaatggagagtgcgtctgcggacagtgtgtttgtaggaagagggataatacaaatgaaatttattctggcaaattctgcgagtgtgataatttcaactgtgatagatccaatggcttaatttgtggaggaaatggtgtttgcaagtgtcgtgtgtgtgagtgcaaccccaactacactggcagtgcatgtgactgttctttggatactagtacttgtgaagccagcaacggacagatctgcaatggccggggcatctgcgagtgtggtgtctgtaagtgtacagatccgaagtttcaagggcaaacgtgtgagatgtgtcagacctgccttggtgtctgtgctgagcataaagaatgtgttcagtgcagagccttcaataaaggagaaaagaaagacacatgcacacaggaatgttcctattttaacattaccaaggtagaaagtcgggacaaattaccccagccggtccaacctgatcctgtgtcccattgtaaggagaaggatgttgacgactgttggttctattttacgtattcagtgaatgggaacaacgaggtcatggttcatgttgtggagaatccagagtgtcccactggtccagacatcgtcgacaagaaagttgagcccaaatcttgtgacaaaactcacacatgcccaccgtgcccagcacctgaactcctggggggaccgtcggtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagcctcgagaaccacaggtgtacaccctgcccccatcccgggatgagctgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 89 - protein, 946 aa, Artificial Sequence
  Synthetic Construct
QTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - DNA, 30 bp, Artificial Sequence
  Synthetic Construct - TBO374
agacccaagcttgctagcttattgcggtag

SEQ ID NO: 91 - DNA, 21 bp, Artificial Sequence
  Synthetic Construct - TBO375
ctcctgggtgacgaagacggc

SEQ ID NO: 92 - protein, 14 aa, Artificial Sequence
  Synthetic Construct - alpha4 N-terminal sequence
YNVDTESALLYQGP

SEQ ID NO: 93 - DNA, 22 bp, Artificial Sequence
  Synthetic Construct - TBO395
atggtggctttgccaatggtcc

SEQ ID NO: 94 - DNA, 23 bp, Artificial Sequence
  Synthetic Construct - TBO396
gggtctcactctgagcacgaccg

SEQ ID NO: 95 - DNA, 45 bp, Artificial Sequence
  Synthetic Construct - TBO395
cttgcataggatccgccgccaccatggtggctttgccaatggtcc

SEQ ID NO: 96 - DNA, 58 bp, Artificial Sequence
  Synthetic Construct - TBO398
actttcttgtcgacgtggtctgctcccttttcttggggtctcactctgagcacgaccg

SEQ ID NO: 97 - DNA, 2823 bp, Artificial Sequence
  Synthetic Construct - beta7-Fc
gaattggacgccaagatcccatccacaggggatgccacagaatggcggaatcctcacctgtccatgctggggtcctgccagccagccccctcctgccagaagtgcatcctctcacaccccagctgtgcatggtgcaagcaactgaacttcaccgcgtcgggagaggcggaggcgcggcgctgcgcccgacgagaggagctgctggctcgaggctgcccgctggaggagctggaggagccccgcggccagcaggaggtgctgcaggaccagccgctcagccagggcgcccgcggagagggtgccacccagctggcgccgcagcgggtccgggtcacgctgcggcctggggagccccagcagctccaggtccgcttccttcgtgctgagggatacccggtggacctgtactaccttatggacctgagctactccatgaaggacgacctggaacgcgtgcgccagctcgggcacgctctgctggtccggctgcaggaagtcacccattctgtgcgcattggttttggttcctttgtggacaaaacggtgctgccctttgtgagcacagtaccctccaaactgcgccacccctgccccacccggctggagcgctgccagtcaccattcagctttcaccatgtgctgtccctgacgggggacgcacaagccttcgagcgggaggtggggcgccagagtgtgtccggcaatctggactcgcctgaaggtggcttcgatgccattctgcaggctgcactctgccaggagcagattggctggagaaatgtgtcccggctgctggtgttcacttcagacgacacattccatacagctggggacgggaagttgggcggcattttcatgcccagtgatgggcactgccacttggacagcaatggcctctacagtcgcagcacagagtttgactacccttctgtgggtcaggtagcccaggccctctctgcagcaaatatccagcccatctttgctgtcaccagtgccgcactgcctgtctaccaggagctgagtaaactgattcctaagtctgcagttggggagctgagtgaggactccagcaacgtggtacagctcatcatggatgcttataatagcctgtcttccaccgtgacccttgaacactcttcactccctcctggggtccacatttcttacgaatcccagtgtgagggtcctgagaagagggagggtaaggctgaggatcgaggacagtgcaaccacgtccgaatcaaccagacggtgactttctgggtttctctccaagccacccactgcctcccagagccccatctcctgaggctccgggcccttggcttctcagaggagctgattgtggagttgcacacgctgtgtgactgtaattgcagtgacacccagccccaggctccccactgcagtgatggccagggacacctacaatgtggtgtatgcagctgtgcccctggccgcctaggtcggctctgtgagtgctctgtggcagagctgtcctccccagacctggaatctgggtgccgggctcccaatggcacagggcccctgtgcagtggaaagggtcactgtcaatgtggacgctgcagctgcagtggacagagctctgggcatctgtgcgagtgtgacgatgccagctgtgagcgacatgagggcatcctctgcggaggctttggtcgctgccaatgtggagtatgtcactgtcatgccaaccgcacgggcagagcatgcgaatgcagtggggacatggacagttgcatcagtcccgagggagggctctgcagtgggcatggacgctgcaaatgcaaccgctgccagtgcttggacggctactatggtgctctatgcgaccaatgcccaggctgcaagacaccatgcgagagacaccgggactgtgcagagtgtggggccttcaggactggcccactggccaccaactgcagtacagcttgtgcccataccaatgtgaccctggccttggcccctatcttggatgatggctggtgcaaagagcggaccctggacaaccagctgttcttcttcttggtggaggatgacgccagaggcacggtcgtgctcagagtgagaccccaagaaaagggagcagaccacgtcgacaagaaagttgagcccaaatcttgtgacaaaactcacacatgcccaccgtgcccagcacctgaactcctggggggaccgtcggtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagcctcgagaaccacaggtgtacaccctgcccccatcccgggatgagctgaccaagaaccaggtcagcctgtactgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 98 - protein, 941 aa, Artificial Sequence [sequence omitted]

SEQ ID NO: 99 - protein, 13 aa, Artificial Sequence
  Synthetic Construct - PLP Peptide 139-151
HSLGKWLGHPDKF

SEQ ID NO: 100 - DNA, 789 bp, Artificial Sequence
  Synthetic Construct - 146R1
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggccgctgctggacagccccctgaacggcaaagtgaccaacgagggcaccacctccaccctgaccatgaaccccgtgtccttcggcaacgagcactcctacctgtgtacagctcggtgcgagagccggtccctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 101 - protein, 261 aa, Artificial Sequence
  Synthetic Construct - 146R1
MPGKMVVILGASNILWIMFAASQAFKIETTPESRYLAQIGDSVSLTCSTTGCESPFFFWRPLLDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 102 - DNA, 789 bp, Artificial Sequence
  Synthetic Construct - 146R2
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccactgttctcttggcggccgctgctggacagccccctgaacggcaaagtgaccaacgagggcaccacctccaccctgaccatgaaccccgtgtccttcggcaacgagcactcctacctgtgtacagctcggtgcgagagccggtccctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 103 - protein, 261 aa, Artificial Sequence
  Synthetic Construct - 146R2
MPGKMVVILGASNILWIMFAASQAFKIETTPESRYLAQIGDSVSLTCSTTGCESPLFSWRPLLDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 104 - DNA, 789 bp, Artificial Sequence
  Synthetic Construct - 146R3
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccactgttcttttggcggaccctgctggacagccccctgaacggcaaagtgaccaacgagggcaccacctccaccctgaccatgaaccccgtgtccttcggcaacgagcactcctacctgtgtacagctcggtgcgagagccggtccctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 105 - protein, 261 aa, Artificial Sequence
  Synthetic Construct - 146R3
MPGKMVVILGASNILWIMFAASQAFKIETTPESRYLAQIGDSVSLTCSTTGCESPLFFWRTLLDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 106 - DNA, 789 bp, Artificial Sequence
  Synthetic Construct - 146R4
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccactgttcttttggcggccgcagctggacagccccctgaacggcaaagtgaccaacgagggcaccacctccaccctgaccatgaaccccgtgtccttcggcaacgagcactcctacctgtgtacagctcggtgcgagagccggtccctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 107 - protein, 261 aa, Artificial Sequence
  Synthetic Construct - 146R4
MPGKMVVILGASNILWIMFAASQAFKIETTPESRYLAQIGDSVSLTCSTTGCESPLFFWRPQLDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 108 - DNA, 789 bp, Artificial Sequence
  Synthetic Construct - 146R5
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccactgttcttttggcggccgctgatcgacagccccctgaacggcaaagtgaccaacgagggcaccacctccaccctgaccatgaaccccgtgtccttcggcaacgagcactcctacctgtgtacagctcggtgcgagagccggtccctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 109 - protein, 261 aa, Artificial Sequence
  Synthetic Construct - 146R5
MPGKMVVILGASNILWIMFAASQAFKIETTPESRYLAQIGDSVSLTCSTTGCESPLFFWRPLIDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 110 - DNA, 789 bp, Artificial Sequence
  Synthetic Construct - 146R6
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccactgttcttttggcggccgctgctggacagccccctgaacggcaaagtgaccaacgagggcaccacctccaccctgaccatgaaccccgtgtccttcggcaacgagcactcctacctgtgtacagctacctgcgagagccggtccctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 111 - protein, 261 aa, Artificial Sequence
  Synthetic Construct - 146R6
MPGKMVVILGASNILWIMFAASQAFKIETTPESRYLAQIGDSVSLTCSTTGCESPLFFWRPLLDSPLNGKVTNEGTTSTLTMNPVSFGNEHSYLCTATCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 112 - DNA, 789 bp, Artificial Sequence
  Synthetic Construct - 146R7
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccactgttcttttggcggccgctgctggacagccccctgaacggcaaagtgaccaacgagggcaccacctccaccctgaccatgaaccccgtgtccttcggcaacgagcactcctacctgtgtacagctcggtgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 113 - protein, 261 aa, Artificial Sequence
  Synthetic Construct - 146R7
MPGKMVVILGASNILWIMFAASQAFKIETTPESRYLAQIGDSVSLTCSTTGCESPLFFWRPLLDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 114 - DNA, 789 bp, Artificial Sequence
  Synthetic Construct - 146R8
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccactgttcttttggcggccgctgctggacagccccctgaacggcaaagtgaccaacgagggcaccacctccaccctgaccatgaaccccgtgtccttcggcaacgagcactcctacctgtgtacagctacctgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 115 - protein, 261 aa, Artificial Sequence
  Synthetic Construct - 146R8
MPGKMVVILGASNILWIMFAASQAFKIETTPESRYLAQIGDSVSLTCSTTGCESPLFFWRPLLDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 116 - DNA, 789 bp, Artificial Sequence
  Synthetic Construct - 146R9
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggccgcagctggacagccccctgaacggcaaagtgaccaacgagggcaccacctccaccctgaccatgaaccccgtgtccttcggcaacgagcactcctacctgtgtacagctcggtgcgagagccggtccctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 117 - protein, 261 aa, Artificial Sequence
  Synthetic Construct - 146R9
MPGKMVVILGASNILWIMFAASQAFKIETTPESRYLAQIGDSVSLTCSTTGCESPFFFWRPQLDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 118 - DNA, 789 bp, Artificial Sequence
  Synthetic Construct - 146R10
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggccgctgctggacagccccctgaacggcaaagtgaccaacgagggcaccacctccaccctgaccatgaaccccgtgtccttcggcaacgagcactcctacctgtgtacagctacctgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 119 - protein, 261 aa, Artificial Sequence
  Synthetic Construct - 146R10
MPGKMVVILGASNILWIMFAASQAFKIETTPESRYLAQIGDSVSLTCSTTGCESPFFFWRPLLDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 120 - DNA, 789 bp, Artificial Sequence
  Synthetic Construct - 146R11
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccactgttcttttggcggccgcagctggacagccccctgaacggcaaagtgaccaacgagggcaccacctccaccctgaccatgaaccccgtgtccttcggcaacgagcactcctacctgtgtacagctacctgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 121 - protein, 261 aa, Artificial Sequence
  Synthetic Construct - 146R11
MPGKMVVILGASNILWIMFAASQAFKIETTPESRYLAQIGDSVSLTCSTTGCESPLFFWRPQLDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 122 - DNA, 789 bp, Artificial Sequence
  Synthetic Construct - 146R12
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttctcttggcggccgctgctggacagccccctgaacggcaaagtgaccaacgagggcaccacctccaccctgaccatgaaccccgtgtccttcggcaacgagcactcctacctgtgtacagctacctgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 123 - protein, 261 aa, Artificial Sequence [sequence omitted]

SEQ ID NO: 124 - DNA, 789 bp, Artificial Sequence
  Synthetic Construct - 146R13
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggccgctgctggacagccccctgaacggcaaagtgaccaacgagggcaccacctccaccctgaccatgaaccccgtgtccttcggcaacgagcactcctacctgtgtacagctcggtgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 125 - protein, 261 aa, Artificial Sequence
  Synthetic Construct - 146R13
MPGKMVVILGASNILWIMFAASQAFKIETTPESRYLAQIGDSVSLTCSTTGCESPFFFWRPLLDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 126 - DNA, 789 bp, Artificial Sequence
  Synthetic Construct - 146R14
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccactgttcttttggcggccgctgatcgacagccccctgaacggcaaagtgaccaacgagggcaccacctccaccctgaccatgaaccccgtgtccttcggcaacgagcactcctacctgtgtacagctcggtgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 127 - protein, 261 aa, Artificial Sequence
  Synthetic Construct - 146R14
MPGKMVVILGASNILWIMFAASQAFKIETTPESRYLAQIGDSVSLTCSTTGCESPLFFWRPLIDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 128 - DNA, 789 bp, Artificial Sequence
  Synthetic Construct - 146R15
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggccgcagctggacagccccctgaacggcaaagtgaccaacgagggcaccacctccaccctgaccatgaaccccgtgtccttcggcaacgagcactcctacctgtgtacagctcggtgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 129 - protein, 261 aa, Artificial Sequence
  Synthetic Construct - 146R15
MPGKMVVILGASNILWIMFAASQAFKIETTPESRYLAQIGDSVSLTCSTTGCESPFFFWRPQLDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 130 - DNA, 789 bp, Artificial Sequence
  Synthetic Construct - 146R16
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggccgctgatcgacagccccctgaacggcaaagtgaccaacgagggcaccacctccaccctgaccatgaaccccgtgtccttcggcaacgagcactcctacctgtgtacagctcggtgcgagagccggtccctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 131 - protein, 261 aa, Artificial Sequence
  Synthetic Construct - 146R16
MPGKMVVILGASNILWIMFAASQAFKIETTPESRYLAQIGDSVSLTCSTTGCESPFFFWRPLIDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 132 - DNA, 789 bp, Artificial Sequence
  Synthetic Construct - 146R17
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggccgctgatcgacagccccctgaacggcaaagtgaccaacgagggcaccacctccaccctgaccatgaaccccgtgtccttcggcaacgagcactcctacctgtgtacagctcggtgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 133 - protein, 261 aa, Artificial Sequence
  Synthetic Construct - 146R17
MPGKMVVILGASNILWIMFAASQAFKIETTPESRYLAQIGDSVSLTCSTTGCESPFFFWRPLIDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 134 - DNA, 789 bp, Artificial Sequence
  Synthetic Construct - 146R18
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggccgctgatcgacagccccctgaacggcaaagtgaccaacgagggcaccacctccaccctgaccatgaaccccgtgtccttcggcaacgagcactcctacctgtgtacagctacctgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 135 - protein, 261 aa, Artificial Sequence
  Synthetic Construct - 146R18
MPGKMVVILGASNILWIMFAASQAFKIETTPESRYLAQIGDSVSLTCSTTGCESPFFFWRPLIDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 136 - DNA, 789 bp, Artificial Sequence
  Synthetic Construct - 146R19
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggccgcagatcgacagccccctgaacggcaaagtgaccaacgagggcaccacctccaccctgaccatgaaccccgtgtccttcggcaacgagcactcctacctgtgtacagctcggtgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 137 - protein, 261 aa, Artificial Sequence
  Synthetic Construct - 146R19
MPGKMVVILGASNILWIMFAASQAFKIETTPESRYLAQIGDSVSLTCSTTGCESPFFFWRPQIDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 138 - DNA, 789 bp, Artificial Sequence
  Synthetic Construct - 146R20
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggccgcagatcgacagccccctgaacggcaaagtgaccaacgagggcaccacctccaccctgaccatgaaccccgtgtccttcggcaacgagcactcctacctgtgtacagctcggtgcgagagccggtccctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 139 - protein, 261 aa, Artificial Sequence
  Synthetic Construct - 146R20
MPGKMVVILGASNILWIMFAASQAFKIETTPESRYLAQIGDSVSLTCSTTGCESPFFFWRPQIDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 140 - DNA, 789 bp, Artificial Sequence
  Synthetic Construct - 146R21
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggaccctgctggacagccccctgaacggcaaagtgaccaacgagggcaccacctccaccctgaccatgaaccccgtgtccttcggcaacgagcactcctacctgtgtacagctcggtgcgagagccggtccctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 141 - protein, 261 aa, Artificial Sequence
  Synthetic Construct - 146R21
MPGKMVVILGASNILWIMFAASQAFKIETTPESRYLAQIGDSVSLTCSTTGCESPFFFWRTLLDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 142 - DNA, 789 bp, Artificial Sequence
  Synthetic Construct - 146R22
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccactgttcttttggcggaccctgctggacagccccctgaacggcaaagtgaccaacgagggcaccacctccaccctgaccatgaaccccgtgtccttcggcaacgagcactcctacctgtgtacagctcggtgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 143 - protein, 261 aa, Artificial Sequence
  Synthetic Construct - 146R22
MPGKMVVILGASNILWIMFAASQAFKIETTPESRYLAQIGDSVSLTCSTTGCESPLFFWRTLLDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISPGGGSDYKDDDDKGGGSDYKDDDDKGGGSDYKDDDDKGS

SEQ ID NO: 144 - DNA, 789 bp, Artificial Sequence
  Synthetic Construct - 146R23
atgcccggcaagatggtggtgatcctgggcgcctccaacatcctgtggatcatgttcgccgcctcccaagcttttaagatcgagacaacccccgagtccagatacctggcccagatcggcgactccgtgtccctgacctgctccaccaccggctgcgagtccccatttttcttttggcggaccctgctggacagccccctgaacggcaaagtgaccaacgagggcaccacctccaccctgaccatgaaccccgtgtccttcggcaacgagcactcctacctgtgtacagctcggtgcgagagccggaagctggaaaagggcatccaggtggaaatctacagcttccccaaggaccccgagatccacctgtccggccctctggaagccggcaagcccatcaccgtgaagtgctccgtggccgacgtgtaccccttcgaccggctggaaatcgacctgctgaagggcgaccacctgatgaagtcccaggaatttctggaggacgccgaccggaagtccctggaaacaaagtctctggaagtgaccttcacccccgtgatcgaggacatcggcaaggtgctggtgtgccgggccaagctgcacatcgacgagatggactccgtgcccaccgtgcggcaggccgtgaaagaactgcaggtctacatctcccctggcggaggatccgactacaaggacgacgacgacaagggcggaggcagcgattacaaggatgatgatgacaaagggggcggaagcgactataaagacgatgatgataagggctcctgatga

SEQ ID NO: 145 - protein, 201 aa, Artificial Sequence
  Synthetic Construct - 146R23
MPGKMVVILGASNILWIMFAASQAFKIETTPESRYLAQIGDSVSLTCSTTGCESPFFFWRTLLDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHI

SEQ ID NO: 146 - DNA, 603 bp, Artificial Sequence
  Synthetic Construct - R1R7
atgtttaaaatcgagaccaccccagaatctcgctatcttgctcagattggtgactccgtctcattgacttgcagcaccacaggctgtgagtccccatttttcttttggcgtccgctgcttgattcgcctctgaatgggaaggtgacgaatgaggggaccacatctacgctgacaatgaatcctgttagttttgggaacgaacactcttacctgtgcacagcacgttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcaccataa

SEQ ID NO: 147 - protein, 200 aa, Artificial Sequence
  Synthetic Construct - R1R7
MFKIETTPESRYLAQIGDSVSLTCSTTGCESPFFFWRPLLDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 148 - DNA, 603 bp, Artificial Sequence
  Synthetic Construct - R1R3
atgtttaaaatcgagaccaccccagaatctcgctatcttgctcagattggtgactccgtctcattgacttgcagcaccacaggctgtgagtccccatttttcttttggcgtacgctgcttgattcgcctctgaatgggaaggtgacgaatgaggggaccacatctacgctgacaatgaatcctgttagttttgggaacgaacactcttacctgtgcacagcacgttgtgaatctcgctctttggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcaccataa

SEQ ID NO: 149 - protein, 200 aa, Artificial Sequence
  Synthetic Construct - R1R3
MFKIETTPESRYLAQIGDSVSLTCSTTGCESPFFFWRTLLDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 150 - DNA, 603 bp, Artificial Sequence
  Synthetic Construct - R5R7
atgtttaaaatcgagaccaccccagaatctcgctatcttgctcagattggtgactccgtctcattgacttgcagcaccacaggctgtgagtccccactgttcttttggcgtccgctgattgattcgcctctgaatgggaaggtgacgaatgaggggaccacatctacgctgacaatgaatcctgttagttttgggaacgaacactcttacctgtgcacagcacgttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcaccataa

SEQ ID NO: 151 - protein, 200 aa, Artificial Sequence
  Synthetic Construct - R5R7
MFKIETTPESRYLAQIGDSVSLTCSTTGCESPLFFWRPLIDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 152 - DNA, 603 bp, Artificial Sequence
  Synthetic Construct - R1R6R7
atgtttaaaatcgagaccaccccagaatctcgctatcttgctcagattggtgactccgtctcattgacttgcagcaccacaggctgtgagtccccatttttcttttggcgtccgctgcttgattcgcctctgaatgggaaggtgacgaatgaggggaccacatctacgctgacaatgaatcctgttagttttgggaacgaacactcttacctgtgcacagcaacttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcaccataa

SEQ ID NO: 153 - protein, 200 aa, Artificial Sequence
  Synthetic Construct - R1R6R7
MFKIETTPESRYLAQIGDSVSLTCSTTGCESPFFFWRPLLDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 154 - DNA, 603 bp, Artificial Sequence
  Synthetic Construct - R3R7
atgtttaaaatcgagaccaccccagaatctcgctatcttgctcagattggtgactccgtctcattgacttgcagcaccacaggctgtgagtccccactgttcttttggcgtacgctgcttgattcgcctctgaatgggaaggtgacgaatgaggggaccacatctacgctgacaatgaatcctgttagttttgggaacgaacactcttacctgtgcacagcacgttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcaccataa

SEQ ID NO: 155 - protein, 200 aa, Artificial Sequence
  Synthetic Construct - R3R7
MFKIETTPESRYLAQIGDSVSLTCSTTGCESPLFFWRTLLDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 156 - DNA, 603 bp, Artificial Sequence
  Synthetic Construct - R1R5
atgtttaaaatcgagaccaccccagaatctcgctatcttgctcagattggtgactccgtctcattgacttgcagcaccacaggctgtgagtccccattcttcttttggcgtccgctgattgattcgcctctgaatgggaaggtgacgaatgaggggaccacatctacgctgacaatgaatcctgttagttttgggaacgaacactcttacctgtgcacagcacgttgtgaatctcgctctttggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcaccataa

SEQ ID NO: 157 - protein, 200 aa, Artificial Sequence
  Synthetic Construct - R1R5
MFKIETTPESRYLAQIGDSVSLTCSTTGCESPFFFWRPLIDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 158 - DNA, 603 bp, Artificial Sequence
  Synthetic Construct - R6R7
atgtttaaaatcgagaccaccccagaatctcgctatcttgctcagattggtgactccgtctcattgacttgcagcaccacaggctgtgagtccccactgttcttttggcgtccgctgcttgattcgcctctgaatgggaaggtgacgaatgaggggaccacatctacgctgacaatgaatcctgttagttttgggaacgaacactcttacctgtgcacagcaacttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcaccataa

SEQ ID NO: 159 - protein, 200 aa, Artificial Sequence
  Synthetic Construct - R6R7
MFKIETTPESRYLAQIGDSVSLTCSTTGCESPLFFWRPLLDSPLNGKVTNEGTTSTLTMNPVSFGNEHSYLCTATCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 160 - DNA, 603 bp, Artificial Sequence
  Synthetic Construct - R3R5R7
atgtttaaaatcgagaccaccccagaatctcgctatcttgctcagattggtgactccgtctcattgacttgcagcaccacaggctgtgagtccccactgttcttttggcgtacgctgattgattcgcctctgaatgggaaggtgacgaatgaggggaccacatctacgctgacaatgaatcctgttagttttgggaacgaacactcttacctgtgcacagcacgttgtgaatctcgcaaattggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcaccataa

SEQ ID NO: 161 - protein, 200 aa, Artificial Sequence
  Synthetic Construct - R3R5R7
MFKIETTPESRYLAQIGDSVSLTCSTTGCESPLFFWRTLIDSPLNGKVTNEGTTSTLTMNPVSFGNEHSYLCTARCESRKLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 162 - DNA, 603 bp, Artificial Sequence
  Synthetic Construct - R1R3R5
atgtttaaaatcgagaccaccccagaatctcgctatcttgctcagattggtgactccgtctcattgacttgcagcaccacaggctgtgagtccccattcttcttttggcgtacgctgattgattcgcctctgaatgggaaggtgacgaatgaggggaccacatctacgctgacaatgaatcctgttagttttgggaacgaacactcttacctgtgcacagcacgttgtgaatctcgctctttggaaaaaggaatccaggtggagatctactcttttcctaaggacccagagattcatttgagtggccctctggaggctgggaagccgatcacagtcaagtgttcagttgctgatgtatacccatttgaccgcctggagattgacttactgaaaggagatcatctcatgaagagtcaggaatttctggaggatgcagaccgcaagtccctggaaaccaagagtttggaagtaacctttactcctgtcattgaggatattggaaaagttcttgtttgccgcgctaaattacacattgatgaaatggattctgtgccaacagtacgccaggctgtaaaagaattgcaagtgtacatttcaccataa

SEQ ID NO: 163 - protein, 200 aa, Artificial Sequence
  Synthetic Construct - R1R3R5
MFKIETTPESRYLAQIGDSVSLTCSTTGCESPFFFWRTLIDSPLNGKVTNEGTTSTLTMNPVSFGNEHSYLCTARCESRSLEKGIQVEIYSFPKDPEIHLSGPLEAGKPITVKCSVADVYPFDRLEIDLLKGDHLMKSQEFLEDADRKSLETKSLEVTFTPVIEDIGKVLVCRAKLHIDEMDSVPTVRQAVKELQVYISP

SEQ ID NO: 164 - protein, 62 aa, Artificial Sequence
  Synthetic Construct - 2D-VCAM-1-CKappa-UGA-V5-G
PAGGKPIPNPLLGLDSTLEPTYCDLAPPAGTTDAAHPGRSVVPALLPLLAGTLLLLETATAP